(19) United States
(12) Patent Application Publication
(10) Pub. No.: US 2009/0171639 A1
(43) Pub. Date: Jul. 2, 2009

(54) Method to identify modulators for human 3alpha-hydroxysteroid dehydrogenase

(76) Inventors: Philipp Floersheim, Basel (CH); Christian Ostermeier, Fischingen (DE); Doncho Uzunov, Basel (CH); Wolfgang Jahnke, Lorrach (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/823,308

(22) Filed: Jun. 26, 2007

Related U.S. Application Data
(63) Continuation of application No. 10/486,660, filed on Feb. 12, 2004, now abandoned.
(60) Provisional application No. 60/314,045, filed on Aug. 22, 2001.

(51) Int. Cl. G06G 7/58; C12N 9/04

(52) U.S. Cl. 703/11; 435/190

(57) ABSTRACT

3 α-hydroxysteroid dehydrogenase (3α-HSD) plays a central role in the metabolism and action of steroid hormones and neurosteroids (steroids synthesized in the central nervous system). The high resolution structure of human type III 3α-HSD crystallized in complex with cofactor NADP is determined by X-ray diffraction. Furthermore the active site is determined. The structure coordinates of the enzyme may be used to design and select novel classes of modulators to human type III 3α-HSD.

### FIELD OF THE INVENTION

The present invention relates to a method to identify modulators for human 3α-hydroxysteroid dehydrogenase (3a-HSD) by using compounds with certain structural, physical and spatial characteristics that allow for the interaction of said compounds with specific residues of the active site of the enzyme. This interaction between the compounds of the invention and the active site inhibits or potentiates the activity of 3a-HSD and these compounds are useful for treating disease in which a deficiency of allopregnanolone, an endogenous neuroactive substrate and the product of the 3a-HSD, is indicated, such as major unipolar depression, premenstrual dysphoric disorder (PMDD, PMS) and other affective disorders. This invention relates also to a novel crystalline structure of type-3 human 3a-HSD, the identification of the detailed catalytic site for the human form of this enzyme and methods enabling the design and selection of inhibitors and potentiators of said active site.

### BACKGROUND OF THE INVENTION

Human 3a-HSD s play central roles in the metabolism and action of steroid hormones and neurosteroids (steroids synthesized in the central nervous system). The 3a-HSD is a member of the aldo-keto reductase (AKR) superfamily. In general, the function of mammalian 3a-HSD s is to convert (reduce) 5α- and 5α,3-ketosteroids into 5α,3α- and 5α,3α-tetrahydrosterolds, respectively, and to further oxidize these 3α-reduced tertrahydrosteroids back to their parent 3-keto steroidal precursors. The steroids that are target substrates of the 3a-HSDs are androgens and progestins. For example, in the prostate the potent androgen 5α-dihydrotestosterone will be converted by the 3a-HSD into the weak androgen 3α-androstanediol. By contrast, in the central nervous system, 3a-HSD can regulate the occupancy of the g-aminobutyric acid (GABA)_{A} receptor by converting 5α-dihydroprogesterone into 3α-hydroxy-5a-pregnan-20-one (allopregnanolone), a potent allosteric effector of the GABA_{A} receptor (K_{d}=10⁻⁹ M) (Majewski, M. D. et al. (1986) Science 232, 1004-1007; Majewski, M. D. (1992) Prog. Neurobiol. 38, 379-395; Lambert, J. J. et al., Trends Pharmacol. Sci. 16, 295-303). In the presence of GABA, allopregnanolone will potentiate GABA_{A}-mediated chloride conductance. As a result 3a-HSD is responsible for the production of anxiolytic steroids, and decreased activity in this pathway has been implicated in the symptoms of pre-menstrual syndrome (Morrow, A. L. et al. (1998) Nature (London) 395, 652-653). Thus 3a-HSD isoforms regulate the occupancy of both a nuclear receptor (androgen receptor) and a membrane-bound chloride-ion gated channel (GABA_{A} receptor) and may have profound effects on receptor function. For these reasons the 3α-HSD is considered a molecular switch turning on and off the function of steroid hormones in the prostate and of neurosteroids in the CNS.

Four human 3a-HSD isoforms have been cloned, sequenced and characterized: type-1 3α-HSD (AKR1C4), type-2 3α(17β)—HSD (AKR1C3), type-3 3α-HSD (AKR1C2), and 20α(3α)-HSD (AKR1C1), sharing at least 84% amino acid and sequence identity. Of these isoforms only type-2 and type-3 are expressed in the brain with type-3 being the predominant form present in the CNS. Types-2 and −3 3α-HSDs share almost 90% nucleotide sequence identity and 88% amino acid homology. Their putative substrate binding pockets and catalytic domains are highly conserved (as are among the other members of the AKR superfamily). The type-3 isoform is believed to be the major form responsible for the oxidation (turning off) of the anxiolytic GABA_{A} receptor-active neurosteroid allopregnanolone in the brain.

All 3a-HSD s are NAD(P)(H) dependent oxido-reductases implying that NAD⁺, NADH, NADP⁺ and NADPH are the cofactors. The oxidative function requires the presence of NAD⁺ or NADP⁺, while NADPH is being utilized for the reduction of 3-ketosteroids. Known inhibitors of the 3a-HSD are the class of the non-steroidal anti-inflamatory drugs (NSAIDs) (Penning et al., PNAS (1983) 80, 4504-4508) and the selective serotonin reuptake inhibitors (SSRIs) that were reported to potentiate and inhibit at the same time the 3a-HSD (Griffin and Mellon, PNAS (1999) 96, 13512-13517.

### SUMMARY OF THE INVENTION

In one aspect the invention comprises the crystalline structure of human type III 3a-HSD and to determine its structure coordinates.

The structure coordinates of a human type III 3a-HSD crystal are used to reveal the atomic details of the active site or the cofactor binding site of the enzyme and to solve the structure of a different human type III 3a-HSD crystal, or a crystal of a mutant, homologue or co-complex, of human type III 3a-HSD.

It is also an object of this invention to use the structure coordinates and atomic details of human type III 3a-HSD, or its mutants or homologues or co-complexes, to provide potentiators or inhibitors of human type III 3a-HSD.

In still a further aspect, the invention provides a method of screening compounds for their ability to modulate the human type III 3a-HSD.

### DETAILED DESCRIPTION

Table 1 lists the atomic structure coordinates of human type III 3a-HSD as derived by X-ray diffreaction from a crystal of human type III 3a-HSD 3α-HSD complexed to the NADP cofactor [Theoretical total number of refl. in resol. range: 56988 (100.0%); number of unobserved reflections (no entry or IFI=0): 1562 (2.7%); number of reflections rejected: 0 (0.0%); total number of reflections used: 55426 (97.3%); number of reflections in working set: 49806 (87.4%); number of reflections in test set: 5620 (9.9%)]. The following abbreviations are used in Table 1. “Atom type” refers to the element whose coordinates are measured. The first letter in the column defines the element. “X, Y, Z” cryptographically defines the atomic position of the element measured. “B” is a thermal factor that measures movement of the atom around its atomic center.

### Abbreviations:

### Å=Angstrom

TIP=represents in the listings of molecule of e.g. active site a water molecule

### DEFINITIONS

The following terms are also used herein:

The term “co-complex” means human type III 3a-HSD or homologue of human type III 3a-HSD in covalent or non-covalent association with a chemical entity or compound. The term “associating with” refers to a condition of proximity between a chemical entity or compound, or portions thereof, and a human type III 3a-HSD molecule or portions thereof. The association may be non-covalent—wherein the juxtaposition is energetically favored by hydrogen bonding or van der Waals or electrostatic interactions—or it may be covalent. The term “active site” or “active site moiety” refers to any or all of the following sites in human type III 3a-HSD: the substrate binding site; and the site where the reduction of the substrate occurs. The active site is characterized by at least amino acid residues TYR 24, ALA 25, ALA 52, VAL 54, TYR 55, LYS 84, TRP 86, HIS 117, ILE 129, ASN 167, GLN 190, TYR 216, HIS 222, GLU 224, PRO 226, TRP 227, LEU 306, LEU 308, ILE 310, PHE 311, TIP 1, TIP 33, TIP 131, TIP 225, TIP 235 using the sequence and numbering according to SEQ ID NO: 1 and Table 1 (for the TIP (=water) molecules).

The term “cofactor” binding site refers to any or all of the following sites in the human type III 3a-HSD: the cofactor binding site (cofactor e.g. NADP). The cofactor binding site (here NADP) is characterized by at least amino acid residues GLY 22, THR 23, TYR 24, ASP 50, TYR 55, LYS 84, HIS 117, SER 166, ASN 167, GLN 190, TYR 216, SER 217, ALA 218, LEU 219, GLY 220, SER 221, HIS 222, LEU 236, ALA 253, LEU 268, ALA 269, LYS 270, SER 271, TYR 272, ASN 273, ARG 276, GLN 279, ASN 280, LEU 306, TIP 17, TIP 27, TIP 53, TIP 93, TIP 122, TIP 207, TIP 219, TIP 224 using the sequence and numbering according to SEQ ID NO: 1 and Table 1 (for the TIP (=water) molecules).

The term “structure coordinates” refers to mathematical coordinates derived from mathematical equations related to the patterns obtained on diffraction of a monochromatic beam of X-rays by the atoms (scattering centers) of a human type III 3a-HSD molecule in crystal form. The diffraction data are used to calculate an electron density map of the repeating unit of the crystal. The electron density maps are used to establish the positions of the individual atoms within the unit cell of the crystal.

Those of skilled in the art understand that a set of structure coordinates determined by X-ray crystallography is not without standard error. For the purpose of this invention, any set of structure coordinates for human type III 3a-HSD or human type III 3a-HSD variants that have a root mean square deviation of protein backbone atoms (N, α-C, C and O) of less than 0.75 Å when superimposed-using backbone atoms-on the structure coordinates listed in Table 1 shall be considered identical.

The term “variant” refers to a polynucleotide or polypeptide that differs from a reference polynucleotide or polypeptide, but retains the essential properties thereof. A typical variant of a polynucleotide differs in nucleotide sequence from the reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from the reference polypeptide. Generally, alterations are limited so that the sequences of the reference polypeptide and the variant are closely similar overall and, in many regions, identical. A variant and reference polypeptide may differ in amino acid sequence by one or more substitutions, insertions, deletions in any combination. A substituted or inserted amino acid residue may or may not be one encoded by the genetic code. Typical conservative substitutions include Gly, Ala; Val, Ile, Leu; Asp, Glu; Asn, Gin, Ser. Thr; Lys, Arg; and Phe and Tyr. A variant of a polynucleotide or polypeptide may be naturally occurring such as an allele, or it may be a variant that is not known to occur naturally. Non-naturally occurring variants of polynucleotides and polypeptides may be made by mutagenesis techniques or by direct synthesis. Also included as variants are polypeptides having one or more post-translational modifications, for instance glycosylation, phosphorylation, methylation, ADIP ribosylation and the like. Embodiments include methylation of the N-terminal amino acid, phosphorylations of serines and threonines and modification of C-terminal glycines.

The term “unit cell” refers to a basic shaped block. The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal.

The term “molecular replacement” refers to a method that involves generating a-preliminary model of a variant of human type III 3a-HSD crystal whose structure coordinates are unknown, by orienting and positioning a molecule whose structure coordinates are known (e.g., human type III 3a-HSD coordinates from Table 1) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. p577 (1985); M. G. Rossmann, ed., “The Molecular Replacement Method”, Int. Sci. Rev. Ser., No. 13, Gordon & Breach, New York, (1972). Using the structure coordinates of human type III 3a-HSD provided by this invention, molecular replacement may be used to determine the structure coordinates of a crystalline variant, e.g co-complexed with a specific inhibitor, or homologue of human type III 3a-HSD or of a different crystal form of human type III 3a-HSD.

### DETAILED DESCRIPTION OF THE INVENTION

In order that the invention described herein may be more fully understood, the following detailed description is set forth.

The present invention relates to crystalline human type III 3a-HSD, the structure of human type III 3a-HSD 3α-HSD as determined by X-ray crystallography, the use of that structure to solve the structure of human type III 3a-HSD homologues and of other crystal forms of human type III 3a-HSD, co-complexes of human type III 3a-HSD, and the use of the human type III 3a-HSD structure and that of its homologues, and co-complexes to design and to select modulators of human type III 3a-HSD.

A. The Structure of Human Type III 3a-HSD in Complex with NADP.

The present invention provides, for the first time, crystals of human type III 3a-HSD grown in the presence of NADP from solutions of polyethylene glycol. The crystals have rhombohedral space group symmetry and reached 0.5×0.5×0.2 mm. The unit cell of said crystals is of the following dimensions: a=b=c=108.5+/−1 Å, α=β=γ=85.1°+/−1°. The structure coordinates of human type III 3a-HSD 3α-HSD, as determined by X-ray crystallography of crystalline human type III 3a-HSD, is listed in Table 1.

Crystal packing reveals that human type III 3a-HSD crystallizes in a single dimmer (data in Table 1 are for the dimer).

The enzyme core is formed by a α/β barrel with a cylindrical core of eight parallel β-strands surrounded by eight α-helices which run anti-parallel to the βsheet. This barrel is formed by repeating the β/α unit eight times with two deviations: First, an additional helix exists between β-strand 7 and helix 8 of the barrel; and a second helix exists between helix 8 and the C-terminal region. At the N-terminus, two additional, anti-parallel β-strands, which are connected by a tight hairpin-loop, form the bottom seal of the barrel.

Our understanding of the structure of human type III 3a-HSD has enabled the identification of the active and cofactor binding sites of the enzyme. The active site moiety is characterized by at least amino add residues TYR 24, ALA 25, ALA 52, VAL 54, TYR 55, LYS 84, TRP 86, HIS 117, ILE 129, ASN 167, GLN 190, TYR 216, HIS 222, GLU 224, PRO 226, TRP 227, LEU 306, LEU 308, ILE 310, PHE 311, TIP 1, TIP 33, TIP 131, TIP 225, TIP 235 using the sequence and numbering according to SEQ ID NO:1. The cofactor binding site (here NADP) is characterized by at least amino acid residues GLY 22, THR 23, TYR 24, ASP 50, TYR 55, LYS 84, HIS 117, SER 166, ASN 167, GLN 190, TYR 216, SER 217, ALA 218, LEU 219, GLY 220, SER 221, HIS 222, LEU 236, ALA 253, LEU 268, ALA 269, LYS 270, SER 271, TYR 272, ASN 273, ARG 276, GLN 279, ASN 280, LEU 306, TIP 17, TIP 27, TIP 53, TIP 93, TIP 122, TIP 207, TIP 219, TIP 224 using the sequence and numbering according to SEQ ID NO:1 and Table 1 (for the TIP (=water) molecules).

B. Uses of the Structure Coordinates of Human Type III 3a-HSD

For the first time, the present invention permits the use of molecular design techniques to design, select and synthesize chemical entities and compounds, including inhibitory compounds, capable of binding to the active site or accessory binding site of human type III 3a-HSD, in whole or in part.

One approach enabled by this invention, is to use the structure coordinates of human type III 3a-HSD to design compounds that bind to the enzyme and alter the physical properties of the compounds in different ways, e.g., solubility. For example, this invention enables the design of compounds that act as competitive inhibitors of the human type III 3a-HSD enzyme by binding to, all or a portion of, the active site of human type III 3a-HSD. A second design approach is to probe a human type III 3a-HSD crystal with molecules composed of a variety of different chemical entities to determine optimal sites for interaction between candidate human type III 3a-HSD inhibitors and the enzyme. For example, high resolution X-ray diffraction data collected from crystals saturated with solvent allows the determination of where each type of solvent molecule sticks. Small molecules that bind tightly to those sites can then be designed and synthesized and tested for their human type III 3a-HSD inhibitor activity. Travis, J., Science, 262, p. 1374 (1993).

This invention also enables the development of compounds that can isomerize to short-lived reaction intermediates in the chemical reaction of a substrate or other compound that binds to human type III 3a-HSD, with human type III 3a-HSD. Thus, the time-dependent analysis of structural changes in human type III 3a-HSD 3α-HSD during its interaction with other molecules is enabled. The reaction intermediates of human type III 3a-HSD can also be deduced from the reaction product in co-complex with human type III 3a-HSD. Such information is useful to design improved analogues of known human type III 3a-HSD potentiators or inhibitors or to design novel classes of potentiators or inhibitors based on the reaction intermediates of the human type III 3a-HSD 3α-HSD enzyme and human type III 3a-HSD-ligand co-complex. This provides a novel route for designing human type III 3a-HSD 3α-HSD inhibitors with both high specificity and stability.

Another approach made possible and enabled by this invention, is to screen computationally small molecule data bases for chemical entities or compounds that can bind in whole, or in part, to the human type III 3a-HSD enzyme. In this screening, the quality of fit of such entities or compounds to the binding site may be judged either by shape complementarity or by estimated interaction energy (Meng, E. C. et al., J. Comp. Chem., 13, pp. 505-524 (1992)).

Because human type III 3a-HSD may crystallize in more than one crystal form, the structure coordinates of human type III 3a-HSD, or portions thereof, as provided by this invention are particularly useful to solve the structure of those other crystal forms of human type III 3a-HSD. They may also be used to solve the structure of human type III 3a-HSD co-complexes, or of the crystalline form of any other protein with significant amino add sequence homology to any functional domain of human type III 3a-HSD.

One method that may be employed for this purpose is molecular replacement. In this method, the unknown crystal structure, whether it is another crystal form of human type III 3a-HSD, human type III 3a-HSD co-complex, or the crystal of some other protein with significant amino acid sequence homology to any functional domain of human type III 3a-HSD, may be determined using the human type III 3a-HSD structure coordinates of this invention as provided in Table 1. This method will provide an accurate structural form for the unknown crystal more quickly and efficiently than attempting to determine such information ab initio.

In addition, in accordance with this invention, human type III 3a-HSD may be crystallized in co-complex with known human type III 3a-HSD inhibitors, as e.g. NSAIDs and SSRIs. The crystal structures of a series of such complexes may then be solved by molecular replacement and compared with that of wild-type human type III 3a-HSD. Potential sites for modification within the binding site of the enzyme may thus be identified. This information provides an additional tool for determining the most efficient binding interactions, for example, increased hydrophobic interactions, between human type III 3a-HSD and a chemical entity or compound.

All of the complexes referred to above may be studied using well-known X-ray diffraction techniques and may be refined versus 1-3 Å resolution X-ray data to an R value of about 0.20 or less using computer software, such as X-PLOR (Yale University, .COPYRGT.1992, distributed by Molecular Simulations, Inc.). See, e.g., Blundel & Johnson, supra; Methods in Enzymology, vol. 114 & 115, H. W. Wyckoff et al., eds., Academic Press (1985). This information may thus be used to optimize known classes of human type III 3a-HSD potentiators and inhibitors, and more importantly, to design and synthesize novel classes of human type III 3a-HSD potentiators and inhibitors.

The design of compounds that bind to human type III 3a-HSD according to this invention generally involves consideration of two factors. First, the compound must be capable of physically and structurally associating with human type III 3a-HSD. Non-covalent molecular interactions important in the association of human type III 3a-HSD with its substrate include hydrogen bonding, van der Waals and hydrophobic interactions.

Second, the compound must be able to assume a conformation that allows it to associate with human type III 3a-HSD. Although certain portions of the compound will not directly participate in this association with human type III 3a-HSD, those portions may still influence the overall conformation of the molecule. This, in turn, may have a significant impact on potency. Such conformational requirements include the overall three-dimensional structure and orientation of the chemical entity or compound in relation to all or a portion of the binding site, e.g., active site or accessory binding site of human type III 3a-HSD, or the spacing between functional groups of a compound comprising several chemical entities that directly interact with human type III 3a-HSD.

The potential inhibitory or binding effect of a chemical compound on human type III 3a-HSD may be analyzed prior to its actual synthesis and testing by the use of computer modelling techniques. If the theoretical structure of the given compound suggests insufficient interaction and association between it and human type III 3a-HSD, synthesis and testing of the compound is obviated. However, if computer modeling indicates a strong interaction, the molecule may then be synthesized and tested for its ability to activate or inhibit the human type-3 by testing it in two functional assays—a spectrophotometric and a modified radiometric assay as described by L. Griffin and S. Mellon in Proc. Natl. Aced. Sci. USA, 1999, 96 (23), 13512-13517, and by T. Penning et al. In Biochem. J., 2000, 351, 67-77. In this manner, synthesis of inoperative compounds may be avoided.

An inhibitory or other binding compound of human type III 3a-HSD may be computationally evaluated and designed by means of a series of steps in which chemical entities or fragments are screened and selected for their ability to associate with the individual binding pockets or other areas of human type III 3a-HSD.

One skilled in the art may use one of several methods to screen chemical entities or fragments for their ability to associate with human type III 3a-HSD and more particularly with the individual binding pockets of the human type III 3a-HSD active site. This process may begin by visual inspection of, for example, the active site on the computer screen based on the human type III 3a-HSD coordinates in Table 1. Selected fragments or chemical entities may then be positioned in a variety of orientations, or docked, within an individual binding pocket of human type III 3a-HSD. Docking may be accomplished using software such as Quanta and Sybyl, followed by energy minimization and molecular dynamics with standard molecular mechanics forcefields, such as CHARMM and AMBER.

Specialized computer programs may also assist in the process of selecting fragments or chemical entities. These include:

1. GRID (Goodford, P. J., “A Computational Procedure for Determining Energetically Favorable Binding Sites on Biologically Important Macromolecules”, J. Med. Chem., 28, 849-857 (1985)). GRID is available from Oxford University, Oxford, UK.
2. MCSS (Miranker, A. and M. Karplus, “Functionality Maps of Binding Sites: A Multiple Copy Simultaneous Search Method.” Proteins: Structure. Function and Genetics, 11, 29-34 (1991)). MCSS is available from Molecular Simulations, Burlington, Mass.
3. AUTODOCK (Goodsell, D. S., and A. J. Olsen, “Automated Docking of Substrates to Proteins by Simulated Annealing”, Proteins: Structure. Function, and Genetics, 8, 195-202 (1990)). AUTODOCK is available from Scripps Research Institute, La Jolla, Calif.
4. DOCK (Kuntz, I. D. et al., “A Geometric Approach to Macromolecule-Ligand Interactions”, J. Mol. Biol., 161, 269-288 (1982)). DOCK is available from University of California, San Francisco, Calif.
5. GOLD (Jones G et al., “Development and Validation of a Genetic Algorithm for Flexible Docking”, J. Mol. Biol. 267, 727-748 (1997))

Once suitable chemical entities or fragments have been selected, they can be assembled into a single compound or inhibitor. Assembly may be proceed by visual inspection of the relationship of the fragments to each other on the three-dimensional image displayed on a computer screen in relation to the structure coordinates of human type III 3a-HSD. This would be followed by manual model building using software such as Quanta or Sybyl.

Useful programs to aid one of skill in the art in connecting the individual chemical entities or fragments include:

1. CAVEAT (Bartlett, P. A. et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.
2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).
3. HOOK (available from Molecular Simulations, Burlington, Mass.).

Instead of proceeding to build an human type III 3a-HSD potentiator or inhibitor in a step-wise fashion one fragment or chemical entity at a time as described above, human type III 3a-HSD binding compounds may be designed as a whole or “de novo” using either an empty active site or optionally including some portion(s) of a known inhibitor(s). These methods include:

1. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Blosym Technologies, San Diego, Calif.
2. LEGEND (Nishibata, Y. and A. Itai, Tetrahedron, 47, p. 8985 (1991)). LEGEND is available from Molecular Simulations, Burlington, Mass.
3. LeapFrog (available from Tripos Associates, St. Louis, Mo.).

Other molecular modelling techniques may also be employed in accordance with this invention. See, e.g., Cohen, N. C. et al., “Molecular Modeling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Once a compound has been designed or selected by the above methods, the efficiency with which that compound may bind to human type III 3a-HSD may be tested and optimized by computational evaluation. For example, a compound that has been designed or selected to function as an human type III 3a-HSD inhibitor must also preferably traverse a volume not overlapping that occupied by the active site when it is bound to the native substrate. An effective human type III 3a-HSD inhibitor must preferably demonstrate a relatively small difference in energy between its bound and free states (i.e., a small deformation energy of binding). Thus, the most efficient human type III 3a-HSD inhibitors should preferably be designed with a deformation energy of binding of not greater than about 10 kcal/mole, preferably, not greater than 7 kcal/mole. Human type III 3a-HSD inhibitors may interact with the enzyme in more than one conformation that is similar in overall binding energy. In those cases, the deformation energy of binding is taken to be the difference between the energy of the free compound and the average energy of the conformations observed when the inhibitor binds to the enzyme.

A compound designed or selected as binding to human type III 3a-HSD may be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the inhibitor and the enzyme when the inhibitor is bound to human type III 3a-HSD, preferably make a neutral or favorable contribution to the enthalpy of binding.

Specific computer software is available in the art to evaluate compound deformation energy and electrostatic interaction. Examples of programs designed for such uses include: Gaussian 92, revision C [M. J. Frisch, Gaussian, Inc., Pittsburgh, Pa. COPYRGT.1992]; AMBER, version 4.0 [P. A. Kollman, University of California at San Francisco, .COPYRGT. 1994]; QUANTA/CHARMM [Molecular Simulations, Inc., Burlington, Mass. COPYRGT.1994]; and insight II/Discover (Biosysm Technologies Inc., San Diego, Calif. .COPYRGT.1994). These programs may be implemented, for instance, using a Silicon Graphics workstation, IRIS 4D/35 or IBM RISC/6000 workstation model 550. Other hardware systems and software packages will be known to those skilled in the art. Once an human type III 3a-HSD-binding compound has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to human type III 3a-HSD by the same computer methods described in detail, above.

The following Examples illustrate the present invention, without in any way limiting the scope thereof.

### EXAMPLE 1

### Structure Determination of Human type III 3a-HSD Complexed with NADP with X-Ray

Cloning: Full length type III 3a-HSD cDNA is amplified by RT-PCR from human fetal brain total RNA (Clontech). Both first strand cDNA synthesis and PCR are performed in a single tube using gene-specific primers with the SuperScript One-Step RT-PCR (GibcoBRL, Life Technologies) kit employing an RT/Platinum Taq polymerase mix. The following reaction components are mixed in a single PCR tube according to the vendor's instructions: 2× Reaction Mix 25 μl, 1 μg template RNA, 0.4 μM final concentration of each gene-specific primer (sense and anti-sense), 1 μl RT/Platinum Taq polymerase mix, and nuclease-free water up to 50 μl final reaction volume.

The sense gene-specific primers are of the following sequences:

For the His-tagged construct: 5′-ATACATATGCACCACCACCACCACCACCTCGAGGT TCTGTTCCAGGGTCCGGATTCGAAATACCAGTGTGTG (SEQ ID NO: 3)

For the non-tagged construct: 5′-TTTCATATGGATTCGAAATACCAGTGTGTG (SEQ ID NO: 4)

The same anti-sense gene-specific primer with the following sequence is used for the full length amplification of both cDNAs: 5′-AGAGCGGCCGCTGTTAAATATTCATCAGAAA (SEQ ID NO: 5) The RT-PCR is performed on a GeneAmp PCR System 9700 (PE Appliad Biosystems). First strand cDNA synthesis is achieved with one cycle at 45° C. for 30 min, followed by RT denaturation at 94° C. for 2 min. PCR is conducted with 40 cycles of: denaturation at 94° C. for 15 sec, primer annealing at 55° C. for 30 sec, and primer extension at 68° C. for 2 min, followed by a single cycle of final extension at 72° C. for 10 min.

The PCR product is digested with NdeI and NotI, purified by agarose gel electrophoresis, and ligated into the NdeI NotI sites of pET26b(+) (Novagen) using standard ligation conditions with T4 DNA ligase (Promega). Both constructs are sequenced (see Appendix).

### Expression

The plasmids are transformed into BL21(DE3) and BL21(DE3)pLysS E. coli cells (Novagen). Small scale (10 ml) cultures are grown to check expression levels and solubility. It is found that both 3a-HSD constructs lead to high levels of inclusion bodies even at 25° C. BL21(DE3) gives better expression levels than BL21 (DE3)pLysS. For production scale, 4 liter shake flask cultures are grown: TBII medium, 37° C., induced with 1 mM IPTG for 4 h.

The usual pellet mass is 30 to 40 g. Due to the fact, that refolding is the method of choice to produce soluble protein, the non-tagged construct is used for the next steps.

### Refolding and Purification

E. coli wet cell pellets are suspended to 15% w/v in lysis buffer (50 mM Tris, 5 mM DTT, 5 mM EDTA, 5 mM Benzamidine-HCl; pH 8.0) using a Heldolph DIAX 800 homogenizer. Cells are lysed by passage twice through a Manton-Gaulin homogenizer (set at 1200 bar). The cell lysate is spun for 30 min at 16,000 g and the supernatant removed. The resulting inclusion body pellets are resuspended in lysis buffer (to approx. 5% w/v) using the Heidolph homogenizer and re-centrifuged. This process is repeated until the resulting supernatant is clear. A final wash using Milli-Q water (containing 5 mM DTT) as solvent is carried out, a sample of the water suspension diluted 10-fold with guanidine buffer (6M guanidine-HCl, 50 mM Tris and 50 mM DTT; pH 8.0.) and analysed using an analytical RP-HPLC system (Thermo Separation Products), fitted with an Orpegen C8 analytical column (HD-gel-RP-7s-300, 150 mm×4 mm). The resulting inclusion body pellet is then solubilized to 14 mg/ml using guanidine buffer and centrifuged.

The guanidine supernatant is diluted with guanidine buffer to a protein concentration of 200 μg/ml and subsequently dialysed at 4° C. vs 3×10 volumes of 50 mM Tris pH 8.5, containing 5 mM DTT and 1 mM EDTA. The rententate is centrifuged (30 min at 16,000 g) and loaded onto a Q-Sepharose HP anlon-exchange column at a flow rate of 8 ml/min, equilibrated with 50 mM Tris pH 8.5 containing 5 mM DTT. After washing the column with 5 column vol (250 ml) of buffer, a linear salt gradient of 0-1 M NaCl in the same buffer is used to elute the bound proteins from the column. The unbound is collected and concentrated using a 10,000 Da cut-off ultrafiltration membrane to approximately 10 mg/ml and loaded onto a gel-filtration column. The column (Superdex 75, XK26/60) is pre-equilibrated with 50 mM Tris pH 8.0 containing 150 mM NaCl and 5 mM DTT. 12 ml of concentrated protein solution is loaded and eluted at a flow-rate of 3 ml/min. The 3a-HSD peak elutes at ˜190 ml, well separated from a small quantity of aggregated material MS: 36736.4 Da (M+H)⁺. (ESI-MS).

Binding of natural ligands is a prerequisite for functionality. One method to detect ligand binding by NMR takes advantage of the increased transverse relaxation rates of the ligand protons when the ligand is bound. Increased line widths in the presence of protein thus indicates binding. Binding experiments of NADP to 3a-HSD are done and check by NMR. In the presence of 3a-HSD, the NADP peaks are broadened so much that they are hardly visible in the spectrum. This proves binding of NADP to 3a-HSD.

Similarly, binding of the presumed natural substrate, allopregnanolone, is proven by observing line broadening of allopregnanolone resonances. Since allopregnanolone is poorly soluble in water, 2% DMSO-d6 and 5% ethanol are added as co-solvents. Even then, the maximum achievable concentration of allo-pregnanolone is only about 20 μM. This is sufficient, however, to see the two upfield shifted methyl resonances of allopregnanolone broadening in the presence of 3a-HSD. Based on the NMR analysis, 3a-HSD appears well-folded and functional in respect to cofactor- and ligand-binding.

Crystallization: 3a-HSD in 50 mM Tris pH 8.0, 150 mM NaCl and 5 mM DTT, is concentrated to 20 mg/ml. For NADP-complexed 3a-HSD, solid NADP-sodium salt is added. Standard crystallization screening using the hanging drop method at 20° C. is used. Commercial screens from Hampton Research and Emerald BioStructures Inc. are used. The following crystallization conditions are found to give promising results:

200 mM Ammonium acetate, 100 mM Sodium citrate, pH 5.6, 30% PEG 4000

200 mM Ammonium sulfate, 100 mM MES, pH 6.0, 30% PEG monomethylether 5000

Other conditions which are found to give crystals:

200 mM Ammonium sulfate, 100 mM MES, pH 6.0, 30% PEG monomethylether 5000, 5% 1,2-Propandiol; 200 mM Ammonium sulfate, 100 mM MES, pH 6.0, 30% PEG monomethylether 5000, 5% Ethanol; 200 mM Ammonium sulfate, 100 mM MES, pH 6.0, 30% PEG monomethylether 5000, 5% 1-Propanol. Thus, in general, the following conditions may lead to crystals:

50-200 mM ammonium sulfate or ammonium acetate, 25-200 mM MES, pH 6.0 or 25-200 mM sodium citrate, 20-30% PEG monomethylether 2000 or 5000 or 20-30% PEG 2000, 4000, 6000, 8000; 0-10% additives as for example glycerin, 1,2-propandiol, ethylene glycol, and 0-20 mM DTT.

The NADP-complexed protein gives much nicer crystals than the apo-protein and crystallization conditions are optimized for the cofactor-3α-HSD complex. Finally, large prisms can be grown which reached 0.5×0.5×0.2 mm. Optimal growth conditions are 25% PEG monomethylether 5000, 5% Glycerol, 100 mM MES, pH 6.0, 200 mM ammoniumsulfate in the precence of 5 mM DTT. Optimal crystal size is reached after 1-2 weeks. A large crystal (3α-HSD in complex with NADP) is mounted in a capillary and diffraction data is collected using a 30 cm MAR imaging plate detector. Those of skill in the art will appreciate that the aforesaid crystallization conditions can be varied. Such variations may be used alone or in combination, and include final protein/inhibitor complex concentrations between 5 mg/ml and 35 mg/ml; all combinations of 3α-HSD/inhibitor to precipitant ratios; citrate concentrations between 1 mM and 200 mM; DTT concentrations between 0 mM and 20 mM; and any concentration of β-mercaptoethanol; pH ranges between 5.5 and 9.5; PEG concentrations between 10% and 25% (gms/100 ml); PEG weights between 2000 and 20000; HEPES concentrations between 5 and 395 mM; and any concentration or type of detergent; any temperature between −5° C. and 100° C.; and crystallization of 3α-HSD co-complexes by batch, liquid bridge, or dialysis method using these conditions or variations thereof.

Structure determination with X-ray: A single crystal of the human type III 3a-HSD complexed with the cofactor NADP is mounted into a glass capillary and X-ray diffraction data is collected at room temperature with a MAR imaging plate system (150 μm pixel size) mounted on a Enraf-Nonius FR591 rotation anode generator equipped with a Cu target, a 0.3 mm×3.0 mm fine focus and Osmic mirrors. Images are collected with 1.0° oscillation each, using an exposure time of 600 sec per frame and a crystal-to-detector distance of 120 mm. Raw diffraction data are processed and scaled with the HKL program suite version 1.96.6 (Otwinowski, Z. and Minor, W. Processing of X-ray Diffraction Data Collected in Oscillation Mode. Methods in Enzymology 1996; 276. C. W. Carter, Jr. and R. M. Sweet, Eds., Academic Press).

The structure is determined by molecular replacement, using the crystal structure of rat type III 3a-HSD (available at the PDB under accession number 1AFS) as search model Molecular replacement is performed with CNX 2000 (Brünger, A T et al. Crystallography & NMR System: A new software suite for macromolecular structure determination. Acta Cryst. 1998; D54: 905-921.), using data between 15 and 4 Å; the “Fastdirect” option is used. At this stage, inspection of the σ_{A}-weighted Fo-Fc electron density map with the program O version 7.0 (Jones, T A et al. Improved Methods for Building Protein Models in Electron Density Maps and the Location of Errors in these Models. Acta Crystallogr. 1991; A47: 110-19) reveals strong density for most of the side chains and all backbone atoms. The model of 3αHSD is built and adjusted to fit the density where necessary. The structure is refined by a number of cycles of torsion angle dynamics and energy minimization, interspersed by model rebuilding steps. For refinement, the “refine.inp” script pf CNX 2000 is used, with the following (non-default) option: Bulk solvent correction (based on the mask method). Cross-validation is used throughout refinement using a test set comprising 5% of the reflections. Water molecules are identified with the CNX script water_pick.inp, and selected based on difference peak height (greater than 3.0σ), hydrogen-bonding and distance criteria. The quality of the final refined model is assessed with the programs CNX 2000. The mean figure of merit for the crystal structure is up to 2 Å resolution (Table 1).

### EXAMPLE 2

### The Use of Molecular Replacement to Solve an Unknown Type III 3a-HSD Crystal Structure

The method of molecular replacement is used to determine the structure coordinates of crystals of human type III 3a-HSD in complex with a ligand (see below Example 4: e.g. 2-acetylbenzofuran) and NADP (as cofactor) in comparison with crystals of human type III 3a-HSD in complex with NADP (as prepared in Example 1). Crystals of human type III 3a-HSD In complex with e.g. 2-acetylbenzofuran are grown under conditions identical to those for crystals of human type III 3a-HSD In complex with the cofactor NADP.

X-ray diffraction data up to 2.0 Å resolution is collected on the 3a-HSD/NADP-2-acetylbenzofuran co-complex. A difference electron density map that combines diffraction data of the 2 results is used to locate structure changes that has occurred. Negative features (electron density) are found in the map wherever localized atoms in the ligand complex are removed or shifted by switching to the new ligand. Positive features are found when localized atoms are introduced into the structure, and indicated the new positions of shifted atoms.

These shifts, plus the new atoms referred to above, are modeled, and the resulting structure is refined against the X-ray data to determine a final picture of the co-complex of NSAIDs with 3a-HSD. The human type III 3a-HSD structure coordinates known for the first time by virtue of this invention may be used to solve the unknown structure of any homologue or co-complex of 3a-HSD using the above-described method. This method may also be used to determine the binding or orientation of a ligand or chemical entity in the active binding site of 3a-HSD. With this approach, we find that the structure of crystals, e.g. In the active site, of human type III 3a-HSD complexed with potential modulators, e.g. inhibitors may be different than the structure of human type III 3a-HSD complexed with NADP alone, e.g. as described in Table 1.

### EXAMPLE 3

### In Silico Gold Docking: Simulation of Binding of Allopregnolone or Other Compounds to Human Type III 3a-HSD

We simulate the binding mode of ligands to the active site of HSD using the X-ray structure of HSD. As a ligand molecule we select allopregnanolone of which we build a molecular model and to which we apply molecular mechanics minimization with the Sybyl (version 6.7.2) molecular modeling program. The model of HSD is obtained by assignment of Sybyl atom types to the atoms of chain A of HSD and co-crystallized NAP. To all 2263 atoms we add in total 2626 missing hydrogen atoms whereby polar amino acids Glu (in total 23), Asp (in total 18), Arg (in total 15), Lys (in total 26) as well as NADP (1) are regarded to be in their ionized state. The GOLD docking program is used to carry out 20 independent runs of docking the ligand model to the HSD model. The radius of the sphere defining the binding site is set to 15 A, and the coordinates of the center of the sphere are such that 1029 atoms (including hydrogen atoms) of the following 90 residues NAP 1, GLY 22, THR 23, TYR 24, ALA 25, PRO26, ALA 27, GLU 28, VAL 29, PRO30, LYS 31, ALA 34, ASP 50, SER 51, ALA 52, HIS 53, VAL 54, TYR 55, ASN 56, ASN 57, GLU 58, GLU 59, GLN 60, VAL 61, TYR 81, THR 82, SER 83, LYS 84, LEU 85, TRP 86, SER 87, ASN 88, SER 89, HIS 90, ALA 98, ARG 101, SER 102, ASN 105, TYR 114, LEU 115, ILE 116, HIS 117, PHE 118, PRO119, VAL 120, SER 121, VAL 122, LYS 123, PRO124, GLY 125, GLU 126, GLU 127, VAL 128, ILE 129, PRO130, LYS 131, ILE 137, LEU 138, PHE 139, SER 166, ASN 167, GLN 190, VAL 191, GLU 192, TYR 216, SER 217, SER 221, HIS 222, ARG 223, GLU 224, GLU 225, PRO226, TRP 227, VAL 228, ASP 229, LEU 268, LYS 270, TYR 272, ARG 304, TYR 305, LEU 306, THR 307, LEU 308, ASP 309, ILE 310, PHE 311, ALA 312, TYR 317, PRO318, and PHE 319 define the binding cavity in the GOLD program. Each of the 20 GOLD runs with our models of HSD and allopregnanolone results in a scored model of the molecule allopregnanolone docked to HSD. The GOLD score or Fitness of the models ranges from 26.94 to 42.54. All residues with atoms within 4 A of docked allopregnanolone are depicted, namely NAP 1, TYR 24, ALA 27, VAL 54, TYR 55, TRP 86, HIS 117, PHE 118, VAL 128, ILE 129, ASN 167, HIS 222, TRP 227, LEU 306, LEU 308 and PHE 311. Atom pair distances smaller than 0.9 Å (Sum of van der Waals radii of atom pair) are found between allopregnanolone (A) and the following 12 atoms of HSD:

### /A/TYR_{—}24/HH=1.967 Å

### /A/VAL_{—}54/HG22=2.131 Å

### /A/VAL_{—}54/HB=1.986 Å

### /A/TRP_{—}86/CZ3=2.504 Å

### /A/TRP_{—}86/CH2=2.460 Å

### /A/HIS_{—}117/NE2=2.427 Å

### /A/HIS_{—}117/NE2=1.799 Å (Hydrogen Bond)

### /A/VAL_{—}128/CG2=2.779 Å

### /A/VAL_{—}128/HG22=2.281 Å

### /A/TRP_{—}227/CZ2=2.576 Å

### /A/TRP_{—}227/HZ2=2.088 Å

### /A/LEU_{—}308/HD22=2.152 Å

In particular, it is seen that the 3-hydroxy group of allopregnanolone makes favorable strong electrostatic contacts with HIS 113, TYR 55 and the carboxamido group of NADP. Examples of regions in the binding site unoccupied by the docked ligand allopregnanolone can be seen within 4.5 A of allopregnanolone and at more than 3 A of HSD. Appropriate substitutions of allopregnanolone or molecules which share the modelled binding mode of allopregnanolone may occupy these regions and confer additional affinity to HSD. Similar Docking Procedures can be performed with other compounds, e.g. with compounds of a virtual library (available for example using MoSELECT (Gillet et al., J. of Molecular Graphics & Modelling (2002) 20(6), 491-498) or with compounds obtained by the methods described herein.

### EXAMPLE 4

### NMR Screening

NMR spectroscopy can be used to discover and design inhibitors of 3a-HSD type III. This is due to the well-known ability of NMR to detect interactions between ligands and a target protein, even if the interactions are only weak and have affinities in the millimolar range (Diercks, T.; Coles, M.; Kessler, H. Applications of NMR in drug discovery. Curr. Opin. Chem. Biol. 2001, 5, 285-291; Hajduk, P. J.; Meadows, R. P.; Fesik, S. W. NMR-based screening in drug discovery. Q. Rev. Biophys. 1999, 32, 211-240; Pellecchia, M.; Sem, D. S.; Wuthrich, K. NMR in drug discovery. Nature Reviews Drug Discovery 2002, 1, 211-219). For this purpose, a solution of 3a-HSD is added to a mixture of compounds, and NMR properties of the compounds, such as their relaxation rates, are measured and compared in the presence and absence of 3a-HSD. An increase in relaxation rates reveals binding affinity between 3a-HSD and said compound. This effect can be enhanced by about a factor of 15 if side chains of 3a-HSD are spin-labeled with paramagnetic centers such as TEMPO (2,2,6,6-Tetramethylpiperidin-N-oxyl) or PROXYL (2,2,5,5-Tetramethylpyrrolidine-N-oxyl) (Jahnke, W.; Ruedisser, S.; Zurini, M. Spin Label Enhanced NMR Screening. J. Am. Chem. Soc. 2001, 123, 3149-3150; Jahnke, W. Spin labels as a tool to identify and characterize protein—ligand interactions by NMR spectroscopy. Chem BioChem 2002, 3, 167-173).

About 200 compounds from an in-house NMR compound library (or library available from ACD Database, MDL Information Systems, Inc) containing a diverse set of small fragments of potential drugs are screened for interactions with 3a-HSD. Several fragments are identified which exhibited affinities to 3a-HSD with dissociation constants below 2 mM, more preferentially 1 mM, even more preferentially 0.5 mM. E.g. 2-acetylbenzofuran is found by this method and had a K_{i ox} of about 132.5 μM (see Example 5).

Although 2-acetylbenzofuran binds to 3a-HSD only weakly, ft is a progressible compound, i.e. it is a very small, soluble and “drug-like” compound that is amenable to chemical modification so that its potency can be improved. There are several ways to achieve this by NMR. One possibility is to select compounds by substructure or similarity search, and test them by NMR. Another possibility is to identify by NMR screening another compound that binds in the vicinity to 2-acetylbenzofuran, and to chemically link both compounds to yield a high-affinity ligand (Shuker, S. B.; Hajduk, P. J.; Meadows, R. P.; Fesik, S. W. Discovering high-affinity ligands for proteins: SAR by NMR. Science 1996, 274, 1531-1534; Jahnke, W.; Flösheimer, A.; Blommers, M.; Paris, C. G.; Helm, J.; Nalin, C. M.; Perez, L. B. Second-site NMR screening and linker design. Current Topics in Medicinal Chemistry 2002, 2, 1337-1348 (in press)). Given the availability of the coordinates from the crystal structure of 3a-HSD, however, the best way is to combine the results from NMR screening with in-silico GOLD docking, as described in example 3. NMR screening hits such as 2-acetylbenzofuran serve as input for GOLD docking experiments and guide the selection of compounds that are docked in silico. Since the docking itself, and the scoring of the results is not fully reliable, docking results need experimental validation by NMR. NMR and docking thus form an iterative cycle which leads to drastically improved compound potencies.

In spite of its low affinity, the dissociation rate of 2-acetylbenzofuran is so slow that severe line broadening is observed by NMR, and more tightly binding compounds cannot be detected by conventional ligand-observe NMR screening (Diercks, T.; Coles, M.; Kessler, H. Applications of NMR in drug discovery. Curr. Opin. Chem. Biol. 2001, 5, 285-291). We therefore had to develop an improved NMR screening method called NMR reporter screening. This method uses 2-acetylbenzofuran as “reporter ligand” and measures the ability of any test compound to displace the reporter ligand. By this technique, higher-affinity compounds can be detected by NMR screening that could previously not be detected (W. Jahnke, P. Floersheim, C. Ostermeier, X. Zhang, R. Hemmig, K. Hurth, D. Uzunov “NMR Reporter Screening for the Detection of High-Affinity Ligands” Angew. Chemie, in press). The combination of NMR reporter screening and GOLD docking lead us to identify sub-micromolar inhibitors within short time with very little chemistry efforts.

### EXAMPLE 5

### Functional In Vitro Assay

Materials: Steroid standards (>98% purity) were purchased from Sigma Chemical Co. ³H-allopregnanolone (64 Ci/mmol, [9, 11, 12-³H(N)]-allopregnanolone, herein after referred to as ³H-ALLO) was purchased from New England Nuclear. 3H-5α-dihydroprogesterone (3H-5α-DHP) was synthesized in our lab using an enzymatic conversion of ³H-ALLO to ³H-5α-DHP by the 3a-HSD (see Enzymatic reactions). The oxidation product ³H-5α-DHP was isolated and purified by semi-preparative HPLC and confirmed by GC/IMS. All solvents were of analytical grade (Merck) and were used without further purification.

Enzymatic reactions (oxidation of 3H-ALLO to ³H-5α-DHP and reduction of ³H-5α-DHP to 3H-ALLO): All enzymatic reactions were performed using pure recombinant type-3 human 3a-HSD expressed in E. coli and refolded and purified from bacterial inclusion bodies. ³H-5α-DHP reduction was conducted in 100 μl reaction systems containing 100 mM sodium phosphate (pH 7.4), 4 μg of recombinant enzyme, 2 mM NADPH and 2.5 μM ³H-5α-DHP (40 000 cpm) in 4% acetonitrile. Similarly, ³H-ALLO oxidation was conducted in 100 μl reaction systems containing 100 mM sodium phosphate (pH 7.4), 4 μg of recombinant enzyme, 2 mM NADP+ and 2.5 μM ³H-ALLO (40 000 cpm) in 4% acetonitrile. Reactions were initiated by the addition of the respective substrate and were incubated at 37° C. for 25 min. The selected reaction conditions afforded a rate of product accumulation within the linear range of the enzymatic conversion. Following the incubation, the reactions were quenched with 400 μl of ice-cold ethyl acetate. The resulting organic extracts were evaporated to dryness under a gentle stream of nitrogen and redissolved in hexane and injected into the HPLC for analysis. Negative control reactions omitting cofactor (NADPH or NADP⁺) or enzyme were carried out in order to exclude any non-enzymatic conversion. In all instances the control reactions resulted in no detectable product formation.

HPLC separation: Chromatographic separation of ³H-ALLO and ³H-5α-DHP was done on an Agilent 1100 series HPLC system (Agilent Technologies) equipped with a vacuum degasser, an autosampler, a quaternary pump and a compartment with a 5 μm normal phase LiChrosorb Diol column 125×4.6 mm (Merck). Chromatographic separation was achieved by isocratic elution with hexane/2-propanol (95:5, v/v). The flow rate was held constant at 1 ml/min. Sample injection volume is 1 ml. Column efflux is directly introduced into the Radioflow scintillation detector (Packard, 500 TR Series).

Candidate-inhibitors for type III human 3a-HSD obtained by methods described above, e.g. Examples 1 to 5 are tested in the functional assay and its K_{i ox} determined (K_{i ox} is the dissociation constant of the inhibitor for the oxidation of ³H-ALLO to ³H-5α-DHP: Kᵢ=IC50/(1+[S]/kₘ), whereas IC50 is the concentration of the inhibitor required to achieve a half-maximal degree of inhibition. IC50 values are determined graphically by plotting inhibitor concentrations versus rate of conversion of substrate to product. [S] is the concentration of the substrate (i.e. ³H-ALLO plus non-radiolabelled ALLO) in the sample and Kₘ is the Michaelis-Menten constant which is 4.9 mM for the oxidation of ALLO by the type-3 human 3a-HSD. E.g. 2-acetylbenzofuran has a K_{i ox} of about 132 μM.

### EXAMPLE 6

### Method to Find an Inhibitor of the Human Type III 3a-HSD Using the Rational Drug Design, the NMR Screening Methods, the in Silico Gold Docking, the Molecular Replacement and the In Vitro Functional Assay as Described Herein

Improved inhibitors of human type III 3a-HSD can be found in combining any of the methods, e.g. as described before, e.g. by using rational drug design techniques as described above in the description, the NMR screening method or NMR reporter screening as e.g. described in example 4, in silico Gold Docking, as e.g. described in example 3, Molecular Replacement, e.g. as described in Example 2 and an in vitro functional assay, e.g. as described in Example 5, in order to validate candidates obtained by any in silico/X-ray method described above. One of the preferred combinations for finding inhibitors to human type III 3a-HSD comprises the following methods:

1.) obtaining first inhibitory candidates by using NMR screening and/or NMR reporter screening; and
2.) co-crystallize the positive molecules (potential inhibitor) with the NADP/human type III 3a HSD complex and determine the difference of the three-dimensional structure of the active site of the inhibitor-NADP-3a_HSD complex with the NADP-3a_HSD complex (as described in Table 1).

TABLE 1

| | Atom | Res- | Num- | | | | | |
|---|---|---|---|---|---|---|---|---|
| Atom | Type | idue | ber | X | Y | Z | OCC | B |
| 1 | CB | SER | 3 | 29.684 | 51.64 | 91.533 | 1 | 53.36 |
| 2 | OG | SER | 3 | 29.417 | 51.09 | 92.819 | 1 | 55.34 |
| 3 | C | SER | 3 | 29.46 | 49.403 | 90.45 | 1 | 47.9 |
| 4 | O | SER | 3 | 28.974 | 48.588 | 91.232 | 1 | 48.75 |
| 5 | N | SER | 3 | 27.49 | 50.911 | 90.626 | 1 | 52.53 |
| 6 | CA | SER | 3 | 28.97 | 50.849 | 90.429 | 1 | 50.93 |
| 7 | N | LYS | 4 | 30.414 | 49.08 | 89.582 | 1 | 43.74 |
| 8 | CA | LYS | 4 | 30.966 | 47.73 | 89.541 | 1 | 38.13 |
| 9 | CB | LYS | 4 | 30.745 | 47.095 | 88.163 | 1 | 38.91 |
| 10 | CG | LYS | 4 | 29.324 | 46.569 | 87.945 | 1 | 40.17 |
| 11 | CD | LYS | 4 | 29.191 | 45.868 | 86.602 | 1 | 41.95 |
| 12 | CE | LYS | 4 | 27.86 | 45.138 | 86.479 | 1 | 43.75 |
| 13 | NZ | LYS | 4 | 26.694 | 46.036 | 86.66 | 1 | 44.35 |
| 14 | C | LYS | 4 | 32.456 | 47.757 | 89.881 | 1 | 34.56 |
| 15 | O | LYS | 4 | 33.145 | 48.739 | 89.612 | 1 | 33.21 |
| 16 | N | TYR | 5 | 32.943 | 46.681 | 90.491 | 1 | 29.37 |
| 17 | CA | TYR | 5 | 34.346 | 46.58 | 90.884 | 1 | 26.52 |
| 18 | CB | TYR | 5 | 34.592 | 47.403 | 92.151 | 1 | 24.65 |
| 19 | CG | TYR | 5 | 33.549 | 47.202 | 93.228 | 1 | 25.8 |
| 20 | CD1 | TYR | 5 | 33.731 | 46.272 | 94.256 | 1 | 24.69 |
| 21 | CE1 | TYR | 5 | 32.756 | 46.091 | 95.246 | 1 | 27.04 |
| 22 | CD2 | TYR | 5 | 32.367 | 47.945 | 93.211 | 1 | 24.77 |
| 23 | CE2 | TYR | 5 | 31.391 | 47.773 | 94.184 | 1 | 26.99 |
| 24 | CZ | TYR | 5 | 31.592 | 46.845 | 95.201 | 1 | 27.76 |
| 25 | OH | TYR | 5 | 30.626 | 46.679 | 96.17 | 1 | 29.56 |
| 26 | C | TYR | 5 | 34.695 | 45.112 | 91.108 | 1 | 24.56 |
| 27 | O | TYR | 5 | 33.812 | 44.299 | 91.378 | 1 | 25.07 |
| 28 | N | GLN | 6 | 35.975 | 44.775 | 90.99 | 1 | 22.99 |
| 29 | CA | GLN | 6 | 36.412 | 43.393 | 91.142 | 1 | 23.88 |
| 30 | CB | GLN | 6 | 37.799 | 43.189 | 90.518 | 1 | 25.49 |
| 31 | CG | GLN | 6 | 37.861 | 43.549 | 89.035 | 1 | 29.27 |
| 32 | CD | GLN | 6 | 39.184 | 43.18 | 88.366 | 1 | 30.43 |
| 33 | OE1 | GLN | 6 | 39.488 | 43.678 | 87.276 | 1 | 30.76 |
| 34 | NE2 | GLN | 6 | 39.967 | 42.295 | 89.003 | 1 | 24.78 |
| 35 | C | GLN | 6 | 36.425 | 42.911 | 92.586 | 1 | 22.92 |
| 36 | O | GLN | 6 | 37.017 | 43.544 | 93.466 | 1 | 19.91 |
| 37 | N | CYS | 7 | 35.765 | 41.772 | 92.8 | 1 | 22.7 |
| 38 | CA | CYS | 7 | 35.646 | 41.126 | 94.106 | 1 | 23.76 |
| 39 | CB | CYS | 7 | 34.227 | 41.255 | 94.655 | 1 | 26.64 |
| 40 | SG | CYS | 7 | 33.651 | 42.899 | 94.969 | 1 | 32.59 |
| 41 | C | CYS | 7 | 35.906 | 39.637 | 93.971 | 1 | 25.06 |
| 42 | O | CYS | 7 | 35.891 | 39.089 | 92.868 | 1 | 24.42 |
| 43 | N | VAL | 8 | 36.144 | 38.99 | 95.108 | 1 | 23.76 |
| 44 | CA | VAL | 8 | 36.326 | 37.546 | 95.155 | 1 | 23.21 |
| 45 | CB | VAL | 8 | 37.743 | 37.12 | 95.601 | 1 | 24.81 |
| 46 | CG1 | VAL | 8 | 38.736 | 37.422 | 94.498 | 1 | 28.43 |
| 47 | CG2 | VAL | 8 | 38.138 | 37.83 | 96.901 | 1 | 23.25 |
| 48 | C | VAL | 8 | 35.312 | 37.022 | 96.163 | 1 | 22.77 |
| 49 | O | VAL | 8 | 34.985 | 37.706 | 97.137 | 1 | 21.39 |
| 50 | N | LYS | 9 | 34.799 | 35.821 | 95.923 | 1 | 22.09 |
| 51 | CA | LYS | 9 | 33.826 | 35.233 | 96.835 | 1 | 22.21 |
| 52 | CB | LYS | 9 | 32.967 | 34.197 | 96.1 | 1 | 22.05 |
| 53 | CG | LYS | 9 | 31.756 | 33.734 | 96.904 | 1 | 23.4 |
| 54 | CD | LYS | 9 | 30.88 | 32.796 | 96.084 | 1 | 25.76 |
| 55 | CE | LYS | 9 | 29.619 | 32.417 | 96.841 | 1 | 27.37 |
| 56 | NZ | LYS | 9 | 28.71 | 31.573 | 96.02 | 1 | 30.87 |
| 57 | C | LYS | 9 | 34.542 | 34.561 | 98.009 | 1 | 21.14 |
| 58 | O | LYS | 9 | 35.465 | 33.777 | 97.809 | 1 | 20.76 |
| 59 | N | LEU | 10 | 34.115 | 34.881 | 99.228 | 1 | 20.25 |
| 60 | CA | LEU | 10 | 34.693 | 34.303 | 100.441 | 1 | 20.25 |
| 61 | CB | LEU | 10 | 34.502 | 35.271 | 101.613 | 1 | 20.7 |
| 62 | CG | LEU | 10 | 35.566 | 36.326 | 101.924 | 1 | 24.07 |
| 63 | CD1 | LEU | 10 | 36.404 | 36.635 | 100.726 | 1 | 23.86 |
| 64 | CD2 | LEU | 10 | 34.896 | 37.572 | 102.491 | 1 | 21.11 |
| 65 | C | LEU | 10 | 34.022 | 32.963 | 100.751 | 1 | 20.4 |
| 66 | O | LEU | 10 | 32.942 | 32.677 | 100.228 | 1 | 21.07 |
| 67 | N | ASN | 11 | 34.641 | 32.146 | 101.604 | 1 | 19.41 |
| 68 | CA | ASN | 11 | 34.065 | 30.838 | 101.913 | 1 | 19.95 |
| 69 | CB | ASN | 11 | 35.099 | 29.916 | 102.589 | 1 | 20.22 |
| 70 | CG | ASN | 11 | 35.601 | 30.448 | 103.931 | 1 | 20.22 |
| 71 | OD1 | ASN | 11 | 34.838 | 30.99 | 104.723 | 1 | 22.84 |
| 72 | ND2 | ASN | 11 | 36.892 | 30.269 | 104.191 | 1 | 17.3 |
| 73 | C | ASN | 11 | 32.768 | 30.888 | 102.724 | 1 | 20.53 |
| 74 | O | ASN | 11 | 32.155 | 29.85 | 102.983 | 1 | 21.53 |
| 75 | N | ASP | 12 | 32.335 | 32.084 | 103.12 | 1 | 19.89 |
| 76 | CA | ASP | 12 | 31.072 | 32.204 | 103.858 | 1 | 19.81 |
| 77 | CB | ASP | 12 | 31.238 | 33.04 | 105.14 | 1 | 19.46 |
| 78 | CG | ASP | 12 | 31.491 | 34.522 | 104.862 | 1 | 20.54 |
| 79 | OD1 | ASP | 12 | 31.606 | 34.92 | 103.688 | 1 | 18.88 |
| 80 | OD2 | ASP | 12 | 31.58 | 35.297 | 105.836 | 1 | 23.31 |
| 81 | C | ASP | 12 | 29.979 | 32.819 | 102.989 | 1 | 20.88 |
| 82 | O | ASP | 12 | 28.908 | 33.182 | 103.486 | 1 | 19.57 |
| 83 | N | GLY | 13 | 30.258 | 32.947 | 101.693 | 1 | 21.36 |
| 84 | CA | GLY | 13 | 29.272 | 33.502 | 100.782 | 1 | 20.73 |
| 85 | C | GLY | 13 | 29.366 | 34.996 | 100.522 | 1 | 21.91 |
| 86 | O | GLY | 13 | 28.736 | 35.487 | 99.587 | 1 | 22.03 |
| 87 | N | HIS | 14 | 30.114 | 35.729 | 101.346 | 1 | 19.06 |
| 88 | CA | HIS | 14 | 30.261 | 37.162 | 101.134 | 1 | 19.56 |
| 89 | CB | HIS | 14 | 30.579 | 37.878 | 102.454 | 1 | 20.87 |
| 90 | CG | HIS | 14 | 29.413 | 37.943 | 103.39 | 1 | 23.26 |
| 91 | CD2 | HIS | 14 | 28.218 | 38.569 | 103.279 | 1 | 23.14 |
| 92 | ND1 | HIS | 14 | 29.379 | 37.266 | 104.591 | 1 | 24.98 |
| 93 | CE1 | HIS | 14 | 28.214 | 37.47 | 105.178 | 1 | 23.12 |
| 94 | NE2 | HIS | 14 | 27.49 | 38.257 | 104.403 | 1 | 25.76 |
| 95 | C | HIS | 14 | 31.341 | 37.459 | 100.091 | 1 | 19.18 |
| 96 | O | HIS | 14 | 32.131 | 36.58 | 99.73 | 1 | 19.71 |
| 97 | N | PHE | 15 | 31.363 | 38.696 | 99.609 | 1 | 18.44 |
| 98 | CA | PHE | 15 | 32.322 | 39.115 | 98.596 | 1 | 20.05 |
| 99 | CB | PHE | 15 | 31.588 | 39.59 | 97.34 | 1 | 20.76 |
| 100 | CG | PHE | 15 | 30.994 | 38.472 | 96.529 | 1 | 22.15 |
| 101 | CD1 | PHE | 15 | 29.79 | 37.879 | 96.903 | 1 | 23.83 |
| 102 | CD2 | PHE | 15 | 31.648 | 38.005 | 95.395 | 1 | 21.78 |
| 103 | CE1 | PHE | 15 | 29.244 | 36.834 | 96.151 | 1 | 23.21 |
| 104 | CE2 | PHE | 15 | 31.113 | 36.961 | 94.637 | 1 | 22.5 |
| 105 | CZ | PHE | 15 | 29.91 | 36.378 | 95.016 | 1 | 22.08 |
| 106 | C | PHE | 15 | 33.236 | 40.219 | 99.103 | 1 | 19.31 |
| 107 | O | PHE | 15 | 32.795 | 41.145 | 99.77 | 1 | 20.45 |
| 108 | N | MET | 16 | 34.513 | 40.108 | 98.77 | 1 | 17.56 |
| 109 | CA | MET | 16 | 35.505 | 41.084 | 99.197 | 1 | 18.31 |
| 110 | CB | MET | 16 | 36.553 | 40.375 | 100.063 | 1 | 16.53 |
| 111 | CG | MET | 16 | 37.762 | 41.217 | 100.464 | 1 | 19.92 |
| 112 | SD | MET | 16 | 39.091 | 40.217 | 101.224 | 1 | 23.07 |
| 113 | CE | MET | 16 | 38.55 | 40.145 | 102.873 | 1 | 20.88 |
| 114 | C | MET | 16 | 36.193 | 41.74 | 97.998 | 1 | 17.1 |
| 115 | O | MET | 16 | 36.657 | 41.043 | 97.097 | 1 | 17.96 |
| 116 | N | PRO | 17 | 36.257 | 43.088 | 97.969 | 1 | 17.41 |
| 117 | CD | PRO | 17 | 35.566 | 44.018 | 98.887 | 1 | 16.2 |
| 118 | CA | PRO | 17 | 36.913 | 43.814 | 96.864 | 1 | 17.6 |
| 119 | CB | PRO | 17 | 36.69 | 45.286 | 97.232 | 1 | 17.75 |
| 120 | CG | PRO | 17 | 35.361 | 45.249 | 98.013 | 1 | 16.66 |
| 121 | C | PRO | 17 | 38.399 | 43.417 | 96.906 | 1 | 19.01 |
| 122 | O | PRO | 17 | 38.993 | 43.38 | 97.984 | 1 | 18.82 |
| 123 | N | VAL | 18 | 38.996 | 43.121 | 95.754 | 1 | 18.52 |
| 124 | CA | VAL | 18 | 40.388 | 42.664 | 95.709 | 1 | 19.91 |
| 125 | CB | VAL | 18 | 40.702 | 41.981 | 94.349 | 1 | 21.31 |
| 126 | CG1 | VAL | 18 | 39.786 | 40.79 | 94.151 | 1 | 21.12 |
| 127 | CG2 | VAL | 18 | 40.527 | 42.983 | 93.206 | 1 | 20.92 |
| 128 | C | VAL | 18 | 41.482 | 43.692 | 95.989 | 1 | 19.98 |
| 129 | O | VAL | 18 | 42.636 | 43.328 | 96.212 | 1 | 20.88 |
| 130 | N | LEU | 19 | 41.133 | 44.97 | 95.946 | 1 | 19.6 |
| 131 | CA | LEU | 19 | 42.101 | 46.024 | 96.227 | 1 | 21.48 |
| 132 | CB | LEU | 19 | 42.226 | 46.994 | 95.041 | 1 | 21.81 |
| 133 | CG | LEU | 19 | 43.07 | 48.259 | 95.308 | 1 | 22.44 |
| 134 | CD1 | LEU | 19 | 44.508 | 47.876 | 95.554 | 1 | 21.46 |
| 135 | CD2 | LEU | 19 | 42.977 | 49.207 | 94.122 | 1 | 23.75 |
| 136 | C | LEU | 19 | 41.59 | 46.769 | 97.449 | 1 | 20.8 |
| 137 | O | LEU | 19 | 40.466 | 47.272 | 97.449 | 1 | 21.52 |
| 138 | N | GLY | 20 | 42.406 | 46.812 | 98.499 | 1 | 20.99 |
| 139 | CA | GLY | 20 | 42.003 | 47.487 | 99.718 | 1 | 20.32 |
| 140 | C | GLY | 20 | 42.889 | 48.67 | 100.076 | 1 | 20.56 |
| 141 | O | GLY | 20 | 44.073 | 48.7 | 99.744 | 1 | 20.63 |
| 142 | N | PHE | 21 | 42.304 | 49.644 | 100.759 | 1 | 19.53 |
| 143 | CA | PHE | 21 | 43.021 | 50.842 | 101.173 | 1 | 21.46 |
| 144 | CB | PHE | 21 | 42.103 | 52.061 | 101.04 | 1 | 22.25 |
| 145 | CG | PHE | 21 | 42.789 | 53.364 | 101.343 | 1 | 23.59 |
| 146 | CD1 | PHE | 21 | 43.777 | 53.859 | 100.494 | 1 | 23.09 |
| 147 | CD2 | PHE | 21 | 42.482 | 54.07 | 102.502 | 1 | 24.76 |
| 148 | CE1 | PHE | 21 | 44.458 | 55.047 | 100.799 | 1 | 25.75 |
| 149 | CE2 | PHE | 21 | 43.149 | 55.25 | 102.822 | 1 | 25.54 |
| 150 | CZ | PHE | 21 | 44.143 | 55.741 | 101.967 | 1 | 24.8 |
| 151 | C | PHE | 21 | 43.508 | 50.717 | 102.625 | 1 | 21.08 |
| 152 | O | PHE | 21 | 42.707 | 50.506 | 103.531 | 1 | 21.23 |
| 153 | N | GLY | 22 | 44.818 | 50.847 | 102.835 | 1 | 21.87 |
| 154 | CA | GLY | 22 | 45.375 | 50.748 | 104.178 | 1 | 22.01 |
| 155 | C | GLY | 22 | 45.302 | 52.092 | 104.88 | 1 | 24.14 |
| 156 | O | GLY | 22 | 45.555 | 53.117 | 104.253 | 1 | 22.23 |
| 157 | N | THR | 23 | 44.972 | 52.107 | 106.172 | 1 | 22.37 |
| 158 | CA | THR | 23 | 44.843 | 53.379 | 106.886 | 1 | 24.15 |
| 159 | CB | THR | 23 | 43.435 | 53.534 | 107.476 | 1 | 23.82 |
| 160 | OG1 | THR | 23 | 43.215 | 52.515 | 108.462 | 1 | 22.83 |
| 161 | CG2 | THR | 23 | 42.388 | 53.408 | 106.382 | 1 | 23.53 |
| 162 | C | THR | 23 | 45.84 | 53.643 | 108.014 | 1 | 25.4 |
| 163 | O | THR | 23 | 45.764 | 54.678 | 108.677 | 1 | 26.47 |
| 164 | N | TYR | 24 | 46.763 | 52.721 | 108.25 | 1 | 25.3 |
| 165 | CA | TYR | 24 | 47.74 | 52.934 | 109.308 | 1 | 28.67 |
| 166 | CB | TYR | 24 | 48.488 | 51.643 | 109.649 | 1 | 29.42 |
| 167 | CG | TYR | 24 | 49.586 | 51.88 | 110.671 | 1 | 31.62 |
| 168 | CD1 | TYR | 24 | 49.275 | 52.095 | 112.013 | 1 | 33.37 |
| 169 | CE1 | TYR | 24 | 50.268 | 52.404 | 112.948 | 1 | 33.58 |
| 170 | CD2 | TYR | 24 | 50.927 | 51.975 | 110.281 | 1 | 32.74 |
| 171 | CE2 | TYR | 24 | 51.931 | 52.286 | 111.206 | 1 | 33.21 |
| 172 | CZ | TYR | 24 | 51.59 | 52.5 | 112.536 | 1 | 35.01 |
| 173 | OH | TYR | 24 | 52.562 | 52.829 | 113.454 | 1 | 36.55 |
| 174 | C | TYR | 24 | 48.779 | 53.977 | 108.904 | 1 | 29.73 |
| 175 | O | TYR | 24 | 49.238 | 53.996 | 107.764 | 1 | 29.54 |
| 176 | N | ALA | 25 | 49.154 | 54.831 | 109.849 | 1 | 30.5 |
| 177 | CA | ALA | 25 | 50.178 | 55.842 | 109.606 | 1 | 33.31 |
| 178 | CB | ALA | 25 | 49.536 | 57.146 | 109.119 | 1 | 32.46 |
| 179 | C | ALA | 25 | 50.934 | 56.078 | 110.913 | 1 | 35.53 |
| 180 | O | ALA | 25 | 50.366 | 55.919 | 111.993 | 1 | 34.49 |
| 181 | N | PRO | 26 | 52.231 | 56.434 | 110.83 | 1 | 38.01 |
| 182 | CD | PRO | 26 | 53.02 | 56.593 | 109.594 | 1 | 39.29 |
| 183 | CA | PRO | 26 | 53.059 | 56.694 | 112.016 | 1 | 39.8 |
| 184 | CB | PRO | 26 | 54.336 | 57.279 | 111.422 | 1 | 40.12 |
| 185 | CG | PRO | 26 | 54.448 | 56.563 | 110.113 | 1 | 40.44 |
| 186 | C | PRO | 26 | 52.369 | 57.677 | 112.957 | 1 | 42.01 |
| 187 | O | PRO | 26 | 51.652 | 58.576 | 112.513 | 1 | 41.02 |
| 188 | N | ALA | 27 | 52.598 | 57.509 | 114.255 | 1 | 45.58 |
| 189 | CA | ALA | 27 | 51.988 | 58.376 | 115.262 | 1 | 48.46 |
| 190 | CB | ALA | 27 | 52.436 | 57.946 | 116.66 | 1 | 49.56 |
| 191 | C | ALA | 27 | 52.302 | 59.856 | 115.043 | 1 | 49.84 |
| 192 | O | ALA | 27 | 51.566 | 60.726 | 115.509 | 1 | 50.69 |
| 193 | N | GLU | 28 | 53.392 | 60.136 | 114.337 | 1 | 50.59 |
| 194 | CA | GLU | 28 | 53.792 | 61.509 | 114.06 | 1 | 52.06 |
| 195 | CB | GLU | 28 | 55.211 | 61.531 | 113.489 | 1 | 54.9 |
| 196 | CG | GLU | 28 | 55.375 | 60.676 | 112.24 | 1 | 59.71 |
| 197 | CD | GLU | 28 | 56.81 | 60.597 | 111.755 | 1 | 62.27 |
| 198 | OE1 | GLU | 28 | 57.347 | 61.635 | 111.299 | 1 | 64.37 |
| 199 | OE2 | GLU | 28 | 57.401 | 59.493 | 111.832 | 1 | 63.33 |
| 200 | C | GLU | 28 | 52.836 | 62.196 | 113.086 | 1 | 51.24 |
| 201 | O | GLU | 28 | 52.762 | 63.424 | 113.044 | 1 | 52.06 |
| 202 | N | VAL | 29 | 52.106 | 61.406 | 112.302 | 1 | 49.06 |
| 203 | CA | VAL | 29 | 51.158 | 61.953 | 111.331 | 1 | 46.23 |
| 204 | CB | VAL | 29 | 50.911 | 60.963 | 110.168 | 1 | 44.91 |
| 205 | CG1 | VAL | 29 | 50.031 | 61.611 | 109.112 | 1 | 44.36 |
| 206 | CG2 | VAL | 29 | 52.231 | 60.519 | 109.564 | 1 | 44.23 |
| 207 | C | VAL | 29 | 49.812 | 62.268 | 111.989 | 1 | 46.13 |
| 208 | O | VAL | 29 | 49.256 | 61.444 | 112.71 | 1 | 45.55 |
| 209 | N | PRO | 30 | 49.273 | 63.475 | 111.747 | 1 | 45.99 |
| 210 | CD | PRO | 30 | 49.877 | 64.564 | 110.958 | 1 | 46.31 |
| 211 | CA | PRO | 30 | 47.987 | 63.895 | 112.318 | 1 | 46.18 |
| 212 | CB | PRO | 30 | 47.788 | 65.294 | 111.735 | 1 | 45.8 |
| 213 | CG | PRO | 30 | 49.191 | 65.784 | 111.527 | 1 | 46.49 |
| 214 | C | PRO | 30 | 46.845 | 62.953 | 111.916 | 1 | 46.96 |
| 215 | O | PRO | 30 | 46.754 | 62.536 | 110.759 | 1 | 45.97 |
| 216 | N | LYS | 31 | 45.974 | 62.637 | 112.87 | 1 | 46.63 |
| 217 | CA | LYS | 31 | 44.844 | 61.753 | 112.609 | 1 | 48.02 |
| 218 | CB | LYS | 31 | 44.114 | 61.41 | 113.915 | 1 | 48.47 |
| 219 | CG | LYS | 31 | 44.941 | 60.604 | 114.911 | 1 | 49.59 |
| 220 | CD | LYS | 31 | 45.462 | 59.309 | 114.3 | 1 | 50.1 |
| 221 | CE | LYS | 31 | 46.34 | 58.554 | 115.287 | 1 | 50.23 |
| 222 | NZ | LYS | 31 | 47.041 | 57.403 | 114.652 | 1 | 52.26 |
| 223 | C | LYS | 31 | 43.86 | 62.375 | 111.622 | 1 | 47.6 |
| 224 | O | LYS | 31 | 42.999 | 61.685 | 111.074 | 1 | 48.62 |
| 225 | N | SER | 32 | 43.992 | 63.677 | 111.394 | 1 | 47.2 |
| 226 | CA | SER | 32 | 43.109 | 64.382 | 110.469 | 1 | 45.71 |
| 227 | CB | SER | 32 | 43.283 | 65.9 | 110.621 | 1 | 46.43 |
| 228 | OG | SER | 32 | 44.594 | 66.315 | 110.261 | 1 | 48.27 |
| 229 | C | SER | 32 | 43.376 | 63.977 | 109.022 | 1 | 44.36 |
| 230 | O | SER | 32 | 42.477 | 64.017 | 108.183 | 1 | 44.55 |
| 231 | N | LYS | 33 | 44.615 | 63.597 | 108.729 | 1 | 43.24 |
| 232 | CA | LYS | 33 | 44.973 | 63.183 | 107.38 | 1 | 42.33 |
| 233 | CB | LYS | 33 | 46.494 | 63.097 | 107.227 | 1 | 45.32 |
| 234 | CG | LYS | 33 | 47.195 | 64.447 | 107.134 | 1 | 49.02 |
| 235 | CD | LYS | 33 | 48.667 | 64.267 | 106.781 | 1 | 52.78 |
| 236 | CE | LYS | 33 | 49.383 | 65.604 | 106.637 | 1 | 55.15 |
| 237 | NZ | LYS | 33 | 50.83 | 65.425 | 106.309 | 1 | 56.11 |
| 238 | C | LYS | 33 | 44.335 | 61.836 | 107.03 | 1 | 39.84 |
| 239 | O | LYS | 33 | 44.162 | 61.517 | 105.858 | 1 | 38.33 |
| 240 | N | ALA | 34 | 43.992 | 61.049 | 108.049 | 1 | 37.92 |
| 241 | CA | ALA | 34 | 43.362 | 59.754 | 107.815 | 1 | 36.35 |
| 242 | CB | ALA | 34 | 43.311 | 58.938 | 109.1 | 1 | 36.8 |
| 243 | C | ALA | 34 | 41.955 | 60.004 | 107.288 | 1 | 34.73 |
| 244 | O | ALA | 34 | 41.474 | 59.295 | 106.404 | 1 | 34.44 |
| 245 | N | LEU | 35 | 41.299 | 61.024 | 107.828 | 1 | 33.55 |
| 246 | CA | LEU | 35 | 39.953 | 61.369 | 107.393 | 1 | 33.63 |
| 247 | CB | LEU | 35 | 39.387 | 62.502 | 108.258 | 1 | 33.49 |
| 248 | CG | LEU | 35 | 38.068 | 63.145 | 107.816 | 1 | 33.7 |
| 249 | CD1 | LEU | 35 | 36.942 | 62.127 | 107.752 | 1 | 34.9 |
| 250 | CD2 | LEU | 35 | 37.728 | 64.246 | 108.801 | 1 | 35.32 |
| 251 | C | LEU | 35 | 39.954 | 61.788 | 105.928 | 1 | 33.63 |
| 252 | O | LEU | 35 | 39.104 | 61.353 | 105.147 | 1 | 32.52 |
| 253 | N | GLU | 36 | 40.914 | 62.627 | 105.553 | 1 | 34.41 |
| 254 | CA | GLU | 36 | 41.01 | 63.102 | 104.175 | 1 | 34.68 |
| 255 | CB | GLU | 36 | 42.034 | 64.231 | 104.081 | 1 | 38.75 |
| 256 | CG | GLU | 36 | 41.765 | 65.357 | 105.062 | 1 | 47.25 |
| 257 | CD | GLU | 36 | 42.812 | 66.459 | 104.996 | 1 | 51.7 |
| 258 | OE1 | GLU | 36 | 44.023 | 66.142 | 105.073 | 1 | 54.07 |
| 259 | OE2 | GLU | 36 | 42.419 | 67.642 | 104.878 | 1 | 54.43 |
| 260 | C | GLU | 36 | 41.405 | 61.981 | 103.23 | 1 | 32.2 |
| 261 | O | GLU | 36 | 40.844 | 61.856 | 102.146 | 1 | 32.14 |
| 262 | N | ALA | 37 | 42.367 | 61.166 | 103.655 | 1 | 29.99 |
| 263 | CA | ALA | 37 | 42.849 | 60.063 | 102.839 | 1 | 28.84 |
| 264 | CB | ALA | 37 | 44.001 | 59.354 | 103.539 | 1 | 27.75 |
| 265 | C | ALA | 37 | 41.747 | 59.066 | 102.487 | 1 | 28.07 |
| 266 | O | ALA | 37 | 41.636 | 58.653 | 101.334 | 1 | 28.97 |
| 267 | N | VAL | 38 | 40.936 | 58.674 | 103.467 | 1 | 26.41 |
| 268 | CA | VAL | 38 | 39.863 | 57.728 | 103.196 | 1 | 26.44 |
| 269 | CB | VAL | 38 | 39.164 | 57.27 | 104.497 | 1 | 25.3 |
| 270 | CG1 | VAL | 38 | 37.989 | 56.356 | 104.167 | 1 | 24.07 |
| 271 | CG2 | VAL | 38 | 40.157 | 56.522 | 105.376 | 1 | 26.35 |
| 272 | C | VAL | 38 | 38.834 | 58.301 | 102.227 | 1 | 27.14 |
| 273 | O | VAL | 38 | 38.334 | 57.588 | 101.359 | 1 | 26 |
| 274 | N | LYS | 39 | 38.523 | 59.587 | 102.362 | 1 | 28.93 |
| 275 | CA | LYS | 39 | 37.56 | 60.217 | 101.461 | 1 | 30.2 |
| 276 | CB | LYS | 39 | 37.327 | 61.675 | 101.852 | 1 | 32.14 |
| 277 | CG | LYS | 39 | 36.432 | 61.861 | 103.054 | 1 | 34.97 |
| 278 | CD | LYS | 39 | 36.169 | 63.343 | 103.281 | 1 | 40.49 |
| 279 | CE | LYS | 39 | 35.184 | 63.567 | 104.412 | 1 | 43.92 |
| 280 | NZ | LYS | 39 | 34.842 | 65.012 | 104.574 | 1 | 46.69 |
| 281 | C | LYS | 39 | 38.06 | 60.156 | 100.025 | 1 | 28.98 |
| 282 | O | LYS | 39 | 37.306 | 59.804 | 99.124 | 1 | 30.66 |
| 283 | N | LEU | 40 | 39.331 | 60.492 | 99.826 | 1 | 29.51 |
| 284 | CA | LEU | 40 | 39.945 | 60.475 | 98.496 | 1 | 30.36 |
| 285 | CB | LEU | 40 | 41.368 | 61.049 | 98.564 | 1 | 31.84 |
| 286 | CG | LEU | 40 | 41.514 | 62.554 | 98.853 | 1 | 35.77 |
| 287 | CD1 | LEU | 40 | 42.989 | 62.908 | 99.07 | 1 | 35.52 |
| 288 | CD2 | LEU | 40 | 40.936 | 63.362 | 97.685 | 1 | 36.06 |
| 289 | C | LEU | 40 | 39.989 | 59.06 | 97.918 | 1 | 29.76 |
| 290 | O | LEU | 40 | 39.742 | 58.86 | 96.729 | 1 | 28.44 |
| 291 | N | ALA | 41 | 40.306 | 58.082 | 98.768 | 1 | 28.87 |
| 292 | CA | ALA | 41 | 40.378 | 56.685 | 98.338 | 1 | 26.84 |
| 293 | CB | ALA | 41 | 40.854 | 55.794 | 99.492 | 1 | 25.05 |
| 294 | C | ALA | 41 | 39.016 | 56.223 | 97.84 | 1 | 25.75 |
| 295 | O | ALA | 41 | 38.914 | 55.526 | 96.829 | 1 | 26.2 |
| 296 | N | ILE | 42 | 37.961 | 56.602 | 98.55 | 1 | 25.35 |
| 297 | CA | ILE | 42 | 36.629 | 56.221 | 98.118 | 1 | 27.2 |
| 298 | CB | ILE | 42 | 35.57 | 56.58 | 99.187 | 1 | 26.66 |
| 299 | CG2 | ILE | 42 | 34.159 | 56.397 | 98.628 | 1 | 27.75 |
| 300 | CG1 | ILE | 42 | 35.754 | 55.661 | 100.406 | 1 | 26.13 |
| 301 | CD1 | ILE | 42 | 34.852 | 55.998 | 101.595 | 1 | 25.97 |
| 302 | C | ILE | 42 | 36.306 | 56.904 | 96.787 | 1 | 28.89 |
| 303 | O | ILE | 42 | 35.74 | 56.287 | 95.892 | 1 | 28.44 |
| 304 | N | GLU | 43 | 36.677 | 58.174 | 96.653 | 1 | 31.16 |
| 305 | CA | GLU | 43 | 36.435 | 58.907 | 95.407 | 1 | 32.94 |
| 306 | CB | GLU | 43 | 36.827 | 60.374 | 95.577 | 1 | 35.59 |
| 307 | CG | GLU | 43 | 35.907 | 61.14 | 96.502 | 1 | 42.17 |
| 308 | CD | GLU | 43 | 36.38 | 62.561 | 96.745 | 1 | 45.48 |
| 309 | OE1 | GLU | 43 | 36.856 | 63.201 | 95.781 | 1 | 47.75 |
| 310 | OE2 | GLU | 43 | 36.262 | 63.044 | 97.892 | 1 | 46.68 |
| 311 | C | GLU | 43 | 37.225 | 58.307 | 94.246 | 1 | 30.98 |
| 312 | O | GLU | 43 | 36.749 | 58.292 | 93.115 | 1 | 32.76 |
| 313 | N | ALA | 44 | 38.428 | 57.816 | 94.532 | 1 | 29.59 |
| 314 | CA | ALA | 44 | 39.284 | 57.224 | 93.504 | 1 | 28.25 |
| 315 | CB | ALA | 44 | 40.728 | 57.115 | 94.014 | 1 | 28.17 |
| 316 | C | ALA | 44 | 38.798 | 55.857 | 93.028 | 1 | 27.79 |
| 317 | O | ALA | 44 | 39.182 | 55.406 | 91.949 | 1 | 26.16 |
| 318 | N | GLY | 45 | 37.97 | 55.187 | 93.828 | 1 | 27.07 |
| 319 | CA | GLY | 45 | 37.474 | 53.88 | 93.421 | 1 | 26.05 |
| 320 | C | GLY | 45 | 37.757 | 52.729 | 94.375 | 1 | 26.58 |
| 321 | O | GLY | 45 | 37.528 | 51.57 | 94.027 | 1 | 26.47 |
| 322 | N | PHE | 46 | 38.269 | 53.031 | 95.565 | 1 | 24.63 |
| 323 | CA | PHE | 46 | 38.539 | 51.989 | 96.56 | 1 | 25.36 |
| 324 | CB | PHE | 46 | 39.503 | 52.495 | 97.638 | 1 | 23.2 |
| 325 | CG | PHE | 46 | 40.949 | 52.423 | 97.253 | 1 | 23.59 |
| 326 | CD1 | PHE | 46 | 41.712 | 51.302 | 97.571 | 1 | 22.81 |
| 327 | CD2 | PHE | 46 | 41.566 | 53.496 | 96.611 | 1 | 23.75 |
| 328 | CE1 | PHE | 46 | 43.069 | 51.248 | 97.267 | 1 | 22.04 |
| 329 | CE2 | PHE | 46 | 42.926 | 53.453 | 96.298 | 1 | 23.33 |
| 330 | CZ | PHE | 46 | 43.681 | 52.325 | 96.629 | 1 | 24.41 |
| 331 | C | PHE | 46 | 37.208 | 51.638 | 97.222 | 1 | 24.15 |
| 332 | O | PHE | 46 | 36.455 | 52.532 | 97.628 | 1 | 25.33 |
| 333 | N | HIS | 47 | 36.91 | 50.345 | 97.321 | 1 | 22.85 |
| 334 | CA | HIS | 47 | 35.667 | 49.882 | 97.952 | 1 | 22.14 |
| 335 | CB | HIS | 47 | 34.85 | 49.062 | 96.952 | 1 | 21.04 |
| 336 | CG | HIS | 47 | 34.209 | 49.888 | 95.882 | 1 | 23.38 |
| 337 | CD2 | HIS | 47 | 34.66 | 50.28 | 94.668 | 1 | 23.45 |
| 338 | ND1 | HIS | 47 | 32.97 | 50.471 | 96.036 | 1 | 24.56 |
| 339 | CE1 | HIS | 47 | 32.685 | 51.189 | 94.964 | 1 | 24.39 |
| 340 | NE2 | HIS | 47 | 33.696 | 51.091 | 94.119 | 1 | 24.67 |
| 341 | C | HIS | 47 | 35.946 | 49.027 | 99.189 | 1 | 22.75 |
| 342 | O | HIS | 47 | 35.022 | 48.521 | 99.83 | 1 | 23.25 |
| 343 | N | HIS | 48 | 37.224 | 48.875 | 99.514 | 1 | 20.59 |
| 344 | CA | HIS | 48 | 37.675 | 48.065 | 100.645 | 1 | 20.27 |
| 345 | CB | HIS | 48 | 38.399 | 46.83 | 100.081 | 1 | 20.69 |
| 346 | CG | HIS | 48 | 39.029 | 45.939 | 101.111 | 1 | 22.81 |
| 347 | CD2 | HIS | 48 | 39.289 | 46.13 | 102.426 | 1 | 21.12 |
| 348 | ND1 | HIS | 48 | 39.532 | 44.692 | 100.798 | 1 | 22.45 |
| 349 | CE1 | HIS | 48 | 40.077 | 44.157 | 101.876 | 1 | 23.25 |
| 350 | NE2 | HIS | 48 | 39.944 | 45.009 | 102.877 | 1 | 23.18 |
| 351 | C | HIS | 48 | 38.603 | 48.949 | 101.489 | 1 | 21.79 |
| 352 | O | HIS | 48 | 39.575 | 49.507 | 100.976 | 1 | 20.72 |
| 353 | N | ILE | 49 | 38.27 | 49.09 | 102.774 | 1 | 19.58 |
| 354 | CA | ILE | 49 | 39.036 | 49.92 | 103.706 | 1 | 19.68 |
| 355 | CB | ILE | 49 | 38.183 | 51.11 | 104.212 | 1 | 20.44 |
| 356 | CG2 | ILE | 49 | 39.011 | 52 | 105.148 | 1 | 21 |
| 357 | CG1 | ILE | 49 | 37.68 | 51.919 | 103.005 | 1 | 24.53 |
| 358 | CD1 | ILE | 49 | 36.588 | 52.902 | 103.333 | 1 | 30.47 |
| 359 | C | ILE | 49 | 39.464 | 49.069 | 104.896 | 1 | 20.16 |
| 360 | O | ILE | 49 | 38.639 | 48.422 | 105.533 | 1 | 19.37 |
| 361 | N | ASP | 50 | 40.752 | 49.102 | 105.199 | 1 | 18.44 |
| 362 | CA | ASP | 50 | 41.313 | 48.303 | 106.271 | 1 | 18.73 |
| 363 | CB | ASP | 50 | 42.475 | 47.462 | 105.728 | 1 | 17.47 |
| 364 | CG | ASP | 50 | 43.06 | 46.52 | 106.784 | 1 | 19.24 |
| 365 | OD1 | ASP | 50 | 42.554 | 45.389 | 106.907 | 1 | 16.55 |
| 366 | OD2 | ASP | 50 | 44.008 | 46.922 | 107.486 | 1 | 17.4 |
| 367 | C | ASP | 50 | 41.81 | 49.131 | 107.448 | 1 | 19.63 |
| 368 | O | ASP | 50 | 42.648 | 50.021 | 107.28 | 1 | 19.78 |
| 369 | N | SER | 51 | 41.277 | 48.861 | 108.636 | 1 | 18.56 |
| 370 | CA | SER | 51 | 41.745 | 49.567 | 109.819 | 1 | 19.98 |
| 371 | CB | SER | 51 | 40.838 | 50.746 | 110.162 | 1 | 18.97 |
| 372 | OG | SER | 51 | 41.521 | 51.606 | 111.062 | 1 | 23.18 |
| 373 | C | SER | 51 | 41.846 | 48.609 | 111.008 | 1 | 18.6 |
| 374 | O | SER | 51 | 41.919 | 47.393 | 110.829 | 1 | 20.07 |
| 375 | N | ALA | 52 | 41.838 | 49.159 | 112.216 | 1 | 18.69 |
| 376 | CA | ALA | 52 | 41.971 | 48.344 | 113.414 | 1 | 17.84 |
| 377 | CB | ALA | 52 | 43.332 | 47.638 | 113.41 | 1 | 14.77 |
| 378 | C | ALA | 52 | 41.878 | 49.243 | 114.632 | 1 | 19.96 |
| 379 | O | ALA | 52 | 42.15 | 50.447 | 114.557 | 1 | 18.84 |
| 380 | N | HIS | 53 | 41.502 | 48.657 | 115.763 | 1 | 19.14 |
| 381 | CA | HIS | 53 | 41.409 | 49.419 | 116.992 | 1 | 20.34 |
| 382 | CB | HIS | 53 | 40.997 | 48.499 | 118.145 | 1 | 20.52 |
| 383 | CG | HIS | 53 | 41.041 | 49.165 | 119.486 | 1 | 23.94 |
| 384 | CD2 | HIS | 53 | 41.803 | 48.92 | 120.579 | 1 | 23.25 |
| 385 | ND1 | HIS | 53 | 40.255 | 50.255 | 119.8 | 1 | 23.72 |
| 386 | CE1 | HIS | 53 | 40.534 | 50.654 | 121.03 | 1 | 25 |
| 387 | NE2 | HIS | 53 | 41.469 | 49.861 | 121.524 | 1 | 24.2 |
| 388 | C | HIS | 53 | 42.755 | 50.095 | 117.312 | 1 | 20.43 |
| 389 | O | HIS | 53 | 42.802 | 51.293 | 117.611 | 1 | 18.98 |
| 390 | N | VAL | 54 | 43.847 | 49.339 | 117.223 | 1 | 21.34 |
| 391 | CA | VAL | 54 | 45.174 | 49.877 | 117.545 | 1 | 23.8 |
| 392 | CB | VAL | 54 | 46.292 | 48.809 | 117.566 | 1 | 26.89 |
| 393 | CG1 | VAL | 54 | 46.201 | 48.001 | 118.817 | 1 | 30.04 |
| 394 | CG2 | VAL | 54 | 46.239 | 47.948 | 116.315 | 1 | 25.21 |
| 395 | C | VAL | 54 | 45.704 | 50.985 | 116.665 | 1 | 24.26 |
| 396 | O | VAL | 54 | 46.69 | 51.62 | 117.028 | 1 | 23.77 |
| 397 | N | TYR | 55 | 45.088 | 51.213 | 115.51 | 1 | 22.57 |
| 398 | CA | TYR | 55 | 45.559 | 52.283 | 114.632 | 1 | 23.31 |
| 399 | CB | TYR | 55 | 45.026 | 52.103 | 113.211 | 1 | 20.13 |
| 400 | CG | TYR | 55 | 45.522 | 50.852 | 112.51 | 1 | 20.5 |
| 401 | CD1 | TYR | 55 | 46.531 | 50.058 | 113.068 | 1 | 19.22 |
| 402 | CE1 | TYR | 55 | 46.958 | 48.886 | 112.438 | 1 | 19.39 |
| 403 | CD2 | TYR | 55 | 44.957 | 50.447 | 111.296 | 1 | 19.69 |
| 404 | CE2 | TYR | 55 | 45.369 | 49.288 | 110.659 | 1 | 18.6 |
| 405 | CZ | TYR | 55 | 46.368 | 48.508 | 111.238 | 1 | 20.53 |
| 406 | OH | TYR | 55 | 46.742 | 47.334 | 110.629 | 1 | 20.19 |
| 407 | C | TYR | 55 | 45.096 | 53.624 | 115.185 | 1 | 24.66 |
| 408 | O | TYR | 55 | 45.559 | 54.683 | 114.756 | 1 | 25.17 |
| 409 | N | ASN | 56 | 44.171 | 53.562 | 116.137 | 1 | 25.36 |
| 410 | CA | ASN | 56 | 43.639 | 54.754 | 116.787 | 1 | 27.21 |
| 411 | CB | ASN | 56 | 44.723 | 55.399 | 117.649 | 1 | 31.46 |
| 412 | CG | ASN | 56 | 44.148 | 56.263 | 118.749 | 1 | 37.58 |
| 413 | OD1 | ASN | 56 | 44.81 | 57.173 | 119.248 | 1 | 42.85 |
| 414 | ND2 | ASN | 56 | 42.912 | 55.976 | 119.145 | 1 | 39.28 |
| 415 | C | ASN | 56 | 43.105 | 55.786 | 115.796 | 1 | 28.09 |
| 416 | O | ASN | 56 | 43.309 | 56.991 | 115.974 | 1 | 27.95 |
| 417 | N | ASN | 57 | 42.419 | 55.322 | 114.754 | 1 | 25.17 |
| 418 | CA | ASN | 57 | 41.873 | 56.237 | 113.756 | 1 | 24.82 |
| 419 | CB | ASN | 57 | 42.783 | 56.263 | 112.513 | 1 | 25.32 |
| 420 | CG | ASN | 57 | 42.839 | 54.916 | 111.793 | 1 | 24.64 |
| 421 | OD1 | ASN | 57 | 42.006 | 54.035 | 112.03 | 1 | 26.13 |
| 422 | ND2 | ASN | 57 | 43.804 | 54.763 | 110.894 | 1 | 25.4 |
| 423 | C | ASN | 57 | 40.447 | 55.888 | 113.326 | 1 | 24.2 |
| 424 | O | ASN | 57 | 39.912 | 56.502 | 112.406 | 1 | 23.01 |
| 425 | N | GLU | 58 | 39.819 | 54.921 | 113.996 | 1 | 23.33 |
| 426 | CA | GLU | 58 | 38.474 | 54.511 | 113.6 | 1 | 23.19 |
| 427 | CB | GLU | 58 | 38.028 | 53.276 | 114.4 | 1 | 25.16 |
| 428 | CG | GLU | 58 | 38.761 | 52.004 | 113.921 | 1 | 23.69 |
| 429 | CD | GLU | 58 | 38.196 | 50.71 | 114.49 | 1 | 23.95 |
| 430 | OE1 | GLU | 58 | 38.121 | 50.579 | 115.731 | 1 | 22.25 |
| 431 | OE2 | GLU | 58 | 37.843 | 49.816 | 113.691 | 1 | 23.43 |
| 432 | C | GLU | 58 | 37.431 | 55.616 | 113.658 | 1 | 25.46 |
| 433 | O | GLU | 58 | 36.455 | 55.599 | 112.908 | 1 | 23.44 |
| 434 | N | GLU | 59 | 37.639 | 56.585 | 114.539 | 1 | 26.95 |
| 435 | CA | GLU | 59 | 36.723 | 57.703 | 114.64 | 1 | 28.91 |
| 436 | CB | GLU | 59 | 37.121 | 58.573 | 115.829 | 1 | 33.46 |
| 437 | CG | GLU | 59 | 36.187 | 59.716 | 116.111 | 1 | 40.91 |
| 438 | CD | GLU | 59 | 36.522 | 60.395 | 117.425 | 1 | 45.45 |
| 439 | OE1 | GLU | 59 | 37.593 | 61.038 | 117.513 | 1 | 47.49 |
| 440 | OE2 | GLU | 59 | 35.715 | 60.264 | 118.371 | 1 | 48.25 |
| 441 | C | GLU | 59 | 36.805 | 58.507 | 113.328 | 1 | 28.63 |
| 442 | O | GLU | 59 | 35.782 | 58.854 | 112.742 | 1 | 27.41 |
| 443 | N | GLN | 60 | 38.022 | 58.78 | 112.866 | 1 | 27.82 |
| 444 | CA | GLN | 60 | 38.206 | 59.534 | 111.627 | 1 | 28.77 |
| 445 | CB | GLN | 60 | 39.669 | 59.966 | 111.455 | 1 | 30.05 |
| 446 | CG | GLN | 60 | 40.163 | 61.032 | 112.434 | 1 | 31.29 |
| 447 | CD | GLN | 60 | 40.225 | 60.531 | 113.862 | 1 | 35.92 |
| 448 | OE1 | GLN | 60 | 40.558 | 59.37 | 114.109 | 1 | 34.47 |
| 449 | NE2 | GLN | 60 | 39.923 | 61.41 | 114.815 | 1 | 36.75 |
| 450 | C | GLN | 60 | 37.782 | 58.714 | 110.41 | 1 | 27.57 |
| 451 | O | GLN | 60 | 37.099 | 59.221 | 109.514 | 1 | 27.1 |
| 452 | N | VAL | 61 | 38.184 | 57.446 | 110.377 | 1 | 25.88 |
| 453 | CA | VAL | 61 | 37.847 | 56.563 | 109.258 | 1 | 24.99 |
| 454 | CB | VAL | 61 | 38.497 | 55.155 | 109.432 | 1 | 24.7 |
| 455 | CG1 | VAL | 61 | 37.992 | 54.204 | 108.344 | 1 | 26.3 |
| 456 | CG2 | VAL | 61 | 40.009 | 55.276 | 109.353 | 1 | 22.58 |
| 457 | C | VAL | 61 | 36.334 | 56.43 | 109.14 | 1 | 24.41 |
| 458 | O | VAL | 61 | 35.781 | 56.514 | 108.044 | 1 | 24.23 |
| 459 | N | GLY | 62 | 35.662 | 56.25 | 110.276 | 1 | 24.84 |
| 460 | CA | GLY | 62 | 34.216 | 56.134 | 110.272 | 1 | 24.97 |
| 461 | C | GLY | 62 | 33.536 | 57.385 | 109.731 | 1 | 28.11 |
| 462 | O | GLY | 62 | 32.566 | 57.307 | 108.968 | 1 | 27.32 |
| 463 | N | LEU | 63 | 34.041 | 58.546 | 110.132 | 1 | 29.01 |
| 464 | CA | LEU | 63 | 33.484 | 59.813 | 109.685 | 1 | 30.07 |
| 465 | CB | LEU | 63 | 34.182 | 60.968 | 110.408 | 1 | 32.74 |
| 466 | CG | LEU | 63 | 33.581 | 62.367 | 110.247 | 1 | 36.32 |
| 467 | CD1 | LEU | 63 | 32.132 | 62.385 | 110.715 | 1 | 36.66 |
| 468 | CD2 | LEU | 63 | 34.412 | 63.346 | 111.059 | 1 | 38.02 |
| 469 | C | LEU | 63 | 33.657 | 59.956 | 108.165 | 1 | 29.27 |
| 470 | O | LEU | 63 | 32.764 | 60.444 | 107.477 | 1 | 28.89 |
| 471 | N | ALA | 64 | 34.801 | 59.521 | 107.643 | 1 | 27.8 |
| 472 | CA | ALA | 64 | 35.041 | 59.616 | 108.209 | 1 | 28.67 |
| 473 | CB | ALA | 64 | 36.453 | 59.141 | 105.872 | 1 | 27.84 |
| 474 | C | ALA | 64 | 34.004 | 58.786 | 105.459 | 1 | 28.56 |
| 475 | O | ALA | 64 | 33.386 | 59.254 | 104.497 | 1 | 28.44 |
| 476 | N | ILE | 65 | 33.803 | 57.556 | 105.917 | 1 | 27.46 |
| 477 | CA | ILE | 65 | 32.842 | 56.647 | 105.304 | 1 | 27.58 |
| 478 | CB | ILE | 65 | 32.883 | 55.244 | 105.993 | 1 | 26.41 |
| 479 | CG2 | ILE | 65 | 31.763 | 54.352 | 105.462 | 1 | 25.23 |
| 480 | CG1 | ILE | 65 | 34.243 | 54.593 | 105.743 | 1 | 26.7 |
| 481 | CD1 | ILE | 65 | 34.441 | 53.259 | 106.467 | 1 | 29.76 |
| 482 | C | ILE | 65 | 31.415 | 57.191 | 105.357 | 1 | 27.92 |
| 483 | O | ILE | 65 | 30.727 | 57.228 | 104.335 | 1 | 27 |
| 484 | N | ARG | 66 | 30.969 | 57.613 | 106.537 | 1 | 28.78 |
| 485 | CA | ARG | 66 | 29.612 | 58.128 | 106.673 | 1 | 31.41 |
| 486 | CB | ARG | 66 | 29.281 | 58.413 | 108.143 | 1 | 33.31 |
| 487 | CG | ARG | 66 | 29.385 | 57.194 | 109.053 | 1 | 37.74 |
| 488 | CD | ARG | 66 | 28.628 | 57.401 | 110.361 | 1 | 40.54 |
| 489 | NE | ARG | 66 | 28.971 | 58.657 | 111.032 | 1 | 45.75 |
| 490 | CZ | ARG | 66 | 30.147 | 58.912 | 111.596 | 1 | 46.01 |
| 491 | NH1 | ARG | 66 | 31.103 | 57.998 | 111.574 | 1 | 49.47 |
| 492 | NH2 | ARG | 66 | 30.366 | 60.078 | 112.183 | 1 | 47.04 |
| 493 | C | ARG | 66 | 29.389 | 59.39 | 105.836 | 1 | 31.9 |
| 494 | O | ARG | 66 | 28.287 | 59.619 | 105.33 | 1 | 31.91 |
| 495 | N | SER | 67 | 30.428 | 60.204 | 105.673 | 1 | 32.21 |
| 496 | CA | SER | 67 | 30.266 | 61.424 | 104.893 | 1 | 34.84 |
| 497 | CB | SER | 67 | 31.432 | 62.394 | 105.146 | 1 | 34.39 |
| 498 | OG | SER | 67 | 32.65 | 61.892 | 104.634 | 1 | 42.99 |
| 499 | C | SER | 67 | 30.126 | 61.103 | 103.4 | 1 | 34.73 |
| 500 | O | SER | 67 | 29.411 | 61.795 | 102.676 | 1 | 34.04 |
| 501 | N | LYS | 68 | 30.784 | 60.04 | 102.942 | 1 | 34.45 |
| 502 | CA | LYS | 68 | 30.695 | 59.654 | 101.54 | 1 | 33.99 |
| 503 | CB | LYS | 68 | 31.876 | 58.766 | 101.159 | 1 | 35.63 |
| 504 | CG | LYS | 68 | 33.135 | 59.563 | 100.891 | 1 | 38.42 |
| 505 | CD | LYS | 68 | 32.923 | 60.445 | 99.667 | 1 | 41.52 |
| 506 | CE | LYS | 68 | 34.016 | 61.477 | 99.507 | 1 | 43.47 |
| 507 | NZ | LYS | 68 | 33.729 | 62.366 | 98.343 | 1 | 44.99 |
| 508 | C | LYS | 68 | 29.373 | 58.953 | 101.248 | 1 | 34.95 |
| 509 | O | LYS | 68 | 28.914 | 58.913 | 100.104 | 1 | 33.66 |
| 510 | N | ILE | 69 | 28.763 | 58.395 | 102.287 | 1 | 34.96 |
| 511 | CA | ILE | 69 | 27.473 | 57.742 | 102.141 | 1 | 34.58 |
| 512 | CB | ILE | 69 | 27.222 | 56.725 | 103.272 | 1 | 33.98 |
| 513 | CG2 | ILE | 69 | 25.756 | 56.314 | 103.298 | 1 | 34.62 |
| 514 | CG1 | ILE | 69 | 28.106 | 55.49 | 103.064 | 1 | 34.34 |
| 515 | CD1 | ILE | 69 | 28.041 | 54.493 | 104.205 | 1 | 32.78 |
| 516 | C | ILE | 69 | 26.395 | 58.824 | 102.195 | 1 | 35.62 |
| 517 | O | ILE | 69 | 25.425 | 58.79 | 101.431 | 1 | 36.68 |
| 518 | N | ALA | 70 | 26.583 | 59.79 | 103.089 | 1 | 34.93 |
| 519 | CA | ALA | 70 | 25.637 | 60.887 | 103.272 | 1 | 36.65 |
| 520 | CB | ALA | 70 | 26.019 | 61.701 | 104.511 | 1 | 35.13 |
| 521 | C | ALA | 70 | 25.522 | 61.812 | 102.063 | 1 | 38.48 |
| 522 | O | ALA | 70 | 24.447 | 62.345 | 101.791 | 1 | 40.31 |
| 523 | N | ASP | 71 | 26.617 | 62.02 | 101.337 | 1 | 39.74 |
| 524 | CA | ASP | 71 | 26.553 | 62.897 | 100.175 | 1 | 40.68 |
| 525 | CB | ASP | 71 | 27.894 | 63.602 | 99.923 | 1 | 40.7 |
| 526 | CG | ASP | 71 | 28.996 | 62.649 | 99.521 | 1 | 42.25 |
| 527 | OD1 | ASP | 71 | 28.7 | 61.584 | 98.939 | 1 | 44.06 |
| 528 | OD2 | ASP | 71 | 30.175 | 62.977 | 99.771 | 1 | 43.7 |
| 529 | C | ASP | 71 | 26.119 | 62.143 | 98.923 | 1 | 41.09 |
| 530 | O | ASP | 71 | 26.115 | 62.696 | 97.823 | 1 | 41.94 |
| 531 | N | GLY | 72 | 25.762 | 60.875 | 99.097 | 1 | 40.42 |
| 532 | CA | GLY | 72 | 25.307 | 60.074 | 97.976 | 1 | 39.8 |
| 533 | C | GLY | 72 | 26.374 | 59.531 | 97.04 | 1 | 40.31 |
| 534 | O | GLY | 72 | 26.058 | 59.056 | 95.95 | 1 | 40.6 |
| 535 | N | SER | 73 | 27.636 | 59.588 | 97.444 | 1 | 38.43 |
| 536 | CA | SER | 73 | 28.703 | 59.079 | 96.593 | 1 | 37.64 |
| 537 | CB | SER | 73 | 30.058 | 59.606 | 97.063 | 1 | 37.67 |
| 538 | OG | SER | 73 | 30.126 | 61.008 | 96.915 | 1 | 39.98 |
| 539 | C | SER | 73 | 28.745 | 57.554 | 96.556 | 1 | 37.46 |
| 540 | O | SER | 73 | 29.1 | 56.959 | 95.538 | 1 | 37.38 |
| 541 | N | VAL | 74 | 28.382 | 56.916 | 97.662 | 1 | 35.43 |
| 542 | CA | VAL | 74 | 28.408 | 55.463 | 97.716 | 1 | 33.58 |
| 543 | CB | VAL | 74 | 29.848 | 54.969 | 98.062 | 1 | 33.69 |
| 544 | CG1 | VAL | 74 | 30.203 | 55.347 | 99.49 | 1 | 32.42 |
| 545 | CG2 | VAL | 74 | 29.965 | 53.479 | 97.845 | 1 | 35.96 |
| 546 | C | VAL | 74 | 27.408 | 54.952 | 98.748 | 1 | 32.69 |
| 547 | O | VAL | 74 | 26.995 | 55.693 | 99.638 | 1 | 32.42 |
| 548 | N | LYS | 75 | 26.998 | 53.695 | 98.61 | 1 | 31.87 |
| 549 | CA | LYS | 75 | 26.064 | 53.097 | 99.556 | 1 | 32.26 |
| 550 | CB | LYS | 75 | 25.018 | 52.269 | 98.812 | 1 | 35.55 |
| 551 | CG | LYS | 75 | 24.183 | 53.097 | 97.852 | 1 | 40.86 |
| 552 | CD | LYS | 75 | 22.981 | 52.332 | 97.316 | 1 | 44.16 |
| 553 | CE | LYS | 75 | 23.38 | 51.169 | 96.417 | 1 | 47.87 |
| 554 | NZ | LYS | 75 | 23.802 | 49.949 | 97.176 | 1 | 51.63 |
| 555 | C | LYS | 75 | 26.846 | 52.212 | 100.527 | 1 | 29.61 |
| 556 | O | LYS | 75 | 27.936 | 51.753 | 100.204 | 1 | 27.7 |
| 557 | N | ARG | 76 | 26.291 | 51.974 | 101.711 | 1 | 28.81 |
| 558 | CA | ARG | 76 | 26.972 | 51.145 | 102.701 | 1 | 28.53 |
| 559 | CB | ARG | 76 | 26.093 | 50.949 | 103.937 | 1 | 28.03 |
| 560 | CG | ARG | 76 | 26.729 | 50.063 | 105.02 | 1 | 26 |
| 561 | CD | ARG | 76 | 27.977 | 50.705 | 105.629 | 1 | 25.59 |
| 562 | NE | ARG | 76 | 28.65 | 49.818 | 106.583 | 1 | 25 |
| 563 | CZ | ARG | 76 | 29.549 | 48.896 | 106.245 | 1 | 26.42 |
| 564 | NH1 | ARG | 76 | 29.886 | 48.744 | 104.972 | 1 | 24.23 |
| 565 | NH2 | ARG | 76 | 30.112 | 48.12 | 107.175 | 1 | 23.44 |
| 566 | C | ARG | 76 | 27.342 | 49.781 | 102.126 | 1 | 27.32 |
| 567 | O | ARG | 76 | 28.436 | 49.28 | 102.366 | 1 | 27.95 |
| 568 | N | GLU | 77 | 26.424 | 49.193 | 101.367 | 1 | 27.83 |
| 569 | CA | GLU | 77 | 26.635 | 47.879 | 100.756 | 1 | 29.8 |
| 570 | CB | GLU | 77 | 25.378 | 47.433 | 99.998 | 1 | 33.42 |
| 571 | CG | GLU | 77 | 24.055 | 47.598 | 100.746 | 1 | 40.95 |
| 572 | CD | GLU | 77 | 23.716 | 49.056 | 101.045 | 1 | 43.36 |
| 573 | OE1 | GLU | 77 | 23.841 | 49.9 | 100.134 | 1 | 45.86 |
| 574 | OE2 | GLU | 77 | 23.315 | 49.35 | 102.188 | 1 | 47.53 |
| 575 | C | GLU | 77 | 27.824 | 47.856 | 99.785 | 1 | 28.52 |
| 576 | O | GLU | 77 | 28.323 | 48.783 | 99.424 | 1 | 27.64 |
| 577 | N | ASP | 78 | 28.276 | 49.027 | 99.345 | 1 | 26.62 |
| 578 | CA | ASP | 78 | 29.396 | 49.061 | 98.409 | 1 | 26.46 |
| 579 | CB | ASP | 78 | 29.087 | 49.988 | 97.235 | 1 | 28.06 |
| 580 | CG | ASP | 78 | 28.002 | 49.419 | 96.333 | 1 | 30.89 |
| 581 | OD1 | ASP | 78 | 28.169 | 48.278 | 95.853 | 1 | 32.5 |
| 582 | OD2 | ASP | 78 | 26.987 | 50.101 | 96.121 | 1 | 32.99 |
| 583 | C | ASP | 78 | 30.712 | 49.427 | 99.055 | 1 | 25.52 |
| 584 | O | ASP | 78 | 31.692 | 49.732 | 98.376 | 1 | 26.85 |
| 585 | N | ILE | 79 | 30.72 | 49.39 | 100.38 | 1 | 23.95 |
| 586 | CA | ILE | 79 | 31.92 | 49.657 | 101.152 | 1 | 23.73 |
| 587 | CB | ILE | 79 | 31.737 | 50.864 | 102.115 | 1 | 26.37 |
| 588 | CG2 | ILE | 79 | 32.917 | 50.952 | 103.084 | 1 | 25.91 |
| 589 | CG1 | ILE | 79 | 31.643 | 52.171 | 101.317 | 1 | 26.85 |
| 590 | CD1 | ILE | 79 | 32.86 | 52.453 | 100.463 | 1 | 30.23 |
| 591 | C | ILE | 79 | 32.173 | 48.398 | 101.977 | 1 | 22.09 |
| 592 | O | ILE | 79 | 31.264 | 47.876 | 102.626 | 1 | 23.32 |
| 593 | N | PHE | 80 | 33.398 | 47.898 | 101.92 | 1 | 21.12 |
| 594 | CA | PHE | 80 | 33.797 | 46.715 | 102.679 | 1 | 21.03 |
| 595 | CB | PHE | 80 | 34.446 | 45.678 | 101.75 | 1 | 19.03 |
| 596 | CG | PHE | 80 | 34.744 | 44.357 | 102.414 | 1 | 17.82 |
| 597 | CD1 | PHE | 80 | 35.864 | 44.205 | 103.236 | 1 | 20.27 |
| 598 | CD2 | PHE | 80 | 33.904 | 43.259 | 102.211 | 1 | 18.83 |
| 599 | CE1 | PHE | 80 | 36.147 | 42.969 | 103.851 | 1 | 20.09 |
| 600 | CE2 | PHE | 80 | 34.172 | 42.016 | 102.816 | 1 | 17.91 |
| 601 | CZ | PHE | 80 | 35.293 | 41.871 | 103.632 | 1 | 18.48 |
| 602 | C | PHE | 80 | 34.802 | 47.243 | 103.705 | 1 | 20.8 |
| 603 | O | PHE | 80 | 35.921 | 47.617 | 103.356 | 1 | 20.58 |
| 604 | N | TYR | 81 | 34.374 | 47.298 | 104.964 | 1 | 19.98 |
| 605 | CA | TYR | 81 | 35.21 | 47.807 | 106.048 | 1 | 20.09 |
| 606 | CB | TYR | 81 | 34.448 | 48.848 | 106.882 | 1 | 19.18 |
| 607 | CG | TYR | 81 | 35.278 | 49.371 | 108.03 | 1 | 19.57 |
| 608 | CD1 | TYR | 81 | 36.36 | 50.217 | 107.796 | 1 | 20.5 |
| 609 | CE1 | TYR | 81 | 37.203 | 50.613 | 108.828 | 1 | 21.7 |
| 610 | CD2 | TYR | 81 | 35.051 | 48.938 | 109.337 | 1 | 19.42 |
| 611 | CE2 | TYR | 81 | 35.894 | 49.322 | 110.379 | 1 | 19.3 |
| 612 | CZ | TYR | 81 | 36.968 | 50.156 | 110.12 | 1 | 21.87 |
| 613 | OH | TYR | 81 | 37.84 | 50.522 | 111.125 | 1 | 21.28 |
| 614 | C | TYR | 81 | 35.692 | 46.695 | 106.978 | 1 | 18.98 |
| 615 | O | TYR | 81 | 34.902 | 45.891 | 107.471 | 1 | 17.3 |
| 616 | N | THR | 82 | 36.997 | 46.671 | 107.217 | 1 | 18.4 |
| 617 | CA | THR | 82 | 37.618 | 45.666 | 108.081 | 1 | 18.46 |
| 618 | CB | THR | 82 | 38.817 | 44.994 | 107.369 | 1 | 18.41 |
| 619 | OG1 | THR | 82 | 38.356 | 44.311 | 106.198 | 1 | 19.05 |
| 620 | CG2 | THR | 82 | 39.527 | 44.007 | 108.304 | 1 | 18.15 |
| 621 | C | THR | 82 | 38.177 | 48.291 | 109.358 | 1 | 18.18 |
| 622 | O | THR | 82 | 38.807 | 47.345 | 109.306 | 1 | 17.43 |
| 623 | N | SER | 83 | 37.921 | 45.665 | 110.5 | 1 | 17.08 |
| 624 | CA | SER | 83 | 38.508 | 46.128 | 111.746 | 1 | 17.19 |
| 625 | CB | SER | 83 | 37.472 | 46.685 | 112.718 | 1 | 18.59 |
| 626 | OG | SER | 83 | 38.145 | 47.278 | 113.832 | 1 | 20.29 |
| 627 | C | SER | 83 | 39.184 | 44.9 | 112.354 | 1 | 17.14 |
| 628 | O | SER | 83 | 38.992 | 43.782 | 111.879 | 1 | 17.04 |
| 629 | N | LYS | 84 | 39.968 | 45.109 | 113.403 | 1 | 16.23 |
| 630 | CA | LYS | 84 | 40.686 | 44.011 | 114.024 | 1 | 16.39 |
| 631 | CB | LYS | 84 | 42.152 | 44.007 | 113.567 | 1 | 15.34 |
| 632 | CG | LYS | 84 | 42.357 | 44.222 | 112.061 | 1 | 16.74 |
| 633 | CD | LYS | 84 | 43.826 | 44.043 | 111.632 | 1 | 16.82 |
| 634 | CE | LYS | 84 | 44.049 | 44.374 | 110.145 | 1 | 16.85 |
| 635 | NZ | LYS | 84 | 44.124 | 45.858 | 109.915 | 1 | 16.41 |
| 636 | C | LYS | 84 | 40.642 | 44.065 | 115.544 | 1 | 16.86 |
| 637 | O | LYS | 84 | 40.701 | 45.142 | 116.154 | 1 | 15.47 |
| 638 | N | LEU | 85 | 40.542 | 42.882 | 116.14 | 1 | 15.88 |
| 639 | CA | LEU | 85 | 40.52 | 42.713 | 117.591 | 1 | 15.47 |
| 640 | CB | LEU | 85 | 39.926 | 41.342 | 117.927 | 1 | 13.95 |
| 641 | CG | LEU | 85 | 39.971 | 40.905 | 119.392 | 1 | 15.62 |
| 642 | CD1 | LEU | 85 | 38.884 | 41.636 | 120.165 | 1 | 13.92 |
| 643 | CD2 | LEU | 85 | 39.764 | 39.395 | 119.477 | 1 | 15.4 |
| 644 | C | LEU | 85 | 41.96 | 42.775 | 118.118 | 1 | 16.61 |
| 645 | O | LEU | 85 | 42.792 | 41.953 | 117.733 | 1 | 16.73 |
| 646 | N | TRP | 86 | 42.268 | 43.73 | 118.995 | 1 | 15.09 |
| 647 | CA | TRP | 86 | 43.631 | 43.81 | 119.515 | 1 | 17.26 |
| 648 | CB | TRP | 86 | 43.965 | 45.195 | 120.094 | 1 | 17.06 |
| 649 | CG | TRP | 86 | 45.414 | 45.287 | 120.479 | 1 | 19.24 |
| 650 | CD2 | TRP | 86 | 46.54 | 44.941 | 119.658 | 1 | 22.26 |
| 651 | CE2 | TRP | 86 | 47.7 | 45.097 | 120.449 | 1 | 23.01 |
| 652 | CE3 | TRP | 86 | 48.677 | 44.512 | 118.33 | 1 | 22.33 |
| 653 | CD1 | TRP | 86 | 45.925 | 45.637 | 121.697 | 1 | 22.03 |
| 654 | NE1 | TRP | 86 | 47.296 | 45.523 | 121.687 | 1 | 22.49 |
| 655 | CZ2 | TRP | 86 | 48.986 | 44.84 | 119.958 | 1 | 24.56 |
| 656 | CZ3 | TRP | 86 | 47.952 | 44.255 | 117.836 | 1 | 24.7 |
| 657 | CH2 | TRP | 86 | 48.095 | 44.419 | 118.654 | 1 | 25.47 |
| 658 | C | TRP | 86 | 43.846 | 42.739 | 120.581 | 1 | 18.13 |
| 659 | O | TRP | 86 | 42.893 | 42.234 | 121.18 | 1 | 16.98 |
| 660 | N | SER | 87 | 45.113 | 42.408 | 120.807 | 1 | 17.74 |
| 661 | CA | SER | 87 | 45.525 | 41.359 | 121.742 | 1 | 17.82 |
| 662 | CB | SER | 87 | 47.019 | 41.107 | 121.557 | 1 | 18.06 |
| 663 | OG | SER | 87 | 47.272 | 40.662 | 120.229 | 1 | 18.53 |
| 664 | C | SER | 87 | 45.215 | 41.516 | 123.222 | 1 | 17.94 |
| 665 | O | SER | 87 | 45.416 | 40.575 | 123.988 | 1 | 17.93 |
| 666 | N | ASN | 88 | 44.744 | 42.691 | 123.637 | 1 | 17.38 |
| 667 | CA | ASN | 88 | 44.392 | 42.886 | 125.034 | 1 | 17.57 |
| 668 | CB | ASN | 88 | 44.656 | 44.335 | 125.473 | 1 | 16.77 |
| 669 | CG | ASN | 88 | 43.895 | 45.366 | 124.645 | 1 | 18.03 |
| 670 | OD1 | ASN | 88 | 43.328 | 45.064 | 123.596 | 1 | 19.23 |
| 671 | ND2 | ASN | 88 | 43.903 | 46.604 | 125.118 | 1 | 16.61 |
| 672 | C | ASN | 88 | 42.927 | 42.504 | 125.264 | 1 | 18.4 |
| 673 | O | ASN | 88 | 42.417 | 42.621 | 126.374 | 1 | 17.59 |
| 674 | N | SER | 89 | 42.262 | 42.025 | 124.212 | 1 | 18.17 |
| 675 | CA | SER | 89 | 40.865 | 41.617 | 124.322 | 1 | 17.2 |
| 676 | CB | SER | 89 | 39.959 | 42.628 | 123.605 | 1 | 17.98 |
| 677 | OG | SER | 89 | 39.98 | 43.887 | 124.274 | 1 | 20.02 |
| 678 | C | SER | 89 | 40.623 | 40.205 | 123.775 | 1 | 18.15 |
| 679 | O | SER | 89 | 39.572 | 39.924 | 123.207 | 1 | 18.42 |
| 680 | N | HIS | 90 | 41.597 | 39.311 | 123.961 | 1 | 17.09 |
| 681 | CA | HIS | 90 | 41.464 | 37.926 | 123.506 | 1 | 17 |
| 682 | CB | HIS | 90 | 42.844 | 37.249 | 123.41 | 1 | 16.29 |
| 683 | CG | HIS | 90 | 43.6 | 37.577 | 122.155 | 1 | 17.98 |
| 684 | CD2 | HIS | 90 | 43.281 | 38.355 | 121.092 | 1 | 18.58 |
| 685 | ND1 | HIS | 90 | 44.858 | 37.073 | 121.897 | 1 | 16.64 |
| 686 | CE1 | HIS | 90 | 45.282 | 37.527 | 120.731 | 1 | 17.97 |
| 687 | NE2 | HIS | 90 | 44.346 | 38.308 | 120.219 | 1 | 17.4 |
| 688 | C | HIS | 90 | 40.565 | 37.064 | 124.403 | 1 | 17.65 |
| 689 | O | HIS | 90 | 40.002 | 36.063 | 123.947 | 1 | 16.1 |
| 690 | N | ARG | 91 | 40.45 | 37.417 | 125.683 | 1 | 17.7 |
| 691 | CA | ARG | 91 | 39.606 | 36.611 | 126.573 | 1 | 18.28 |
| 692 | CB | ARG | 91 | 39.629 | 37.173 | 127.993 | 1 | 17.65 |
| 693 | CG | ARG | 91 | 41.002 | 37.069 | 128.64 | 1 | 17.56 |
| 694 | CD | ARG | 91 | 40.984 | 37.566 | 130.079 | 1 | 18.35 |
| 695 | NE | ARG | 91 | 42.319 | 37.549 | 130.67 | 1 | 19.75 |
| 696 | CZ | ARG | 91 | 42.586 | 37.926 | 131.914 | 1 | 22.75 |
| 697 | NH1 | ARG | 91 | 41.602 | 38.354 | 132.709 | 1 | 20.62 |
| 698 | NH2 | ARG | 91 | 43.832 | 37.882 | 132.364 | 1 | 20.67 |
| 699 | C | ARG | 91 | 38.2 | 36.625 | 125.983 | 1 | 16.86 |
| 700 | O | ARG | 91 | 37.715 | 37.669 | 125.57 | 1 | 17.21 |
| 701 | N | PRO | 92 | 37.528 | 35.462 | 125.937 | 1 | 19.49 |
| 702 | CD | PRO | 92 | 37.924 | 34.203 | 126.592 | 1 | 18.57 |
| 703 | CA | PRO | 92 | 36.177 | 35.334 | 125.37 | 1 | 18.62 |
| 704 | CB | PRO | 92 | 35.735 | 33.947 | 125.834 | 1 | 20.65 |
| 705 | CG | PRO | 92 | 37.025 | 33.202 | 125.912 | 1 | 24.15 |
| 706 | C | PRO | 92 | 35.153 | 36.4 | 125.748 | 1 | 19.04 |
| 707 | O | PRO | 92 | 34.416 | 36.896 | 124.889 | 1 | 16.63 |
| 708 | N | GLU | 93 | 35.098 | 36.743 | 127.032 | 1 | 17.82 |
| 709 | CA | GLU | 93 | 34.128 | 37.733 | 127.508 | 1 | 18.94 |
| 710 | CB | GLU | 93 | 33.979 | 37.639 | 129.034 | 1 | 19.61 |
| 711 | CG | GLU | 93 | 35.209 | 38.107 | 129.818 | 1 | 21.01 |
| 712 | CD | GLU | 93 | 36.133 | 36.963 | 130.236 | 1 | 25.88 |
| 713 | OE1 | GLU | 93 | 36.331 | 36.003 | 129.462 | 1 | 24.23 |
| 714 | OE2 | GLU | 93 | 36.682 | 37.035 | 131.348 | 1 | 28.77 |
| 715 | C | GLU | 93 | 34.491 | 39.172 | 127.126 | 1 | 17.69 |
| 716 | O | GLU | 93 | 33.717 | 40.09 | 127.382 | 1 | 15.74 |
| 717 | N | LEU | 94 | 35.665 | 39.365 | 126.523 | 1 | 17.28 |
| 718 | CA | LEU | 94 | 36.098 | 40.698 | 126.112 | 1 | 17.92 |
| 719 | CB | LEU | 94 | 37.549 | 40.943 | 126.547 | 1 | 17.98 |
| 720 | CG | LEU | 94 | 37.873 | 40.883 | 128.051 | 1 | 19.7 |
| 721 | CD1 | LEU | 94 | 39.362 | 41.156 | 128.265 | 1 | 20.48 |
| 722 | CD2 | LEU | 94 | 37.047 | 41.92 | 128.792 | 1 | 21.4 |
| 723 | C | LEU | 94 | 35.996 | 40.959 | 124.614 | 1 | 17.5 |
| 724 | O | LEU | 94 | 36.132 | 42.102 | 124.176 | 1 | 17.02 |
| 725 | N | VAL | 95 | 35.75 | 39.911 | 123.832 | 1 | 16.76 |
| 726 | CA | VAL | 95 | 35.688 | 40.054 | 122.378 | 1 | 16.55 |
| 727 | CB | VAL | 95 | 35.65 | 38.663 | 121.664 | 1 | 17.34 |
| 728 | CG1 | VAL | 95 | 35.582 | 38.847 | 120.134 | 1 | 15.37 |
| 729 | CG2 | VAL | 95 | 36.901 | 37.849 | 122.05 | 1 | 16.26 |
| 730 | C | VAL | 95 | 34.524 | 40.911 | 121.881 | 1 | 16.68 |
| 731 | O | VAL | 95 | 34.737 | 41.832 | 121.085 | 1 | 15.22 |
| 732 | N | ARG | 96 | 33.303 | 40.63 | 122.326 | 1 | 14.23 |
| 733 | CA | ARG | 96 | 32.187 | 41.434 | 121.848 | 1 | 17.06 |
| 734 | CB | ARG | 96 | 30.839 | 40.829 | 122.268 | 1 | 18.39 |
| 735 | CG | ARG | 96 | 29.679 | 41.649 | 121.738 | 1 | 21.14 |
| 736 | CD | ARG | 96 | 28.351 | 40.922 | 121.808 | 1 | 22.25 |
| 737 | NE | ARG | 96 | 27.264 | 41.861 | 121.562 | 1 | 23.91 |
| 738 | CZ | ARG | 96 | 26.017 | 41.514 | 121.249 | 1 | 23.83 |
| 739 | NH1 | ARG | 96 | 25.684 | 40.236 | 121.135 | 1 | 22.82 |
| 740 | NH2 | ARG | 96 | 25.104 | 42.453 | 121.05 | 1 | 23.05 |
| 741 | C | ARG | 96 | 32.283 | 42.9 | 122.292 | 1 | 17.26 |
| 742 | O | ARG | 96 | 31.963 | 43.809 | 121.523 | 1 | 17.44 |
| 743 | N | PRO | 97 | 32.703 | 43.149 | 123.548 | 1 | 16.36 |
| 744 | CD | PRO | 97 | 32.777 | 42.22 | 124.692 | 1 | 16.82 |
| 745 | CA | PRO | 97 | 32.816 | 44.539 | 123.997 | 1 | 15.62 |
| 746 | CB | PRO | 97 | 33.264 | 44.395 | 125.452 | 1 | 16.84 |
| 747 | CG | PRO | 97 | 32.551 | 43.144 | 125.879 | 1 | 18.16 |
| 748 | C | PRO | 97 | 33.837 | 45.318 | 123.142 | 1 | 16.16 |
| 749 | O | PRO | 97 | 33.645 | 46.499 | 122.875 | 1 | 13.69 |
| 750 | N | ALA | 98 | 34.919 | 44.66 | 122.716 | 1 | 14.1 |
| 751 | CA | ALA | 98 | 35.927 | 45.329 | 121.88 | 1 | 16.12 |
| 752 | CB | ALA | 98 | 37.178 | 44.431 | 121.709 | 1 | 15.85 |
| 753 | C | ALA | 98 | 35.345 | 45.684 | 120.507 | 1 | 16.76 |
| 754 | O | ALA | 98 | 35.65 | 46.743 | 119.948 | 1 | 17.68 |
| 755 | N | LEU | 99 | 34.525 | 44.792 | 119.956 | 1 | 15.77 |
| 756 | CA | LEU | 99 | 33.913 | 45.055 | 118.66 | 1 | 17.8 |
| 757 | CB | LEU | 99 | 33.219 | 43.794 | 118.114 | 1 | 16.25 |
| 758 | CG | LEU | 99 | 32.394 | 43.934 | 116.815 | 1 | 18.18 |
| 759 | CD1 | LEU | 99 | 33.206 | 44.632 | 115.715 | 1 | 16.77 |
| 760 | CD2 | LEU | 99 | 31.969 | 42.54 | 116.352 | 1 | 19.62 |
| 761 | C | LEU | 99 | 32.908 | 46.208 | 118.812 | 1 | 18.26 |
| 762 | O | LEU | 99 | 32.893 | 47.139 | 118.003 | 1 | 16.49 |
| 763 | N | GLU | 100 | 32.088 | 46.157 | 119.861 | 1 | 17.72 |
| 764 | CA | GLU | 100 | 31.108 | 47.218 | 120.113 | 1 | 18.21 |
| 765 | CB | GLU | 100 | 30.275 | 46.89 | 121.354 | 1 | 20.01 |
| 766 | CG | GLU | 100 | 29.413 | 45.656 | 121.131 | 1 | 25.55 |
| 767 | CD | GLU | 100 | 28.605 | 45.256 | 122.347 | 1 | 29.61 |
| 768 | OE1 | GLU | 100 | 29.11 | 45.388 | 123.484 | 1 | 33.34 |
| 769 | OE2 | GLU | 100 | 27.475 | 44.778 | 122.157 | 1 | 32.17 |
| 770 | C | GLU | 100 | 31.778 | 48.58 | 120.279 | 1 | 18.89 |
| 771 | O | GLU | 100 | 31.232 | 49.599 | 119.86 | 1 | 19.15 |
| 772 | N | ARG | 101 | 32.959 | 48.596 | 120.888 | 1 | 18.88 |
| 773 | CA | ARG | 101 | 33.696 | 49.838 | 121.08 | 1 | 19.61 |
| 774 | CB | ARG | 101 | 34.917 | 49.606 | 121.965 | 1 | 20.04 |
| 775 | CG | ARG | 101 | 35.718 | 50.871 | 122.224 | 1 | 22.92 |
| 776 | CD | ARG | 101 | 36.952 | 50.596 | 123.061 | 1 | 22.13 |
| 777 | NE | ARG | 101 | 37.562 | 51.834 | 123.54 | 1 | 20.45 |
| 778 | CZ | ARG | 101 | 38.721 | 51.893 | 124.19 | 1 | 23.07 |
| 779 | NH1 | ARG | 101 | 39.403 | 50.781 | 124.434 | 1 | 21.59 |
| 780 | NH2 | ARG | 101 | 39.191 | 53.06 | 124.617 | 1 | 22.84 |
| 781 | C | ARG | 101 | 34.14 | 50.395 | 119.725 | 1 | 20.18 |
| 782 | O | ARG | 101 | 34.029 | 51.597 | 119.478 | 1 | 19.44 |
| 783 | N | SER | 102 | 34.654 | 49.525 | 118.854 | 1 | 19.39 |
| 784 | CA | SER | 102 | 35.066 | 49.952 | 117.521 | 1 | 18.23 |
| 785 | CB | SER | 102 | 35.684 | 48.787 | 116.733 | 1 | 18.96 |
| 786 | OG | SER | 102 | 37.08 | 48.656 | 116.98 | 1 | 19.01 |
| 787 | C | SER | 102 | 33.821 | 50.469 | 116.787 | 1 | 18.98 |
| 788 | O | SER | 102 | 33.85 | 51.536 | 116.182 | 1 | 18.92 |
| 789 | N | LEU | 103 | 32.729 | 49.713 | 116.848 | 1 | 19.27 |
| 790 | CA | LEU | 103 | 31.492 | 50.124 | 116.185 | 1 | 21.56 |
| 791 | CB | LEU | 103 | 30.397 | 49.056 | 116.363 | 1 | 21.46 |
| 792 | CG | LEU | 103 | 30.63 | 47.777 | 115.554 | 1 | 20.28 |
| 793 | CD1 | LEU | 103 | 29.546 | 46.75 | 115.841 | 1 | 22.01 |
| 794 | CD2 | LEU | 103 | 30.661 | 48.131 | 114.071 | 1 | 21.19 |
| 795 | C | LEU | 103 | 30.986 | 51.479 | 116.678 | 1 | 22.36 |
| 796 | O | LEU | 103 | 30.456 | 52.27 | 115.897 | 1 | 23.53 |
| 797 | N | LYS | 104 | 31.143 | 51.751 | 117.966 | 1 | 23.04 |
| 798 | CA | LYS | 104 | 30.694 | 53.023 | 118.52 | 1 | 24.98 |
| 799 | CB | LYS | 104 | 30.738 | 52.986 | 120.049 | 1 | 29.66 |
| 800 | CG | LYS | 104 | 30.214 | 54.246 | 120.706 | 1 | 34.22 |
| 801 | CD | LYS | 104 | 29.922 | 54.011 | 122.17 | 1 | 40.01 |
| 802 | CE | LYS | 104 | 29.267 | 55.238 | 122.803 | 1 | 43.84 |
| 803 | NZ | LYS | 104 | 28.94 | 55.015 | 124.255 | 1 | 47.11 |
| 804 | C | LYS | 104 | 31.558 | 54.165 | 117.998 | 1 | 25.4 |
| 805 | O | LYS | 104 | 31.047 | 55.241 | 117.696 | 1 | 24.95 |
| 806 | N | ASN | 105 | 32.864 | 53.93 | 117.887 | 1 | 25.13 |
| 807 | CA | ASN | 105 | 33.774 | 54.946 | 117.37 | 1 | 24.96 |
| 808 | CB | ASN | 105 | 35.235 | 54.511 | 117.539 | 1 | 25.12 |
| 809 | CG | ASN | 105 | 35.729 | 54.683 | 118.961 | 1 | 28.69 |
| 810 | OD1 | ASN | 105 | 35.288 | 55.581 | 119.669 | 1 | 28.82 |
| 811 | ND2 | ASN | 105 | 36.657 | 53.831 | 119.379 | 1 | 27.74 |
| 812 | C | ASN | 105 | 33.501 | 55.228 | 115.896 | 1 | 25.76 |
| 813 | O | ASN | 105 | 33.613 | 56.367 | 115.438 | 1 | 24.28 |
| 814 | N | LEU | 106 | 33.142 | 54.179 | 115.162 | 1 | 23.88 |
| 815 | CA | LEU | 106 | 32.843 | 54.282 | 113.736 | 1 | 25.15 |
| 816 | CB | LEU | 106 | 32.941 | 52.905 | 113.086 | 1 | 22.08 |
| 817 | CG | LEU | 106 | 34.331 | 52.29 | 112.934 | 1 | 22.12 |
| 818 | CD1 | LEU | 106 | 34.233 | 50.762 | 112.937 | 1 | 21.11 |
| 819 | CD2 | LEU | 106 | 34.958 | 52.797 | 111.64 | 1 | 21.78 |
| 820 | C | LEU | 106 | 31.453 | 54.829 | 113.452 | 1 | 25.59 |
| 821 | O | LEU | 106 | 31.217 | 55.437 | 112.407 | 1 | 25.31 |
| 822 | N | GLN | 107 | 30.539 | 54.608 | 114.39 | 1 | 26.92 |
| 823 | CA | GLN | 107 | 29.149 | 55.011 | 114.232 | 1 | 29.28 |
| 824 | CB | GLN | 107 | 29.033 | 56.505 | 113.922 | 1 | 32.87 |
| 825 | CG | GLN | 107 | 29.232 | 57.377 | 115.148 | 1 | 40.8 |
| 826 | CD | GLN | 107 | 28.179 | 57.107 | 116.208 | 1 | 46.03 |
| 827 | OE1 | GLN | 107 | 26.99 | 57.377 | 116.005 | 1 | 50.08 |
| 828 | NE2 | GLN | 107 | 28.606 | 56.559 | 117.346 | 1 | 48.27 |
| 829 | C | GLN | 107 | 28.531 | 54.183 | 113.111 | 1 | 28.78 |
| 830 | O | GLN | 107 | 27.671 | 54.658 | 112.375 | 1 | 26.96 |
| 831 | N | LEU | 108 | 29 | 52.943 | 112.975 | 1 | 26.63 |
| 832 | CA | LEU | 108 | 28.461 | 52.025 | 111.978 | 1 | 25.41 |
| 833 | CB | LEU | 108 | 29.575 | 51.426 | 111.112 | 1 | 26.3 |
| 834 | CG | LEU | 108 | 30.287 | 52.412 | 110.175 | 1 | 28.35 |
| 835 | CD1 | LEU | 108 | 31.452 | 51.723 | 109.498 | 1 | 28.35 |
| 836 | CD2 | LEU | 108 | 29.301 | 52.944 | 109.154 | 1 | 29.34 |
| 837 | C | LEU | 108 | 27.744 | 50.914 | 112.728 | 1 | 24.32 |
| 838 | O | LEU | 108 | 28.076 | 50.619 | 113.875 | 1 | 23.03 |
| 839 | N | ASP | 109 | 26.767 | 50.294 | 112.081 | 1 | 24.81 |
| 840 | CA | ASP | 109 | 26.005 | 49.218 | 112.709 | 1 | 27.01 |
| 841 | CB | ASP | 109 | 24.634 | 49.087 | 112.04 | 1 | 31.65 |
| 842 | CG | ASP | 109 | 23.81 | 50.363 | 112.14 | 1 | 36.75 |
| 843 | OD1 | ASP | 109 | 23.676 | 50.904 | 113.26 | 1 | 39.05 |
| 844 | OD2 | ASP | 109 | 23.292 | 50.817 | 111.1 | 1 | 41.28 |
| 845 | C | ASP | 109 | 26.731 | 47.867 | 112.67 | 1 | 25.66 |
| 846 | O | ASP | 109 | 26.429 | 46.969 | 113.454 | 1 | 24.54 |
| 847 | N | TYR | 110 | 27.691 | 47.719 | 111.763 | 1 | 23.81 |
| 848 | CA | TYR | 110 | 28.428 | 46.465 | 111.669 | 1 | 22.04 |
| 849 | CB | TYR | 110 | 27.544 | 45.378 | 111.026 | 1 | 23.87 |
| 850 | CG | TYR | 110 | 27.186 | 45.676 | 109.576 | 1 | 25.91 |
| 851 | CD1 | TYR | 110 | 25.993 | 46.333 | 109.234 | 1 | 28.25 |
| 852 | CE1 | TYR | 110 | 25.709 | 46.666 | 107.888 | 1 | 29.75 |
| 853 | CD2 | TYR | 110 | 28.077 | 45.356 | 108.552 | 1 | 25.45 |
| 854 | CE2 | TYR | 110 | 27.812 | 45.678 | 107.23 | 1 | 27.56 |
| 855 | CZ | TYR | 110 | 26.631 | 46.334 | 106.901 | 1 | 29.12 |
| 856 | OH | TYR | 110 | 26.414 | 46.661 | 105.585 | 1 | 31.81 |
| 857 | C | TYR | 110 | 29.684 | 46.65 | 110.833 | 1 | 22.34 |
| 858 | O | TYR | 110 | 29.802 | 47.633 | 110.096 | 1 | 21.73 |
| 859 | N | VAL | 111 | 30.634 | 45.723 | 110.967 | 1 | 19.67 |
| 860 | CA | VAL | 111 | 31.84 | 45.773 | 110.149 | 1 | 19.49 |
| 861 | CB | VAL | 111 | 33.166 | 45.587 | 110.963 | 1 | 20.04 |
| 862 | CG1 | VAL | 111 | 33.339 | 46.742 | 111.957 | 1 | 18 |
| 863 | CG2 | VAL | 111 | 33.173 | 44.23 | 111.681 | 1 | 16.54 |
| 864 | C | VAL | 111 | 31.688 | 44.613 | 109.176 | 1 | 19.37 |
| 865 | O | VAL | 111 | 31.033 | 43.62 | 109.483 | 1 | 19.55 |
| 866 | N | ASP | 112 | 32.27 | 44.741 | 107.99 | 1 | 17.64 |
| 867 | CA | ASP | 112 | 32.175 | 43.672 | 107.013 | 1 | 17.89 |
| 868 | CB | ASP | 112 | 32.5 | 44.229 | 105.629 | 1 | 16.76 |
| 869 | CG | ASP | 112 | 31.501 | 45.28 | 105.202 | 1 | 17.24 |
| 870 | OD1 | ASP | 112 | 30.485 | 44.906 | 104.592 | 1 | 18.14 |
| 871 | OD2 | ASP | 112 | 31.711 | 46.469 | 105.507 | 1 | 17.83 |
| 872 | C | ASP | 112 | 33.103 | 42.522 | 107.357 | 1 | 17.41 |
| 873 | O | ASP | 112 | 32.824 | 41.372 | 107.019 | 1 | 19.21 |
| 874 | N | LEU | 113 | 34.192 | 42.838 | 108.043 | 1 | 17.31 |
| 875 | CA | LEU | 113 | 35.172 | 41.832 | 108.423 | 1 | 17.56 |
| 876 | CB | LEU | 113 | 36.258 | 41.717 | 107.344 | 1 | 17.25 |
| 877 | CG | LEU | 113 | 37.397 | 40.707 | 107.527 | 1 | 17.91 |
| 878 | CD1 | LEU | 113 | 36.846 | 39.286 | 107.672 | 1 | 16.18 |
| 879 | CD2 | LEU | 113 | 38.336 | 40.788 | 106.317 | 1 | 13.46 |
| 880 | C | LEU | 113 | 35.826 | 42.178 | 109.746 | 1 | 17.46 |
| 881 | O | LEU | 113 | 36.238 | 43.321 | 109.97 | 1 | 17.2 |
| 882 | N | TYR | 114 | 35.924 | 41.182 | 110.623 | 1 | 16.2 |
| 883 | CA | TYR | 114 | 36.56 | 41.378 | 111.921 | 1 | 16.4 |
| 884 | CB | TYR | 114 | 35.544 | 41.235 | 113.056 | 1 | 16.09 |
| 885 | CG | TYR | 114 | 36.015 | 41.87 | 114.342 | 1 | 16.96 |
| 886 | CD1 | TYR | 114 | 36.565 | 43.158 | 114.339 | 1 | 16.89 |
| 887 | CE1 | TYR | 114 | 36.935 | 43.792 | 115.519 | 1 | 16.7 |
| 888 | CD2 | TYR | 114 | 35.853 | 41.227 | 115.562 | 1 | 15.54 |
| 889 | CE2 | TYR | 114 | 36.219 | 41.854 | 116.757 | 1 | 16.92 |
| 890 | CZ | TYR | 114 | 36.753 | 43.131 | 116.73 | 1 | 17.55 |
| 891 | OH | TYR | 114 | 37.094 | 43.762 | 117.905 | 1 | 17.16 |
| 892 | C | TYR | 114 | 37.627 | 40.314 | 112.042 | 1 | 15.59 |
| 893 | O | TYR | 114 | 37.346 | 39.131 | 111.875 | 1 | 15.09 |
| 894 | N | LEU | 115 | 38.845 | 40.741 | 112.343 | 1 | 16.26 |
| 895 | CA | LEU | 115 | 39.974 | 39.83 | 112.433 | 1 | 18.24 |
| 896 | CB | LEU | 115 | 41.055 | 40.247 | 111.431 | 1 | 16.82 |
| 897 | CG | LEU | 115 | 40.689 | 40.523 | 109.969 | 1 | 17.32 |
| 898 | CD1 | LEU | 115 | 41.931 | 41.003 | 109.231 | 1 | 16.49 |
| 899 | CD2 | LEU | 115 | 40.124 | 39.261 | 109.327 | 1 | 13.58 |
| 900 | C | LEU | 115 | 40.663 | 39.777 | 113.772 | 1 | 17.71 |
| 901 | O | LEU | 115 | 40.703 | 40.772 | 114.494 | 1 | 17.3 |
| 902 | N | ILE | 116 | 41.21 | 38.611 | 114.102 | 1 | 16.66 |
| 903 | CA | ILE | 116 | 42.032 | 38.533 | 115.297 | 1 | 17.19 |
| 904 | CB | ILE | 116 | 42.314 | 37.079 | 115.72 | 1 | 19.47 |
| 905 | CG2 | ILE | 116 | 43.477 | 37.042 | 116.72 | 1 | 17.88 |
| 906 | CG1 | ILE | 116 | 41.037 | 36.456 | 116.295 | 1 | 19.53 |
| 907 | CD1 | ILE | 116 | 41.17 | 34.985 | 116.718 | 1 | 20.36 |
| 908 | C | ILE | 116 | 43.308 | 39.146 | 114.686 | 1 | 16.44 |
| 909 | O | ILE | 116 | 43.847 | 38.633 | 113.696 | 1 | 16.7 |
| 910 | N | HIS | 117 | 43.787 | 40.233 | 115.272 | 1 | 16.1 |
| 911 | CA | HIS | 117 | 44.952 | 40.963 | 114.763 | 1 | 17.06 |
| 912 | CB | HIS | 117 | 45.02 | 42.32 | 115.469 | 1 | 17.25 |
| 913 | CG | HIS | 117 | 45.752 | 43.379 | 114.7 | 1 | 18.13 |
| 914 | CD2 | HIS | 117 | 46.722 | 43.296 | 113.757 | 1 | 19.98 |
| 915 | ND1 | HIS | 117 | 45.517 | 44.726 | 114.886 | 1 | 19.09 |
| 916 | CE1 | HIS | 117 | 46.311 | 45.426 | 114.094 | 1 | 19.36 |
| 917 | NE2 | HIS | 117 | 47.053 | 44.583 | 113.399 | 1 | 19.51 |
| 918 | C | HIS | 117 | 46.319 | 40.275 | 114.863 | 1 | 19.74 |
| 919 | O | HIS | 117 | 47.11 | 40.305 | 113.913 | 1 | 16.67 |
| 920 | N | PHE | 118 | 46.584 | 39.658 | 116.011 | 1 | 18.7 |
| 921 | CA | PHE | 118 | 47.857 | 38.999 | 116.275 | 1 | 19.93 |
| 922 | CB | PHE | 118 | 48.848 | 40 | 116.869 | 1 | 18.32 |
| 923 | CG | PHE | 118 | 50.284 | 39.587 | 116.726 | 1 | 21.12 |
| 924 | CD1 | PHE | 118 | 50.87 | 39.518 | 115.462 | 1 | 22.69 |
| 925 | CD2 | PHE | 118 | 51.04 | 39.227 | 117.837 | 1 | 20.64 |
| 926 | CE1 | PHE | 118 | 52.195 | 39.09 | 115.299 | 1 | 23.73 |
| 927 | CE2 | PHE | 118 | 52.375 | 38.794 | 117.692 | 1 | 21.43 |
| 928 | CZ | PHE | 118 | 52.948 | 38.725 | 116.424 | 1 | 22.01 |
| 929 | C | PHE | 118 | 47.56 | 37.895 | 117.283 | 1 | 18.66 |
| 930 | O | PHE | 118 | 46.855 | 38.118 | 118.25 | 1 | 19.59 |
| 931 | N | PRO | 119 | 48.108 | 36.692 | 117.063 | 1 | 19.24 |
| 932 | CD | PRO | 119 | 49.001 | 36.354 | 115.938 | 1 | 18.33 |
| 933 | CA | PRO | 119 | 47.895 | 35.531 | 117.938 | 1 | 19.19 |
| 934 | CB | PRO | 119 | 48.368 | 34.359 | 117.075 | 1 | 18.53 |
| 935 | CG | PRO | 119 | 49.486 | 34.963 | 116.282 | 1 | 18 |
| 936 | C | PRO | 119 | 48.513 | 35.527 | 119.329 | 1 | 17.99 |
| 937 | O | PRO | 119 | 48.215 | 34.637 | 120.115 | 1 | 21.68 |
| 938 | N | VAL | 120 | 49.355 | 36.504 | 119.646 | 1 | 18.15 |
| 939 | CA | VAL | 120 | 49.945 | 36.562 | 120.988 | 1 | 18.91 |
| 940 | CB | VAL | 120 | 51.391 | 37.115 | 120.937 | 1 | 19.49 |
| 941 | CG1 | VAL | 120 | 51.929 | 37.309 | 122.356 | 1 | 17.66 |
| 942 | CG2 | VAL | 120 | 52.288 | 36.135 | 120.162 | 1 | 18.69 |
| 943 | C | VAL | 120 | 49.065 | 37.48 | 121.857 | 1 | 18.14 |
| 944 | O | VAL | 120 | 48.751 | 38.592 | 121.456 | 1 | 17.51 |
| 945 | N | SER | 121 | 48.678 | 37.021 | 123.043 | 1 | 17.59 |
| 946 | CA | SER | 121 | 47.817 | 37.822 | 123.916 | 1 | 17.32 |
| 947 | CB | SER | 121 | 46.881 | 36.905 | 124.71 | 1 | 15.31 |
| 948 | OG | SER | 121 | 46.364 | 35.884 | 123.876 | 1 | 18.77 |
| 949 | C | SER | 121 | 48.586 | 38.679 | 124.902 | 1 | 16.53 |
| 950 | O | SER | 121 | 49.671 | 38.289 | 125.339 | 1 | 18.72 |
| 951 | N | VAL | 122 | 48.046 | 39.85 | 125.246 | 1 | 16.49 |
| 952 | CA | VAL | 122 | 48.696 | 40.698 | 126.253 | 1 | 17.36 |
| 953 | CB | VAL | 122 | 49.271 | 42.039 | 125.671 | 1 | 18.28 |
| 954 | CG1 | VAL | 122 | 50.271 | 41.728 | 124.564 | 1 | 20.07 |
| 955 | CG2 | VAL | 122 | 48.179 | 42.954 | 125.157 | 1 | 15.48 |
| 956 | C | VAL | 122 | 47.673 | 40.971 | 127.359 | 1 | 18.29 |
| 957 | O | VAL | 122 | 46.476 | 40.699 | 127.196 | 1 | 16.66 |
| 958 | N | LYS | 123 | 48.142 | 41.474 | 128.492 | 1 | 18.31 |
| 959 | CA | LYS | 123 | 47.27 | 41.747 | 129.635 | 1 | 20.99 |
| 960 | CB | LYS | 123 | 48.106 | 42.344 | 130.785 | 1 | 24.66 |
| 961 | CG | LYS | 123 | 47.307 | 42.812 | 131.988 | 1 | 32.3 |
| 962 | CD | LYS | 123 | 48.235 | 43.421 | 133.051 | 1 | 38.57 |
| 963 | CE | LYS | 123 | 47.469 | 43.943 | 134.271 | 1 | 42.41 |
| 964 | NZ | LYS | 123 | 46.502 | 45.024 | 133.898 | 1 | 46.41 |
| 965 | C | LYS | 123 | 46.101 | 42.683 | 129.314 | 1 | 19.29 |
| 966 | O | LYS | 123 | 46.277 | 43.694 | 128.65 | 1 | 20.06 |
| 967 | N | PRO | 124 | 44.882 | 42.339 | 129.768 | 1 | 20.15 |
| 968 | CD | PRO | 124 | 44.467 | 41.12 | 130.489 | 1 | 19.25 |
| 969 | CA | PRO | 124 | 43.731 | 43.208 | 129.495 | 1 | 20.69 |
| 970 | CB | PRO | 124 | 42.565 | 42.476 | 130.17 | 1 | 21.24 |
| 971 | CG | PRO | 124 | 42.995 | 41.04 | 130.159 | 1 | 21.45 |
| 972 | C | PRO | 124 | 43.962 | 44.587 | 130.117 | 1 | 22.18 |
| 973 | O | PRO | 124 | 44.658 | 44.711 | 131.13 | 1 | 21.98 |
| 974 | N | GLY | 125 | 43.369 | 45.614 | 129.519 | 1 | 23.23 |
| 975 | CA | GLY | 125 | 43.537 | 46.961 | 130.033 | 1 | 25.26 |
| 976 | C | GLY | 125 | 43.556 | 47.962 | 128.893 | 1 | 26.59 |
| 977 | O | GLY | 125 | 43.563 | 47.567 | 127.729 | 1 | 26.11 |
| 978 | N | GLU | 126 | 43.584 | 49.254 | 129.21 | 1 | 27.64 |
| 979 | CA | GLU | 126 | 43.579 | 50.267 | 128.16 | 1 | 29.99 |
| 980 | CB | GLU | 126 | 43.293 | 51.658 | 128.746 | 1 | 31.39 |
| 981 | CG | GLU | 126 | 43.076 | 52.729 | 127.676 | 1 | 34.06 |
| 982 | CD | GLU | 126 | 41.855 | 52.452 | 126.794 | 1 | 35.58 |
| 983 | OE1 | GLU | 126 | 41.789 | 53.014 | 125.681 | 1 | 37.56 |
| 984 | OE2 | GLU | 126 | 40.959 | 51.689 | 127.217 | 1 | 36.34 |
| 985 | C | GLU | 126 | 44.868 | 50.306 | 127.336 | 1 | 29.78 |
| 986 | O | GLU | 126 | 44.819 | 50.591 | 126.141 | 1 | 30.29 |
| 987 | N | GLU | 127 | 46.01 | 50.002 | 127.955 | 1 | 28.44 |
| 988 | CA | GLU | 127 | 47.289 | 50.03 | 127.235 | 1 | 30.75 |
| 989 | CB | GLU | 127 | 48.465 | 49.912 | 128.204 | 1 | 33.73 |
| 990 | CG | GLU | 127 | 48.672 | 51.126 | 129.078 | 1 | 42.95 |
| 991 | CD | GLU | 127 | 49.823 | 50.947 | 130.051 | 1 | 47.41 |
| 992 | OE1 | GLU | 127 | 49.723 | 50.076 | 130.949 | 1 | 49.7 |
| 993 | OE2 | GLU | 127 | 50.831 | 51.681 | 129.919 | 1 | 51.13 |
| 994 | C | GLU | 127 | 47.392 | 48.915 | 126.208 | 1 | 28.97 |
| 995 | O | GLU | 127 | 47.241 | 47.742 | 126.544 | 1 | 27.85 |
| 996 | N | VAL | 128 | 47.674 | 49.27 | 124.96 | 1 | 26.71 |
| 997 | CA | VAL | 128 | 47.767 | 48.247 | 123.928 | 1 | 26.17 |
| 998 | CB | VAL | 128 | 47.54 | 48.85 | 122.522 | 1 | 26.28 |
| 999 | CG1 | VAL | 128 | 46.132 | 49.421 | 122.462 | 1 | 27.24 |
| 1000 | CG2 | VAL | 128 | 48.563 | 49.931 | 122.207 | 1 | 26.18 |
| 1001 | C | VAL | 128 | 49.07 | 47.448 | 123.985 | 1 | 25.98 |
| 1002 | O | VAL | 128 | 49.126 | 46.314 | 123.508 | 1 | 25.13 |
| 1003 | N | ILE | 129 | 50.11 | 48.026 | 124.578 | 1 | 23.42 |
| 1004 | CA | ILE | 129 | 51.378 | 47.318 | 124.728 | 1 | 24.86 |
| 1005 | CB | ILE | 129 | 52.487 | 47.88 | 123.817 | 1 | 28 |
| 1006 | CG2 | ILE | 129 | 53.739 | 47.041 | 123.974 | 1 | 27.42 |
| 1007 | CG1 | ILE | 129 | 52.034 | 47.895 | 122.353 | 1 | 29.89 |
| 1008 | CD1 | ILE | 129 | 51.761 | 46.543 | 121.76 | 1 | 31.44 |
| 1009 | C | ILE | 129 | 51.827 | 47.492 | 126.172 | 1 | 24.28 |
| 1010 | O | ILE | 129 | 52.717 | 48.289 | 126.464 | 1 | 24.94 |
| 1011 | N | PRO | 130 | 51.207 | 46.749 | 127.102 | 1 | 22.67 |
| 1012 | CD | PRO | 130 | 50.149 | 45.736 | 126.905 | 1 | 21.84 |
| 1013 | CA | PRO | 130 | 51.579 | 46.866 | 128.513 | 1 | 22.3 |
| 1014 | CB | PRO | 130 | 50.444 | 46.137 | 129.231 | 1 | 20.51 |
| 1015 | CG | PRO | 130 | 50.08 | 45.046 | 128.265 | 1 | 19.52 |
| 1016 | C | PRO | 130 | 52.945 | 46.244 | 128.768 | 1 | 22.31 |
| 1017 | O | PRO | 130 | 53.245 | 45.177 | 128.241 | 1 | 19.83 |
| 1018 | N | LYS | 131 | 53.766 | 46.928 | 129.562 | 1 | 22.4 |
| 1019 | CA | LYS | 131 | 55.114 | 46.469 | 129.88 | 1 | 25.57 |
| 1020 | CB | LYS | 131 | 56.155 | 47.367 | 129.196 | 1 | 28.25 |
| 1021 | CG | LYS | 131 | 56.134 | 47.33 | 127.676 | 1 | 31.78 |
| 1022 | CD | LYS | 131 | 57.074 | 48.379 | 127.09 | 1 | 37.23 |
| 1023 | CE | LYS | 131 | 56.971 | 48.45 | 125.575 | 1 | 39.52 |
| 1024 | NZ | LYS | 131 | 57.799 | 49.567 | 125.021 | 1 | 44.65 |
| 1025 | C | LYS | 131 | 55.377 | 46.465 | 131.383 | 1 | 25.7 |
| 1026 | O | LYS | 131 | 54.735 | 47.201 | 132.137 | 1 | 22.82 |
| 1027 | N | ASP | 132 | 56.317 | 45.631 | 131.819 | 1 | 26.15 |
| 1028 | CA | ASP | 132 | 56.652 | 45.567 | 133.235 | 1 | 29.24 |
| 1029 | CB | ASP | 132 | 57.032 | 44.142 | 133.66 | 1 | 28.46 |
| 1030 | CG | ASP | 132 | 58.196 | 43.568 | 132.853 | 1 | 29.04 |
| 1031 | OD1 | ASP | 132 | 59.076 | 44.338 | 132.417 | 1 | 29.33 |
| 1032 | OD2 | ASP | 132 | 58.222 | 42.335 | 132.671 | 1 | 28.3 |
| 1033 | C | ASP | 132 | 57.802 | 46.516 | 133.557 | 1 | 30.65 |
| 1034 | O | ASP | 132 | 58.279 | 47.257 | 132.694 | 1 | 29.64 |
| 1035 | N | GLU | 133 | 58.237 | 46.473 | 134.809 | 1 | 34.55 |
| 1036 | CA | GLU | 133 | 59.313 | 47.323 | 135.303 | 1 | 38.41 |
| 1037 | CB | GLU | 133 | 59.566 | 47.015 | 136.784 | 1 | 41.71 |
| 1038 | CG | GLU | 133 | 59.922 | 45.552 | 137.058 | 1 | 48.53 |
| 1039 | CD | GLU | 133 | 60.018 | 45.225 | 138.548 | 1 | 53.12 |
| 1040 | OE1 | GLU | 133 | 60.742 | 45.944 | 139.28 | 1 | 56.07 |
| 1041 | OE2 | GLU | 133 | 59.379 | 44.238 | 138.989 | 1 | 54.63 |
| 1042 | C | GLU | 133 | 60.605 | 47.163 | 134.512 | 1 | 39.06 |
| 1043 | O | GLU | 133 | 61.351 | 48.123 | 134.33 | 1 | 40.02 |
| 1044 | N | ASN | 134 | 60.856 | 45.952 | 134.026 | 1 | 38.55 |
| 1045 | CA | ASN | 134 | 62.072 | 45.672 | 133.273 | 1 | 37.71 |
| 1046 | CB | ASN | 134 | 62.454 | 44.204 | 133.445 | 1 | 37.78 |
| 1047 | CG | ASN | 134 | 62.665 | 43.834 | 134.891 | 1 | 37.39 |
| 1048 | OD1 | ASN | 134 | 62.221 | 42.781 | 135.35 | 1 | 39.84 |
| 1049 | ND2 | ASN | 134 | 63.346 | 44.703 | 135.625 | 1 | 39.06 |
| 1050 | C | ASN | 134 | 61.935 | 45.996 | 131.802 | 1 | 36.69 |
| 1051 | O | ASN | 134 | 62.848 | 45.742 | 131.022 | 1 | 38.22 |
| 1052 | N | GLY | 135 | 60.796 | 46.562 | 131.42 | 1 | 35.14 |
| 1053 | CA | GLY | 135 | 60.581 | 46.896 | 130.024 | 1 | 32.83 |
| 1054 | C | GLY | 135 | 60.104 | 45.722 | 129.18 | 1 | 31.48 |
| 1055 | O | GLY | 135 | 60.066 | 45.818 | 127.96 | 1 | 31.99 |
| 1056 | N | LYS | 136 | 59.742 | 44.612 | 129.816 | 1 | 29.85 |
| 1057 | CA | LYS | 136 | 59.267 | 43.441 | 129.076 | 1 | 29.54 |
| 1058 | CB | LYS | 136 | 59.692 | 42.158 | 129.789 | 1 | 32.26 |
| 1059 | CG | LYS | 136 | 61.175 | 42.088 | 130.14 | 1 | 36.49 |
| 1060 | CD | LYS | 136 | 62.056 | 42.186 | 128.905 | 1 | 38.71 |
| 1061 | CE | LYS | 136 | 63.539 | 42.114 | 129.266 | 1 | 40.88 |
| 1062 | NZ | LYS | 136 | 64.392 | 42.228 | 128.048 | 1 | 44.11 |
| 1063 | C | LYS | 136 | 57.742 | 43.467 | 128.947 | 1 | 29.07 |
| 1064 | O | LYS | 136 | 57.034 | 43.889 | 129.862 | 1 | 26.85 |
| 1065 | N | ILE | 137 | 57.228 | 43.004 | 127.816 | 1 | 27.92 |
| 1066 | CA | ILE | 137 | 55.788 | 43.008 | 127.618 | 1 | 27.14 |
| 1067 | CB | ILE | 137 | 55.447 | 42.61 | 126.171 | 1 | 29.57 |
| 1068 | CG2 | ILE | 137 | 53.945 | 42.462 | 125.995 | 1 | 27.1 |
| 1069 | CG1 | ILE | 137 | 55.97 | 43.696 | 125.223 | 1 | 31.39 |
| 1070 | CD1 | ILE | 137 | 55.665 | 43.437 | 123.778 | 1 | 35.74 |
| 1071 | C | ILE | 137 | 55.06 | 42.097 | 128.605 | 1 | 24.95 |
| 1072 | O | ILE | 137 | 55.551 | 41.026 | 128.955 | 1 | 24.12 |
| 1073 | N | LEU | 138 | 53.902 | 42.551 | 129.079 | 1 | 22.65 |
| 1074 | CA | LEU | 138 | 53.083 | 41.771 | 130.003 | 1 | 21.66 |
| 1075 | CB | LEU | 138 | 52.241 | 42.693 | 130.894 | 1 | 21.35 |
| 1076 | CG | LEU | 138 | 52.985 | 43.488 | 131.982 | 1 | 23.73 |
| 1077 | CD1 | LEU | 138 | 52.012 | 44.465 | 132.654 | 1 | 23.73 |
| 1078 | CD2 | LEU | 138 | 53.584 | 42.532 | 133.014 | 1 | 20.64 |
| 1079 | C | LEU | 138 | 52.156 | 40.879 | 129.187 | 1 | 21.5 |
| 1080 | O | LEU | 138 | 51.053 | 41.284 | 128.823 | 1 | 20.11 |
| 1081 | N | PHE | 139 | 52.608 | 39.669 | 128.89 | 1 | 19.99 |
| 1082 | CA | PHE | 139 | 51.811 | 38.727 | 128.113 | 1 | 21.48 |
| 1083 | CB | PHE | 139 | 52.693 | 37.577 | 127.629 | 1 | 21.3 |
| 1084 | CG | PHE | 139 | 53.817 | 38.005 | 126.737 | 1 | 23.78 |
| 1085 | CD1 | PHE | 139 | 55.137 | 37.933 | 127.173 | 1 | 22.62 |
| 1086 | CD2 | PHE | 139 | 53.555 | 38.441 | 125.44 | 1 | 22.45 |
| 1087 | CE1 | PHE | 139 | 56.185 | 38.284 | 126.322 | 1 | 24.11 |
| 1088 | CE2 | PHE | 139 | 54.589 | 38.796 | 124.582 | 1 | 23.09 |
| 1089 | CZ | PHE | 139 | 55.909 | 38.717 | 125.019 | 1 | 23.08 |
| 1090 | C | PHE | 139 | 50.672 | 38.133 | 128.94 | 1 | 21.65 |
| 1091 | O | PHE | 139 | 50.707 | 38.157 | 130.167 | 1 | 22.63 |
| 1092 | N | ASP | 140 | 49.668 | 37.592 | 128.258 | 1 | 20.57 |
| 1093 | CA | ASP | 140 | 48.542 | 36.941 | 128.923 | 1 | 20.45 |
| 1094 | CB | ASP | 140 | 47.237 | 37.733 | 128.711 | 1 | 20.67 |
| 1095 | CG | ASP | 140 | 46.088 | 37.25 | 129.614 | 1 | 23.36 |
| 1096 | OD1 | ASP | 140 | 46.366 | 36.708 | 130.692 | 1 | 23.17 |
| 1097 | OD2 | ASP | 140 | 44.903 | 37.43 | 129.256 | 1 | 23.79 |
| 1098 | C | ASP | 140 | 48.457 | 35.576 | 128.24 | 1 | 20.96 |
| 1099 | O | ASP | 140 | 49.043 | 35.382 | 127.174 | 1 | 22.89 |
| 1100 | N | THR | 141 | 47.761 | 34.633 | 128.862 | 1 | 18.57 |
| 1101 | CA | THR | 141 | 47.607 | 33.305 | 128.291 | 1 | 18.74 |
| 1102 | CB | THR | 141 | 48.14 | 32.235 | 129.248 | 1 | 20.47 |
| 1103 | OG1 | THR | 141 | 49.555 | 32.405 | 129.374 | 1 | 21.87 |
| 1104 | CG2 | THR | 141 | 47.827 | 30.829 | 128.731 | 1 | 15.71 |
| 1105 | C | THR | 141 | 46.127 | 33.082 | 128.033 | 1 | 18.17 |
| 1106 | O | THR | 141 | 45.315 | 33.138 | 128.949 | 1 | 17.06 |
| 1107 | N | VAL | 142 | 45.781 | 32.842 | 126.775 | 1 | 18.08 |
| 1108 | CA | VAL | 142 | 44.393 | 32.641 | 126.38 | 1 | 18.65 |
| 1109 | CB | VAL | 142 | 43.789 | 33.949 | 125.8 | 1 | 19.5 |
| 1110 | CG1 | VAL | 142 | 42.308 | 33.755 | 125.49 | 1 | 18.74 |
| 1111 | CG2 | VAL | 142 | 43.993 | 35.099 | 126.772 | 1 | 20.06 |
| 1112 | C | VAL | 142 | 44.322 | 31.588 | 125.291 | 1 | 18.64 |
| 1113 | O | VAL | 142 | 45.118 | 31.614 | 124.355 | 1 | 16.35 |
| 1114 | N | ASP | 143 | 43.373 | 30.672 | 125.43 | 1 | 18.87 |
| 1115 | CA | ASP | 143 | 43.129 | 29.624 | 124.448 | 1 | 19.44 |
| 1116 | CB | ASP | 143 | 42.149 | 28.608 | 125.044 | 1 | 20.43 |
| 1117 | CG | ASP | 143 | 41.763 | 27.512 | 124.064 | 1 | 22.27 |
| 1118 | OD1 | ASP | 143 | 42.212 | 27.556 | 122.905 | 1 | 19.67 |
| 1119 | OD2 | ASP | 143 | 41.007 | 26.614 | 124.474 | 1 | 22.89 |
| 1120 | C | ASP | 143 | 42.488 | 30.326 | 123.234 | 1 | 18.39 |
| 1121 | O | ASP | 143 | 41.353 | 30.799 | 123.317 | 1 | 16.12 |
| 1122 | N | LEU | 144 | 43.211 | 30.41 | 122.119 | 1 | 18.24 |
| 1123 | CA | LEU | 144 | 42.673 | 31.07 | 120.929 | 1 | 18.62 |
| 1124 | CB | LEU | 144 | 43.73 | 31.161 | 119.817 | 1 | 21.51 |
| 1125 | CG | LEU | 144 | 44.895 | 32.131 | 120.073 | 1 | 24.41 |
| 1126 | CD1 | LEU | 144 | 45.867 | 32.132 | 118.906 | 1 | 23.81 |
| 1127 | CD2 | LEU | 144 | 44.332 | 33.524 | 120.285 | 1 | 25.19 |
| 1128 | C | LEU | 144 | 41.421 | 30.395 | 120.394 | 1 | 18.64 |
| 1129 | O | LEU | 144 | 40.64 | 31.017 | 119.674 | 1 | 17.42 |
| 1130 | N | CYS | 145 | 41.221 | 29.117 | 120.706 | 1 | 18.25 |
| 1131 | CA | CYS | 145 | 39.994 | 28.458 | 120.237 | 1 | 19.32 |
| 1132 | CB | CYS | 145 | 40.044 | 26.945 | 120.5 | 1 | 19.66 |
| 1133 | SG | CYS | 145 | 41.139 | 26.087 | 119.368 | 1 | 25.77 |
| 1134 | C | CYS | 145 | 38.795 | 29.091 | 120.943 | 1 | 19.56 |
| 1135 | O | CYS | 145 | 37.71 | 29.184 | 120.362 | 1 | 19.58 |
| 1136 | N | ALA | 146 | 38.991 | 29.541 | 122.185 | 1 | 18.68 |
| 1137 | CA | ALA | 146 | 37.906 | 30.192 | 122.939 | 1 | 18.93 |
| 1138 | CB | ALA | 146 | 38.276 | 30.292 | 124.428 | 1 | 19.44 |
| 1139 | C | ALA | 146 | 37.645 | 31.59 | 122.353 | 1 | 17.77 |
| 1140 | O | ALA | 146 | 36.505 | 32.05 | 122.28 | 1 | 18.91 |
| 1141 | N | THR | 147 | 38.716 | 32.262 | 121.939 | 1 | 17.75 |
| 1142 | CA | THR | 147 | 38.603 | 33.575 | 121.316 | 1 | 17.1 |
| 1143 | CB | THR | 147 | 39.995 | 34.145 | 120.941 | 1 | 19.35 |
| 1144 | OG1 | THR | 147 | 40.825 | 34.171 | 122.107 | 1 | 17.02 |
| 1145 | CG2 | THR | 147 | 39.873 | 35.567 | 120.38 | 1 | 18.51 |
| 1146 | C | THR | 147 | 37.796 | 33.402 | 120.023 | 1 | 17.22 |
| 1147 | O | THR | 147 | 36.89 | 34.184 | 119.743 | 1 | 15.66 |
| 1148 | N | TRP | 148 | 38.131 | 32.373 | 119.242 | 1 | 16.44 |
| 1149 | CA | TRP | 148 | 37.429 | 32.129 | 117.98 | 1 | 16.96 |
| 1150 | CB | TRP | 148 | 38.051 | 30.956 | 117.207 | 1 | 15.95 |
| 1151 | CG | TRP | 148 | 37.484 | 30.862 | 115.818 | 1 | 17.93 |
| 1152 | CD2 | TRP | 148 | 37.688 | 31.799 | 114.751 | 1 | 17.54 |
| 1153 | CE2 | TRP | 148 | 36.892 | 31.372 | 113.658 | 1 | 18.38 |
| 1154 | CE3 | TRP | 148 | 38.46 | 32.961 | 114.614 | 1 | 17.37 |
| 1155 | CD1 | TRP | 148 | 36.599 | 29.926 | 115.342 | 1 | 17.42 |
| 1156 | NE1 | TRP | 148 | 36.239 | 30.23 | 114.046 | 1 | 18.41 |
| 1157 | CZ2 | TRP | 148 | 36.851 | 32.071 | 112.438 | 1 | 17.82 |
| 1158 | CZ3 | TRP | 148 | 38.415 | 33.661 | 113.391 | 1 | 18.03 |
| 1159 | CH2 | TRP | 148 | 37.617 | 33.21 | 112.329 | 1 | 16.09 |
| 1160 | C | TRP | 148 | 35.937 | 31.884 | 118.2 | 1 | 17.16 |
| 1161 | O | TRP | 148 | 35.1 | 32.384 | 117.447 | 1 | 16.61 |
| 1162 | N | GLU | 149 | 35.597 | 31.114 | 119.229 | 1 | 17.78 |
| 1163 | CA | GLU | 149 | 34.191 | 30.865 | 119.528 | 1 | 19.7 |
| 1164 | CB | GLU | 149 | 34.064 | 29.939 | 120.748 | 1 | 22.43 |
| 1165 | CG | GLU | 149 | 34.121 | 28.467 | 120.381 | 1 | 27.26 |
| 1166 | CD | GLU | 149 | 34.365 | 27.553 | 121.579 | 1 | 33.05 |
| 1167 | OE1 | GLU | 149 | 33.912 | 27.883 | 122.694 | 1 | 35.61 |
| 1168 | OE2 | GLU | 149 | 34.998 | 26.498 | 121.391 | 1 | 33.21 |
| 1169 | C | GLU | 149 | 33.444 | 32.174 | 119.774 | 1 | 18.35 |
| 1170 | O | GLU | 149 | 32.312 | 32.35 | 119.333 | 1 | 17.6 |
| 1171 | N | ALA | 150 | 34.083 | 33.092 | 120.488 | 1 | 18.08 |
| 1172 | CA | ALA | 150 | 33.477 | 34.391 | 120.768 | 1 | 18.24 |
| 1173 | CB | ALA | 150 | 34.336 | 35.176 | 121.771 | 1 | 16.07 |
| 1174 | C | ALA | 150 | 33.331 | 35.183 | 119.456 | 1 | 17.9 |
| 1175 | O | ALA | 150 | 32.357 | 35.906 | 119.28 | 1 | 16.93 |
| 1176 | N | MET | 151 | 34.3 | 35.047 | 118.545 | 1 | 16.76 |
| 1177 | CA | MET | 151 | 34.241 | 35.728 | 117.244 | 1 | 16.67 |
| 1178 | CB | MET | 151 | 35.519 | 35.466 | 116.431 | 1 | 18.01 |
| 1179 | CG | MET | 151 | 36.805 | 36.086 | 117.001 | 1 | 20.11 |
| 1180 | SD | MET | 151 | 36.915 | 37.856 | 116.729 | 1 | 24.45 |
| 1181 | CE | MET | 151 | 37.436 | 37.875 | 114.95 | 1 | 23.58 |
| 1182 | C | MET | 151 | 33.041 | 35.209 | 116.448 | 1 | 17.22 |
| 1183 | O | MET | 151 | 32.336 | 35.986 | 115.8 | 1 | 18.3 |
| 1184 | N | GLU | 152 | 32.817 | 33.897 | 116.486 | 1 | 15.7 |
| 1185 | CA | GLU | 152 | 31.693 | 33.289 | 115.761 | 1 | 17.57 |
| 1186 | CB | GLU | 152 | 31.704 | 31.768 | 115.928 | 1 | 18.48 |
| 1187 | CG | GLU | 152 | 32.923 | 31.092 | 115.302 | 1 | 17.62 |
| 1188 | CD | GLU | 152 | 32.923 | 29.577 | 115.515 | 1 | 20.58 |
| 1189 | OE1 | GLU | 152 | 32.559 | 29.154 | 116.627 | 1 | 18.71 |
| 1190 | OE2 | GLU | 152 | 33.294 | 28.832 | 114.585 | 1 | 18.38 |
| 1191 | C | GLU | 152 | 30.369 | 33.859 | 116.259 | 1 | 18.9 |
| 1192 | O | GLU | 152 | 29.448 | 34.104 | 115.473 | 1 | 17.35 |
| 1193 | N | LYS | 153 | 30.276 | 34.067 | 117.569 | 1 | 18.75 |
| 1194 | CA | LYS | 153 | 29.068 | 34.641 | 118.157 | 1 | 19.9 |
| 1195 | CB | LYS | 153 | 29.146 | 34.588 | 119.681 | 1 | 22.18 |
| 1196 | CG | LYS | 153 | 28.977 | 33.186 | 120.229 | 1 | 25.13 |
| 1197 | CD | LYS | 153 | 29.123 | 33.157 | 121.732 | 1 | 30.14 |
| 1198 | CE | LYS | 153 | 28.915 | 31.753 | 122.274 | 1 | 33.98 |
| 1199 | NZ | LYS | 153 | 29.057 | 31.725 | 123.755 | 1 | 36.09 |
| 1200 | C | LYS | 153 | 28.837 | 36.084 | 117.696 | 1 | 20.24 |
| 1201 | O | LYS | 153 | 27.688 | 36.541 | 117.608 | 1 | 19.49 |
| 1202 | N | CYS | 154 | 29.916 | 36.809 | 117.407 | 1 | 19.47 |
| 1203 | CA | CYS | 154 | 29.765 | 38.183 | 116.92 | 1 | 20.4 |
| 1204 | CB | CYS | 154 | 31.104 | 38.926 | 116.933 | 1 | 22.16 |
| 1205 | SG | CYS | 154 | 31.681 | 39.373 | 118.591 | 1 | 22.96 |
| 1206 | C | CYS | 154 | 29.195 | 38.154 | 115.502 | 1 | 19.84 |
| 1207 | O | CYS | 154 | 28.421 | 39.033 | 115.138 | 1 | 18.69 |
| 1208 | N | LYS | 155 | 29.588 | 37.159 | 114.706 | 1 | 19.14 |
| 1209 | CA | LYS | 155 | 29.055 | 37.022 | 113.351 | 1 | 20.04 |
| 1210 | CB | LYS | 155 | 29.812 | 35.937 | 112.576 | 1 | 19.99 |
| 1211 | CG | LYS | 155 | 29.231 | 35.627 | 111.197 | 1 | 21.91 |
| 1212 | CD | LYS | 155 | 30.122 | 34.669 | 110.424 | 1 | 22.3 |
| 1213 | CE | LYS | 155 | 29.422 | 34.106 | 109.196 | 1 | 24.2 |
| 1214 | NZ | LYS | 155 | 28.968 | 35.161 | 108.261 | 1 | 24.25 |
| 1215 | C | LYS | 155 | 27.563 | 36.653 | 113.447 | 1 | 21.9 |
| 1216 | O | LYS | 155 | 26.723 | 37.229 | 112.752 | 1 | 22.03 |
| 1217 | N | ASP | 156 | 27.231 | 35.694 | 114.303 | 1 | 22.09 |
| 1218 | CA | ASP | 156 | 25.83 | 35.307 | 114.45 | 1 | 23.73 |
| 1219 | CB | ASP | 156 | 25.69 | 34.124 | 115.401 | 1 | 24.59 |
| 1220 | CG | ASP | 156 | 26.094 | 32.82 | 114.754 | 1 | 27.27 |
| 1221 | OD1 | ASP | 156 | 26.001 | 32.729 | 113.513 | 1 | 27.59 |
| 1222 | OD2 | ASP | 156 | 26.487 | 31.895 | 115.482 | 1 | 27.25 |
| 1223 | C | ASP | 156 | 24.959 | 36.457 | 114.941 | 1 | 23.15 |
| 1224 | O | ASP | 156 | 23.79 | 36.549 | 114.581 | 1 | 23.39 |
| 1225 | N | ALA | 157 | 25.534 | 37.326 | 115.766 | 1 | 21.56 |
| 1226 | CA | ALA | 157 | 24.809 | 38.474 | 116.293 | 1 | 22.11 |
| 1227 | CB | ALA | 157 | 25.534 | 39.031 | 117.512 | 1 | 23.07 |
| 1228 | C | ALA | 157 | 24.658 | 39.566 | 115.227 | 1 | 22.43 |
| 1229 | O | ALA | 157 | 23.962 | 40.559 | 115.442 | 1 | 23.8 |
| 1230 | N | GLY | 158 | 25.322 | 39.388 | 114.091 | 1 | 21.85 |
| 1231 | CA | GLY | 158 | 25.234 | 40.365 | 113.016 | 1 | 21.14 |
| 1232 | C | GLY | 158 | 26.175 | 41.553 | 113.135 | 1 | 22.19 |
| 1233 | O | GLY | 158 | 26.092 | 42.466 | 112.322 | 1 | 22.57 |
| 1234 | N | LEU | 159 | 27.073 | 41.552 | 114.121 | 1 | 20.77 |
| 1235 | CA | LEU | 159 | 28.002 | 42.675 | 114.307 | 1 | 20.11 |
| 1236 | CB | LEU | 159 | 28.557 | 42.678 | 115.736 | 1 | 21.83 |
| 1237 | CG | LEU | 159 | 27.519 | 42.778 | 116.859 | 1 | 25.56 |
| 1238 | CD1 | LEU | 159 | 28.177 | 42.514 | 118.221 | 1 | 27.21 |
| 1239 | CD2 | LEU | 159 | 26.88 | 44.15 | 116.823 | 1 | 26.46 |
| 1240 | C | LEU | 159 | 29.169 | 42.65 | 113.311 | 1 | 19.47 |
| 1241 | O | LEU | 159 | 29.758 | 43.688 | 113.006 | 1 | 18.75 |
| 1242 | N | ALA | 160 | 29.511 | 41.458 | 112.838 | 1 | 17.69 |
| 1243 | CA | ALA | 160 | 30.577 | 41.272 | 111.854 | 1 | 19.37 |
| 1244 | CB | ALA | 160 | 31.801 | 40.615 | 112.502 | 1 | 18.51 |
| 1245 | C | ALA | 160 | 29.986 | 40.348 | 110.797 | 1 | 19.78 |
| 1246 | O | ALA | 160 | 29.504 | 39.269 | 111.131 | 1 | 21.12 |
| 1247 | N | LYS | 161 | 30 | 40.769 | 109.534 | 1 | 18.95 |
| 1248 | CA | LYS | 161 | 29.451 | 39.946 | 108.454 | 1 | 19.09 |
| 1249 | CB | LYS | 161 | 29.339 | 40.769 | 107.164 | 1 | 20.12 |
| 1250 | CG | LYS | 161 | 28.32 | 41.893 | 107.236 | 1 | 24.07 |
| 1251 | CD | LYS | 161 | 26.908 | 41.356 | 107.425 | 1 | 27.39 |
| 1252 | CE | LYS | 161 | 25.872 | 42.478 | 107.298 | 1 | 33.77 |
| 1253 | NZ | LYS | 161 | 24.462 | 41.972 | 107.365 | 1 | 35.4 |
| 1254 | C | LYS | 161 | 30.316 | 38.716 | 108.219 | 1 | 17.87 |
| 1255 | O | LYS | 161 | 29.796 | 37.625 | 107.976 | 1 | 17.04 |
| 1256 | N | SER | 162 | 31.633 | 38.902 | 108.295 | 1 | 16.04 |
| 1257 | CA | SER | 162 | 32.609 | 37.815 | 108.119 | 1 | 16.14 |
| 1258 | CB | SER | 162 | 33.281 | 37.907 | 106.742 | 1 | 15.8 |
| 1259 | OG | SER | 162 | 32.334 | 37.755 | 105.688 | 1 | 16.86 |
| 1260 | C | SER | 162 | 33.69 | 37.943 | 109.197 | 1 | 17.65 |
| 1261 | O | SER | 162 | 33.892 | 39.019 | 109.765 | 1 | 17.35 |
| 1262 | N | ILE | 163 | 34.379 | 36.846 | 109.489 | 1 | 16.91 |
| 1263 | CA | ILE | 163 | 35.438 | 36.874 | 110.484 | 1 | 16.36 |
| 1264 | CB | ILE | 163 | 34.99 | 36.216 | 111.834 | 1 | 16.11 |
| 1265 | CG2 | ILE | 163 | 33.944 | 37.095 | 112.523 | 1 | 15.46 |
| 1266 | CG1 | ILE | 163 | 34.428 | 34.811 | 111.58 | 1 | 15.57 |
| 1267 | CD1 | ILE | 163 | 34.054 | 34.06 | 112.868 | 1 | 18.24 |
| 1268 | C | ILE | 163 | 36.647 | 36.139 | 109.926 | 1 | 16.61 |
| 1269 | O | ILE | 163 | 36.504 | 35.203 | 109.148 | 1 | 15.65 |
| 1270 | N | GLY | 164 | 37.839 | 36.576 | 110.316 | 1 | 15.81 |
| 1271 | CA | GLY | 164 | 39.042 | 35.931 | 109.825 | 1 | 16.03 |
| 1272 | C | GLY | 164 | 40.187 | 36.153 | 110.784 | 1 | 17.05 |
| 1273 | O | GLY | 164 | 39.973 | 36.541 | 111.938 | 1 | 13.78 |
| 1274 | N | VAL | 165 | 41.407 | 35.918 | 110.317 | 1 | 16.41 |
| 1275 | CA | VAL | 165 | 42.562 | 36.093 | 111.183 | 1 | 16.57 |
| 1276 | CB | VAL | 165 | 43.152 | 34.73 | 111.6 | 1 | 15.92 |
| 1277 | CG1 | VAL | 165 | 42.113 | 33.949 | 112.409 | 1 | 17.39 |
| 1278 | CG2 | VAL | 165 | 43.571 | 33.923 | 110.346 | 1 | 16.45 |
| 1279 | C | VAL | 165 | 43.642 | 36.901 | 110.471 | 1 | 17.39 |
| 1280 | O | VAL | 165 | 43.553 | 37.185 | 109.273 | 1 | 18.21 |
| 1281 | N | SER | 166 | 44.667 | 37.269 | 111.223 | 1 | 17.77 |
| 1282 | CA | SER | 166 | 45.773 | 38.023 | 110.679 | 1 | 17.79 |
| 1283 | CB | SER | 166 | 45.571 | 39.517 | 110.937 | 1 | 17.65 |
| 1284 | OG | SER | 166 | 46.616 | 40.274 | 110.34 | 1 | 19.57 |
| 1285 | C | SER | 166 | 47.026 | 37.53 | 111.374 | 1 | 18.06 |
| 1286 | O | SER | 166 | 46.995 | 37.211 | 112.559 | 1 | 18.54 |
| 1287 | N | ASN | 167 | 48.125 | 37.448 | 110.633 | 1 | 17.64 |
| 1288 | CA | ASN | 167 | 49.387 | 36.999 | 111.202 | 1 | 19.25 |
| 1289 | CB | ASN | 167 | 49.867 | 38.006 | 112.256 | 1 | 19.41 |
| 1290 | CG | ASN | 167 | 50.185 | 39.348 | 111.641 | 1 | 20.11 |
| 1291 | OD1 | ASN | 167 | 50.968 | 39.409 | 110.695 | 1 | 21.27 |
| 1292 | ND2 | ASN | 167 | 49.578 | 40.422 | 112.153 | 1 | 17.29 |
| 1293 | C | ASN | 167 | 49.372 | 35.589 | 111.784 | 1 | 19.5 |
| 1294 | O | ASN | 167 | 50.066 | 35.296 | 112.76 | 1 | 20.27 |
| 1295 | N | PHE | 168 | 48.578 | 34.714 | 111.181 | 1 | 18.79 |
| 1296 | CA | PHE | 168 | 48.525 | 33.33 | 111.615 | 1 | 18.69 |
| 1297 | CB | PHE | 168 | 47.102 | 32.766 | 111.518 | 1 | 19.54 |
| 1298 | CG | PHE | 168 | 46.287 | 32.928 | 112.777 | 1 | 17.92 |
| 1299 | CD1 | PHE | 168 | 46.274 | 34.131 | 113.473 | 1 | 17.75 |
| 1300 | CD2 | PHE | 168 | 45.506 | 31.877 | 113.243 | 1 | 17.13 |
| 1301 | CE1 | PHE | 168 | 45.486 | 34.287 | 114.624 | 1 | 17.96 |
| 1302 | CE2 | PHE | 168 | 44.718 | 32.02 | 114.384 | 1 | 18.1 |
| 1303 | CZ | PHE | 168 | 44.711 | 33.229 | 115.072 | 1 | 17.59 |
| 1304 | C | PHE | 168 | 49.434 | 32.513 | 110.707 | 1 | 20.98 |
| 1305 | O | PHE | 168 | 49.583 | 32.82 | 109.519 | 1 | 18.73 |
| 1306 | N | ASN | 169 | 50.053 | 31.481 | 111.275 | 1 | 20.45 |
| 1307 | CA | ASN | 169 | 50.909 | 30.6 | 110.5 | 1 | 22.66 |
| 1308 | CB | ASN | 169 | 52.233 | 30.316 | 111.23 | 1 | 20.98 |
| 1309 | CG | ASN | 169 | 52.028 | 29.731 | 112.617 | 1 | 23.01 |
| 1310 | OD1 | ASN | 169 | 50.971 | 29.177 | 112.921 | 1 | 23.96 |
| 1311 | ND2 | ASN | 169 | 53.051 | 29.837 | 113.463 | 1 | 21.34 |
| 1312 | C | ASN | 169 | 50.13 | 29.31 | 110.276 | 1 | 22.19 |
| 1313 | O | ASN | 169 | 48.97 | 29.195 | 110.685 | 1 | 20.94 |
| 1314 | N | HIS | 170 | 50.773 | 28.355 | 109.613 | 1 | 22.88 |
| 1315 | CA | HIS | 170 | 50.187 | 27.053 | 109.294 | 1 | 24.29 |
| 1316 | CB | HIS | 170 | 51.263 | 26.184 | 108.618 | 1 | 28.3 |
| 1317 | CG | HIS | 170 | 50.883 | 24.745 | 108.455 | 1 | 31.8 |
| 1318 | CD2 | HIS | 170 | 51.495 | 23.61 | 108.869 | 1 | 34.28 |
| 1319 | ND1 | HIS | 170 | 49.749 | 24.344 | 107.784 | 1 | 35.5 |
| 1320 | CE1 | HIS | 170 | 49.675 | 23.024 | 107.793 | 1 | 34.89 |
| 1321 | NE2 | HIS | 170 | 50.723 | 22.555 | 108.446 | 1 | 36.77 |
| 1322 | C | HIS | 170 | 49.616 | 26.333 | 110.52 | 1 | 23.9 |
| 1323 | O | HIS | 170 | 48.469 | 25.882 | 110.511 | 1 | 21.22 |
| 1324 | N | ARG | 171 | 50.427 | 26.232 | 111.567 | 1 | 24.03 |
| 1325 | CA | ARG | 171 | 50.033 | 25.567 | 112.806 | 1 | 24.73 |
| 1326 | CB | ARG | 171 | 51.224 | 25.576 | 113.779 | 1 | 28.13 |
| 1327 | CG | ARG | 171 | 50.912 | 25.161 | 115.213 | 1 | 36.51 |
| 1328 | CD | ARG | 171 | 52.165 | 25.229 | 116.091 | 1 | 41.26 |
| 1329 | NE | ARG | 171 | 51.874 | 25.777 | 117.415 | 1 | 47.16 |
| 1330 | CZ | ARG | 171 | 51.533 | 27.042 | 117.639 | 1 | 48.77 |
| 1331 | NH1 | ARG | 171 | 51.443 | 27.89 | 116.625 | 1 | 53.05 |
| 1332 | NH2 | ARG | 171 | 51.276 | 27.462 | 118.871 | 1 | 50.54 |
| 1333 | C | ARG | 171 | 48.8 | 26.215 | 113.456 | 1 | 23.52 |
| 1334 | O | ARG | 171 | 47.85 | 25.527 | 113.821 | 1 | 22.84 |
| 1335 | N | LEU | 172 | 48.811 | 27.536 | 113.594 | 1 | 21.13 |
| 1336 | CA | LEU | 172 | 47.685 | 28.236 | 114.207 | 1 | 21.39 |
| 1337 | CB | LEU | 172 | 48.047 | 29.705 | 114.426 | 1 | 20.14 |
| 1338 | CG | LEU | 172 | 49.114 | 29.918 | 115.512 | 1 | 22.06 |
| 1339 | CD1 | LEU | 172 | 49.559 | 31.366 | 115.531 | 1 | 21.5 |
| 1340 | CD2 | LEU | 172 | 48.543 | 29.512 | 116.877 | 1 | 24.46 |
| 1341 | C | LEU | 172 | 46.405 | 28.117 | 113.368 | 1 | 21.98 |
| 1342 | O | LEU | 172 | 45.292 | 27.999 | 113.9 | 1 | 20.69 |
| 1343 | N | LEU | 173 | 46.561 | 28.15 | 112.052 | 1 | 21.14 |
| 1344 | CA | LEU | 173 | 45.413 | 28.014 | 111.176 | 1 | 23.89 |
| 1345 | CB | LEU | 173 | 45.843 | 28.24 | 109.723 | 1 | 25.01 |
| 1346 | CG | LEU | 173 | 45.296 | 29.426 | 108.917 | 1 | 29.42 |
| 1347 | CD1 | LEU | 173 | 44.975 | 30.622 | 109.772 | 1 | 30.46 |
| 1348 | CD2 | LEU | 173 | 46.322 | 29.768 | 107.856 | 1 | 29.01 |
| 1349 | C | LEU | 173 | 44.822 | 26.608 | 111.362 | 1 | 23.96 |
| 1350 | O | LEU | 173 | 43.61 | 26.444 | 111.482 | 1 | 23.79 |
| 1351 | N | GLU | 174 | 45.689 | 25.601 | 111.405 | 1 | 24.75 |
| 1352 | CA | GLU | 174 | 45.247 | 24.224 | 111.576 | 1 | 26.43 |
| 1353 | CB | GLU | 174 | 46.449 | 23.282 | 111.549 | 1 | 29.69 |
| 1354 | CG | GLU | 174 | 46.127 | 21.906 | 111.02 | 1 | 40.76 |
| 1355 | CD | GLU | 174 | 46.01 | 21.876 | 109.506 | 1 | 44.29 |
| 1356 | OE1 | GLU | 174 | 47.062 | 21.767 | 108.832 | 1 | 47.58 |
| 1357 | OE2 | GLU | 174 | 44.87 | 21.976 | 108.995 | 1 | 45.18 |
| 1358 | C | GLU | 174 | 44.491 | 24.067 | 112.899 | 1 | 25.16 |
| 1359 | O | GLU | 174 | 43.476 | 23.374 | 112.966 | 1 | 23.12 |
| 1360 | N | MET | 175 | 44.993 | 24.717 | 113.946 | 1 | 24.01 |
| 1361 | CA | MET | 175 | 44.364 | 24.663 | 115.26 | 1 | 25.32 |
| 1362 | CB | MET | 175 | 45.1 | 25.591 | 116.233 | 1 | 26.63 |
| 1363 | CG | MET | 175 | 44.524 | 25.62 | 117.633 | 1 | 30.63 |
| 1364 | SD | MET | 175 | 45.217 | 26.949 | 118.657 | 1 | 34.03 |
| 1365 | CE | MET | 175 | 46.64 | 26.091 | 119.374 | 1 | 39.82 |
| 1366 | C | MET | 175 | 42.886 | 25.086 | 115.167 | 1 | 24.83 |
| 1367 | O | MET | 175 | 42 | 24.436 | 115.733 | 1 | 22.66 |
| 1368 | N | ILE | 176 | 42.627 | 26.176 | 114.45 | 1 | 21.34 |
| 1369 | CA | ILE | 176 | 41.26 | 26.675 | 114.281 | 1 | 22.39 |
| 1370 | CB | ILE | 176 | 41.236 | 28.101 | 113.643 | 1 | 21.64 |
| 1371 | CG2 | ILE | 176 | 39.787 | 28.566 | 113.442 | 1 | 22.69 |
| 1372 | CG1 | ILE | 176 | 41.976 | 29.096 | 114.53 | 1 | 22.22 |
| 1373 | CD1 | ILE | 176 | 41.449 | 29.174 | 115.944 | 1 | 25.11 |
| 1374 | C | ILE | 176 | 40.427 | 25.749 | 113.396 | 1 | 22.36 |
| 1375 | O | ILE | 176 | 39.304 | 25.383 | 113.75 | 1 | 19.6 |
| 1376 | N | LEU | 177 | 40.979 | 25.376 | 112.245 | 1 | 22.2 |
| 1377 | CA | LEU | 177 | 40.271 | 24.513 | 111.313 | 1 | 25.16 |
| 1378 | CB | LEU | 177 | 41.096 | 24.301 | 110.038 | 1 | 25.81 |
| 1379 | CG | LEU | 177 | 41.358 | 25.551 | 109.191 | 1 | 27.74 |
| 1380 | CD1 | LEU | 177 | 42.259 | 25.198 | 108.013 | 1 | 26.85 |
| 1381 | CD2 | LEU | 177 | 40.03 | 26.125 | 108.704 | 1 | 28.04 |
| 1382 | C | LEU | 177 | 39.892 | 23.16 | 111.894 | 1 | 26.29 |
| 1383 | O | LEU | 177 | 38.852 | 22.607 | 111.543 | 1 | 26.05 |
| 1384 | N | ASN | 178 | 40.726 | 22.627 | 112.779 | 1 | 25.93 |
| 1385 | CA | ASN | 178 | 40.455 | 21.328 | 113.385 | 1 | 29.26 |
| 1386 | CB | ASN | 178 | 41.757 | 20.537 | 113.549 | 1 | 31.42 |
| 1387 | CG | ASN | 178 | 42.41 | 20.222 | 112.22 | 1 | 33.25 |
| 1388 | OD1 | ASN | 178 | 41.756 | 19.752 | 111.298 | 1 | 37.3 |
| 1389 | ND2 | ASN | 178 | 43.706 | 20.476 | 112.119 | 1 | 37.97 |
| 1390 | C | ASN | 178 | 39.751 | 21.441 | 114.732 | 1 | 29.32 |
| 1391 | O | ASN | 178 | 39.544 | 20.445 | 115.417 | 1 | 29.78 |
| 1392 | N | LYS | 179 | 39.377 | 22.658 | 115.103 | 1 | 27.59 |
| 1393 | CA | LYS | 179 | 38.697 | 22.881 | 116.368 | 1 | 27.88 |
| 1394 | CB | LYS | 179 | 38.444 | 24.366 | 116.571 | 1 | 27.48 |
| 1395 | CG | LYS | 179 | 37.618 | 24.689 | 117.79 | 1 | 28.61 |
| 1396 | CD | LYS | 179 | 37.127 | 26.12 | 117.718 | 1 | 30.71 |
| 1397 | CE | LYS | 179 | 36.13 | 26.396 | 118.795 | 1 | 32.98 |
| 1398 | NZ | LYS | 179 | 34.946 | 25.52 | 118.666 | 1 | 31.58 |
| 1399 | C | LYS | 179 | 37.365 | 22.139 | 116.409 | 1 | 27.87 |
| 1400 | O | LYS | 179 | 36.573 | 22.204 | 115.474 | 1 | 26.45 |
| 1401 | N | PRO | 180 | 37.107 | 21.411 | 117.499 | 1 | 28.33 |
| 1402 | CD | PRO | 180 | 37.971 | 21.14 | 118.66 | 1 | 28.78 |
| 1403 | CA | PRO | 180 | 35.84 | 20.683 | 117.597 | 1 | 28.01 |
| 1404 | CB | PRO | 180 | 36.029 | 19.827 | 118.851 | 1 | 29.62 |
| 1405 | CG | PRO | 180 | 36.981 | 20.63 | 119.676 | 1 | 31.19 |
| 1406 | C | PRO | 180 | 34.671 | 21.667 | 117.717 | 1 | 26.76 |
| 1407 | O | PRO | 180 | 34.764 | 22.676 | 118.424 | 1 | 24.04 |
| 1408 | N | GLY | 181 | 33.588 | 21.385 | 117.002 | 1 | 24 |
| 1409 | CA | GLY | 181 | 32.426 | 22.253 | 117.059 | 1 | 24.33 |
| 1410 | C | GLY | 181 | 32.545 | 23.52 | 116.232 | 1 | 24.58 |
| 1411 | O | GLY | 181 | 31.699 | 24.407 | 116.34 | 1 | 23.81 |
| 1412 | N | LEU | 182 | 33.584 | 23.6 | 115.401 | 1 | 23.25 |
| 1413 | CA | LEU | 182 | 33.817 | 24.766 | 114.547 | 1 | 23.59 |
| 1414 | CB | LEU | 182 | 34.961 | 24.488 | 113.565 | 1 | 24.45 |
| 1415 | CG | LEU | 182 | 35.326 | 25.629 | 112.611 | 1 | 23.77 |
| 1416 | CD1 | LEU | 182 | 35.98 | 26.754 | 113.405 | 1 | 23.78 |
| 1417 | CD2 | LEU | 182 | 36.281 | 25.128 | 111.524 | 1 | 25.03 |
| 1418 | C | LEU | 182 | 32.571 | 25.132 | 113.75 | 1 | 24.35 |
| 1419 | O | LEU | 182 | 31.982 | 24.288 | 113.08 | 1 | 22.97 |
| 1420 | N | LYS | 183 | 32.17 | 25.393 | 113.811 | 1 | 23.3 |
| 1421 | CA | LYS | 183 | 30.997 | 28.818 | 113.063 | 1 | 24.46 |
| 1422 | CB | LYS | 183 | 30.119 | 27.703 | 113.942 | 1 | 25.98 |
| 1423 | CG | LYS | 183 | 28.748 | 27.971 | 113.374 | 1 | 32.36 |
| 1424 | CD | LYS | 183 | 27.84 | 28.643 | 114.406 | 1 | 35.98 |
| 1425 | CE | LYS | 183 | 26.432 | 28.846 | 113.847 | 1 | 37.3 |
| 1426 | NZ | LYS | 183 | 25.525 | 29.477 | 114.843 | 1 | 36.68 |
| 1427 | C | LYS | 183 | 31.385 | 27.569 | 111.789 | 1 | 24.49 |
| 1428 | O | LYS | 183 | 30.812 | 27.337 | 110.727 | 1 | 23.6 |
| 1429 | N | TYR | 184 | 32.362 | 28.464 | 111.894 | 1 | 21.68 |
| 1430 | CA | TYR | 184 | 32.796 | 29.251 | 110.745 | 1 | 21.19 |
| 1431 | CB | TYR | 184 | 32.35 | 30.71 | 110.902 | 1 | 20.85 |
| 1432 | CG | TYR | 184 | 30.863 | 30.894 | 111.11 | 1 | 21.79 |
| 1433 | CD1 | TYR | 184 | 29.956 | 30.495 | 110.137 | 1 | 21.73 |
| 1434 | CE1 | TYR | 184 | 28.591 | 30.68 | 110.306 | 1 | 24.81 |
| 1435 | CD2 | TYR | 184 | 30.363 | 31.49 | 112.275 | 1 | 21.6 |
| 1436 | CE2 | TYR | 184 | 28.986 | 31.685 | 112.452 | 1 | 22.27 |
| 1437 | CZ | TYR | 184 | 28.112 | 31.276 | 111.461 | 1 | 24.98 |
| 1438 | OH | TYR | 184 | 26.753 | 31.457 | 111.604 | 1 | 27.58 |
| 1439 | C | TYR | 184 | 34.304 | 29.236 | 110.619 | 1 | 22.22 |
| 1440 | O | TYR | 184 | 35.011 | 29.551 | 111.581 | 1 | 20.83 |
| 1441 | N | LYS | 185 | 34.839 | 28.864 | 109.466 | 1 | 20.22 |
| 1442 | CA | LYS | 185 | 36.285 | 28.939 | 109.415 | 1 | 24.29 |
| 1443 | CB | LYS | 185 | 36.874 | 27.868 | 108.492 | 1 | 28.55 |
| 1444 | CG | LYS | 185 | 36.238 | 27.715 | 107.162 | 1 | 31.16 |
| 1445 | CD | LYS | 185 | 36.773 | 26.439 | 106.534 | 1 | 33.15 |
| 1446 | CE | LYS | 185 | 36.692 | 26.5 | 105.036 | 1 | 34.09 |
| 1447 | NZ | LYS | 185 | 37.438 | 25.381 | 104.39 | 1 | 36 |
| 1448 | C | LYS | 185 | 36.698 | 30.347 | 109.003 | 1 | 22.03 |
| 1449 | O | LYS | 185 | 35.871 | 31.138 | 108.553 | 1 | 20.79 |
| 1450 | N | PRO | 186 | 37.972 | 30.702 | 109.206 | 1 | 21.24 |
| 1451 | CD | PRO | 186 | 39.062 | 29.974 | 109.88 | 1 | 20.76 |
| 1452 | CA | PRO | 186 | 38.387 | 32.054 | 108.815 | 1 | 20.4 |
| 1453 | CB | PRO | 186 | 39.864 | 32.083 | 109.193 | 1 | 21.52 |
| 1454 | CG | PRO | 186 | 39.954 | 31.103 | 110.335 | 1 | 23.92 |
| 1455 | C | PRO | 186 | 38.189 | 32.27 | 107.314 | 1 | 18.7 |
| 1456 | O | PRO | 186 | 38.472 | 31.376 | 106.522 | 1 | 15.86 |
| 1457 | N | VAL | 187 | 37.702 | 33.446 | 106.92 | 1 | 18.54 |
| 1458 | CA | VAL | 187 | 37.517 | 33.706 | 105.497 | 1 | 18.05 |
| 1459 | CB | VAL | 187 | 36.461 | 34.824 | 105.196 | 1 | 17.24 |
| 1460 | CG1 | VAL | 187 | 35.097 | 34.444 | 105.764 | 1 | 17.86 |
| 1461 | CG2 | VAL | 187 | 36.924 | 36.161 | 105.764 | 1 | 14.04 |
| 1462 | C | VAL | 187 | 38.847 | 34.134 | 104.891 | 1 | 18.78 |
| 1463 | O | VAL | 187 | 39.049 | 34.005 | 103.679 | 1 | 19.13 |
| 1464 | N | CYS | 188 | 39.756 | 34.641 | 105.725 | 1 | 17.42 |
| 1465 | CA | CYS | 188 | 41.055 | 35.1 | 105.227 | 1 | 17.53 |
| 1466 | CB | CYS | 188 | 40.941 | 36.539 | 104.703 | 1 | 15.97 |
| 1467 | SG | CYS | 188 | 40.627 | 37.798 | 106.032 | 1 | 21.11 |
| 1468 | C | CYS | 188 | 42.142 | 35.095 | 106.29 | 1 | 17.11 |
| 1469 | O | CYS | 188 | 41.878 | 34.943 | 107.476 | 1 | 17.34 |
| 1470 | N | ASN | 189 | 43.373 | 35.273 | 105.832 | 1 | 16.95 |
| 1471 | CA | ASN | 189 | 44.522 | 35.377 | 106.715 | 1 | 17.15 |
| 1472 | CB | ASN | 189 | 45.385 | 34.108 | 106.681 | 1 | 14.74 |
| 1473 | CG | ASN | 189 | 46.527 | 34.16 | 107.677 | 1 | 16.7 |
| 1474 | OD1 | ASN | 189 | 46.525 | 34.987 | 108.587 | 1 | 18.43 |
| 1475 | ND2 | ASN | 189 | 47.516 | 33.28 | 107.508 | 1 | 17.17 |
| 1476 | C | ASN | 189 | 45.289 | 36.56 | 106.142 | 1 | 17.14 |
| 1477 | O | ASN | 189 | 45.848 | 36.464 | 105.052 | 1 | 16.91 |
| 1478 | N | GLN | 190 | 45.279 | 37.682 | 106.859 | 1 | 17.05 |
| 1479 | CA | GLN | 190 | 45.98 | 38.885 | 106.409 | 1 | 17.47 |
| 1480 | CB | GLN | 190 | 45.261 | 40.152 | 106.897 | 1 | 16.49 |
| 1481 | CG | GLN | 190 | 45.905 | 41.437 | 106.373 | 1 | 18.11 |
| 1482 | CD | GLN | 190 | 45.299 | 42.705 | 106.951 | 1 | 17.19 |
| 1483 | OE1 | GLN | 190 | 46.012 | 43.546 | 107.504 | 1 | 17.23 |
| 1484 | NE2 | GLN | 190 | 43.985 | 42.861 | 106.806 | 1 | 17.87 |
| 1485 | C | GLN | 190 | 47.411 | 38.838 | 106.948 | 1 | 18.81 |
| 1486 | O | GLN | 190 | 47.631 | 38.895 | 108.158 | 1 | 17.72 |
| 1487 | N | VAL | 191 | 48.377 | 38.709 | 106.038 | 1 | 18.5 |
| 1488 | CA | VAL | 191 | 49.784 | 38.618 | 106.414 | 1 | 18.2 |
| 1489 | CB | VAL | 191 | 50.301 | 37.16 | 106.312 | 1 | 19.63 |
| 1490 | CG1 | VAL | 191 | 49.566 | 36.259 | 107.306 | 1 | 17.52 |
| 1491 | CG2 | VAL | 191 | 50.105 | 36.634 | 104.888 | 1 | 16.96 |
| 1492 | C | VAL | 191 | 50.671 | 39.47 | 105.514 | 1 | 20.88 |
| 1493 | O | VAL | 191 | 50.231 | 39.973 | 104.483 | 1 | 19.09 |
| 1494 | N | GLU | 192 | 51.93 | 39.629 | 105.908 | 1 | 19.94 |
| 1495 | CA | GLU | 192 | 52.872 | 40.398 | 105.1 | 1 | 20.74 |
| 1496 | CB | GLU | 192 | 54.2 | 40.567 | 105.843 | 1 | 22.62 |
| 1497 | CG | GLU | 192 | 55.229 | 41.419 | 105.094 | 1 | 23.94 |
| 1498 | CD | GLU | 192 | 56.525 | 41.599 | 105.873 | 1 | 27.59 |
| 1499 | OE1 | GLU | 192 | 56.49 | 41.569 | 107.129 | 1 | 25.12 |
| 1500 | OE2 | GLU | 192 | 57.577 | 41.792 | 105.226 | 1 | 26.79 |
| 1501 | C | GLU | 192 | 53.118 | 39.609 | 103.814 | 1 | 19.96 |
| 1502 | O | GLU | 192 | 53.343 | 38.403 | 103.866 | 1 | 18.25 |
| 1503 | N | CYS | 193 | 53.086 | 40.281 | 102.666 | 1 | 20.13 |
| 1504 | CA | CYS | 193 | 53.326 | 39.593 | 101.406 | 1 | 21.76 |
| 1505 | CB | CYS | 193 | 52.079 | 38.819 | 100.987 | 1 | 19.37 |
| 1506 | SG | CYS | 193 | 52.408 | 37.619 | 99.683 | 1 | 23.32 |
| 1507 | C | CYS | 193 | 53.736 | 40.568 | 100.31 | 1 | 21.4 |
| 1508 | O | CYS | 193 | 53.053 | 41.557 | 100.055 | 1 | 21.27 |
| 1509 | N | HIS | 194 | 54.865 | 40.279 | 99.671 | 1 | 23.08 |
| 1510 | CA | HIS | 194 | 55.399 | 41.124 | 98.609 | 1 | 24.27 |
| 1511 | CB | HIS | 194 | 56.004 | 42.402 | 99.206 | 1 | 23.92 |
| 1512 | CG | HIS | 194 | 56.972 | 42.143 | 100.321 | 1 | 25.35 |
| 1513 | CD2 | HIS | 194 | 58.256 | 41.72 | 100.303 | 1 | 23.07 |
| 1514 | ND1 | HIS | 194 | 56.621 | 42.246 | 101.652 | 1 | 27.77 |
| 1515 | CE1 | HIS | 194 | 57.648 | 41.892 | 102.404 | 1 | 24.38 |
| 1516 | NE2 | HIS | 194 | 58.653 | 41.567 | 101.611 | 1 | 27.71 |
| 1517 | C | HIS | 194 | 56.464 | 40.321 | 97.864 | 1 | 24.71 |
| 1518 | O | HIS | 194 | 56.789 | 39.198 | 98.254 | 1 | 24.63 |
| 1519 | N | PRO | 195 | 57.021 | 40.882 | 96.773 | 1 | 26.31 |
| 1520 | CD | PRO | 195 | 56.625 | 42.142 | 96.115 | 1 | 26.37 |
| 1521 | CA | PRO | 195 | 58.048 | 40.183 | 95.989 | 1 | 25.75 |
| 1522 | CB | PRO | 195 | 58.438 | 41.22 | 94.937 | 1 | 25.95 |
| 1523 | CG | PRO | 195 | 57.132 | 41.94 | 94.692 | 1 | 28.17 |
| 1524 | C | PRO | 195 | 59.263 | 39.645 | 96.762 | 1 | 26.5 |
| 1525 | O | PRO | 195 | 59.886 | 38.667 | 96.336 | 1 | 27.97 |
| 1526 | N | TYR | 196 | 59.617 | 40.274 | 97.88 | 1 | 26.19 |
| 1527 | CA | TYR | 196 | 60.76 | 39.791 | 98.658 | 1 | 28.47 |
| 1528 | CB | TYR | 196 | 61.483 | 40.944 | 99.348 | 1 | 30.42 |
| 1529 | CG | TYR | 196 | 62.294 | 41.826 | 98.416 | 1 | 32.54 |
| 1530 | CD1 | TYR | 196 | 62.64 | 41.401 | 97.131 | 1 | 33.58 |
| 1531 | CE1 | TYR | 196 | 63.42 | 42.205 | 96.292 | 1 | 34.32 |
| 1532 | CD2 | TYR | 196 | 62.744 | 43.076 | 98.84 | 1 | 33.82 |
| 1533 | CE2 | TYR | 196 | 63.517 | 43.884 | 98.017 | 1 | 35.16 |
| 1534 | CZ | TYR | 196 | 63.851 | 43.441 | 96.743 | 1 | 35.05 |
| 1535 | OH | TYR | 196 | 64.612 | 44.254 | 95.939 | 1 | 35.28 |
| 1536 | C | TYR | 196 | 60.361 | 38.749 | 99.697 | 1 | 29.4 |
| 1537 | O | TYR | 196 | 61.217 | 38.175 | 100.374 | 1 | 29.07 |
| 1538 | N | PHE | 197 | 59.059 | 38.507 | 99.816 | 1 | 28.6 |
| 1539 | CA | PHE | 197 | 58.539 | 37.518 | 100.756 | 1 | 28.65 |
| 1540 | CB | PHE | 197 | 58.412 | 38.154 | 102.143 | 1 | 29.99 |
| 1541 | CG | PHE | 197 | 58.385 | 37.157 | 103.267 | 1 | 34.19 |
| 1542 | CD1 | PHE | 197 | 59.536 | 36.462 | 103.626 | 1 | 35.26 |
| 1543 | CD2 | PHE | 197 | 57.205 | 36.897 | 103.954 | 1 | 34.95 |
| 1544 | CE1 | PHE | 197 | 59.513 | 35.519 | 104.651 | 1 | 36.46 |
| 1545 | CE2 | PHE | 197 | 57.169 | 35.955 | 104.983 | 1 | 35.86 |
| 1546 | CZ | PHE | 197 | 58.326 | 35.263 | 105.332 | 1 | 36.97 |
| 1547 | C | PHE | 197 | 57.164 | 37.14 | 100.203 | 1 | 27.15 |
| 1548 | O | PHE | 197 | 56.142 | 37.528 | 100.765 | 1 | 25.95 |
| 1549 | N | ASN | 198 | 57.152 | 36.376 | 99.108 | 1 | 26.53 |
| 1550 | CA | ASN | 198 | 55.91 | 36.01 | 98.437 | 1 | 25.85 |
| 1551 | CB | ASN | 198 | 56.187 | 35.616 | 96.97 | 1 | 26.42 |
| 1552 | CG | ASN | 198 | 56.943 | 34.315 | 96.831 | 1 | 26.28 |
| 1553 | OD1 | ASN | 198 | 56.717 | 33.361 | 97.57 | 1 | 27.54 |
| 1554 | ND2 | ASN | 198 | 57.836 | 34.262 | 95.85 | 1 | 28.12 |
| 1555 | C | ASN | 198 | 54.992 | 34.982 | 99.097 | 1 | 24.9 |
| 1556 | O | ASN | 198 | 53.934 | 34.674 | 98.559 | 1 | 23.53 |
| 1557 | N | GLN | 199 | 55.393 | 34.443 | 100.244 | 1 | 24.33 |
| 1558 | CA | GLN | 199 | 54.543 | 33.513 | 100.991 | 1 | 25.12 |
| 1559 | CB | GLN | 199 | 53.378 | 34.302 | 101.612 | 1 | 23.81 |
| 1560 | CG | GLN | 199 | 53.817 | 35.412 | 102.578 | 1 | 23.35 |
| 1561 | CD | GLN | 199 | 53.912 | 34.925 | 104.018 | 1 | 23.03 |
| 1562 | OE1 | GLN | 199 | 54.128 | 33.744 | 104.256 | 1 | 23.69 |
| 1563 | NE2 | GLN | 199 | 53.759 | 35.839 | 104.981 | 1 | 21.88 |
| 1564 | C | GLN | 199 | 53.969 | 32.347 | 100.195 | 1 | 26.39 |
| 1565 | O | GLN | 199 | 52.883 | 31.851 | 100.51 | 1 | 25.84 |
| 1566 | N | ARG | 200 | 54.693 | 31.89 | 99.18 | 1 | 26.76 |
| 1567 | CA | ARG | 200 | 54.211 | 30.797 | 98.339 | 1 | 26.95 |
| 1568 | CB | ARG | 200 | 55.335 | 30.346 | 97.39 | 1 | 31.47 |
| 1569 | CG | ARG | 200 | 54.882 | 29.526 | 96.184 | 1 | 38.93 |
| 1570 | CD | ARG | 200 | 55.38 | 28.086 | 96.268 | 1 | 44.51 |
| 1571 | NE | ARG | 200 | 54.485 | 27.254 | 97.075 | 1 | 48.92 |
| 1572 | CZ | ARG | 200 | 54.768 | 26.022 | 97.494 | 1 | 49.45 |
| 1573 | NH1 | ARG | 200 | 55.935 | 25.465 | 97.188 | 1 | 51.27 |
| 1574 | NH2 | ARG | 200 | 53.879 | 25.346 | 98.21 | 1 | 47.89 |
| 1575 | C | ARG | 200 | 53.658 | 29.598 | 99.12 | 1 | 25.86 |
| 1576 | O | ARG | 200 | 52.563 | 29.112 | 98.823 | 1 | 23.36 |
| 1577 | N | LYS | 201 | 54.393 | 29.117 | 100.121 | 1 | 23.87 |
| 1578 | CA | LYS | 201 | 53.913 | 27.959 | 100.884 | 1 | 25.87 |
| 1579 | CB | LYS | 201 | 54.977 | 27.474 | 101.876 | 1 | 29.05 |
| 1580 | CG | LYS | 201 | 56.172 | 26.75 | 101.257 | 1 | 36.51 |
| 1581 | CD | LYS | 201 | 57.085 | 26.212 | 102.358 | 1 | 40.48 |
| 1582 | CE | LYS | 201 | 58.328 | 25.532 | 101.8 | 1 | 44.89 |
| 1583 | NZ | LYS | 201 | 58.001 | 24.347 | 100.958 | 1 | 47.66 |
| 1584 | C | LYS | 201 | 52.61 | 28.227 | 101.648 | 1 | 23.98 |
| 1585 | O | LYS | 201 | 51.687 | 27.411 | 101.622 | 1 | 23.69 |
| 1586 | N | LEU | 202 | 52.551 | 29.358 | 102.34 | 1 | 22.85 |
| 1587 | CA | LEU | 202 | 51.362 | 29.717 | 103.097 | 1 | 23.18 |
| 1588 | CB | LEU | 202 | 51.636 | 30.954 | 103.956 | 1 | 20.86 |
| 1589 | CG | LEU | 202 | 50.474 | 31.425 | 104.855 | 1 | 22.77 |
| 1590 | CD1 | LEU | 202 | 50.004 | 30.272 | 105.741 | 1 | 21.21 |
| 1591 | CD2 | LEU | 202 | 50.918 | 32.606 | 105.717 | 1 | 22.29 |
| 1592 | C | LEU | 202 | 50.186 | 29.975 | 102.15 | 1 | 21.97 |
| 1593 | O | LEU | 202 | 49.043 | 29.602 | 102.439 | 1 | 22.71 |
| 1594 | N | LEU | 203 | 50.466 | 30.608 | 101.012 | 1 | 21.34 |
| 1595 | CA | LEU | 203 | 49.415 | 30.893 | 100.035 | 1 | 20.14 |
| 1596 | CB | LEU | 203 | 49.969 | 31.739 | 98.873 | 1 | 19.23 |
| 1597 | CG | LEU | 203 | 49.06 | 32.017 | 97.659 | 1 | 19.8 |
| 1598 | CD1 | LEU | 203 | 47.774 | 32.712 | 98.087 | 1 | 18.79 |
| 1599 | CD2 | LEU | 203 | 49.826 | 32.891 | 96.657 | 1 | 17.97 |
| 1600 | C | LEU | 203 | 48.785 | 29.607 | 99.515 | 1 | 20.34 |
| 1601 | O | LEU | 203 | 47.556 | 29.501 | 99.451 | 1 | 21.16 |
| 1602 | N | ASP | 204 | 49.602 | 28.62 | 99.149 | 1 | 21.43 |
| 1603 | CA | ASP | 204 | 49.046 | 27.353 | 98.658 | 1 | 21.76 |
| 1604 | CB | ASP | 204 | 50.147 | 26.4 | 98.163 | 1 | 24.09 |
| 1605 | CG | ASP | 204 | 50.626 | 26.742 | 96.753 | 1 | 27.58 |
| 1606 | OD1 | ASP | 204 | 49.828 | 27.309 | 95.975 | 1 | 27.53 |
| 1607 | OD2 | ASP | 204 | 51.785 | 26.431 | 96.416 | 1 | 28.62 |
| 1608 | C | ASP | 204 | 48.242 | 26.661 | 99.746 | 1 | 21.55 |
| 1609 | O | ASP | 204 | 47.205 | 26.059 | 99.464 | 1 | 21.62 |
| 1610 | N | PHE | 205 | 48.718 | 26.733 | 100.987 | 1 | 21 |
| 1611 | CA | PHE | 205 | 47.979 | 26.105 | 102.074 | 1 | 21.06 |
| 1612 | CB | PHE | 205 | 48.706 | 26.247 | 103.41 | 1 | 22.19 |
| 1613 | CG | PHE | 205 | 47.909 | 25.714 | 104.577 | 1 | 24.78 |
| 1614 | CD1 | PHE | 205 | 47.711 | 24.347 | 104.733 | 1 | 25.01 |
| 1615 | CD2 | PHE | 205 | 47.303 | 26.582 | 105.478 | 1 | 24.02 |
| 1616 | CE1 | PHE | 205 | 48.913 | 23.846 | 105.771 | 1 | 27.56 |
| 1617 | CE2 | PHE | 205 | 46.505 | 26.091 | 106.517 | 1 | 26.08 |
| 1618 | CZ | PHE | 205 | 46.311 | 24.722 | 106.661 | 1 | 25.41 |
| 1619 | C | PHE | 205 | 46.6 | 26.764 | 102.185 | 1 | 19.7 |
| 1620 | O | PHE | 205 | 45.582 | 26.077 | 102.26 | 1 | 19.85 |
| 1621 | N | CYS | 206 | 46.568 | 28.092 | 102.207 | 1 | 18.87 |
| 1622 | CA | CYS | 206 | 45.295 | 28.801 | 102.301 | 1 | 21.52 |
| 1623 | CB | CYS | 206 | 45.524 | 30.318 | 102.374 | 1 | 21.11 |
| 1624 | SG | CYS | 206 | 46.169 | 30.88 | 103.968 | 1 | 23.47 |
| 1625 | C | CYS | 206 | 44.35 | 28.46 | 101.147 | 1 | 20.35 |
| 1626 | O | CYS | 206 | 43.146 | 28.246 | 101.355 | 1 | 21.75 |
| 1627 | N | LYS | 207 | 44.879 | 28.415 | 99.93 | 1 | 20.83 |
| 1628 | CA | LYS | 207 | 44.049 | 28.079 | 98.771 | 1 | 21.69 |
| 1629 | CB | LYS | 207 | 44.87 | 28.139 | 97.481 | 1 | 22.56 |
| 1630 | CG | LYS | 207 | 45.175 | 29.545 | 97.019 | 1 | 23.24 |
| 1631 | CD | LYS | 207 | 46.032 | 29.545 | 95.777 | 1 | 25.16 |
| 1632 | CE | LYS | 207 | 46.236 | 30.963 | 95.275 | 1 | 27.78 |
| 1633 | NZ | LYS | 207 | 47.12 | 31.017 | 94.087 | 1 | 31.64 |
| 1634 | C | LYS | 207 | 43.441 | 26.693 | 98.905 | 1 | 21.5 |
| 1635 | O | LYS | 207 | 42.29 | 26.484 | 98.526 | 1 | 20.72 |
| 1636 | N | SER | 208 | 44.217 | 25.747 | 99.433 | 1 | 22.01 |
| 1637 | CA | SER | 208 | 43.725 | 24.385 | 99.597 | 1 | 23.65 |
| 1638 | CB | SER | 208 | 44.854 | 23.454 | 100.062 | 1 | 23.05 |
| 1639 | OG | SER | 208 | 45.144 | 23.632 | 101.437 | 1 | 24.76 |
| 1640 | C | SER | 208 | 42.559 | 24.339 | 100.59 | 1 | 22.4 |
| 1641 | O | SER | 208 | 41.764 | 23.407 | 100.563 | 1 | 23.6 |
| 1642 | N | LYS | 209 | 42.446 | 25.348 | 101.449 | 1 | 22.91 |
| 1643 | CA | LYS | 209 | 41.357 | 25.391 | 102.442 | 1 | 24.13 |
| 1644 | CB | LYS | 209 | 41.932 | 25.673 | 103.837 | 1 | 25.7 |
| 1645 | CG | LYS | 209 | 42.998 | 24.683 | 104.294 | 1 | 28.43 |
| 1646 | CD | LYS | 209 | 42.431 | 23.266 | 104.387 | 1 | 31.36 |
| 1647 | CE | LYS | 209 | 43.501 | 22.265 | 104.799 | 1 | 33.97 |
| 1648 | NZ | LYS | 209 | 42.959 | 20.882 | 104.989 | 1 | 36.69 |
| 1649 | C | LYS | 209 | 40.321 | 26.465 | 102.104 | 1 | 23.79 |
| 1650 | O | LYS | 209 | 39.426 | 26.744 | 102.899 | 1 | 23.5 |
| 1651 | N | ASP | 210 | 40.448 | 27.043 | 100.911 | 1 | 23.78 |
| 1652 | CA | ASP | 210 | 39.579 | 28.119 | 100.432 | 1 | 23.96 |
| 1653 | CB | ASP | 210 | 38.115 | 27.677 | 100.257 | 1 | 26.36 |
| 1654 | CG | ASP | 210 | 37.301 | 28.697 | 99.448 | 1 | 29.34 |
| 1655 | OD1 | ASP | 210 | 36.07 | 28.818 | 99.644 | 1 | 30.37 |
| 1656 | OD2 | ASP | 210 | 37.911 | 29.385 | 98.597 | 1 | 28.41 |
| 1657 | C | ASP | 210 | 39.611 | 29.325 | 101.365 | 1 | 23.52 |
| 1658 | O | ASP | 210 | 38.579 | 29.943 | 101.643 | 1 | 23.64 |
| 1659 | N | ILE | 211 | 40.802 | 29.641 | 101.857 | 1 | 20.71 |
| 1660 | CA | ILE | 211 | 41.008 | 30.798 | 102.716 | 1 | 19.71 |
| 1661 | CB | ILE | 211 | 41.887 | 30.443 | 103.936 | 1 | 20.89 |
| 1662 | CG2 | ILE | 211 | 42.266 | 31.718 | 104.699 | 1 | 18.01 |
| 1663 | CG1 | ILE | 211 | 41.145 | 29.441 | 104.835 | 1 | 20.41 |
| 1664 | CD1 | ILE | 211 | 41.958 | 28.975 | 106.045 | 1 | 21.68 |
| 1665 | C | ILE | 211 | 41.75 | 31.809 | 101.847 | 1 | 21.09 |
| 1666 | O | ILE | 211 | 42.75 | 31.465 | 101.217 | 1 | 19 |
| 1667 | N | VAL | 212 | 41.251 | 33.04 | 101.792 | 1 | 20.06 |
| 1668 | CA | VAL | 212 | 41.882 | 34.076 | 100.977 | 1 | 21.03 |
| 1669 | CB | VAL | 212 | 40.867 | 35.191 | 100.604 | 1 | 22.91 |
| 1670 | CG1 | VAL | 212 | 41.592 | 36.368 | 99.972 | 1 | 22.76 |
| 1671 | CG2 | VAL | 212 | 39.806 | 34.639 | 99.644 | 1 | 22.98 |
| 1672 | C | VAL | 212 | 43.046 | 34.725 | 101.712 | 1 | 21.05 |
| 1673 | O | VAL | 212 | 42.932 | 35.058 | 102.889 | 1 | 20.92 |
| 1674 | N | LEU | 213 | 44.171 | 34.9 | 101.025 | 1 | 20.26 |
| 1675 | CA | LEU | 213 | 45.32 | 35.546 | 101.645 | 1 | 20.09 |
| 1676 | CB | LEU | 213 | 46.633 | 34.929 | 101.13 | 1 | 20.99 |
| 1677 | CG | LEU | 213 | 47.907 | 35.315 | 101.904 | 1 | 21.62 |
| 1678 | CD1 | LEU | 213 | 48.979 | 34.25 | 101.737 | 1 | 25.02 |
| 1579 | CD2 | LEU | 213 | 48.407 | 35.657 | 101.421 | 1 | 24.82 |
| 1680 | C | LEU | 213 | 45.245 | 37.033 | 101.307 | 1 | 20.03 |
| 1681 | O | LEU | 213 | 45.068 | 37.407 | 100.149 | 1 | 22.02 |
| 1682 | N | VAL | 214 | 45.339 | 37.883 | 102.323 | 1 | 20.42 |
| 1683 | CA | VAL | 214 | 45.287 | 39.326 | 102.119 | 1 | 17.7 |
| 1684 | CB | VAL | 214 | 44.221 | 39.99 | 103.026 | 1 | 18.15 |
| 1685 | CG1 | VAL | 214 | 44.234 | 41.504 | 102.834 | 1 | 15.93 |
| 1686 | CG2 | VAL | 214 | 42.834 | 39.416 | 102.69 | 1 | 17.11 |
| 1687 | C | VAL | 214 | 46.675 | 39.85 | 102.459 | 1 | 19.11 |
| 1688 | O | VAL | 214 | 47.179 | 39.646 | 103.57 | 1 | 17.5 |
| 1689 | N | ALA | 215 | 47.29 | 40.526 | 101.495 | 1 | 17.91 |
| 1690 | CA | ALA | 215 | 48.643 | 41.03 | 101.659 | 1 | 19.16 |
| 1691 | CB | ALA | 215 | 49.387 | 40.953 | 100.312 | 1 | 18.93 |
| 1692 | C | ALA | 215 | 48.773 | 42.426 | 102.203 | 1 | 19.94 |
| 1693 | O | ALA | 215 | 48.172 | 43.361 | 101.672 | 1 | 20.38 |
| 1694 | N | TYR | 216 | 49.556 | 42.572 | 103.269 | 1 | 19.96 |
| 1695 | CA | TYR | 216 | 49.815 | 43.899 | 103.795 | 1 | 20.9 |
| 1696 | CB | TYR | 216 | 49.424 | 44.028 | 105.277 | 1 | 19.49 |
| 1697 | CG | TYR | 216 | 50.232 | 43.251 | 106.286 | 1 | 18.59 |
| 1698 | CD1 | TYR | 216 | 51.564 | 43.577 | 106.551 | 1 | 17.27 |
| 1699 | CE1 | TYR | 216 | 52.262 | 42.984 | 107.618 | 1 | 20.63 |
| 1700 | CD2 | TYR | 216 | 49.613 | 42.293 | 107.096 | 1 | 19.09 |
| 1701 | CE2 | TYR | 216 | 50.295 | 41.689 | 108.164 | 1 | 19.94 |
| 1702 | CZ | TYR | 216 | 51.615 | 42.047 | 108.423 | 1 | 20.81 |
| 1703 | OH | TYR | 216 | 52.26 | 41.511 | 109.518 | 1 | 20.35 |
| 1704 | C | TYR | 216 | 51.3 | 44.174 | 103.546 | 1 | 20.4 |
| 1705 | O | TYR | 216 | 52.064 | 43.251 | 103.249 | 1 | 20.03 |
| 1706 | N | SER | 217 | 51.69 | 45.441 | 103.632 | 1 | 22.14 |
| 1707 | CA | SER | 217 | 53.06 | 45.857 | 103.352 | 1 | 24.39 |
| 1708 | CB | SER | 217 | 54.022 | 45.31 | 104.411 | 1 | 26.95 |
| 1709 | OG | SER | 217 | 53.768 | 45.944 | 105.663 | 1 | 30.12 |
| 1710 | C | SER | 217 | 53.454 | 45.373 | 101.958 | 1 | 24.21 |
| 1711 | O | SER | 217 | 54.61 | 45.034 | 101.702 | 1 | 25.59 |
| 1712 | N | ALA | 218 | 52.476 | 45.361 | 101.053 | 1 | 24.73 |
| 1713 | CA | ALA | 218 | 52.685 | 44.903 | 99.676 | 1 | 24.91 |
| 1714 | CB | ALA | 218 | 51.339 | 44.762 | 98.965 | 1 | 25.44 |
| 1715 | C | ALA | 218 | 53.597 | 45.83 | 98.88 | 1 | 25.61 |
| 1716 | O | ALA | 218 | 54.077 | 45.459 | 97.808 | 1 | 27.04 |
| 1717 | N | LEU | 219 | 53.822 | 47.031 | 99.405 | 1 | 26.13 |
| 1718 | CA | LEU | 219 | 54.694 | 48.013 | 98.768 | 1 | 27.81 |
| 1719 | CB | LEU | 219 | 53.979 | 49.364 | 98.652 | 1 | 25.58 |
| 1720 | CG | LEU | 219 | 52.701 | 49.368 | 97.802 | 1 | 26.17 |
| 1721 | CD1 | LEU | 219 | 52.123 | 50.779 | 97.73 | 1 | 25.96 |
| 1722 | CD2 | LEU | 219 | 53.013 | 48.848 | 96.406 | 1 | 25.65 |
| 1723 | C | LEU | 219 | 56.011 | 48.191 | 99.53 | 1 | 28.63 |
| 1724 | O | LEU | 219 | 56.749 | 49.148 | 99.287 | 1 | 29.16 |
| 1725 | N | GLY | 220 | 56.302 | 47.281 | 100.459 | 1 | 29.75 |
| 1726 | CA | GLY | 220 | 57.547 | 47.374 | 101.207 | 1 | 30.62 |
| 1727 | C | GLY | 220 | 57.427 | 47.906 | 102.624 | 1 | 31.28 |
| 1728 | O | GLY | 220 | 58.443 | 48.092 | 103.303 | 1 | 29.88 |
| 1729 | N | SER | 221 | 56.187 | 45.166 | 103.046 | 1 | 29.92 |
| 1730 | CA | SER | 221 | 55.846 | 48.667 | 104.383 | 1 | 32.01 |
| 1731 | CB | SER | 221 | 56.544 | 47.841 | 105.474 | 1 | 31.88 |
| 1732 | OG | SER | 221 | 57.806 | 48.401 | 105.804 | 1 | 31.34 |
| 1733 | C | SER | 221 | 56.161 | 50.138 | 104.61 | 1 | 33.56 |
| 1734 | O | SER | 221 | 56.877 | 50.762 | 103.832 | 1 | 32.52 |
| 1735 | N | HIS | 222 | 55.61 | 50.675 | 105.694 | 1 | 35.54 |
| 1736 | CA | HIS | 222 | 55.82 | 52.065 | 106.073 | 1 | 37.26 |
| 1737 | CB | HIS | 222 | 54.874 | 52.434 | 107.215 | 1 | 38.35 |
| 1738 | CG | HIS | 222 | 54.804 | 51.4 | 108.297 | 1 | 40.7 |
| 1739 | CD2 | HIS | 222 | 55.685 | 51.064 | 109.27 | 1 | 40.28 |
| 1740 | ND1 | HIS | 222 | 53.726 | 50.552 | 108.448 | 1 | 42.24 |
| 1741 | CE1 | HIS | 222 | 53.944 | 49.742 | 109.469 | 1 | 41.92 |
| 1742 | NE2 | HIS | 222 | 55.126 | 50.032 | 109.985 | 1 | 42.06 |
| 1743 | C | HIS | 222 | 57.262 | 52.323 | 106.513 | 1 | 38.38 |
| 1744 | O | HIS | 222 | 57.695 | 53.471 | 106.587 | 1 | 39.38 |
| 1745 | N | ARG | 223 | 58 | 51.254 | 106.805 | 1 | 39.44 |
| 1746 | CA | ARG | 223 | 59.388 | 51.368 | 107.243 | 1 | 40.59 |
| 1747 | CB | ARG | 223 | 60.265 | 51.922 | 106.112 | 1 | 40.47 |
| 1748 | CG | ARG | 223 | 60.263 | 51.072 | 104.84 | 1 | 40.92 |
| 1749 | CD | ARG | 223 | 61.224 | 51.637 | 103.806 | 1 | 40.98 |
| 1750 | NE | ARG | 223 | 62.611 | 51.502 | 104.244 | 1 | 40.62 |
| 1751 | CZ | ARG | 223 | 63.344 | 50.401 | 104.096 | 1 | 41.19 |
| 1752 | NH1 | ARG | 223 | 62.828 | 49.329 | 103.509 | 1 | 39.72 |
| 1753 | NH2 | ARG | 223 | 64.593 | 50.362 | 104.551 | 1 | 40.52 |
| 1754 | C | ARG | 223 | 59.529 | 52.255 | 108.477 | 1 | 41.44 |
| 1755 | O | ARG | 223 | 60.501 | 52.995 | 108.604 | 1 | 42.07 |
| 1756 | N | GLU | 224 | 58.563 | 52.178 | 109.388 | 1 | 42.47 |
| 1757 | CA | GLU | 224 | 58.599 | 52.982 | 110.609 | 1 | 44.5 |
| 1758 | CB | GLU | 224 | 57.18 | 53.191 | 111.146 | 1 | 45.56 |
| 1759 | CG | GLU | 224 | 57.075 | 54.202 | 112.277 | 1 | 47.34 |
| 1760 | CD | GLU | 224 | 55.732 | 54.142 | 112.987 | 1 | 49.12 |
| 1761 | OE1 | GLU | 224 | 54.743 | 53.719 | 112.357 | 1 | 50.19 |
| 1762 | OE2 | GLU | 224 | 55.658 | 54.524 | 114.172 | 1 | 49.07 |
| 1763 | C | GLU | 224 | 59.443 | 52.312 | 111.695 | 1 | 44.7 |
| 1764 | O | GLU | 224 | 59.279 | 51.127 | 111.978 | 1 | 42.69 |
| 1765 | N | GLU | 225 | 60.35 | 53.075 | 112.296 | 1 | 46.25 |
| 1766 | CA | GLU | 225 | 61.194 | 52.558 | 113.368 | 1 | 47.31 |
| 1767 | CB | GLU | 225 | 62.481 | 53.371 | 113.466 | 1 | 49.21 |
| 1768 | CG | GLU | 225 | 63.417 | 53.203 | 112.286 | 1 | 53.19 |
| 1769 | CD | GLU | 225 | 64.614 | 54.13 | 112.369 | 1 | 55.54 |
| 1770 | OE1 | GLU | 225 | 64.472 | 55.323 | 112.018 | 1 | 56.97 |
| 1771 | OE2 | GLU | 225 | 65.691 | 53.668 | 112.799 | 1 | 56.42 |
| 1772 | C | GLU | 225 | 60.416 | 52.68 | 114.673 | 1 | 46.84 |
| 1773 | O | GLU | 225 | 59.602 | 53.587 | 114.825 | 1 | 45.7 |
| 1774 | N | PRO | 226 | 60.659 | 51.765 | 115.629 | 1 | 47.12 |
| 1775 | CD | PRO | 226 | 60.318 | 52.004 | 117.037 | 1 | 48.37 |
| 1776 | CA | PRO | 226 | 61.613 | 50.653 | 115.53 | 1 | 47.43 |
| 1777 | CB | PRO | 226 | 62.056 | 50.435 | 116.981 | 1 | 40.28 |
| 1778 | CG | PRO | 226 | 61.623 | 51.685 | 117.707 | 1 | 49.15 |
| 1779 | C | PRO | 226 | 60.983 | 49.385 | 114.967 | 1 | 46.53 |
| 1780 | O | PRO | 226 | 61.581 | 48.315 | 115.036 | 1 | 47.34 |
| 1781 | N | TRP | 227 | 59.779 | 49.504 | 114.42 | 1 | 45.2 |
| 1782 | CA | TRP | 227 | 59.074 | 48.347 | 113.875 | 1 | 44.16 |
| 1783 | CB | TRP | 227 | 57.657 | 48.744 | 113.468 | 1 | 46.38 |
| 1784 | CG | TRP | 227 | 56.908 | 49.418 | 114.561 | 1 | 48.83 |
| 1785 | CD2 | TRP | 227 | 56.443 | 48.819 | 115.776 | 1 | 49.79 |
| 1786 | CE2 | TRP | 227 | 55.815 | 49.836 | 116.528 | 1 | 50.43 |
| 1787 | CE3 | TRP | 227 | 56.495 | 47.52 | 116.302 | 1 | 50.77 |
| 1788 | CD1 | TRP | 227 | 56.555 | 50.734 | 114.622 | 1 | 49.43 |
| 1789 | NE1 | TRP | 227 | 55.899 | 50.994 | 115.8 | 1 | 50.06 |
| 1790 | CZ2 | TRP | 227 | 55.243 | 49.597 | 117.784 | 1 | 51.29 |
| 1791 | CZ3 | TRP | 227 | 55.925 | 47.281 | 117.553 | 1 | 51.95 |
| 1792 | CH2 | TRP | 227 | 55.307 | 48.318 | 118.278 | 1 | 51.69 |
| 1793 | C | TRP | 227 | 59.78 | 47.705 | 112.686 | 1 | 42.72 |
| 1794 | O | TRP | 227 | 59.835 | 48.48 | 112.577 | 1 | 41.39 |
| 1795 | N | VAL | 228 | 60.311 | 48.532 | 111.794 | 1 | 40.57 |
| 1796 | CA | VAL | 228 | 61.006 | 48.029 | 110.619 | 1 | 40.35 |
| 1797 | CB | VAL | 228 | 60.378 | 48.583 | 109.323 | 1 | 39.03 |
| 1798 | CG1 | VAL | 228 | 61.076 | 47.991 | 108.105 | 1 | 37.37 |
| 1799 | CG2 | VAL | 228 | 58.894 | 48.274 | 109.295 | 1 | 37.04 |
| 1800 | C | VAL | 228 | 62.475 | 48.425 | 110.653 | 1 | 41.11 |
| 1801 | O | VAL | 228 | 62.807 | 49.579 | 110.918 | 1 | 41.12 |
| 1802 | N | ASP | 229 | 63.344 | 47.46 | 110.375 | 1 | 42.45 |
| 1803 | CA | ASP | 229 | 64.783 | 47.686 | 110.366 | 1 | 44.15 |
| 1804 | CB | ASP | 229 | 65.518 | 46.342 | 110.326 | 1 | 44.85 |
| 1805 | CG | ASP | 229 | 67.017 | 46.501 | 110.153 | 1 | 46.51 |
| 1806 | OD1 | ASP | 229 | 67.565 | 47.529 | 110.597 | 1 | 48.44 |
| 1807 | OD2 | ASP | 229 | 67.651 | 45.591 | 109.588 | 1 | 46.66 |
| 1808 | C | ASP | 229 | 65.206 | 48.552 | 109.182 | 1 | 44.5 |
| 1809 | O | ASP | 229 | 65.016 | 48.171 | 108.026 | 1 | 43.42 |
| 1810 | N | PRO | 230 | 65.783 | 49.735 | 109.463 | 1 | 45.57 |
| 1811 | CD | PRO | 230 | 65.977 | 50.287 | 110.814 | 1 | 46.21 |
| 1812 | CA | PRO | 230 | 65.245 | 50.69 | 108.447 | 1 | 45.92 |
| 1813 | CB | PRO | 230 | 66.863 | 51.809 | 109.281 | 1 | 46.03 |
| 1814 | CG | PRO | 230 | 66.057 | 51.775 | 110.535 | 1 | 47.39 |
| 1815 | C | PRO | 230 | 67.241 | 50.088 | 107.461 | 1 | 45.87 |
| 1816 | O | PRO | 230 | 67.399 | 50.586 | 106.349 | 1 | 46.92 |
| 1817 | N | ASN | 231 | 67.912 | 49.019 | 107.87 | 1 | 45.84 |
| 1818 | CA | ASN | 231 | 68.878 | 48.371 | 106.999 | 1 | 46.56 |
| 1819 | CB | ASN | 231 | 69.892 | 47.581 | 107.821 | 1 | 49.47 |
| 1820 | CG | ASN | 231 | 70.792 | 48.478 | 108.642 | 1 | 52.53 |
| 1821 | OD1 | ASN | 231 | 71.419 | 49.395 | 108.106 | 1 | 53.82 |
| 1822 | ND2 | ASN | 231 | 70.864 | 48.22 | 109.948 | 1 | 53.65 |
| 1823 | C | ASN | 231 | 68.229 | 47.448 | 105.98 | 1 | 45.65 |
| 1824 | O | ASN | 231 | 68.899 | 46.96 | 105.072 | 1 | 45.76 |
| 1825 | N | SER | 232 | 66.932 | 47.196 | 106.133 | 1 | 43.44 |
| 1826 | CA | SER | 232 | 66.228 | 45.324 | 105.196 | 1 | 42.14 |
| 1827 | CB | SER | 232 | 64.802 | 48.046 | 105.686 | 1 | 41.81 |
| 1828 | OG | SER | 232 | 64.045 | 47.238 | 105.793 | 1 | 40.94 |
| 1829 | C | SER | 232 | 66.188 | 48.976 | 103.815 | 1 | 40.57 |
| 1830 | O | SER | 232 | 66.055 | 48.196 | 103.691 | 1 | 39.09 |
| 1831 | N | PRO | 233 | 66.308 | 48.168 | 102.756 | 1 | 39.8 |
| 1832 | CD | PRO | 233 | 66.411 | 44.698 | 102.748 | 1 | 39.27 |
| 1833 | CA | PRO | 233 | 66.283 | 45.707 | 101.393 | 1 | 39.82 |
| 1834 | CB | PRO | 233 | 66.588 | 45.48 | 100.531 | 1 | 39.77 |
| 1835 | CG | PRO | 233 | 65.995 | 44.35 | 101.336 | 1 | 40.75 |
| 1836 | C | PRO | 233 | 64.95 | 47.361 | 101.041 | 1 | 39.89 |
| 1837 | O | PRO | 233 | 63.902 | 46.978 | 101.56 | 1 | 39.22 |
| 1838 | N | VAL | 234 | 65.003 | 48.36 | 100.167 | 1 | 38.87 |
| 1839 | CA | VAL | 234 | 63.806 | 49.065 | 99.736 | 1 | 38.86 |
| 1840 | CB | VAL | 234 | 64.137 | 50.51 | 99.331 | 1 | 40.06 |
| 1841 | CG1 | VAL | 234 | 62.857 | 51.279 | 99.03 | 1 | 39.32 |
| 1842 | CG2 | VAL | 234 | 64.914 | 51.185 | 100.451 | 1 | 40.19 |
| 1843 | C | VAL | 234 | 63.189 | 48.332 | 98.551 | 1 | 38.15 |
| 1844 | O | VAL | 234 | 63.743 | 48.328 | 97.448 | 1 | 37.93 |
| 1845 | N | LEU | 235 | 62.037 | 47.712 | 98.787 | 1 | 36.78 |
| 1846 | CA | LEU | 235 | 61.35 | 46.95 | 97.748 | 1 | 36.19 |
| 1847 | CB | LEU | 235 | 59.983 | 46.465 | 98.253 | 1 | 34.15 |
| 1848 | CG | LEU | 235 | 59.17 | 45.628 | 97.254 | 1 | 33.96 |
| 1849 | CD1 | LEU | 235 | 59.821 | 44.265 | 97.082 | 1 | 32.16 |
| 1850 | CD2 | LEU | 235 | 57.733 | 45.471 | 97.75 | 1 | 32.57 |
| 1851 | C | LEU | 235 | 61.147 | 47.71 | 96.442 | 1 | 35.2 |
| 1852 | O | LEU | 235 | 61.53 | 47.226 | 95.384 | 1 | 35.48 |
| 1853 | N | LEU | 236 | 60.55 | 48.895 | 96.521 | 1 | 35.43 |
| 1854 | CA | LEU | 236 | 60.259 | 49.681 | 95.328 | 1 | 37.25 |
| 1855 | CB | LEU | 236 | 59.364 | 50.868 | 95.691 | 1 | 37.23 |
| 1856 | CG | LEU | 236 | 57.987 | 50.481 | 96.25 | 1 | 38.48 |
| 1857 | CD1 | LEU | 236 | 57.144 | 51.728 | 96.456 | 1 | 38.3 |
| 1858 | CD2 | LEU | 236 | 57.295 | 49.523 | 95.288 | 1 | 37.67 |
| 1859 | C | LEU | 236 | 61.475 | 50.167 | 94.542 | 1 | 39.23 |
| 1860 | O | LEU | 236 | 61.335 | 50.8 | 93.49 | 1 | 39.02 |
| 1861 | N | GLU | 237 | 62.666 | 49.867 | 95.046 | 1 | 39.37 |
| 1862 | CA | GLU | 237 | 63.891 | 50.263 | 94.369 | 1 | 40.97 |
| 1863 | CB | GLU | 237 | 64.886 | 50.854 | 95.372 | 1 | 42.53 |
| 1864 | CG | GLU | 237 | 64.709 | 52.347 | 95.595 | 1 | 45.84 |
| 1865 | CD | GLU | 237 | 65.614 | 52.888 | 96.685 | 1 | 49.27 |
| 1866 | OE1 | GLU | 237 | 66.772 | 52.434 | 96.778 | 1 | 50.58 |
| 1867 | OE2 | GLU | 237 | 65.172 | 53.778 | 97.445 | 1 | 51.15 |
| 1868 | C | GLU | 237 | 64.513 | 49.078 | 93.649 | 1 | 40.7 |
| 1869 | O | GLU | 237 | 65.563 | 49.203 | 93.023 | 1 | 40.04 |
| 1870 | N | ASP | 238 | 63.857 | 47.926 | 93.728 | 1 | 39.55 |
| 1871 | CA | ASP | 238 | 64.368 | 46.726 | 93.077 | 1 | 39.87 |
| 1872 | CB | ASP | 238 | 63.452 | 45.541 | 93.379 | 1 | 38.76 |
| 1873 | CG | ASP | 238 | 63.947 | 44.256 | 92.771 | 1 | 38.72 |
| 1874 | OD1 | ASP | 238 | 63.671 | 44.016 | 91.576 | 1 | 38.63 |
| 1875 | OD2 | ASP | 238 | 64.622 | 43.481 | 93.483 | 1 | 39.32 |
| 1876 | C | ASP | 238 | 64.49 | 46.943 | 91.566 | 1 | 39.9 |
| 1877 | O | ASP | 238 | 63.568 | 47.451 | 90.929 | 1 | 39.8 |
| 1878 | N | PRO | 239 | 65.64 | 46.562 | 90.977 | 1 | 39.5 |
| 1879 | CD | PRO | 239 | 66.812 | 45.988 | 91.662 | 1 | 40.04 |
| 1880 | CA | PRO | 239 | 65.906 | 46.712 | 89.541 | 1 | 39.48 |
| 1881 | CB | PRO | 239 | 67.265 | 46.034 | 89.366 | 1 | 40.14 |
| 1882 | CG | PRO | 239 | 67.931 | 46.275 | 90.676 | 1 | 41.52 |
| 1883 | C | PRO | 239 | 64.853 | 46.086 | 88.636 | 1 | 38.81 |
| 1884 | O | PRO | 239 | 64.346 | 46.736 | 87.718 | 1 | 38.72 |
| 1885 | N | VAL | 240 | 64.537 | 44.82 | 88.893 | 1 | 37.76 |
| 1886 | CA | VAL | 240 | 63.554 | 44.094 | 88.098 | 1 | 36.76 |
| 1887 | CB | VAL | 240 | 63.412 | 42.645 | 88.603 | 1 | 36.48 |
| 1888 | CG1 | VAL | 240 | 62.382 | 41.888 | 87.766 | 1 | 37.11 |
| 1889 | CG2 | VAL | 240 | 64.759 | 41.949 | 88.527 | 1 | 35.46 |
| 1890 | C | VAL | 240 | 62.196 | 44.791 | 88.107 | 1 | 36.79 |
| 1891 | O | VAL | 240 | 61.568 | 44.961 | 87.057 | 1 | 35.48 |
| 1892 | N | LEU | 241 | 61.757 | 45.207 | 89.291 | 1 | 36.18 |
| 1893 | CA | LEU | 241 | 60.484 | 45.903 | 89.436 | 1 | 36.29 |
| 1894 | CB | LEU | 241 | 60.203 | 46.186 | 90.92 | 1 | 35.86 |
| 1895 | CG | LEU | 241 | 59.057 | 45.418 | 91.589 | 1 | 36.72 |
| 1896 | CD1 | LEU | 241 | 59.078 | 43.946 | 91.193 | 1 | 36.56 |
| 1897 | CD2 | LEU | 241 | 59.166 | 45.581 | 93.098 | 1 | 36.14 |
| 1898 | C | LEU | 241 | 60.488 | 47.211 | 88.652 | 1 | 36.13 |
| 1899 | O | LEU | 241 | 59.503 | 47.56 | 88.008 | 1 | 35.34 |
| 1900 | N | CYS | 242 | 61.596 | 47.94 | 88.708 | 1 | 37.21 |
| 1901 | CA | CYS | 242 | 61.683 | 49.206 | 87.988 | 1 | 38.27 |
| 1902 | CB | CYS | 242 | 62.909 | 49.998 | 88.457 | 1 | 39.33 |
| 1903 | SG | CYS | 242 | 62.797 | 50.558 | 90.196 | 1 | 44.22 |
| 1904 | C | CYS | 242 | 61.732 | 48.981 | 86.472 | 1 | 37.44 |
| 1905 | O | CYS | 242 | 61.162 | 49.757 | 85.704 | 1 | 37.1 |
| 1906 | N | ALA | 243 | 62.398 | 47.91 | 86.054 | 1 | 37.04 |
| 1907 | CA | ALA | 243 | 62.516 | 47.577 | 84.641 | 1 | 37.85 |
| 1908 | CB | ALA | 243 | 63.483 | 46.406 | 84.457 | 1 | 38.26 |
| 1909 | C | ALA | 243 | 61.153 | 47.227 | 84.051 | 1 | 38.69 |
| 1910 | O | ALA | 243 | 60.783 | 47.714 | 82.98 | 1 | 38.51 |
| 1911 | N | LEU | 244 | 60.409 | 46.371 | 84.751 | 1 | 37.63 |
| 1912 | CA | LEU | 244 | 59.084 | 45.964 | 84.295 | 1 | 36.83 |
| 1913 | CB | LEU | 244 | 58.525 | 44.86 | 85.2 | 1 | 36.85 |
| 1914 | CG | LEU | 244 | 59.199 | 43.488 | 85.065 | 1 | 37.84 |
| 1915 | CD1 | LEU | 244 | 58.739 | 42.554 | 86.183 | 1 | 38.32 |
| 1916 | CD2 | LEU | 244 | 58.864 | 42.898 | 83.71 | 1 | 38.61 |
| 1917 | C | LEU | 244 | 58.143 | 47.161 | 84.281 | 1 | 36.98 |
| 1918 | O | LEU | 244 | 57.247 | 47.241 | 83.444 | 1 | 36.39 |
| 1919 | N | ALA | 245 | 58.355 | 48.094 | 85.206 | 1 | 36.97 |
| 1920 | CA | ALA | 245 | 57.525 | 49.291 | 85.282 | 1 | 38.57 |
| 1921 | CB | ALA | 245 | 57.863 | 50.082 | 86.541 | 1 | 36.81 |
| 1922 | C | ALA | 245 | 57.717 | 50.172 | 84.046 | 1 | 40.02 |
| 1923 | O | ALA | 245 | 56.749 | 50.659 | 83.466 | 1 | 40.15 |
| 1924 | N | LYS | 246 | 58.97 | 50.379 | 83.655 | 1 | 42.4 |
| 1925 | CA | LYS | 246 | 59.276 | 51.202 | 82.49 | 1 | 44.83 |
| 1926 | CB | LYS | 246 | 60.788 | 51.396 | 82.368 | 1 | 47.34 |
| 1927 | CG | LYS | 246 | 61.387 | 52.148 | 83.545 | 1 | 50.83 |
| 1928 | CD | LYS | 246 | 62.881 | 52.372 | 83.384 | 1 | 53.82 |
| 1929 | CE | LYS | 246 | 63.469 | 53.041 | 84.623 | 1 | 54.6 |
| 1930 | NZ | LYS | 246 | 64.949 | 53.184 | 84.523 | 1 | 56.72 |
| 1931 | C | LYS | 246 | 58.722 | 50.564 | 81.223 | 1 | 44.53 |
| 1932 | O | LYS | 246 | 58.207 | 51.25 | 80.345 | 1 | 45.4 |
| 1933 | N | LYS | 247 | 58.826 | 49.244 | 81.145 | 1 | 45.11 |
| 1934 | CA | LYS | 247 | 58.33 | 48.497 | 80.001 | 1 | 45.55 |
| 1935 | CB | LYS | 247 | 58.658 | 47.015 | 80.183 | 1 | 46.31 |
| 1936 | CG | LYS | 247 | 58.091 | 46.099 | 79.119 | 1 | 49 |
| 1937 | CD | LYS | 247 | 58.414 | 44.643 | 79.445 | 1 | 51.05 |
| 1938 | CE | LYS | 247 | 57.768 | 43.677 | 78.461 | 1 | 52.33 |
| 1939 | NZ | LYS | 247 | 58.061 | 42.251 | 78.803 | 1 | 52.42 |
| 1940 | C | LYS | 247 | 56.821 | 48.686 | 79.835 | 1 | 45.9 |
| 1941 | O | LYS | 247 | 56.328 | 48.847 | 78.718 | 1 | 45.84 |
| 1942 | N | HIS | 248 | 56.094 | 48.669 | 80.948 | 1 | 44.47 |
| 1943 | CA | HIS | 248 | 54.642 | 48.835 | 80.931 | 1 | 43.29 |
| 1944 | CB | HIS | 248 | 54.006 | 48.026 | 82.067 | 1 | 42.91 |
| 1945 | CG | HIS | 248 | 53.815 | 46.578 | 81.749 | 1 | 42.56 |
| 1946 | CD2 | HIS | 248 | 54.581 | 45.495 | 82.022 | 1 | 42.78 |
| 1947 | ND1 | HIS | 248 | 52.716 | 46.106 | 81.062 | 1 | 42.35 |
| 1948 | CE1 | HIS | 248 | 52.812 | 44.795 | 80.929 | 1 | 43.28 |
| 1949 | NE2 | HIS | 248 | 53.935 | 44.399 | 81.502 | 1 | 43.23 |
| 1950 | C | HIS | 248 | 54.236 | 50.295 | 81.072 | 1 | 41.93 |
| 1951 | O | HIS | 248 | 53.05 | 50.613 | 81.078 | 1 | 41.29 |
| 1952 | N | LYS | 249 | 55.226 | 51.175 | 81.183 | 1 | 42.25 |
| 1953 | CA | LYS | 249 | 54.969 | 52.599 | 81.339 | 1 | 43.15 |
| 1954 | CB | LYS | 249 | 54.24 | 53.152 | 80.108 | 1 | 47.39 |
| 1955 | CG | LYS | 249 | 55.026 | 54.211 | 79.341 | 1 | 50.86 |
| 1956 | CD | LYS | 249 | 56.278 | 53.632 | 78.696 | 1 | 54.03 |
| 1957 | CE | LYS | 249 | 55.933 | 52.775 | 77.482 | 1 | 57.3 |
| 1958 | NZ | LYS | 249 | 55.299 | 53.585 | 76.396 | 1 | 58.33 |
| 1959 | C | LYS | 249 | 54.134 | 52.85 | 82.597 | 1 | 42.46 |
| 1960 | O | LYS | 249 | 53.218 | 53.675 | 82.607 | 1 | 41.54 |
| 1961 | N | ARG | 250 | 54.452 | 52.119 | 83.659 | 1 | 40.73 |
| 1962 | CA | ARG | 250 | 53.751 | 52.268 | 84.928 | 1 | 38.52 |
| 1963 | CB | ARG | 250 | 53.04 | 50.962 | 85.3 | 1 | 37.53 |
| 1964 | CG | ARG | 250 | 52.011 | 50.489 | 84.281 | 1 | 37.01 |
| 1965 | CD | ARG | 250 | 50.801 | 51.413 | 84.237 | 1 | 35.26 |
| 1966 | NE | ARG | 250 | 49.873 | 51.028 | 83.178 | 1 | 34.52 |
| 1967 | CZ | ARG | 250 | 48.673 | 51.571 | 82.998 | 1 | 34.48 |
| 1968 | NH1 | ARG | 250 | 48.243 | 52.528 | 83.81 | 1 | 34.48 |
| 1969 | NH2 | ARG | 250 | 47.905 | 51.159 | 82.001 | 1 | 34.14 |
| 1970 | C | ARG | 250 | 54.777 | 52.619 | 86 | 1 | 38.11 |
| 1971 | O | ARG | 250 | 55.777 | 53.28 | 85.719 | 1 | 37.87 |
| 1972 | N | THR | 251 | 54.529 | 52.171 | 87.226 | 1 | 36.4 |
| 1973 | CA | THR | 251 | 55.443 | 52.432 | 88.33 | 1 | 34.98 |
| 1974 | CB | THR | 251 | 54.832 | 53.42 | 89.338 | 1 | 35.26 |
| 1975 | OG1 | THR | 251 | 53.65 | 52.844 | 89.905 | 1 | 34.94 |
| 1976 | CG2 | THR | 251 | 54.465 | 54.739 | 88.654 | 1 | 34.78 |
| 1977 | C | THR | 251 | 55.742 | 51.122 | 89.057 | 1 | 34.17 |
| 1978 | O | THR | 251 | 55.002 | 50.144 | 88.921 | 1 | 34.37 |
| 1979 | N | PRO | 252 | 56.841 | 51.08 | 89.827 | 1 | 33.46 |
| 1980 | CD | PRO | 252 | 57.871 | 52.123 | 89.995 | 1 | 33.42 |
| 1981 | CA | PRO | 252 | 57.204 | 49.868 | 90.566 | 1 | 31.79 |
| 1982 | CB | PRO | 252 | 58.446 | 50.296 | 91.345 | 1 | 33.43 |
| 1983 | CG | PRO | 252 | 59.063 | 51.318 | 90.453 | 1 | 34.85 |
| 1984 | C | PRO | 252 | 56.073 | 49.418 | 91.494 | 1 | 29.43 |
| 1985 | O | PRO | 252 | 55.82 | 48.226 | 91.638 | 1 | 28.84 |
| 1986 | N | ALA | 253 | 55.405 | 50.379 | 92.124 | 1 | 28.12 |
| 1987 | CA | ALA | 253 | 54.305 | 50.069 | 93.036 | 1 | 28.73 |
| 1988 | CB | ALA | 253 | 53.743 | 51.347 | 93.65 | 1 | 26.03 |
| 1989 | C | ALA | 253 | 53.2 | 49.321 | 92.301 | 1 | 28.76 |
| 1990 | O | ALA | 253 | 52.707 | 48.299 | 92.785 | 1 | 27.92 |
| 1991 | N | LEU | 254 | 52.807 | 49.828 | 91.133 | 1 | 27.76 |
| 1992 | CA | LEU | 254 | 51.754 | 49.182 | 90.357 | 1 | 27.06 |
| 1993 | CB | LEU | 254 | 51.413 | 50.018 | 89.122 | 1 | 27.29 |
| 1994 | CG | LEU | 254 | 50.529 | 51.229 | 89.432 | 1 | 28.73 |
| 1995 | CD1 | LEU | 254 | 50.36 | 52.099 | 88.181 | 1 | 28.92 |
| 1996 | CD2 | LEU | 254 | 49.169 | 50.742 | 89.919 | 1 | 27.78 |
| 1997 | C | LEU | 254 | 52.131 | 47.764 | 89.958 | 1 | 25.42 |
| 1998 | O | LEU | 254 | 51.29 | 46.863 | 89.964 | 1 | 25.92 |
| 1999 | N | ILE | 255 | 53.399 | 47.559 | 89.618 | 1 | 24.15 |
| 2000 | CA | ILE | 255 | 53.857 | 46.227 | 89.244 | 1 | 24.33 |
| 2001 | CB | ILE | 255 | 55.351 | 46.241 | 88.806 | 1 | 26.07 |
| 2002 | CG2 | ILE | 255 | 55.826 | 44.812 | 88.551 | 1 | 22.96 |
| 2003 | CG1 | ILE | 255 | 55.542 | 47.128 | 87.557 | 1 | 26.67 |
| 2004 | CD1 | ILE | 255 | 54.781 | 46.648 | 86.311 | 1 | 28.41 |
| 2005 | C | ILE | 255 | 53.703 | 45.284 | 90.454 | 1 | 24.72 |
| 2006 | O | ILE | 255 | 53.214 | 44.159 | 90.321 | 1 | 23.96 |
| 2007 | N | ALA | 256 | 54.117 | 45.756 | 91.63 | 1 | 23.68 |
| 2008 | CA | ALA | 256 | 54.049 | 44.953 | 92.852 | 1 | 25.5 |
| 2009 | CB | ALA | 256 | 54.716 | 45.696 | 94.01 | 1 | 24.12 |
| 2010 | C | ALA | 256 | 52.616 | 44.596 | 93.217 | 1 | 24.66 |
| 2011 | O | ALA | 256 | 52.348 | 43.494 | 93.689 | 1 | 24.59 |
| 2012 | N | LEU | 257 | 51.699 | 45.533 | 93.004 | 1 | 25.64 |
| 2013 | CA | LEU | 257 | 50.286 | 45.3 | 93.299 | 1 | 24.19 |
| 2014 | CB | LEU | 257 | 49.517 | 46.628 | 93.248 | 1 | 24.73 |
| 2015 | CG | LEU | 257 | 49.19 | 47.386 | 94.531 | 1 | 27.37 |
| 2016 | CD1 | LEU | 257 | 50.18 | 47.092 | 95.627 | 1 | 28.38 |
| 2017 | CD2 | LEU | 257 | 49.133 | 48.867 | 94.212 | 1 | 26.24 |
| 2018 | C | LEU | 257 | 49.678 | 44.312 | 92.303 | 1 | 24.13 |
| 2019 | O | LEU | 257 | 48.98 | 43.375 | 92.7 | 1 | 23.36 |
| 2020 | N | ARG | 258 | 49.948 | 44.516 | 91.013 | 1 | 21.76 |
| 2021 | CA | ARG | 258 | 49.396 | 43.638 | 89.976 | 1 | 21.32 |
| 2022 | CB | ARG | 258 | 49.793 | 44.131 | 88.579 | 1 | 20.62 |
| 2023 | CG | ARG | 258 | 49.115 | 43.356 | 87.444 | 1 | 21.27 |
| 2024 | CD | ARG | 258 | 47.62 | 43.63 | 87.383 | 1 | 19.89 |
| 2025 | NE | ARG | 258 | 46.898 | 42.562 | 85.687 | 1 | 21.41 |
| 2026 | CZ | ARG | 258 | 45.602 | 42.602 | 86.396 | 1 | 20.8 |
| 2027 | NH1 | ARG | 258 | 44.881 | 43.664 | 86.733 | 1 | 21.02 |
| 2028 | NH2 | ARG | 258 | 45.025 | 41.571 | 85.789 | 1 | 21.49 |
| 2029 | C | ARG | 258 | 49.856 | 42.2 | 90.156 | 1 | 20.44 |
| 2030 | O | ARG | 258 | 49.112 | 41.254 | 89.907 | 1 | 19.9 |
| 2031 | N | TYR | 259 | 51.105 | 42.048 | 90.575 | 1 | 20.01 |
| 2032 | CA | TYR | 259 | 51.675 | 40.736 | 90.823 | 1 | 22.19 |
| 2033 | CB | TYR | 259 | 53.06 | 40.913 | 91.447 | 1 | 21.54 |
| 2034 | CG | TYR | 259 | 53.676 | 39.663 | 92.022 | 1 | 22.47 |
| 2035 | CD1 | TYR | 259 | 54.087 | 38.617 | 91.197 | 1 | 21.09 |
| 2036 | CE1 | TYR | 259 | 54.673 | 37.468 | 91.731 | 1 | 24.15 |
| 2037 | CD2 | TYR | 259 | 53.864 | 39.535 | 93.4 | 1 | 21.46 |
| 2038 | CE2 | TYR | 259 | 54.448 | 38.392 | 93.944 | 1 | 23.27 |
| 2039 | CZ | TYR | 259 | 54.851 | 37.365 | 93.111 | 1 | 24.07 |
| 2040 | OH | TYR | 259 | 55.433 | 36.23 | 93.646 | 1 | 25.03 |
| 2041 | C | TYR | 259 | 50.768 | 39.944 | 91.773 | 1 | 22.3 |
| 2042 | O | TYR | 259 | 50.43 | 38.785 | 91.503 | 1 | 24.44 |
| 2043 | N | GLN | 260 | 50.364 | 40.572 | 92.873 | 1 | 21.31 |
| 2044 | CA | GLN | 260 | 49.517 | 39.891 | 93.859 | 1 | 21.5 |
| 2045 | CB | GLN | 260 | 49.363 | 40.755 | 95.123 | 1 | 20.4 |
| 2046 | CG | GLN | 260 | 50.692 | 41.128 | 95.788 | 1 | 20.56 |
| 2047 | CD | GLN | 260 | 51.391 | 39.942 | 96.438 | 1 | 23.73 |
| 2048 | OE1 | GLN | 260 | 50.998 | 38.787 | 96.242 | 1 | 22.78 |
| 2049 | NE2 | GLN | 260 | 52.446 | 40.219 | 97.206 | 1 | 22.67 |
| 2050 | C | GLN | 260 | 48.149 | 39.536 | 93.298 | 1 | 21.05 |
| 2051 | O | GLN | 260 | 47.642 | 38.43 | 93.526 | 1 | 20.53 |
| 2052 | N | LEU | 261 | 47.539 | 40.46 | 92.562 | 1 | 20.98 |
| 2053 | CA | LEU | 261 | 46.221 | 40.174 | 91.994 | 1 | 22.92 |
| 2054 | CB | LEU | 261 | 45.687 | 41.388 | 91.234 | 1 | 23.96 |
| 2055 | CG | LEU | 261 | 45.351 | 42.607 | 92.089 | 1 | 25.12 |
| 2056 | CD1 | LEU | 261 | 44.726 | 43.66 | 91.186 | 1 | 26.75 |
| 2057 | CD2 | LEU | 261 | 44.383 | 42.231 | 93.212 | 1 | 23.31 |
| 2058 | C | LEU | 261 | 46.219 | 38.961 | 91.065 | 1 | 23.12 |
| 2059 | O | LEU | 261 | 45.28 | 38.167 | 91.08 | 1 | 23.7 |
| 2060 | N | GLN | 262 | 47.267 | 38.802 | 90.264 | 1 | 22.38 |
| 2061 | CA | GLN | 262 | 47.297 | 37.675 | 89.34 | 1 | 22.84 |
| 2062 | CB | GLN | 262 | 48.264 | 37.959 | 68.187 | 1 | 22.63 |
| 2063 | CG | GLN | 262 | 47.873 | 39.221 | 87.462 | 1 | 23.55 |
| 2064 | CD | GLN | 262 | 48.477 | 39.386 | 86.07 | 1 | 24.5 |
| 2065 | OE1 | GLN | 262 | 48.43 | 40.484 | 85.518 | 1 | 24.63 |
| 2066 | NE2 | GLN | 262 | 49.02 | 38.308 | 85.492 | 1 | 22.08 |
| 2067 | C | GLN | 262 | 47.595 | 36.331 | 89.984 | 1 | 23.25 |
| 2068 | O | GLN | 262 | 47.433 | 35.284 | 89.35 | 1 | 23.72 |
| 2069 | N | ARG | 263 | 48.035 | 36.342 | 91.238 | 1 | 22.12 |
| 2070 | CA | ARG | 263 | 48.283 | 35.077 | 91.918 | 1 | 23.73 |
| 2071 | CB | ARG | 263 | 49.647 | 35.093 | 92.639 | 1 | 25.01 |
| 2072 | CG | ARG | 263 | 49.89 | 36.244 | 93.574 | 1 | 26.67 |
| 2073 | CD | ARG | 263 | 51.395 | 36.514 | 93.732 | 1 | 27.18 |
| 2074 | NE | ARG | 263 | 52.15 | 35.356 | 94.211 | 1 | 25.96 |
| 2075 | CZ | ARG | 263 | 52.567 | 35.183 | 95.462 | 1 | 25.36 |
| 2076 | NH1 | ARG | 263 | 52.309 | 36.096 | 96.391 | 1 | 25.36 |
| 2077 | NH2 | ARG | 263 | 53.249 | 34.089 | 95.783 | 1 | 24.76 |
| 2078 | C | ARG | 263 | 47.137 | 34.751 | 92.879 | 1 | 23.22 |
| 2079 | O | ARG | 263 | 47.246 | 33.846 | 93.702 | 1 | 24.73 |
| 2080 | N | GLY | 264 | 46.034 | 35.492 | 92.756 | 1 | 23.03 |
| 2081 | CA | GLY | 264 | 44.859 | 35.25 | 93.578 | 1 | 21.99 |
| 2082 | C | GLY | 264 | 44.865 | 35.854 | 94.967 | 1 | 22.05 |
| 2083 | O | GLY | 264 | 44.065 | 35.465 | 95.817 | 1 | 21.8 |
| 2084 | N | VAL | 265 | 45.761 | 36.807 | 95.196 | 1 | 20.76 |
| 2085 | CA | VAL | 265 | 45.886 | 37.458 | 96.493 | 1 | 20.75 |
| 2086 | CB | VAL | 265 | 47.395 | 37.712 | 96.809 | 1 | 20.54 |
| 2087 | CG1 | VAL | 265 | 47.546 | 38.622 | 98.01 | 1 | 19.37 |
| 2088 | CG2 | VAL | 265 | 48.112 | 36.382 | 97.065 | 1 | 20.48 |
| 2089 | C | VAL | 265 | 45.11 | 38.786 | 96.526 | 1 | 21.62 |
| 2090 | O | VAL | 265 | 45.072 | 39.508 | 95.534 | 1 | 22.39 |
| 2091 | N | VAL | 266 | 44.45 | 39.074 | 97.647 | 1 | 21.11 |
| 2092 | CA | VAL | 266 | 43.745 | 40.343 | 97.807 | 1 | 20.94 |
| 2093 | CB | VAL | 266 | 42.58 | 40.236 | 98.807 | 1 | 20.65 |
| 2094 | CG1 | VAL | 266 | 42.082 | 41.633 | 99.176 | 1 | 19.84 |
| 2095 | CG2 | VAL | 266 | 41.441 | 39.423 | 98.178 | 1 | 19.17 |
| 2096 | C | VAL | 266 | 44.851 | 41.241 | 98.359 | 1 | 21.24 |
| 2097 | O | VAL | 266 | 45.578 | 40.847 | 99.273 | 1 | 21.37 |
| 2098 | N | VAL | 267 | 44.992 | 42.443 | 97.814 | 1 | 20.32 |
| 2099 | CA | VAL | 267 | 46.092 | 43.295 | 98.236 | 1 | 19.96 |
| 2100 | CB | VAL | 267 | 47.071 | 43.491 | 97.019 | 1 | 22.82 |
| 2101 | CG1 | VAL | 267 | 46.396 | 44.342 | 95.928 | 1 | 21.52 |
| 2102 | CG2 | VAL | 267 | 48.377 | 44.121 | 97.47 | 1 | 21.72 |
| 2103 | C | VAL | 267 | 45.701 | 44.641 | 98.631 | 1 | 19.92 |
| 2104 | O | VAL | 267 | 44.755 | 45.291 | 98.383 | 1 | 21.24 |
| 2105 | N | LEU | 268 | 46.425 | 45.045 | 99.87 | 1 | 20.4 |
| 2106 | CA | LEU | 268 | 46.171 | 46.332 | 100.506 | 1 | 21.21 |
| 2107 | CB | LEU | 268 | 46.276 | 46.209 | 102.033 | 1 | 21.71 |
| 2108 | CG | LEU | 268 | 45.316 | 45.226 | 102.716 | 1 | 22.01 |
| 2109 | CD1 | LEU | 268 | 45.492 | 45.28 | 104.237 | 1 | 21.04 |
| 2110 | CD2 | LEU | 268 | 43.893 | 45.586 | 102.349 | 1 | 19.01 |
| 2111 | C | LEU | 268 | 47.24 | 47.306 | 100.006 | 1 | 21.6 |
| 2112 | O | LEU | 268 | 48.311 | 46.893 | 99.569 | 1 | 22.1 |
| 2113 | N | ALA | 269 | 46.927 | 48.592 | 100.054 | 1 | 22.37 |
| 2114 | CA | ALA | 269 | 47.859 | 49.636 | 99.654 | 1 | 23.5 |
| 2115 | CB | ALA | 269 | 47.747 | 49.941 | 98.154 | 1 | 24.65 |
| 2116 | C | ALA | 269 | 47.5 | 50.866 | 100.46 | 1 | 21.63 |
| 2117 | O | ALA | 269 | 46.365 | 51.336 | 100.432 | 1 | 20.76 |
| 2118 | N | LYS | 270 | 48.473 | 51.371 | 101.201 | 1 | 22.49 |
| 2119 | CA | LYS | 270 | 48.259 | 52.555 | 102.006 | 1 | 23.31 |
| 2120 | CB | LYS | 270 | 48.854 | 52.366 | 103.403 | 1 | 24.43 |
| 2121 | CG | LYS | 270 | 48.799 | 53.615 | 104.287 | 1 | 25.8 |
| 2122 | CD | LYS | 270 | 49.942 | 54.593 | 103.998 | 1 | 27.08 |
| 2123 | CE | LYS | 270 | 49.876 | 55.81 | 104.909 | 1 | 28.38 |
| 2124 | NZ | LYS | 270 | 51.044 | 56.73 | 104.709 | 1 | 29.11 |
| 2125 | C | LYS | 270 | 48.941 | 53.744 | 101.355 | 1 | 23.74 |
| 2126 | O | LYS | 270 | 50.076 | 53.637 | 100.899 | 1 | 23.72 |
| 2127 | N | SER | 271 | 48.237 | 54.867 | 101.316 | 1 | 24.86 |
| 2128 | CA | SER | 271 | 48.783 | 56.115 | 100.802 | 1 | 26.56 |
| 2129 | CB | SER | 271 | 48.802 | 56.15 | 99.278 | 1 | 27.2 |
| 2130 | OG | SER | 271 | 49.375 | 57.377 | 98.845 | 1 | 27.96 |
| 2131 | C | SER | 271 | 47.951 | 57.284 | 101.3 | 1 | 26.81 |
| 2132 | O | SER | 271 | 46.723 | 57.232 | 101.287 | 1 | 26.35 |
| 2133 | N | TYR | 272 | 48.62 | 58.336 | 101.758 | 1 | 27.32 |
| 2134 | CA | TYR | 272 | 47.909 | 59.52 | 102.214 | 1 | 29.84 |
| 2135 | CB | TYR | 272 | 48.415 | 59.954 | 103.588 | 1 | 30.58 |
| 2136 | CG | TYR | 272 | 47.739 | 59.233 | 104.735 | 1 | 33.04 |
| 2137 | CD1 | TYR | 272 | 47.133 | 57.99 | 104.546 | 1 | 32.63 |
| 2138 | CE1 | TYR | 272 | 46.53 | 57.315 | 105.605 | 1 | 34.22 |
| 2139 | CD2 | TYR | 272 | 47.726 | 59.785 | 106.018 | 1 | 32.82 |
| 2140 | CE2 | TYR | 272 | 47.128 | 59.121 | 107.086 | 1 | 33.95 |
| 2141 | CZ | TYR | 272 | 46.532 | 57.887 | 106.875 | 1 | 35.22 |
| 2142 | OH | TYR | 272 | 45.938 | 57.227 | 107.928 | 1 | 35.13 |
| 2143 | C | TYR | 272 | 48.111 | 60.642 | 101.203 | 1 | 31.07 |
| 2144 | O | TYR | 272 | 47.744 | 61.784 | 101.458 | 1 | 31.91 |
| 2145 | N | ASN | 273 | 48.685 | 60.301 | 100.052 | 1 | 31.74 |
| 2146 | CA | ASN | 273 | 48.943 | 61.273 | 98.99 | 1 | 32.79 |
| 2147 | CB | ASN | 273 | 50.365 | 61.082 | 98.46 | 1 | 33.57 |
| 2148 | CG | ASN | 273 | 50.713 | 62.062 | 97.356 | 1 | 36.73 |
| 2149 | OD1 | ASN | 273 | 50.345 | 61.869 | 95.194 | 1 | 36.9 |
| 2150 | ND2 | ASN | 273 | 51.419 | 63.129 | 97.718 | 1 | 34.92 |
| 2151 | C | ASN | 273 | 47.932 | 61.11 | 97.855 | 1 | 33.61 |
| 2152 | O | ASN | 273 | 47.805 | 60.029 | 97.288 | 1 | 32.66 |
| 2153 | N | GLU | 274 | 47.224 | 62.184 | 97.519 | 1 | 34.9 |
| 2154 | CA | GLU | 274 | 46.21 | 62.137 | 96.468 | 1 | 37.68 |
| 2155 | CB | GLU | 274 | 45.654 | 63.534 | 96.204 | 1 | 40.27 |
| 2156 | CG | GLU | 274 | 44.286 | 63.518 | 95.55 | 1 | 45.62 |
| 2157 | CD | GLU | 274 | 43.744 | 64.91 | 95.286 | 1 | 49.14 |
| 2158 | OE1 | GLU | 274 | 43.877 | 65.781 | 96.175 | 1 | 51.78 |
| 2159 | OE2 | GLU | 274 | 43.172 | 65.122 | 94.197 | 1 | 51.55 |
| 2160 | C | GLU | 274 | 46.689 | 61.532 | 95.15 | 1 | 37.73 |
| 2161 | O | GLU | 274 | 46.005 | 60.693 | 94.565 | 1 | 38.29 |
| 2162 | N | GLN | 275 | 47.856 | 61.964 | 94.684 | 1 | 37.55 |
| 2163 | CA | GLN | 275 | 48.418 | 61.466 | 93.433 | 1 | 38.35 |
| 2164 | CB | GLN | 275 | 49.774 | 62.134 | 93.156 | 1 | 41.05 |
| 2165 | CG | GLN | 275 | 50.53 | 61.549 | 91.96 | 1 | 47.47 |
| 2166 | CD | GLN | 275 | 52.015 | 61.897 | 91.974 | 1 | 50.47 |
| 2167 | OE1 | GLN | 275 | 52.72 | 61.617 | 92.946 | 1 | 54.12 |
| 2168 | NE2 | GLN | 275 | 52.495 | 62.5 | 90.893 | 1 | 51.74 |
| 2169 | C | GLN | 275 | 48.605 | 59.952 | 93.465 | 1 | 36.21 |
| 2170 | O | GLN | 275 | 48.182 | 59.239 | 92.557 | 1 | 36.07 |
| 2171 | N | ARG | 276 | 49.245 | 59.461 | 94.519 | 1 | 34.21 |
| 2172 | CA | ARG | 276 | 49.496 | 58.035 | 94.63 | 1 | 32.82 |
| 2173 | CB | ARG | 276 | 50.544 | 57.779 | 95.713 | 1 | 32.52 |
| 2174 | CG | ARG | 276 | 51.914 | 58.352 | 95.336 | 1 | 31.92 |
| 2175 | CD | ARG | 276 | 53.003 | 57.977 | 96.328 | 1 | 32.43 |
| 2176 | NE | ARG | 276 | 53.012 | 58.823 | 97.515 | 1 | 36.32 |
| 2177 | CZ | ARG | 276 | 53.549 | 60.039 | 97.565 | 1 | 39.2 |
| 2178 | NH1 | ARG | 276 | 54.127 | 60.555 | 96.484 | 1 | 37.68 |
| 2179 | NH2 | ARG | 276 | 53.517 | 60.734 | 98.699 | 1 | 38.21 |
| 2180 | C | ARG | 276 | 48.221 | 57.217 | 94.869 | 1 | 32.1 |
| 2181 | O | ARG | 276 | 48.105 | 56.093 | 94.379 | 1 | 31.08 |
| 2182 | N | ILE | 277 | 47.266 | 57.78 | 95.603 | 1 | 29.08 |
| 2183 | CA | ILE | 277 | 46.007 | 57.094 | 95.846 | 1 | 30.23 |
| 2184 | CB | ILE | 277 | 45.068 | 57.946 | 96.733 | 1 | 29.19 |
| 2185 | CG2 | ILE | 277 | 43.645 | 57.385 | 96.686 | 1 | 28.24 |
| 2186 | CG1 | ILE | 277 | 45.606 | 57.979 | 98.165 | 1 | 28.81 |
| 2187 | CD1 | ILE | 277 | 44.878 | 58.939 | 99.09 | 1 | 30.07 |
| 2188 | C | ILE | 277 | 45.32 | 56.823 | 94.502 | 1 | 31.49 |
| 2189 | O | ILE | 277 | 44.871 | 55.705 | 94.229 | 1 | 29.95 |
| 2190 | N | ARG | 278 | 45.254 | 57.854 | 93.663 | 1 | 32.62 |
| 2191 | CA | ARG | 278 | 44.637 | 57.745 | 92.343 | 1 | 33.3 |
| 2192 | CB | ARG | 278 | 44.586 | 59.124 | 91.677 | 1 | 37.93 |
| 2193 | CG | ARG | 278 | 43.538 | 60.072 | 92.247 | 1 | 42.19 |
| 2194 | CD | ARG | 278 | 42.168 | 59.771 | 91.662 | 1 | 48.23 |
| 2195 | NE | ARG | 278 | 41.132 | 60.665 | 92.182 | 1 | 53.2 |
| 2196 | CZ | ARG | 278 | 39.866 | 60.662 | 91.77 | 1 | 54.66 |
| 2197 | NH1 | ARG | 278 | 39.477 | 59.812 | 90.829 | 1 | 56.28 |
| 2198 | NH2 | ARG | 278 | 38.989 | 61.501 | 92.305 | 1 | 56.34 |
| 2199 | C | ARG | 278 | 45.426 | 56.783 | 91.464 | 1 | 32.02 |
| 2200 | O | ARG | 278 | 44.855 | 56.039 | 90.673 | 1 | 33.73 |
| 2201 | N | GLN | 279 | 46.744 | 56.795 | 91.613 | 1 | 30.29 |
| 2202 | CA | GLN | 279 | 47.609 | 55.93 | 90.824 | 1 | 30.03 |
| 2203 | CB | GLN | 279 | 49.07 | 56.324 | 91.04 | 1 | 31.01 |
| 2204 | CG | GLN | 279 | 50.064 | 55.42 | 90.322 | 1 | 34.41 |
| 2205 | CD | GLN | 279 | 51.495 | 55.658 | 90.766 | 1 | 36.75 |
| 2206 | OE1 | GLN | 279 | 51.999 | 56.78 | 90.694 | 1 | 39.52 |
| 2207 | NE2 | GLN | 279 | 52.16 | 54.601 | 91.227 | 1 | 35.02 |
| 2208 | C | GLN | 279 | 47.446 | 54.438 | 91.133 | 1 | 29.57 |
| 2209 | O | GLN | 279 | 47.482 | 53.603 | 90.226 | 1 | 28.68 |
| 2210 | N | ASN | 280 | 47.276 | 54.099 | 92.41 | 1 | 27.58 |
| 2211 | CA | ASN | 280 | 47.141 | 52.702 | 92.8 | 1 | 26.53 |
| 2212 | CB | ASN | 280 | 47.177 | 52.569 | 94.331 | 1 | 27.16 |
| 2213 | CG | ASN | 280 | 48.537 | 52.944 | 94.918 | 1 | 29.46 |
| 2214 | OD1 | ASN | 280 | 49.559 | 52.914 | 94.224 | 1 | 30.7 |
| 2215 | ND2 | ASN | 280 | 48.556 | 53.287 | 96.201 | 1 | 28.8 |
| 2216 | C | ASN | 280 | 45.918 | 51.978 | 92.23 | 1 | 25.59 |
| 2217 | O | ASN | 280 | 45.976 | 50.768 | 92.005 | 1 | 25.49 |
| 2218 | N | VAL | 281 | 44.825 | 52.696 | 91.984 | 1 | 25.25 |
| 2219 | CA | VAL | 281 | 43.644 | 52.05 | 91.424 | 1 | 28.01 |
| 2220 | CB | VAL | 281 | 42.366 | 52.943 | 91.522 | 1 | 29.55 |
| 2221 | CG1 | VAL | 281 | 42.003 | 53.182 | 92.978 | 1 | 31.31 |
| 2222 | CG2 | VAL | 281 | 42.584 | 54.256 | 90.81 | 1 | 29.72 |
| 2223 | C | VAL | 281 | 43.861 | 51.663 | 89.963 | 1 | 26.71 |
| 2224 | O | VAL | 281 | 43.069 | 50.922 | 89.394 | 1 | 26.49 |
| 2225 | N | GLN | 282 | 44.939 | 52.152 | 89.361 | 1 | 27.5 |
| 2226 | CA | GLN | 282 | 45.228 | 51.832 | 87.964 | 1 | 28.61 |
| 2227 | CB | GLN | 282 | 46.247 | 52.818 | 87.395 | 1 | 30.28 |
| 2228 | CG | GLN | 282 | 45.711 | 54.232 | 87.279 | 1 | 34.49 |
| 2229 | CD | GLN | 282 | 46.75 | 55.214 | 86.773 | 1 | 39.88 |
| 2230 | OE1 | GLN | 282 | 46.492 | 56.414 | 86.692 | 1 | 43.4 |
| 2231 | NE2 | GLN | 282 | 47.935 | 54.708 | 86.431 | 1 | 41.09 |
| 2232 | C | GLN | 282 | 45.744 | 50.409 | 87.808 | 1 | 28.72 |
| 2233 | O | GLN | 282 | 46.041 | 49.97 | 86.698 | 1 | 29 |
| 2234 | N | VAL | 283 | 45.846 | 49.69 | 88.923 | 1 | 26.48 |
| 2235 | CA | VAL | 283 | 46.311 | 48.314 | 88.898 | 1 | 24.82 |
| 2236 | CB | VAL | 283 | 46.386 | 47.727 | 90.337 | 1 | 24.36 |
| 2237 | CG1 | VAL | 283 | 44.992 | 47.641 | 90.93 | 1 | 22.65 |
| 2238 | CG2 | VAL | 283 | 47.064 | 46.355 | 90.312 | 1 | 24.34 |
| 2239 | C | VAL | 283 | 45.371 | 47.453 | 88.037 | 1 | 24.17 |
| 2240 | O | VAL | 283 | 45.757 | 46.392 | 87.553 | 1 | 24.72 |
| 2241 | N | PHE | 284 | 44.145 | 47.92 | 87.835 | 1 | 23.25 |
| 2242 | CA | PHE | 284 | 43.182 | 47.177 | 87.024 | 1 | 23.62 |
| 2243 | CB | PHE | 284 | 41.757 | 47.473 | 87.5 | 1 | 23.6 |
| 2244 | CG | PHE | 284 | 41.496 | 47.06 | 88.923 | 1 | 24.18 |
| 2245 | CD1 | PHE | 284 | 41.483 | 45.711 | 89.282 | 1 | 22.53 |
| 2246 | CD2 | PHE | 284 | 41.273 | 48.024 | 89.909 | 1 | 22.51 |
| 2247 | CE1 | PHE | 284 | 41.251 | 45.329 | 90.607 | 1 | 23.77 |
| 2248 | CE2 | PHE | 284 | 41.043 | 47.654 | 91.23 | 1 | 22.18 |
| 2249 | CZ | PHE | 284 | 41.031 | 46.303 | 91.584 | 1 | 22.16 |
| 2250 | C | PHE | 284 | 43.283 | 47.504 | 85.531 | 1 | 25.17 |
| 2251 | O | PHE | 284 | 42.581 | 46.905 | 84.705 | 1 | 24.92 |
| 2252 | N | GLU | 285 | 44.163 | 48.439 | 85.184 | 1 | 27.11 |
| 2253 | CA | GLU | 285 | 44.312 | 48.874 | 83.798 | 1 | 29.76 |
| 2254 | CB | GLU | 285 | 44.623 | 50.367 | 83.757 | 1 | 32.45 |
| 2255 | CG | GLU | 285 | 43.413 | 51.25 | 83.992 | 1 | 39.53 |
| 2256 | CD | GLU | 285 | 43.792 | 52.711 | 84.138 | 1 | 45.17 |
| 2257 | OE1 | GLU | 285 | 44.736 | 53.159 | 83.441 | 1 | 48.18 |
| 2258 | OE2 | GLU | 285 | 43.138 | 53.414 | 84.943 | 1 | 49.47 |
| 2259 | C | GLU | 285 | 45.328 | 48.131 | 82.948 | 1 | 29.29 |
| 2260 | O | GLU | 285 | 45.457 | 48.412 | 81.758 | 1 | 29.9 |
| 2261 | N | PHE | 286 | 46.052 | 47.189 | 83.535 | 1 | 27.1 |
| 2262 | CA | PHE | 286 | 47.017 | 46.435 | 82.755 | 1 | 26.36 |
| 2263 | CB | PHE | 286 | 48.375 | 47.143 | 82.727 | 1 | 26.81 |
| 2264 | CG | PHE | 286 | 49.09 | 47.159 | 84.056 | 1 | 27.15 |
| 2265 | CD1 | PHE | 286 | 48.684 | 48.014 | 85.078 | 1 | 27.28 |
| 2266 | CD2 | PHE | 286 | 50.161 | 46.304 | 84.284 | 1 | 27.07 |
| 2267 | CE1 | PHE | 286 | 49.338 | 48.015 | 86.317 | 1 | 27.05 |
| 2268 | CE2 | PHE | 286 | 50.82 | 46.295 | 85.515 | 1 | 28.67 |
| 2269 | CZ | PHE | 286 | 50.407 | 47.151 | 86.531 | 1 | 26.96 |
| 2270 | C | PHE | 286 | 47.173 | 45.03 | 83.315 | 1 | 27.5 |
| 2271 | O | PHE | 286 | 46.636 | 44.713 | 84.379 | 1 | 27.55 |
| 2272 | N | GLN | 287 | 47.911 | 44.191 | 82.593 | 1 | 25.8 |
| 2273 | CA | GLN | 287 | 48.135 | 42.815 | 83.008 | 1 | 26.97 |
| 2274 | CB | GLN | 287 | 47.224 | 41.873 | 82.215 | 1 | 26.77 |
| 2275 | CG | GLN | 287 | 45.742 | 42.192 | 82.333 | 1 | 29.69 |
| 2276 | CD | GLN | 287 | 44.893 | 41.246 | 81.52 | 1 | 30.19 |
| 2277 | OE1 | GLN | 287 | 44.981 | 40.029 | 81.682 | 1 | 32.01 |
| 2278 | NE2 | GLN | 287 | 44.066 | 41.795 | 80.637 | 1 | 31.32 |
| 2279 | C | GLN | 287 | 49.588 | 42.423 | 82.783 | 1 | 27.79 |
| 2280 | O | GLN | 287 | 50.289 | 43.044 | 81.982 | 1 | 28.2 |
| 2281 | N | LEU | 288 | 50.039 | 41.393 | 83.494 | 1 | 27.7 |
| 2282 | CA | LEU | 288 | 51.411 | 40.9 | 83.363 | 1 | 27.07 |
| 2283 | CB | LEU | 288 | 52.063 | 40.785 | 84.743 | 1 | 27.06 |
| 2284 | CG | LEU | 288 | 52.234 | 42.062 | 85.571 | 1 | 28.71 |
| 2285 | CD1 | LEU | 288 | 52.724 | 41.709 | 86.985 | 1 | 28.37 |
| 2286 | CD2 | LEU | 288 | 53.226 | 42.991 | 84.885 | 1 | 28.27 |
| 2287 | C | LEU | 288 | 51.388 | 39.526 | 82.689 | 1 | 27.12 |
| 2288 | O | LEU | 288 | 50.478 | 38.735 | 82.928 | 1 | 24.97 |
| 2289 | N | THR | 289 | 52.387 | 39.244 | 81.853 | 1 | 26.53 |
| 2290 | CA | THR | 289 | 52.455 | 37.957 | 81.158 | 1 | 27.17 |
| 2291 | CB | THR | 289 | 53.336 | 38.047 | 79.897 | 1 | 27.63 |
| 2292 | OG1 | THR | 289 | 54.682 | 38.364 | 80.278 | 1 | 26.9 |
| 2293 | CG2 | THR | 289 | 52.812 | 39.136 | 78.959 | 1 | 25.8 |
| 2294 | C | THR | 289 | 53.038 | 36.883 | 82.077 | 1 | 27.96 |
| 2295 | O | THR | 289 | 53.555 | 37.197 | 83.147 | 1 | 27.68 |
| 2296 | N | SER | 290 | 52.951 | 35.622 | 81.662 | 1 | 28.4 |
| 2297 | CA | SER | 290 | 53.469 | 34.528 | 82.47 | 1 | 30.6 |
| 2298 | CB | SER | 290 | 53.229 | 33.179 | 81.778 | 1 | 30.42 |
| 2299 | OG | SER | 290 | 53.976 | 33.073 | 80.576 | 1 | 31.38 |
| 2300 | C | SER | 290 | 54.963 | 34.725 | 82.732 | 1 | 32.77 |
| 2301 | O | SER | 290 | 55.459 | 34.428 | 83.823 | 1 | 30.89 |
| 2302 | N | GLU | 291 | 55.679 | 35.242 | 81.735 | 1 | 33.92 |
| 2303 | CA | GLU | 291 | 57.117 | 35.472 | 81.871 | 1 | 34.91 |
| 2304 | CB | GLU | 291 | 57.731 | 35.831 | 80.516 | 1 | 39.23 |
| 2305 | CG | GLU | 291 | 57.841 | 34.657 | 79.563 | 1 | 45.97 |
| 2306 | CD | GLU | 291 | 58.255 | 35.077 | 78.161 | 1 | 50.17 |
| 2307 | OE1 | GLU | 291 | 59.283 | 35.783 | 78.024 | 1 | 51.85 |
| 2308 | OE2 | GLU | 291 | 57.553 | 34.692 | 77.197 | 1 | 52.8 |
| 2309 | C | GLU | 291 | 57.436 | 36.569 | 82.874 | 1 | 33.19 |
| 2310 | O | GLU | 291 | 58.407 | 36.473 | 83.625 | 1 | 31.81 |
| 2311 | N | GLU | 292 | 56.631 | 37.621 | 82.883 | 1 | 31.47 |
| 2312 | CA | GLU | 292 | 56.864 | 38.703 | 83.825 | 1 | 31.8 |
| 2313 | CB | GLU | 292 | 56.025 | 39.923 | 83.444 | 1 | 31.61 |
| 2314 | CG | GLU | 292 | 56.276 | 40.389 | 82.013 | 1 | 33.47 |
| 2315 | CD | GLU | 292 | 55.423 | 41.572 | 81.617 | 1 | 32.37 |
| 2316 | OE1 | GLU | 292 | 54.201 | 41.539 | 81.864 | 1 | 31.84 |
| 2317 | OE2 | GLU | 292 | 55.972 | 42.534 | 81.043 | 1 | 36.21 |
| 2318 | C | GLU | 292 | 56.523 | 38.221 | 85.245 | 1 | 31.56 |
| 2319 | O | GLU | 292 | 57.179 | 38.61 | 86.209 | 1 | 32.01 |
| 2320 | N | MET | 293 | 55.504 | 37.375 | 85.37 | 1 | 30.5 |
| 2321 | CA | MET | 293 | 55.13 | 36.846 | 86.675 | 1 | 29.87 |
| 2322 | CB | MET | 293 | 53.841 | 36.015 | 86.579 | 1 | 28.77 |
| 2323 | CG | MET | 293 | 52.554 | 36.849 | 86.431 | 1 | 26.63 |
| 2324 | SD | MET | 293 | 52.246 | 37.9 | 87.874 | 1 | 26.45 |
| 2325 | CE | MET | 293 | 51.638 | 36.608 | 89.044 | 1 | 25.3 |
| 2326 | C | MET | 293 | 56.273 | 35.981 | 87.206 | 1 | 32.23 |
| 2327 | O | MET | 293 | 56.614 | 36.05 | 88.389 | 1 | 31.14 |
| 2328 | N | LYS | 294 | 56.863 | 35.164 | 86.332 | 1 | 32.57 |
| 2329 | CA | LYS | 294 | 57.974 | 34.299 | 86.733 | 1 | 35.38 |
| 2330 | CB | LYS | 294 | 58.431 | 33.41 | 85.57 | 1 | 39.68 |
| 2331 | CG | LYS | 294 | 57.96 | 31.965 | 85.65 | 1 | 45.54 |
| 2332 | CD | LYS | 294 | 56.448 | 31.852 | 85.514 | 1 | 49.88 |
| 2333 | CE | LYS | 294 | 55.999 | 30.391 | 85.456 | 1 | 52.47 |
| 2334 | NZ | LYS | 294 | 56.305 | 29.635 | 86.714 | 1 | 54.82 |
| 2335 | C | LYS | 294 | 59.159 | 35.127 | 87.207 | 1 | 34.03 |
| 2336 | O | LYS | 294 | 59.832 | 34.768 | 88.172 | 1 | 33.36 |
| 2337 | N | ALA | 295 | 59.409 | 36.236 | 86.52 | 1 | 32.69 |
| 2338 | CA | ALA | 295 | 60.509 | 37.114 | 86.876 | 1 | 33.18 |
| 2339 | CB | ALA | 295 | 60.597 | 38.286 | 85.898 | 1 | 33.31 |
| 2340 | C | ALA | 295 | 60.326 | 37.637 | 88.292 | 1 | 33.5 |
| 2341 | O | ALA | 295 | 61.28 | 37.688 | 89.069 | 1 | 32.63 |
| 2342 | N | ILE | 296 | 59.099 | 38.027 | 88.628 | 1 | 31.97 |
| 2343 | CA | ILE | 296 | 58.828 | 38.561 | 89.956 | 1 | 31.01 |
| 2344 | CB | ILE | 296 | 57.451 | 39.252 | 90 | 1 | 29.14 |
| 2345 | CG2 | ILE | 296 | 57.227 | 39.865 | 91.379 | 1 | 27.21 |
| 2346 | CG1 | ILE | 296 | 57.406 | 40.352 | 88.931 | 1 | 28.02 |
| 2347 | CD1 | ILE | 296 | 56.034 | 40.958 | 88.686 | 1 | 25.89 |
| 2348 | C | ILE | 296 | 58.932 | 37.483 | 91.033 | 1 | 31.54 |
| 2349 | O | ILE | 296 | 59.401 | 37.759 | 92.139 | 1 | 31.57 |
| 2350 | N | ASP | 297 | 58.517 | 36.261 | 90.705 | 1 | 31.84 |
| 2351 | CA | ASP | 297 | 58.613 | 35.149 | 91.652 | 1 | 33.86 |
| 2352 | CB | ASP | 297 | 58.059 | 33.85 | 91.061 | 1 | 34.57 |
| 2353 | CG | ASP | 297 | 56.55 | 33.81 | 91.032 | 1 | 36.81 |
| 2354 | OD1 | ASP | 297 | 55.902 | 34.511 | 91.839 | 1 | 39.31 |
| 2355 | OD2 | ASP | 297 | 56.008 | 33.049 | 90.206 | 1 | 38.82 |
| 2356 | C | ASP | 297 | 60.068 | 34.895 | 92.025 | 1 | 34.35 |
| 2357 | O | ASP | 297 | 60.363 | 34.396 | 93.111 | 1 | 34.05 |
| 2358 | N | GLY | 298 | 60.976 | 35.229 | 91.114 | 1 | 34.64 |
| 2359 | CA | GLY | 298 | 62.39 | 35.009 | 91.366 | 1 | 34.61 |
| 2360 | C | GLY | 298 | 63.035 | 36.004 | 92.312 | 1 | 35.12 |
| 2361 | O | GLY | 298 | 64.189 | 35.829 | 92.697 | 1 | 35.46 |
| 2362 | N | LEU | 299 | 62.3 | 37.045 | 92.691 | 1 | 34.08 |
| 2363 | CA | LEU | 299 | 62.829 | 38.062 | 93.591 | 1 | 34.15 |
| 2364 | CB | LEU | 299 | 62.093 | 39.387 | 93.386 | 1 | 33.2 |
| 2365 | CG | LEU | 299 | 62.196 | 40.02 | 92.004 | 1 | 33.51 |
| 2366 | CD1 | LEU | 299 | 61.387 | 41.306 | 91.968 | 1 | 32.39 |
| 2367 | CD2 | LEU | 299 | 63.658 | 40.295 | 91.679 | 1 | 34.46 |
| 2368 | C | LEU | 299 | 62.73 | 37.663 | 95.06 | 1 | 34.53 |
| 2369 | O | LEU | 299 | 63.289 | 38.33 | 95.931 | 1 | 34.06 |
| 2370 | N | ASN | 300 | 62.02 | 36.577 | 95.335 | 1 | 34.76 |
| 2371 | CA | ASN | 300 | 61.847 | 36.127 | 96.705 | 1 | 36.44 |
| 2372 | CB | ASN | 300 | 61.079 | 34.812 | 96.737 | 1 | 35.58 |
| 2373 | CG | ASN | 300 | 60.647 | 34.433 | 98.135 | 1 | 36.05 |
| 2374 | OD1 | ASN | 300 | 60.01 | 35.222 | 98.836 | 1 | 36.46 |
| 2375 | ND2 | ASN | 300 | 60.982 | 33.221 | 98.547 | 1 | 37.41 |
| 2376 | C | ASN | 300 | 63.187 | 35.959 | 97.411 | 1 | 38.47 |
| 2377 | O | ASN | 300 | 64.067 | 35.248 | 96.931 | 1 | 38.65 |
| 2378 | N | ARG | 301 | 63.339 | 36.614 | 98.554 | 1 | 39.52 |
| 2379 | CA | ARG | 301 | 64.586 | 36.526 | 99.298 | 1 | 41.71 |
| 2380 | CB | ARG | 301 | 65.442 | 37.765 | 99.016 | 1 | 43.08 |
| 2381 | CG | ARG | 301 | 64.727 | 39.072 | 99.283 | 1 | 46.16 |
| 2382 | CD | ARG | 301 | 65.672 | 40.256 | 99.172 | 1 | 49.91 |
| 2383 | NE | ARG | 301 | 66.08 | 40.526 | 97.798 | 1 | 52.67 |
| 2384 | CZ | ARG | 301 | 66.937 | 41.484 | 97.45 | 1 | 54.39 |
| 2385 | NH1 | ARG | 301 | 67.483 | 42.268 | 98.376 | 1 | 54.14 |
| 2386 | NH2 | ARG | 301 | 67.244 | 41.662 | 96.171 | 1 | 54.23 |
| 2387 | C | ARG | 301 | 64.342 | 36.386 | 100.798 | 1 | 42.2 |
| 2388 | O | ARG | 301 | 65.248 | 36.593 | 101.605 | 1 | 41.63 |
| 2389 | N | ASN | 302 | 63.111 | 36.036 | 101.157 | 1 | 41.8 |
| 2390 | CA | ASN | 302 | 62.72 | 35.858 | 102.549 | 1 | 42.57 |
| 2391 | CB | ASN | 302 | 63.375 | 34.604 | 103.129 | 1 | 48.14 |
| 2392 | CG | ASN | 302 | 62.704 | 33.328 | 102.656 | 1 | 49.75 |
| 2393 | OD1 | ASN | 302 | 62.655 | 33.037 | 101.458 | 1 | 51.99 |
| 2394 | ND2 | ASN | 302 | 62.179 | 32.559 | 103.601 | 1 | 53.11 |
| 2395 | C | ASN | 302 | 63.029 | 37.052 | 103.441 | 1 | 40.58 |
| 2396 | O | ASN | 302 | 63.645 | 36.909 | 104.493 | 1 | 40.69 |
| 2397 | N | VAL | 303 | 62.599 | 38.23 | 103.012 | 1 | 38.42 |
| 2398 | CA | VAL | 303 | 62.807 | 39.445 | 103.78 | 1 | 36.2 |
| 2399 | CB | VAL | 303 | 63.283 | 40.61 | 102.887 | 1 | 36.8 |
| 2400 | CG1 | VAL | 303 | 63.207 | 41.927 | 103.652 | 1 | 37.98 |
| 2401 | CG2 | VAL | 303 | 64.711 | 40.359 | 102.434 | 1 | 38.8 |
| 2402 | C | VAL | 303 | 61.49 | 39.845 | 104.439 | 1 | 34.67 |
| 2403 | O | VAL | 303 | 60.564 | 40.315 | 103.77 | 1 | 33.77 |
| 2404 | N | ARG | 304 | 61.416 | 39.655 | 105.751 | 1 | 31.64 |
| 2405 | CA | ARG | 304 | 60.223 | 40.003 | 106.513 | 1 | 30.43 |
| 2406 | CB | ARG | 304 | 59.967 | 38.938 | 107.596 | 1 | 30.41 |
| 2407 | CG | ARG | 304 | 58.654 | 39.11 | 108.348 | 1 | 29.74 |
| 2408 | CD | ARG | 304 | 58.533 | 38.137 | 109.519 | 1 | 28.02 |
| 2409 | NE | ARG | 304 | 58.301 | 36.74 | 109.147 | 1 | 25.39 |
| 2410 | CZ | ARG | 304 | 57.155 | 36.254 | 108.684 | 1 | 27.16 |
| 2411 | NH1 | ARG | 304 | 56.109 | 37.05 | 108.514 | 1 | 28.29 |
| 2412 | NH2 | ARG | 304 | 57.047 | 34.958 | 108.417 | 1 | 25.72 |
| 2413 | C | ARG | 304 | 60.473 | 41.365 | 107.151 | 1 | 29.91 |
| 2414 | O | ARG | 304 | 61.386 | 41.51 | 107.967 | 1 | 29.76 |
| 2415 | N | TYR | 305 | 59.689 | 42.369 | 106.764 | 1 | 28.58 |
| 2416 | CA | TYR | 305 | 59.842 | 43.712 | 107.326 | 1 | 29.92 |
| 2417 | CB | TYR | 305 | 59.175 | 44.759 | 106.43 | 1 | 30.86 |
| 2418 | CG | TYR | 305 | 59.829 | 44.92 | 105.075 | 1 | 32.58 |
| 2419 | CD1 | TYR | 305 | 61.124 | 45.423 | 104.958 | 1 | 33.72 |
| 2420 | CE1 | TYR | 305 | 61.73 | 45.566 | 103.708 | 1 | 34.63 |
| 2421 | CD2 | TYR | 305 | 59.153 | 44.563 | 103.911 | 1 | 32.87 |
| 2422 | CE2 | TYR | 305 | 59.749 | 44.7 | 102.657 | 1 | 35.12 |
| 2423 | CZ | TYR | 305 | 61.036 | 45.199 | 102.564 | 1 | 35.52 |
| 2424 | OH | TYR | 305 | 61.624 | 45.304 | 101.327 | 1 | 37.64 |
| 2425 | C | TYR | 305 | 59.266 | 43.849 | 108.735 | 1 | 30.66 |
| 2426 | O | TYR | 305 | 59.838 | 44.542 | 109.576 | 1 | 30.76 |
| 2427 | N | LEU | 306 | 58.128 | 43.205 | 108.988 | 1 | 29.79 |
| 2428 | CA | LEU | 306 | 57.491 | 43.28 | 110.298 | 1 | 29.5 |
| 2429 | CB | LEU | 306 | 56.014 | 43.638 | 110.145 | 1 | 32.3 |
| 2430 | CG | LEU | 306 | 55.756 | 45.115 | 109.854 | 1 | 35.29 |
| 2431 | CD1 | LEU | 306 | 54.351 | 45.289 | 109.342 | 1 | 39.03 |
| 2432 | CD2 | LEU | 306 | 55.975 | 45.929 | 111.126 | 1 | 37 |
| 2433 | C | LEU | 306 | 57.625 | 41.984 | 111.072 | 1 | 29.03 |
| 2434 | O | LEU | 306 | 56.808 | 41.078 | 110.935 | 1 | 25.39 |
| 2435 | N | THR | 307 | 58.665 | 41.905 | 111.893 | 1 | 29.26 |
| 2436 | CA | THR | 307 | 58.907 | 40.713 | 112.688 | 1 | 30.49 |
| 2437 | CB | THR | 307 | 60.402 | 40.537 | 112.984 | 1 | 31.61 |
| 2438 | OG1 | THR | 307 | 60.868 | 41.676 | 113.714 | 1 | 33.59 |
| 2439 | CG2 | THR | 307 | 61.194 | 40.402 | 111.689 | 1 | 33.13 |
| 2440 | C | THR | 307 | 58.172 | 40.776 | 114.017 | 1 | 29.79 |
| 2441 | O | THR | 307 | 57.867 | 39.737 | 114.598 | 1 | 29.68 |
| 2442 | N | LEU | 308 | 57.9 | 41.989 | 114.492 | 1 | 28.32 |
| 2443 | CA | LEU | 308 | 57.207 | 42.176 | 115.767 | 1 | 30.8 |
| 2444 | CB | LEU | 308 | 55.725 | 41.79 | 115.623 | 1 | 31.6 |
| 2445 | CG | LEU | 308 | 54.873 | 42.714 | 114.74 | 1 | 34.08 |
| 2446 | CD1 | LEU | 308 | 53.54 | 42.067 | 114.422 | 1 | 35.47 |
| 2447 | CD2 | LEU | 308 | 54.662 | 44.035 | 115.458 | 1 | 34.47 |
| 2448 | C | LEU | 308 | 57.87 | 41.314 | 116.844 | 1 | 30.56 |
| 2449 | O | LEU | 308 | 57.192 | 40.717 | 117.684 | 1 | 27.97 |
| 2450 | N | ASP | 309 | 59.198 | 41.256 | 116.813 | 1 | 30.42 |
| 2451 | CA | ASP | 309 | 59.955 | 40.453 | 117.769 | 1 | 32.55 |
| 2452 | CB | ASP | 309 | 61.439 | 40.467 | 117.41 | 1 | 36.27 |
| 2453 | CG | ASP | 309 | 61.919 | 41.832 | 117.016 | 1 | 39.6 |
| 2454 | OD1 | ASP | 309 | 61.657 | 42.797 | 117.765 | 1 | 42.38 |
| 2455 | OD2 | ASP | 309 | 62.555 | 41.943 | 115.948 | 1 | 44.74 |
| 2456 | C | ASP | 309 | 59.789 | 40.827 | 119.239 | 1 | 31.54 |
| 2457 | O | ASP | 309 | 60.25 | 40.096 | 120.109 | 1 | 31.57 |
| 2458 | N | ILE | 310 | 59.152 | 41.96 | 119.525 | 1 | 31.29 |
| 2459 | CA | ILE | 310 | 58.935 | 42.336 | 120.915 | 1 | 31.99 |
| 2460 | CB | ILE | 310 | 58.413 | 43.782 | 121.057 | 1 | 33.71 |
| 2461 | CG2 | ILE | 310 | 59.488 | 44.76 | 120.617 | 1 | 36.9 |
| 2462 | CG1 | ILE | 310 | 57.134 | 43.961 | 120.245 | 1 | 35.24 |
| 2463 | CD1 | ILE | 310 | 56.419 | 45.26 | 120.535 | 1 | 38.78 |
| 2464 | C | ILE | 310 | 57.901 | 41.378 | 121.514 | 1 | 30.39 |
| 2465 | O | ILE | 310 | 57.754 | 41.304 | 122.729 | 1 | 30.07 |
| 2466 | N | PHE | 311 | 57.2 | 40.647 | 120.645 | 1 | 27.74 |
| 2467 | CA | PHE | 311 | 56.185 | 39.686 | 121.066 | 1 | 25.41 |
| 2468 | CB | PHE | 311 | 54.937 | 39.79 | 120.169 | 1 | 26.66 |
| 2469 | CG | PHE | 311 | 54.149 | 41.063 | 120.354 | 1 | 29.51 |
| 2470 | CD1 | PHE | 311 | 53.505 | 41.339 | 121.556 | 1 | 31.8 |
| 2471 | CD2 | PHE | 311 | 54.066 | 41.996 | 119.327 | 1 | 31.54 |
| 2472 | CE1 | PHE | 311 | 52.79 | 42.532 | 121.735 | 1 | 32.67 |
| 2473 | CE2 | PHE | 311 | 53.354 | 43.189 | 119.498 | 1 | 33.98 |
| 2474 | CZ | PHE | 311 | 52.717 | 43.455 | 120.703 | 1 | 31.44 |
| 2475 | C | PHE | 311 | 56.718 | 38.25 | 121.026 | 1 | 24.05 |
| 2476 | O | PHE | 311 | 55.969 | 37.315 | 121.262 | 1 | 22.96 |
| 2477 | N | ALA | 312 | 58.005 | 38.076 | 120.715 | 1 | 22.35 |
| 2478 | CA | ALA | 312 | 58.616 | 36.743 | 120.651 | 1 | 22.48 |
| 2479 | CB | ALA | 312 | 60.019 | 36.827 | 120.035 | 1 | 24.12 |
| 2480 | C | ALA | 312 | 58.706 | 36.108 | 122.038 | 1 | 21.84 |
| 2481 | O | ALA | 312 | 58.876 | 36.807 | 123.035 | 1 | 21.95 |
| 2482 | N | GLY | 313 | 58.584 | 34.784 | 122.101 | 1 | 21.93 |
| 2483 | CA | GLY | 313 | 58.662 | 34.1 | 123.385 | 1 | 21.35 |
| 2484 | C | GLY | 313 | 57.513 | 33.138 | 123.608 | 1 | 22.17 |
| 2485 | O | GLY | 313 | 57.735 | 31.956 | 123.844 | 1 | 21.77 |
| 2486 | N | PRO | 314 | 56.263 | 33.627 | 123.562 | 1 | 21.99 |
| 2487 | CD | PRO | 314 | 55.906 | 35.057 | 123.499 | 1 | 22.26 |
| 2488 | CA | PRO | 314 | 55.068 | 32.798 | 123.753 | 1 | 21.99 |
| 2489 | CB | PRO | 314 | 53.923 | 33.812 | 123.648 | 1 | 23.79 |
| 2490 | CG | PRO | 314 | 54.544 | 35.078 | 124.16 | 1 | 21.52 |
| 2491 | C | PRO | 314 | 54.959 | 31.725 | 122.669 | 1 | 22.73 |
| 2492 | O | PRO | 314 | 55.431 | 31.905 | 121.546 | 1 | 20.06 |
| 2493 | N | PRO | 315 | 54.332 | 30.589 | 122.994 | 1 | 23.89 |
| 2494 | CD | PRO | 315 | 53.725 | 30.2 | 124.278 | 1 | 23.5 |
| 2495 | CA | PRO | 315 | 54.195 | 29.528 | 121.995 | 1 | 24.17 |
| 2496 | CB | PRO | 315 | 53.477 | 28.409 | 122.762 | 1 | 25.56 |
| 2497 | CG | PRO | 315 | 52.733 | 29.146 | 123.844 | 1 | 26.38 |
| 2498 | C | PRO | 315 | 53.434 | 29.99 | 120.75 | 1 | 23.75 |
| 2499 | O | PRO | 315 | 53.656 | 29.467 | 119.66 | 1 | 21.44 |
| 2500 | N | ASN | 316 | 52.564 | 30.988 | 120.891 | 1 | 22.99 |
| 2501 | CA | ASN | 316 | 51.823 | 31.448 | 119.719 | 1 | 23.53 |
| 2502 | CB | ASN | 316 | 50.456 | 32.017 | 120.114 | 1 | 24.15 |
| 2503 | CG | ASN | 316 | 49.496 | 30.953 | 120.616 | 1 | 26.61 |
| 2504 | OD1 | ASN | 316 | 49.741 | 29.752 | 120.475 | 1 | 25.81 |
| 2505 | ND2 | ASN | 316 | 48.379 | 31.395 | 121.191 | 1 | 27.23 |
| 2506 | C | ASN | 316 | 52.554 | 32.466 | 118.829 | 1 | 22.97 |
| 2507 | O | ASN | 316 | 51.985 | 32.899 | 117.828 | 1 | 22.03 |
| 2508 | N | TYR | 317 | 53.777 | 32.877 | 119.185 | 1 | 22.46 |
| 2509 | CA | TYR | 317 | 54.514 | 33.809 | 118.322 | 1 | 21.8 |
| 2510 | CB | TYR | 317 | 55.935 | 34.053 | 118.833 | 1 | 22.35 |
| 2511 | CG | TYR | 317 | 56.73 | 34.969 | 117.926 | 1 | 23.24 |
| 2512 | CD1 | TYR | 317 | 56.404 | 36.325 | 117.813 | 1 | 22.38 |
| 2513 | CE1 | TYR | 317 | 57.108 | 37.169 | 116.945 | 1 | 22.18 |
| 2514 | CD2 | TYR | 317 | 57.784 | 34.476 | 117.151 | 1 | 22.97 |
| 2515 | CE2 | TYR | 317 | 58.491 | 35.314 | 116.28 | 1 | 22.18 |
| 2516 | CZ | TYR | 317 | 58.148 | 36.648 | 116.182 | 1 | 23.21 |
| 2517 | OH | TYR | 317 | 58.846 | 37.451 | 115.314 | 1 | 24.39 |
| 2518 | C | TYR | 317 | 54.534 | 33.067 | 116.976 | 1 | 22.49 |
| 2519 | O | TYR | 317 | 55.036 | 31.946 | 116.878 | 1 | 22.27 |
| 2520 | N | PRO | 318 | 53.981 | 33.688 | 115.924 | 1 | 22.42 |
| 2521 | CD | PRO | 318 | 53.57 | 35.098 | 115.948 | 1 | 21.67 |
| 2522 | CA | PRO | 318 | 53.87 | 33.138 | 114.567 | 1 | 23.78 |
| 2523 | CB | PRO | 318 | 52.764 | 33.986 | 113.926 | 1 | 23.9 |
| 2524 | CG | PRO | 318 | 52.502 | 35.136 | 114.898 | 1 | 24.92 |
| 2525 | C | PRO | 318 | 55.035 | 33.025 | 113.605 | 1 | 24.17 |
| 2526 | O | PRO | 318 | 55.026 | 32.133 | 112.753 | 1 | 24.57 |
| 2527 | N | PHE | 319 | 56.017 | 33.908 | 113.723 | 1 | 24.31 |
| 2528 | CA | PHE | 319 | 57.105 | 33.929 | 112.764 | 1 | 27.99 |
| 2529 | CB | PHE | 319 | 57.528 | 35.386 | 112.531 | 1 | 25.41 |
| 2530 | CG | PHE | 319 | 56.36 | 36.332 | 112.331 | 1 | 24.36 |
| 2531 | CD1 | PHE | 319 | 55.244 | 35.949 | 111.584 | 1 | 24.42 |
| 2532 | CD2 | PHE | 319 | 56.387 | 37.606 | 112.874 | 1 | 25.1 |
| 2533 | CE1 | PHE | 319 | 54.167 | 36.833 | 111.38 | 1 | 22.49 |
| 2534 | CE2 | PHE | 319 | 55.326 | 38.496 | 112.679 | 1 | 26.18 |
| 2535 | CZ | PHE | 319 | 54.218 | 38.107 | 111.932 | 1 | 23.79 |
| 2536 | C | PHE | 319 | 58.312 | 33.053 | 113.042 | 1 | 30.78 |
| 2537 | O | PHE | 319 | 59.367 | 33.255 | 112.454 | 1 | 34.17 |
| 2538 | N | SER | 320 | 58.15 | 32.069 | 113.912 | 1 | 32.82 |
| 2539 | CA | SER | 320 | 59.237 | 31.156 | 114.228 | 1 | 35.99 |
| 2540 | CB | SER | 320 | 59.107 | 30.649 | 115.664 | 1 | 35.8 |
| 2541 | OG | SER | 320 | 59.553 | 31.627 | 116.577 | 1 | 39.46 |
| 2542 | C | SER | 320 | 59.25 | 29.968 | 113.273 | 1 | 36.75 |
| 2543 | O | SER | 320 | 60.308 | 29.547 | 112.814 | 1 | 37.09 |
| 2544 | N | ASP | 321 | 58.07 | 29.433 | 112.972 | 1 | 36.63 |
| 2545 | CA | ASP | 321 | 57.969 | 28.286 | 112.081 | 1 | 37.7 |
| 2546 | CB | ASP | 321 | 56.557 | 27.699 | 112.12 | 1 | 37.98 |
| 2547 | CG | ASP | 321 | 56.151 | 27.257 | 113.504 | 1 | 39.08 |
| 2548 | OD1 | ASP | 321 | 57.04 | 27.127 | 114.371 | 1 | 41.4 |
| 2549 | OD2 | ASP | 321 | 54.944 | 27.03 | 113.719 | 1 | 37.81 |
| 2550 | C | ASP | 321 | 58.318 | 28.641 | 110.641 | 1 | 38.03 |
| 2551 | O | ASP | 321 | 58.362 | 29.814 | 110.274 | 1 | 36.77 |
| 2552 | N | GLU | 322 | 58.566 | 27.62 | 109.829 | 1 | 38.02 |
| 2553 | CA | GLU | 322 | 58.9 | 27.839 | 108.429 | 1 | 40.14 |
| 2554 | CB | GLU | 322 | 59.163 | 26.509 | 107.728 | 1 | 43.27 |
| 2555 | CG | GLU | 322 | 59.509 | 26.666 | 106.259 | 1 | 49.62 |
| 2556 | CD | GLU | 322 | 59.682 | 25.334 | 105.562 | 1 | 53.4 |
| 2557 | OE1 | GLU | 322 | 58.738 | 24.509 | 105.602 | 1 | 55.39 |
| 2558 | OE2 | GLU | 322 | 60.76 | 25.112 | 104.966 | 1 | 56.35 |
| 2559 | C | GLU | 322 | 57.772 | 28.586 | 107.723 | 1 | 38.44 |
| 2560 | O | GLU | 322 | 58.02 | 29.42 | 106.857 | 1 | 38.44 |
| 2561 | N | TYR | 323 | 56.532 | 28.273 | 108.084 | 1 | 35.34 |
| 2562 | CA | TYR | 323 | 55.373 | 28.951 | 107.507 | 1 | 34.25 |
| 2563 | CB | TYR | 323 | 55.209 | 28.587 | 106.02 | 1 | 34.15 |
| 2564 | CG | TYR | 323 | 54.545 | 27.256 | 105.747 | 1 | 34.6 |
| 2565 | CD1 | TYR | 323 | 53.197 | 27.191 | 105.389 | 1 | 35.02 |
| 2566 | CE1 | TYR | 323 | 52.568 | 25.97 | 105.156 | 1 | 36.34 |
| 2567 | CD2 | TYR | 323 | 55.254 | 26.063 | 105.866 | 1 | 36.3 |
| 2568 | CE2 | TYR | 323 | 54.637 | 24.834 | 105.64 | 1 | 38.59 |
| 2569 | CZ | TYR | 323 | 53.293 | 24.797 | 105.286 | 1 | 39.03 |
| 2570 | OH | TYR | 323 | 52.681 | 23.586 | 105.079 | 1 | 40.66 |
| 2571 | C | TYR | 323 | 54.126 | 28.568 | 108.304 | 1 | 33.55 |
| 2572 | O | TYR | 323 | 53.076 | 29.211 | 108.12 | 1 | 32.2 |
| 2573 | OXT | TYR | 323 | 54.225 | 27.623 | 109.114 | 1 | 33.11 |
| 2574 | TYR | A | | | | | | |
| 2575 | CB | SER | 3 | 23.11 | 40.285 | 92.904 | 1 | 51.49 |
| 2576 | OG | SER | 3 | 23.122 | 40.992 | 91.671 | 1 | 52.76 |
| 2577 | C | SER | 3 | 25.24 | 41.36 | 93.638 | 1 | 46.66 |
| 2578 | O | SER | 3 | 25.53 | 42.299 | 92.899 | 1 | 47.86 |
| 2579 | N | SER | 3 | 23.054 | 42.379 | 94.244 | 1 | 50.69 |
| 2580 | CA | SER | 3 | 23.787 | 41.092 | 94.021 | 1 | 49.5 |
| 2581 | N | LYS | 4 | 26.154 | 40.54 | 94.149 | 1 | 42.8 |
| 2582 | CA | LYS | 4 | 27.568 | 40.693 | 93.824 | 1 | 37.17 |
| 2583 | CB | LYS | 4 | 28.391 | 40.953 | 95.092 | 1 | 39.55 |
| 2584 | CG | LYS | 4 | 28.317 | 42.395 | 95.597 | 1 | 41.23 |
| 2585 | CD | LYS | 4 | 29.222 | 42.614 | 96.796 | 1 | 43.05 |
| 2586 | CE | LYS | 4 | 29.335 | 44.087 | 97.159 | 1 | 44.48 |
| 2587 | NZ | LYS | 4 | 28.017 | 44.716 | 97.433 | 1 | 46.38 |
| 2588 | C | LYS | 4 | 28.084 | 39.452 | 93.099 | 1 | 33.85 |
| 2589 | O | LYS | 4 | 27.602 | 38.349 | 93.333 | 1 | 32.85 |
| 2590 | N | TYR | 5 | 29.054 | 39.64 | 92.206 | 1 | 29.33 |
| 2591 | CA | TYR | 5 | 29.628 | 38.536 | 91.442 | 1 | 26.37 |
| 2592 | CB | TYR | 5 | 28.686 | 38.139 | 90.295 | 1 | 24.54 |
| 2593 | CG | TYR | 5 | 28.12 | 39.312 | 89.526 | 1 | 24.48 |
| 2594 | CD1 | TYR | 5 | 28.732 | 39.775 | 88.355 | 1 | 24.71 |
| 2595 | CE1 | TYR | 5 | 28.218 | 40.871 | 87.656 | 1 | 25.46 |
| 2596 | CD2 | TYR | 5 | 26.98 | 39.974 | 89.981 | 1 | 23.74 |
| 2597 | CE2 | TYR | 5 | 26.456 | 41.068 | 89.292 | 1 | 25.68 |
| 2598 | CZ | TYR | 5 | 27.077 | 41.512 | 88.133 | 1 | 27.64 |
| 2599 | OH | TYR | 5 | 26.558 | 42.591 | 87.449 | 1 | 27.65 |
| 2600 | C | TYR | 5 | 30.99 | 38.956 | 90.908 | 1 | 25.38 |
| 2601 | O | TYR | 5 | 31.26 | 40.149 | 90.754 | 1 | 24.54 |
| 2602 | N | GLN | 6 | 31.85 | 37.983 | 90.623 | 1 | 23.54 |
| 2603 | CA | GLN | 6 | 33.191 | 38.284 | 90.14 | 1 | 23.98 |
| 2604 | CB | GLN | 6 | 34.113 | 37.066 | 90.316 | 1 | 24.98 |
| 2605 | CG | GLN | 6 | 34.23 | 36.605 | 91.762 | 1 | 29.43 |
| 2606 | CD | GLN | 6 | 35.292 | 35.541 | 91.992 | 1 | 30.13 |
| 2607 | OE1 | GLN | 6 | 35.286 | 34.875 | 93.035 | 1 | 33.67 |
| 2608 | NE2 | GLN | 6 | 36.214 | 35.379 | 91.033 | 1 | 23.36 |
| 2609 | C | GLN | 6 | 33.223 | 38.751 | 88.689 | 1 | 23.14 |
| 2610 | O | GLN | 6 | 32.671 | 38.097 | 87.804 | 1 | 21.74 |
| 2611 | N | CYS | 7 | 33.881 | 39.889 | 88.478 | 1 | 22.96 |
| 2612 | CA | CYS | 7 | 34.047 | 40.52 | 87.162 | 1 | 25.27 |
| 2613 | CB | CYS | 7 | 33.195 | 41.784 | 87.036 | 1 | 27.29 |
| 2614 | SG | CYS | 7 | 31.453 | 41.58 | 87.206 | 1 | 33.83 |
| 2615 | C | CYS | 7 | 35.48 | 40.981 | 86.989 | 1 | 24.65 |
| 2616 | O | CYS | 7 | 36.238 | 41.08 | 87.958 | 1 | 24.06 |
| 2617 | N | VAL | 8 | 35.839 | 41.277 | 85.745 | 1 | 24.96 |
| 2618 | CA | VAL | 8 | 37.156 | 41.81 | 85.423 | 1 | 23.89 |
| 2619 | CB | VAL | 8 | 37.998 | 40.855 | 84.539 | 1 | 25.46 |
| 2620 | CG1 | VAL | 8 | 38.426 | 39.652 | 85.36 | 1 | 29.75 |
| 2621 | CG2 | VAL | 8 | 37.203 | 40.418 | 83.303 | 1 | 24.53 |
| 2622 | C | VAL | 8 | 36.913 | 43.096 | 84.649 | 1 | 24.28 |
| 2623 | O | VAL | 8 | 35.929 | 43.208 | 83.915 | 1 | 22.61 |
| 2624 | N | LYS | 9 | 37.799 | 44.071 | 84.821 | 1 | 22.76 |
| 2625 | CA | LYS | 9 | 37.661 | 45.341 | 84.12 | 1 | 24.08 |
| 2626 | CB | LYS | 9 | 38.384 | 46.451 | 84.885 | 1 | 22.1 |
| 2627 | CG | LYS | 9 | 38.062 | 47.846 | 84.377 | 1 | 24.27 |
| 2628 | CD | LYS | 9 | 38.73 | 48.914 | 85.236 | 1 | 28.1 |
| 2629 | CE | LYS | 9 | 38.286 | 50.312 | 84.831 | 1 | 29.63 |
| 2630 | NZ | LYS | 9 | 38.878 | 51.354 | 85.715 | 1 | 33.48 |
| 2631 | C | LYS | 9 | 38.237 | 45.241 | 82.705 | 1 | 23.39 |
| 2632 | O | LYS | 9 | 39.359 | 44.767 | 82.517 | 1 | 26.48 |
| 2633 | N | LEU | 10 | 37.462 | 45.676 | 81.717 | 1 | 23.77 |
| 2634 | CA | LEU | 10 | 37.883 | 45.646 | 80.313 | 1 | 23.05 |
| 2635 | CB | LEU | 10 | 36.649 | 45.544 | 79.405 | 1 | 22.2 |
| 2636 | CG | LEU | 10 | 38.108 | 44.165 | 78.997 | 1 | 25.16 |
| 2637 | CD1 | LEU | 10 | 36.524 | 43.11 | 79.969 | 1 | 25.8 |
| 2638 | CD2 | LEU | 10 | 34.586 | 44.232 | 78.843 | 1 | 23.3 |
| 2639 | C | LEU | 10 | 38.682 | 46.904 | 79.99 | 1 | 24.13 |
| 2640 | O | LEU | 10 | 38.622 | 47.885 | 80.735 | 1 | 21.46 |
| 2641 | N | ASN | 11 | 39.423 | 46.889 | 78.882 | 1 | 23.72 |
| 2642 | CA | ASN | 11 | 40.231 | 48.055 | 78.532 | 1 | 24.5 |
| 2643 | CB | ASN | 11 | 41.281 | 47.716 | 77.452 | 1 | 23.61 |
| 2644 | CG | ASN | 11 | 40.668 | 47.251 | 76.143 | 1 | 23.15 |
| 2645 | OD1 | ASN | 11 | 39.653 | 47.777 | 75.692 | 1 | 26.36 |
| 2646 | ND2 | ASN | 11 | 41.302 | 46.27 | 75.514 | 1 | 24.55 |
| 2647 | C | ASN | 11 | 39.422 | 49.283 | 78.117 | 1 | 23.94 |
| 2648 | O | ASN | 11 | 40.001 | 50.326 | 77.844 | 1 | 24.73 |
| 2649 | N | ASP | 12 | 38.093 | 46.172 | 78.076 | 1 | 23.89 |
| 2650 | CA | ASP | 12 | 37.265 | 50.336 | 77.73 | 1 | 23.72 |
| 2851 | CB | ASP | 12 | 36.27 | 50.01 | 76.603 | 1 | 22.85 |
| 2652 | CG | ASP | 12 | 35.165 | 49.063 | 77.039 | 1 | 22.92 |
| 2653 | OD1 | ASP | 12 | 35.21 | 48.55 | 78.176 | 1 | 23.93 |
| 2654 | OD2 | ASP | 12 | 34.243 | 48.82 | 76.228 | 1 | 25.82 |
| 2655 | C | ASP | 12 | 36.511 | 50.836 | 78.958 | 1 | 24.19 |
| 2656 | O | ASP | 12 | 35.613 | 51.672 | 78.854 | 1 | 24.59 |
| 2657 | N | GLY | 13 | 36.864 | 50.303 | 80.124 | 1 | 24.62 |
| 2658 | CA | GLY | 13 | 36.209 | 50.734 | 81.346 | 1 | 23.34 |
| 2659 | C | GLY | 13 | 35.017 | 49.907 | 81.796 | 1 | 25.14 |
| 2660 | O | GLY | 13 | 34.554 | 50.066 | 82.933 | 1 | 25.24 |
| 2661 | N | HIS | 14 | 34.505 | 49.029 | 80.932 | 1 | 21.75 |
| 2662 | CA | HIS | 14 | 33.358 | 48.213 | 81.325 | 1 | 21.95 |
| 2663 | CB | HIS | 14 | 32.501 | 47.825 | 80.102 | 1 | 23.02 |
| 2664 | CG | HIS | 14 | 31.706 | 48.964 | 79.54 | 1 | 24.58 |
| 2665 | CD2 | HIS | 14 | 30.676 | 49.674 | 80.061 | 1 | 25.5 |
| 2666 | ND1 | HIS | 14 | 31.975 | 49.528 | 78.311 | 1 | 27.89 |
| 2667 | CE1 | HIS | 14 | 31.145 | 50.535 | 78.097 | 1 | 26 |
| 2668 | NE2 | HIS | 14 | 30.347 | 50.646 | 79.145 | 1 | 27.75 |
| 2669 | C | HIS | 14 | 33.818 | 46.972 | 82.07 | 1 | 21.49 |
| 2670 | O | HIS | 14 | 35.009 | 46.653 | 82.071 | 1 | 21.38 |
| 2671 | N | PHE | 15 | 32.875 | 46.285 | 82.713 | 1 | 21.42 |
| 2672 | CA | PHE | 15 | 33.188 | 45.091 | 83.481 | 1 | 21.62 |
| 2673 | CB | PHE | 15 | 32.81 | 45.284 | 84.954 | 1 | 23.2 |
| 2674 | CG | PHE | 15 | 33.742 | 46.194 | 85.705 | 1 | 23.74 |
| 2675 | CD1 | PHE | 15 | 33.649 | 47.581 | 85.576 | 1 | 25.06 |
| 2676 | CD2 | PHE | 15 | 34.726 | 45.655 | 86.536 | 1 | 23.38 |
| 2677 | CE1 | PHE | 15 | 34.529 | 48.428 | 86.269 | 1 | 24.03 |
| 2678 | CE2 | PHE | 15 | 35.611 | 46.482 | 87.232 | 1 | 23.84 |
| 2679 | CZ | PHE | 15 | 35.511 | 47.877 | 87.099 | 1 | 25.29 |
| 2680 | C | PHE | 15 | 32.491 | 43.858 | 82.931 | 1 | 20.99 |
| 2681 | O | PHE | 15 | 31.321 | 43.902 | 82.571 | 1 | 23.17 |
| 2682 | N | MET | 16 | 33.217 | 42.751 | 82.88 | 1 | 18.67 |
| 2683 | CA | MET | 16 | 32.675 | 41.511 | 82.359 | 1 | 19.2 |
| 2684 | CB | MET | 16 | 33.5 | 41.083 | 81.135 | 1 | 20.21 |
| 2685 | CG | MET | 16 | 33.149 | 39.711 | 80.564 | 1 | 21.12 |
| 2686 | SD | MET | 16 | 34.37 | 39.145 | 79.317 | 1 | 26.36 |
| 2687 | CE | MET | 16 | 33.778 | 39.965 | 77.887 | 1 | 23.05 |
| 2688 | C | MET | 16 | 32.716 | 40.408 | 83.413 | 1 | 18.36 |
| 2689 | O | MET | 16 | 33.756 | 40.19 | 84.038 | 1 | 18.24 |
| 2690 | N | PRO | 17 | 31.581 | 39.72 | 83.638 | 1 | 17.86 |
| 2691 | CD | PRO | 17 | 30.237 | 40.048 | 83.11 | 1 | 17.68 |
| 2692 | CA | PRO | 17 | 31.52 | 38.625 | 84.624 | 1 | 18.07 |
| 2693 | CB | PRO | 17 | 30.054 | 38.194 | 84.577 | 1 | 17.06 |
| 2694 | CG | PRO | 17 | 29.31 | 39.5 | 84.196 | 1 | 17.51 |
| 2695 | C | PRO | 17 | 32.474 | 37.532 | 84.117 | 1 | 19.83 |
| 2696 | O | PRO | 17 | 32.456 | 37.21 | 82.931 | 1 | 20.42 |
| 2697 | N | VAL | 18 | 33.292 | 36.966 | 85.001 | 1 | 20.07 |
| 2698 | CA | VAL | 18 | 34.288 | 35.969 | 84.597 | 1 | 20.44 |
| 2699 | CB | VAL | 18 | 35.369 | 35.783 | 85.686 | 1 | 21.53 |
| 2700 | CG1 | VAL | 18 | 36.081 | 37.108 | 85.935 | 1 | 23.77 |
| 2701 | CG2 | VAL | 18 | 34.738 | 35.272 | 86.979 | 1 | 22.46 |
| 2702 | C | VAL | 18 | 33.789 | 34.587 | 84.199 | 1 | 20.34 |
| 2703 | O | VAL | 18 | 34.549 | 33.798 | 83.632 | 1 | 19.6 |
| 2704 | N | LEU | 19 | 32.537 | 34.277 | 84.523 | 1 | 19.12 |
| 2705 | CA | LEU | 19 | 31.954 | 32.989 | 84.169 | 1 | 20.34 |
| 2706 | CB | LEU | 19 | 31.479 | 32.223 | 85.417 | 1 | 20.44 |
| 2707 | CG | LEU | 19 | 30.669 | 30.939 | 85.143 | 1 | 20.87 |
| 2708 | CD1 | LEU | 19 | 31.551 | 29.909 | 84.464 | 1 | 22.15 |
| 2709 | CD2 | LEU | 19 | 30.118 | 30.378 | 86.442 | 1 | 22.51 |
| 2710 | C | LEU | 19 | 30.77 | 33.291 | 83.26 | 1 | 19.76 |
| 2711 | O | LEU | 19 | 29.862 | 34.036 | 83.629 | 1 | 19.31 |
| 2712 | N | GLY | 20 | 30.796 | 32.73 | 82.061 | 1 | 18.97 |
| 2713 | CA | GLY | 20 | 29.718 | 32.965 | 81.119 | 1 | 19.62 |
| 2714 | C | GLY | 20 | 28.978 | 31.702 | 80.727 | 1 | 20.48 |
| 2715 | O | GLY | 20 | 29.547 | 30.609 | 80.731 | 1 | 19.33 |
| 2716 | N | PHE | 21 | 27.705 | 31.861 | 80.383 | 1 | 20.13 |
| 2717 | CA | PHE | 21 | 26.856 | 30.741 | 79.99 | 1 | 21.82 |
| 2718 | CB | PHE | 21 | 25.444 | 30.939 | 80.566 | 1 | 20.86 |
| 2719 | CG | PHE | 21 | 24.515 | 29.787 | 80.303 | 1 | 22.06 |
| 2720 | CD1 | PHE | 21 | 24.73 | 28.554 | 80.912 | 1 | 22.01 |
| 2721 | CD2 | PHE | 21 | 23.455 | 29.919 | 79.411 | 1 | 23.33 |
| 2722 | CE1 | PHE | 21 | 23.902 | 27.462 | 80.634 | 1 | 21.96 |
| 2723 | CE2 | PHE | 21 | 22.617 | 28.835 | 79.122 | 1 | 24.29 |
| 2724 | CZ | PHE | 21 | 22.845 | 27.608 | 79.736 | 1 | 23.8 |
| 2725 | C | PHE | 21 | 26.781 | 30.636 | 78.466 | 1 | 21.4 |
| 2726 | O | PHE | 21 | 26.399 | 31.595 | 77.789 | 1 | 20.17 |
| 2727 | N | GLY | 22 | 27.153 | 29.474 | 77.932 | 1 | 21.02 |
| 2728 | CA | GLY | 22 | 27.099 | 29.268 | 76.496 | 1 | 22.2 |
| 2729 | C | GLY | 22 | 25.72 | 28.796 | 76.071 | 1 | 22.97 |
| 2730 | O | GLY | 22 | 25.119 | 27.968 | 76.754 | 1 | 22.67 |
| 2731 | N | THR | 23 | 25.216 | 29.293 | 74.944 | 1 | 23.62 |
| 2732 | CA | THR | 23 | 23.871 | 28.924 | 74.502 | 1 | 24.19 |
| 2733 | CB | THR | 23 | 22.993 | 30.172 | 74.335 | 1 | 22.21 |
| 2734 | OG1 | THR | 23 | 23.536 | 31.009 | 73.302 | 1 | 21.01 |
| 2735 | CG2 | THR | 23 | 22.948 | 30.959 | 75.642 | 1 | 22.76 |
| 2736 | C | THR | 23 | 23.771 | 28.114 | 73.207 | 1 | 26.42 |
| 2737 | O | THR | 23 | 22.666 | 27.767 | 72.778 | 1 | 27.73 |
| 2738 | N | TYR | 24 | 24.9 | 27.81 | 72.58 | 1 | 26.09 |
| 2739 | CA | TYR | 24 | 24.849 | 27.03 | 71.348 | 1 | 29.15 |
| 2740 | CB | TYR | 24 | 26.19 | 27.049 | 70.614 | 1 | 29.87 |
| 2741 | CG | TYR | 24 | 26.169 | 26.159 | 69.379 | 1 | 33.24 |
| 2742 | CD1 | TYR | 24 | 25.503 | 26.561 | 68.217 | 1 | 34.26 |
| 2743 | CE1 | TYR | 24 | 25.405 | 25.712 | 67.108 | 1 | 35.36 |
| 2744 | CD2 | TYR | 24 | 26.741 | 24.886 | 69.401 | 1 | 32.89 |
| 2745 | CE2 | TYR | 24 | 26.648 | 24.031 | 68.299 | 1 | 34.4 |
| 2746 | CZ | TYR | 24 | 25.975 | 24.453 | 67.158 | 1 | 35.12 |
| 2747 | OH | TYR | 24 | 25.859 | 23.611 | 66.075 | 1 | 36.53 |
| 2748 | C | TYR | 24 | 24.486 | 25.579 | 71.614 | 1 | 28.8 |
| 2749 | O | TYR | 24 | 24.953 | 24.983 | 72.582 | 1 | 29.48 |
| 2750 | N | ALA | 25 | 23.665 | 25.009 | 70.742 | 1 | 29.91 |
| 2751 | CA | ALA | 25 | 23.28 | 23.606 | 70.856 | 1 | 32.21 |
| 2752 | CB | ALA | 25 | 22.016 | 23.46 | 71.694 | 1 | 30.83 |
| 2753 | C | ALA | 25 | 23.045 | 23.062 | 69.445 | 1 | 34.07 |
| 2754 | O | ALA | 25 | 22.658 | 23.812 | 68.551 | 1 | 31.89 |
| 2755 | N | PRO | 26 | 23.294 | 21.752 | 69.226 | 1 | 36.72 |
| 2756 | CD | PRO | 26 | 23.816 | 20.788 | 70.211 | 1 | 37.06 |
| 2757 | CA | PRO | 26 | 23.105 | 21.108 | 67.919 | 1 | 39.09 |
| 2758 | CB | PRO | 26 | 23.298 | 19.63 | 68.233 | 1 | 39.11 |
| 2759 | CG | PRO | 26 | 24.31 | 19.66 | 69.327 | 1 | 39.34 |
| 2760 | C | PRO | 26 | 21.712 | 21.394 | 67.374 | 1 | 41.24 |
| 2761 | O | PRO | 26 | 20.754 | 21.5 | 68.142 | 1 | 40.63 |
| 2762 | N | ALA | 27 | 21.604 | 21.506 | 66.054 | 1 | 44.55 |
| 2763 | CA | ALA | 27 | 20.326 | 21.795 | 65.403 | 1 | 47.37 |
| 2764 | CB | ALA | 27 | 20.514 | 21.845 | 63.885 | 1 | 48.56 |
| 2765 | C | ALA | 27 | 19.234 | 20.792 | 65.76 | 1 | 48.57 |
| 2766 | O | ALA | 27 | 18.048 | 21.097 | 65.654 | 1 | 49.96 |
| 2767 | N | GLU | 28 | 19.638 | 19.6 | 66.184 | 1 | 49.36 |
| 2768 | CA | GLU | 28 | 18.686 | 18.56 | 66.555 | 1 | 50.9 |
| 2769 | CB | GLU | 28 | 19.413 | 17.225 | 66.717 | 1 | 53.65 |
| 2770 | CG | GLU | 28 | 20.564 | 17.283 | 67.712 | 1 | 58.93 |
| 2771 | CD | GLU | 28 | 21.368 | 15.996 | 67.776 | 1 | 61.61 |
| 2772 | OE1 | GLU | 28 | 20.822 | 14.966 | 68.233 | 1 | 64.23 |
| 2773 | OE2 | GLU | 28 | 22.549 | 16.018 | 67.365 | 1 | 63.26 |
| 2774 | C | GLU | 28 | 17.95 | 18.9 | 67.847 | 1 | 49.9 |
| 2775 | O | GLU | 28 | 16.866 | 18.378 | 68.105 | 1 | 50.83 |
| 2776 | N | VAL | 29 | 18.54 | 19.77 | 68.662 | 1 | 47.83 |
| 2777 | CA | VAL | 29 | 17.928 | 20.168 | 69.93 | 1 | 44.9 |
| 2778 | CB | VAL | 29 | 18.994 | 20.656 | 70.941 | 1 | 44.63 |
| 2779 | CG1 | VAL | 29 | 18.346 | 20.945 | 72.287 | 1 | 43.33 |
| 2780 | CG2 | VAL | 29 | 20.095 | 19.619 | 71.084 | 1 | 43.27 |
| 2781 | C | VAL | 29 | 16.914 | 21.295 | 69.727 | 1 | 44.17 |
| 2782 | O | VAL | 29 | 17.207 | 22.292 | 69.073 | 1 | 43.92 |
| 2783 | N | PRO | 30 | 15.704 | 21.148 | 70.292 | 1 | 43.3 |
| 2784 | CD | PRO | 30 | 15.223 | 19.984 | 71.056 | 1 | 43.06 |
| 2785 | CA | PRO | 30 | 14.647 | 22.16 | 70.17 | 1 | 43.44 |
| 2786 | CB | PRO | 30 | 13.507 | 21.57 | 71.002 | 1 | 42.61 |
| 2787 | CG | PRO | 30 | 13.728 | 20.1 | 70.898 | 1 | 42.89 |
| 2788 | C | PRO | 30 | 15.091 | 23.528 | 70.707 | 1 | 42.98 |
| 2789 | O | PRO | 30 | 15.723 | 23.614 | 71.762 | 1 | 40.95 |
| 2790 | N | LYS | 31 | 14.748 | 24.59 | 69.986 | 1 | 43.38 |
| 2791 | CA | LYS | 31 | 15.111 | 25.941 | 70.402 | 1 | 44.42 |
| 2792 | CB | LYS | 31 | 14.745 | 26.956 | 69.309 | 1 | 45.21 |
| 2793 | CG | LYS | 31 | 15.524 | 26.795 | 68.008 | 1 | 46.67 |
| 2794 | CD | LYS | 31 | 17.029 | 26.847 | 68.241 | 1 | 47.63 |
| 2795 | CE | LYS | 31 | 17.794 | 26.603 | 66.949 | 1 | 48.38 |
| 2796 | NZ | LYS | 31 | 19.256 | 26.429 | 67.172 | 1 | 50.44 |
| 2797 | C | LYS | 31 | 14.421 | 26.326 | 71.71 | 1 | 44.37 |
| 2798 | O | LYS | 31 | 14.805 | 27.295 | 72.358 | 1 | 45.04 |
| 2799 | N | SER | 32 | 13.404 | 25.56 | 72.097 | 1 | 43.67 |
| 2800 | CA | SER | 32 | 12.667 | 25.833 | 73.325 | 1 | 42.65 |
| 2801 | CB | SER | 32 | 11.387 | 24.991 | 73.373 | 1 | 42.69 |
| 2802 | OG | SER | 32 | 11.674 | 23.606 | 73.413 | 1 | 41.36 |
| 2803 | C | SER | 32 | 13.52 | 25.551 | 74.563 | 1 | 42.86 |
| 2804 | O | SER | 32 | 13.341 | 26.173 | 75.615 | 1 | 43.23 |
| 2805 | N | LYS | 33 | 14.449 | 24.61 | 74.442 | 1 | 41.51 |
| 2806 | CA | LYS | 33 | 15.317 | 24.271 | 75.559 | 1 | 40.07 |
| 2807 | CB | LYS | 33 | 16.071 | 22.971 | 75.276 | 1 | 43.25 |
| 2808 | CG | LYS | 33 | 15.219 | 21.716 | 75.38 | 1 | 47.42 |
| 2809 | CD | LYS | 33 | 16.087 | 20.468 | 75.296 | 1 | 52.09 |
| 2810 | CE | LYS | 33 | 15.265 | 19.196 | 75.452 | 1 | 54.57 |
| 2811 | NZ | LYS | 33 | 16.114 | 17.974 | 75.331 | 1 | 56.46 |
| 2812 | C | LYS | 33 | 16.309 | 25.399 | 75.845 | 1 | 37.74 |
| 2813 | O | LYS | 33 | 16.826 | 25.507 | 76.957 | 1 | 36.52 |
| 2814 | N | ALA | 34 | 16.575 | 26.235 | 74.846 | 1 | 34.59 |
| 2815 | CA | ALA | 34 | 17.49 | 27.356 | 75.035 | 1 | 34.06 |
| 2816 | CB | ALA | 34 | 17.826 | 28.005 | 73.697 | 1 | 34.5 |
| 2817 | C | ALA | 34 | 16.814 | 28.368 | 75.964 | 1 | 33.31 |
| 2818 | O | ALA | 34 | 17.457 | 28.969 | 76.828 | 1 | 31.23 |
| 2819 | N | LEU | 35 | 15.509 | 28.546 | 75.789 | 1 | 32.19 |
| 2820 | CA | LEU | 35 | 14.761 | 29.475 | 76.624 | 1 | 31.46 |
| 2821 | CB | LEU | 35 | 13.312 | 29.576 | 76.142 | 1 | 32.96 |
| 2822 | CG | LEU | 35 | 12.317 | 30.341 | 77.022 | 1 | 33.43 |
| 2823 | CD1 | LEU | 35 | 12.749 | 31.78 | 77.22 | 1 | 34.56 |
| 2824 | CD2 | LEU | 35 | 10.959 | 30.294 | 76.348 | 1 | 37.14 |
| 2825 | C | LEU | 35 | 14.794 | 29.021 | 78.081 | 1 | 31.43 |
| 2826 | O | LEU | 35 | 15.036 | 29.817 | 78.988 | 1 | 29.27 |
| 2827 | N | GLU | 36 | 14.556 | 27.735 | 78.305 | 1 | 31.35 |
| 2828 | CA | GLU | 36 | 14.561 | 27.195 | 79.661 | 1 | 33.31 |
| 2829 | CB | GLU | 36 | 14.043 | 25.76 | 79.649 | 1 | 37.09 |
| 2830 | CG | GLU | 36 | 12.7 | 25.633 | 78.965 | 1 | 45.94 |
| 2831 | CD | GLU | 36 | 12.192 | 24.203 | 78.925 | 1 | 50.64 |
| 2832 | OE1 | GLU | 36 | 12.944 | 23.315 | 78.456 | 1 | 53.15 |
| 2833 | OE2 | GLU | 36 | 11.036 | 23.974 | 79.353 | 1 | 53.3 |
| 2834 | C | GLU | 36 | 15.953 | 27.224 | 80.274 | 1 | 31.27 |
| 2835 | O | GLU | 36 | 16.12 | 27.582 | 81.445 | 1 | 30.65 |
| 2836 | N | ALA | 37 | 16.951 | 26.851 | 79.479 | 1 | 28.97 |
| 2837 | CA | ALA | 37 | 18.333 | 26.825 | 79.954 | 1 | 27.45 |
| 2838 | CB | ALA | 37 | 19.259 | 26.281 | 78.852 | 1 | 26.57 |
| 2839 | C | ALA | 37 | 18.822 | 28.2 | 80.427 | 1 | 26.24 |
| 2840 | O | ALA | 37 | 19.428 | 28.31 | 81.49 | 1 | 25.18 |
| 2841 | N | VAL | 38 | 18.564 | 29.245 | 79.645 | 1 | 25.53 |
| 2842 | CA | VAL | 38 | 19.007 | 30.574 | 80.041 | 1 | 25.16 |
| 2843 | CB | VAL | 38 | 18.754 | 31.612 | 78.921 | 1 | 26.09 |
| 2844 | CG1 | VAL | 38 | 19.134 | 33.008 | 79.396 | 1 | 24.8 |
| 2845 | CG2 | VAL | 38 | 19.585 | 31.252 | 77.696 | 1 | 24.23 |
| 2846 | C | VAL | 38 | 18.324 | 31.016 | 81.342 | 1 | 25.72 |
| 2847 | O | VAL | 38 | 18.959 | 31.624 | 82.201 | 1 | 23.17 |
| 2848 | N | LYS | 39 | 17.039 | 30.704 | 81.499 | 1 | 25.85 |
| 2849 | CA | LYS | 39 | 16.336 | 31.083 | 82.72 | 1 | 26.89 |
| 2850 | CB | LYS | 39 | 14.87 | 30.668 | 82.651 | 1 | 28.97 |
| 2851 | CG | LYS | 39 | 14.01 | 31.552 | 81.768 | 1 | 31.93 |
| 2852 | CD | LYS | 39 | 12.548 | 31.133 | 81.871 | 1 | 38.8 |
| 2853 | CE | LYS | 39 | 11.646 | 32.084 | 81.103 | 1 | 42.32 |
| 2854 | NZ | LYS | 39 | 10.197 | 31.748 | 81.265 | 1 | 46.18 |
| 2855 | C | LYS | 39 | 16.985 | 30.432 | 83.941 | 1 | 26.88 |
| 2856 | O | LYS | 39 | 17.199 | 31.085 | 84.965 | 1 | 26.39 |
| 2857 | N | LEU | 40 | 17.294 | 29.144 | 83.827 | 1 | 26.1 |
| 2858 | CA | LEU | 40 | 17.921 | 28.403 | 84.915 | 1 | 26.99 |
| 2859 | CB | LEU | 40 | 17.991 | 26.907 | 84.562 | 1 | 29.26 |
| 2860 | CG | LEU | 40 | 16.664 | 26.138 | 84.492 | 1 | 30.91 |
| 2861 | CD1 | LEU | 40 | 16.894 | 24.739 | 83.936 | 1 | 33.02 |
| 2862 | CD2 | LEU | 40 | 16.036 | 25.063 | 85.885 | 1 | 32 |
| 2863 | C | LEU | 40 | 19.323 | 28.93 | 85.219 | 1 | 26.7 |
| 2864 | O | LEU | 40 | 19.715 | 29.03 | 86.377 | 1 | 26.63 |
| 2865 | N | ALA | 41 | 20.083 | 29.255 | 84.175 | 1 | 25.67 |
| 2866 | CA | ALA | 41 | 21.435 | 29.786 | 84.35 | 1 | 24.31 |
| 2867 | CB | ALA | 41 | 22.101 | 29.997 | 82.98 | 1 | 24.12 |
| 2868 | C | ALA | 41 | 21.385 | 31.106 | 85.126 | 1 | 23.7 |
| 2869 | O | ALA | 41 | 22.207 | 31.347 | 86.009 | 1 | 23.75 |
| 2870 | N | ILE | 42 | 20.424 | 31.965 | 84.798 | 1 | 23.4 |
| 2871 | CA | ILE | 42 | 20.304 | 33.23 | 85.504 | 1 | 24.4 |
| 2872 | CB | ILE | 42 | 19.26 | 34.157 | 84.83 | 1 | 23.89 |
| 2873 | CG2 | ILE | 42 | 18.979 | 35.361 | 85.713 | 1 | 23.17 |
| 2874 | CG1 | ILE | 42 | 19.801 | 34.624 | 83.47 | 1 | 24.07 |
| 2875 | CD1 | ILE | 42 | 18.826 | 35.454 | 82.645 | 1 | 24.15 |
| 2876 | C | ILE | 42 | 19.926 | 32.963 | 86.964 | 1 | 26.63 |
| 2877 | O | ILE | 42 | 20.459 | 33.596 | 87.875 | 1 | 25.63 |
| 2878 | N | GLU | 43 | 19.023 | 32.014 | 87.191 | 1 | 27.75 |
| 2879 | CA | GLU | 43 | 18.619 | 31.67 | 88.557 | 1 | 29.96 |
| 2880 | CB | GLU | 43 | 17.464 | 30.666 | 88.529 | 1 | 32.5 |
| 2881 | CG | GLU | 43 | 16.163 | 31.253 | 88.02 | 1 | 39.88 |
| 2882 | CD | GLU | 43 | 15.071 | 30.212 | 87.883 | 1 | 44.77 |
| 2883 | OE1 | GLU | 43 | 14.978 | 29.336 | 88.772 | 1 | 48.08 |
| 2884 | OE2 | GLU | 43 | 14.299 | 30.275 | 86.898 | 1 | 46.33 |
| 2885 | C | GLU | 43 | 19.784 | 31.078 | 89.353 | 1 | 29.45 |
| 2886 | O | GLU | 43 | 19.901 | 31.314 | 90.552 | 1 | 29.01 |
| 2887 | N | ALA | 44 | 20.642 | 30.316 | 88.678 | 1 | 27.71 |
| 2888 | CA | ALA | 44 | 21.784 | 29.679 | 89.318 | 1 | 26.97 |
| 2889 | CB | ALA | 44 | 22.332 | 28.57 | 88.417 | 1 | 25.65 |
| 2890 | C | ALA | 44 | 22.9 | 30.665 | 89.674 | 1 | 27.05 |
| 2891 | O | ALA | 44 | 23.745 | 30.369 | 90.519 | 1 | 26.75 |
| 2892 | N | GLY | 45 | 22.915 | 31.831 | 89.033 | 1 | 25.47 |
| 2893 | CA | GLY | 45 | 23.957 | 32.8 | 89.346 | 1 | 26.2 |
| 2894 | C | GLY | 45 | 24.823 | 33.257 | 88.179 | 1 | 24.8 |
| 2895 | O | GLY | 45 | 25.822 | 33.941 | 88.383 | 1 | 24.11 |
| 2896 | N | PHE | 46 | 24.463 | 32.865 | 86.96 | 1 | 22.81 |
| 2897 | CA | PHE | 46 | 25.206 | 33.299 | 85.774 | 1 | 23.85 |
| 2898 | CB | PHE | 46 | 24.873 | 32.41 | 84.577 | 1 | 21.71 |
| 2899 | CG | PHE | 46 | 25.639 | 31.126 | 84.536 | 1 | 21.41 |
| 2900 | CD1 | PHE | 46 | 26.841 | 31.046 | 83.844 | 1 | 21.32 |
| 2901 | CD2 | PHE | 46 | 25.149 | 29.986 | 85.159 | 1 | 20.85 |
| 2902 | CE1 | PHE | 46 | 27.553 | 29.838 | 83.764 | 1 | 22.22 |
| 2903 | CE2 | PHE | 46 | 25.853 | 28.771 | 85.085 | 1 | 22.8 |
| 2904 | CZ | PHE | 46 | 27.054 | 28.701 | 84.387 | 1 | 21.79 |
| 2905 | C | PHE | 46 | 24.772 | 34.728 | 85.449 | 1 | 23.48 |
| 2906 | O | PHE | 46 | 23.58 | 35.016 | 85.402 | 1 | 24.97 |
| 2907 | N | HIS | 47 | 25.738 | 35.617 | 85.224 | 1 | 22.89 |
| 2908 | CA | HIS | 47 | 25.435 | 37.01 | 84.899 | 1 | 21.75 |
| 2909 | CB | HIS | 47 | 26.064 | 37.937 | 85.94 | 1 | 20.72 |
| 2910 | CG | HIS | 47 | 25.362 | 37.921 | 87.259 | 1 | 22.19 |
| 2911 | CD2 | HIS | 47 | 25.554 | 37.162 | 88.364 | 1 | 23.04 |
| 2912 | ND1 | HIS | 47 | 24.289 | 38.74 | 87.538 | 1 | 24.37 |
| 2913 | CE1 | HIS | 47 | 23.851 | 38.487 | 88.758 | 1 | 23.52 |
| 2914 | NE2 | HIS | 47 | 24.6 | 37.533 | 89.28 | 1 | 22.33 |
| 2915 | C | HIS | 47 | 25.971 | 37.379 | 83.514 | 1 | 22.95 |
| 2916 | O | HIS | 47 | 25.86 | 38.524 | 83.082 | 1 | 23.41 |
| 2917 | N | HIS | 48 | 26.547 | 36.394 | 82.834 | 1 | 21.99 |
| 2918 | CA | HIS | 48 | 27.142 | 36.575 | 81.509 | 1 | 21.77 |
| 2919 | CB | HIS | 48 | 28.669 | 36.428 | 81.644 | 1 | 21.18 |
| 2920 | CG | HIS | 48 | 29.429 | 36.499 | 80.351 | 1 | 21.68 |
| 2921 | CD2 | HIS | 48 | 29.025 | 36.382 | 79.064 | 1 | 21.78 |
| 2922 | ND1 | HIS | 48 | 30.802 | 36.624 | 80.309 | 1 | 22.13 |
| 2923 | CE1 | HIS | 48 | 31.21 | 36.572 | 79.053 | 1 | 23.16 |
| 2924 | NE2 | HIS | 48 | 30.15 | 36.423 | 78.278 | 1 | 22.2 |
| 2925 | C | HIS | 48 | 26.53 | 35.507 | 80.593 | 1 | 21.54 |
| 2926 | O | HIS | 48 | 26.568 | 34.317 | 80.9 | 1 | 20.56 |
| 2927 | N | ILE | 49 | 25.934 | 35.95 | 79.488 | 1 | 20.27 |
| 2928 | CA | ILE | 49 | 25.29 | 35.048 | 78.53 | 1 | 20.69 |
| 2929 | CB | ILE | 49 | 23.758 | 35.303 | 78.48 | 1 | 22.42 |
| 2930 | CG2 | ILE | 49 | 23.084 | 34.324 | 77.507 | 1 | 22.61 |
| 2931 | CG1 | ILE | 49 | 23.177 | 35.155 | 79.894 | 1 | 23.66 |
| 2932 | CD1 | ILE | 49 | 21.782 | 35.697 | 80.036 | 1 | 31 |
| 2933 | C | ILE | 49 | 25.898 | 35.272 | 77.145 | 1 | 20.39 |
| 2934 | O | ILE | 49 | 25.953 | 36.399 | 76.651 | 1 | 20.55 |
| 2935 | N | ASP | 50 | 26.329 | 34.186 | 76.518 | 1 | 18.83 |
| 2936 | CA | ASP | 50 | 26.974 | 34.263 | 75.22 | 1 | 19.92 |
| 2937 | CB | ASP | 50 | 28.332 | 33.549 | 75.283 | 1 | 19.2 |
| 2938 | CG | ASP | 50 | 29.119 | 33.669 | 73.984 | 1 | 20.93 |
| 2939 | OD1 | ASP | 50 | 29.888 | 34.643 | 73.832 | 1 | 17.84 |
| 2940 | OD2 | ASP | 50 | 28.959 | 32.79 | 73.105 | 1 | 21.3 |
| 2941 | C | ASP | 50 | 26.142 | 33.658 | 74.099 | 1 | 19.88 |
| 2942 | O | ASP | 50 | 25.757 | 32.491 | 74.165 | 1 | 19.89 |
| 2943 | N | SER | 51 | 25.846 | 34.449 | 73.075 | 1 | 19.81 |
| 2944 | CA | SER | 51 | 25.107 | 33.915 | 71.94 | 1 | 20.16 |
| 2945 | CB | SER | 51 | 23.614 | 34.207 | 72.041 | 1 | 21.03 |
| 2946 | OG | SER | 51 | 22.906 | 33.362 | 71.124 | 1 | 24.72 |
| 2947 | C | SER | 51 | 25.655 | 34.488 | 70.636 | 1 | 21.26 |
| 2948 | O | SER | 51 | 26.767 | 35.009 | 70.598 | 1 | 21.14 |
| 2949 | N | ALA | 52 | 24.865 | 34.4 | 69.573 | 1 | 20.99 |
| 2950 | CA | ALA | 52 | 25.298 | 34.872 | 68.273 | 1 | 22.57 |
| 2951 | CB | ALA | 52 | 26.491 | 34.036 | 67.792 | 1 | 20.12 |
| 2952 | C | ALA | 52 | 24.156 | 34.734 | 67.286 | 1 | 22.63 |
| 2953 | O | ALA | 52 | 23.255 | 33.919 | 67.48 | 1 | 24.12 |
| 2954 | N | HIS | 53 | 24.197 | 35.527 | 66.227 | 1 | 22.43 |
| 2955 | CA | HIS | 53 | 23.164 | 35.461 | 65.206 | 1 | 24.21 |
| 2956 | CB | HIS | 53 | 23.492 | 36.441 | 64.07 | 1 | 24.8 |
| 2957 | CG | HIS | 53 | 22.581 | 36.325 | 62.892 | 1 | 27.19 |
| 2958 | CD2 | HIS | 53 | 22.826 | 35.955 | 61.613 | 1 | 28.16 |
| 2959 | ND1 | HIS | 53 | 21.226 | 36.552 | 62.974 | 1 | 28.2 |
| 2960 | CE1 | HIS | 53 | 20.672 | 36.322 | 61.796 | 1 | 29.25 |
| 2961 | NE2 | HIS | 53 | 21.623 | 35.958 | 60.953 | 1 | 30.19 |
| 2962 | C | HIS | 53 | 23.067 | 34.031 | 64.661 | 1 | 24.84 |
| 2963 | O | HIS | 53 | 21.979 | 33.469 | 64.598 | 1 | 24.37 |
| 2964 | N | VAL | 54 | 24.206 | 33.432 | 64.305 | 1 | 25.97 |
| 2965 | CA | VAL | 54 | 24.209 | 32.079 | 63.733 | 1 | 27.38 |
| 2966 | CB | VAL | 54 | 25.607 | 31.625 | 63.235 | 1 | 29.06 |
| 2967 | CG1 | VAL | 54 | 25.938 | 32.303 | 61.941 | 1 | 31.84 |
| 2968 | CG2 | VAL | 54 | 26.663 | 31.884 | 64.292 | 1 | 28.59 |
| 2969 | C | VAL | 54 | 23.713 | 30.95 | 64.601 | 1 | 27.43 |
| 2970 | O | VAL | 54 | 23.506 | 29.85 | 64.099 | 1 | 26.2 |
| 2971 | N | TYR | 55 | 23.545 | 31.187 | 65.897 | 1 | 25.66 |
| 2972 | CA | TYR | 55 | 23.063 | 30.116 | 66.767 | 1 | 24.57 |
| 2973 | CB | TYR | 55 | 23.375 | 30.412 | 68.242 | 1 | 23.08 |
| 2974 | CG | TYR | 55 | 24.858 | 30.477 | 68.572 | 1 | 20.87 |
| 2975 | CD1 | TYR | 55 | 25.825 | 30.083 | 67.644 | 1 | 20.85 |
| 2976 | CE1 | TYR | 55 | 27.19 | 30.168 | 67.934 | 1 | 19.98 |
| 2977 | CD2 | TYR | 55 | 25.292 | 30.953 | 69.81 | 1 | 22.02 |
| 2978 | CE2 | TYR | 55 | 26.652 | 31.041 | 70.114 | 1 | 20.05 |
| 2979 | CZ | TYR | 55 | 27.592 | 30.651 | 69.168 | 1 | 20.66 |
| 2980 | OH | TYR | 55 | 28.928 | 30.786 | 69.449 | 1 | 20.36 |
| 2981 | C | TYR | 55 | 21.56 | 29.975 | 66.578 | 1 | 25.61 |
| 2982 | O | TYR | 55 | 20.949 | 29.005 | 67.023 | 1 | 26.05 |
| 2983 | N | ASN | 56 | 20.971 | 30.964 | 65.919 | 1 | 26.33 |
| 2984 | CA | ASN | 56 | 19.543 | 30.97 | 65.65 | 1 | 28.12 |
| 2985 | CB | ASN | 56 | 19.199 | 29.875 | 64.64 | 1 | 32.95 |
| 2986 | CG | ASN | 56 | 17.921 | 30.166 | 63.883 | 1 | 38.01 |
| 2987 | OD1 | ASN | 56 | 17.27 | 29.255 | 63.371 | 1 | 43.36 |
| 2988 | ND2 | ASN | 56 | 17.558 | 31.444 | 63.796 | 1 | 40.52 |
| 2989 | C | ASN | 56 | 18.69 | 30.78 | 66.904 | 1 | 27.75 |
| 2990 | O | ASN | 56 | 17.686 | 30.067 | 66.879 | 1 | 27.99 |
| 2991 | N | ASN | 57 | 19.078 | 31.421 | 68.001 | 1 | 26.13 |
| 2992 | CA | ASN | 57 | 18.322 | 31.298 | 69.239 | 1 | 25.98 |
| 2993 | CB | ASN | 57 | 19.011 | 30.296 | 70.185 | 1 | 25.2 |
| 2994 | CG | ASN | 57 | 20.402 | 30.755 | 70.617 | 1 | 26.97 |
| 2995 | OD1 | ASN | 57 | 20.771 | 31.917 | 70.432 | 1 | 27.15 |
| 2996 | ND2 | ASN | 57 | 21.175 | 29.845 | 71.206 | 1 | 26.44 |
| 2997 | C | ASN | 57 | 18.141 | 32.625 | 69.977 | 1 | 25.42 |
| 2998 | O | ASN | 57 | 17.644 | 32.638 | 71.099 | 1 | 25.94 |
| 2999 | N | GLU | 58 | 18.526 | 33.735 | 69.357 | 1 | 25.6 |
| 3000 | CA | GLU | 58 | 18.432 | 35.027 | 70.031 | 1 | 26.34 |
| 3001 | CB | GLU | 58 | 19.104 | 36.121 | 69.183 | 1 | 25.7 |
| 3002 | CG | GLU | 58 | 20.645 | 36.006 | 69.244 | 1 | 25.77 |
| 3003 | CD | GLU | 58 | 21.387 | 37.199 | 68.658 | 1 | 25.84 |
| 3004 | OE1 | GLU | 58 | 21.144 | 37.541 | 67.478 | 1 | 24.8 |
| 3005 | OE2 | GLU | 58 | 22.231 | 37.78 | 69.377 | 1 | 24.97 |
| 3006 | C | GLU | 58 | 17.017 | 35.414 | 70.437 | 1 | 27.34 |
| 3007 | O | GLU | 58 | 16.815 | 36.135 | 71.418 | 1 | 25.53 |
| 3008 | N | GLU | 59 | 16.034 | 34.917 | 69.698 | 1 | 27.98 |
| 3009 | CA | GLU | 59 | 14.651 | 35.199 | 70.025 | 1 | 30 |
| 3010 | CB | GLU | 59 | 13.744 | 34.652 | 68.93 | 1 | 34.43 |
| 3011 | CG | GLU | 59 | 12.286 | 34.967 | 69.106 | 1 | 41.73 |
| 3012 | CD | GLU | 59 | 11.488 | 34.592 | 67.875 | 1 | 46.56 |
| 3013 | OE1 | GLU | 59 | 11.354 | 33.38 | 67.597 | 1 | 49.54 |
| 3014 | OE2 | GLU | 59 | 11.013 | 35.512 | 57.175 | 1 | 49.46 |
| 3015 | C | GLU | 59 | 14.328 | 34.539 | 71.371 | 1 | 29.4 |
| 3016 | O | GLU | 59 | 13.74 | 35.167 | 72.255 | 1 | 28.42 |
| 3017 | N | GLN | 60 | 14.731 | 33.279 | 71.535 | 1 | 27.4 |
| 3018 | CA | GLN | 60 | 14.48 | 32.552 | 72.782 | 1 | 27.54 |
| 3019 | CB | GLN | 60 | 14.765 | 31.052 | 72.619 | 1 | 29.68 |
| 3020 | CG | GLN | 60 | 13.806 | 30.284 | 71.714 | 1 | 30.38 |
| 3021 | CD | GLN | 60 | 13.884 | 30.716 | 70.269 | 1 | 32.32 |
| 3022 | OE1 | GLN | 60 | 14.957 | 31.036 | 69.756 | 1 | 33.54 |
| 3023 | NE2 | GLN | 60 | 12.744 | 30.71 | 69.593 | 1 | 36.39 |
| 3024 | C | GLN | 60 | 15.345 | 33.085 | 73.925 | 1 | 26.83 |
| 3025 | O | GLN | 60 | 14.865 | 33.246 | 75.047 | 1 | 26.42 |
| 3026 | N | VAL | 61 | 16.621 | 33.341 | 73.641 | 1 | 25.34 |
| 3027 | CA | VAL | 61 | 17.533 | 33.863 | 74.655 | 1 | 25.63 |
| 3028 | CB | VAL | 61 | 18.982 | 33.995 | 74.093 | 1 | 26.32 |
| 3029 | CG1 | VAL | 61 | 19.892 | 34.682 | 75.117 | 1 | 26.57 |
| 3030 | CG2 | VAL | 61 | 19.536 | 32.609 | 73.774 | 1 | 27.75 |
| 3031 | C | VAL | 61 | 17.024 | 35.22 | 75.15 | 1 | 25.49 |
| 3032 | O | VAL | 61 | 16.978 | 35.471 | 76.359 | 1 | 24.8 |
| 3033 | N | GLY | 62 | 16.612 | 36.076 | 74.215 | 1 | 24.34 |
| 3034 | CA | GLY | 62 | 16.105 | 37.388 | 74.583 | 1 | 25.51 |
| 3035 | C | GLY | 62 | 14.885 | 37.29 | 75.484 | 1 | 26.43 |
| 3036 | O | GLY | 62 | 14.747 | 38.033 | 76.459 | 1 | 26.03 |
| 3037 | N | LEU | 63 | 13.991 | 36.364 | 75.156 | 1 | 25.93 |
| 3038 | CA | LEU | 63 | 12.779 | 36.171 | 75.941 | 1 | 28.5 |
| 3039 | CB | LEU | 63 | 11.874 | 35.149 | 75.251 | 1 | 30.77 |
| 3040 | CG | LEU | 63 | 10.448 | 34.996 | 75.777 | 1 | 34.72 |
| 3041 | CD1 | LEU | 63 | 9.702 | 36.324 | 75.714 | 1 | 36.85 |
| 3042 | CD2 | LEU | 63 | 9.734 | 33.955 | 74.932 | 1 | 36.99 |
| 3043 | C | LEU | 63 | 13.122 | 35.707 | 77.365 | 1 | 28.11 |
| 3044 | O | LEU | 63 | 12.504 | 36.139 | 78.344 | 1 | 26.23 |
| 3045 | N | ALA | 64 | 14.108 | 34.826 | 77.48 | 1 | 26.08 |
| 3046 | CA | ALA | 64 | 14.516 | 34.347 | 78.795 | 1 | 26.61 |
| 3047 | CB | ALA | 64 | 15.625 | 33.303 | 78.66 | 1 | 25.41 |
| 3048 | C | ALA | 64 | 15.003 | 35.522 | 79.645 | 1 | 26.26 |
| 3049 | O | ALA | 64 | 14.605 | 35.674 | 80.804 | 1 | 26.32 |
| 3050 | N | ILE | 65 | 15.861 | 36.353 | 79.06 | 1 | 25.07 |
| 3051 | CA | ILE | 65 | 16.417 | 37.511 | 79.757 | 1 | 26 |
| 3052 | CB | ILE | 65 | 17.457 | 38.248 | 78.869 | 1 | 25.98 |
| 3053 | CG2 | ILE | 65 | 17.907 | 39.549 | 79.542 | 1 | 25.07 |
| 3054 | CG1 | ILE | 65 | 18.66 | 37.332 | 78.619 | 1 | 25.28 |
| 3055 | CD1 | ILE | 65 | 19.699 | 37.916 | 77.673 | 1 | 27.03 |
| 3056 | C | ILE | 65 | 15.326 | 38.497 | 80.177 | 1 | 26.74 |
| 3057 | O | ILE | 65 | 15.264 | 38.902 | 81.34 | 1 | 25.91 |
| 3058 | N | ARG | 66 | 14.467 | 38.883 | 79.237 | 1 | 25.35 |
| 3059 | CA | ARG | 66 | 13.406 | 39.828 | 79.561 | 1 | 28.96 |
| 3060 | CB | ARG | 66 | 12.628 | 40.222 | 78.305 | 1 | 31.16 |
| 3061 | CG | ARG | 66 | 13.486 | 40.877 | 77.24 | 1 | 36.86 |
| 3062 | CD | ARG | 66 | 12.646 | 41.655 | 76.242 | 1 | 42.52 |
| 3063 | NE | ARG | 66 | 11.525 | 40.876 | 75.708 | 1 | 48.22 |
| 3064 | CZ | ARG | 66 | 11.651 | 39.833 | 74.89 | 1 | 48.95 |
| 3065 | NH1 | ARG | 66 | 12.85 | 39.43 | 74.5 | 1 | 49.65 |
| 3066 | NH2 | ARG | 66 | 10.574 | 39.195 | 74.459 | 1 | 50.89 |
| 3067 | C | ARG | 66 | 12.441 | 39.299 | 80.621 | 1 | 29.09 |
| 3068 | O | ARG | 66 | 11.915 | 40.07 | 81.427 | 1 | 29.74 |
| 3069 | N | SER | 67 | 12.205 | 37.991 | 80.632 | 1 | 29.24 |
| 3070 | CA | SER | 67 | 11.297 | 37.425 | 81.617 | 1 | 32.23 |
| 3071 | CB | SER | 67 | 10.886 | 35.993 | 81.229 | 1 | 33.45 |
| 3072 | OG | SER | 67 | 11.973 | 35.095 | 81.312 | 1 | 41.58 |
| 3073 | C | SER | 67 | 11.925 | 37.45 | 83.014 | 1 | 32.16 |
| 3074 | O | SER | 67 | 11.221 | 37.622 | 84.01 | 1 | 32.1 |
| 3075 | N | LYS | 68 | 13.246 | 37.298 | 83.093 | 1 | 31.73 |
| 3076 | CA | LYS | 68 | 13.925 | 37.32 | 84.39 | 1 | 32.39 |
| 3077 | CB | LYS | 68 | 15.296 | 36.65 | 84.288 | 1 | 33.23 |
| 3078 | CG | LYS | 68 | 15.212 | 35.135 | 84.342 | 1 | 34.93 |
| 3079 | CD | LYS | 68 | 14.706 | 34.702 | 85.715 | 1 | 39.94 |
| 3080 | CE | LYS | 68 | 14.278 | 33.253 | 85.734 | 1 | 42.89 |
| 3081 | NZ | LYS | 68 | 13.7 | 32.885 | 87.06 | 1 | 44.66 |
| 3082 | C | LYS | 68 | 14.056 | 38.746 | 84.918 | 1 | 32.83 |
| 3083 | O | LYS | 68 | 14.207 | 38.971 | 86.123 | 1 | 33.07 |
| 3084 | N | ILE | 69 | 14.003 | 39.711 | 84.009 | 1 | 31.55 |
| 3085 | CA | ILE | 69 | 14.062 | 41.111 | 84.4 | 1 | 32.25 |
| 3086 | CB | ILE | 69 | 14.544 | 42.002 | 83.232 | 1 | 30.9 |
| 3087 | CG2 | ILE | 69 | 14.284 | 43.473 | 83.544 | 1 | 31.96 |
| 3088 | CG1 | ILE | 69 | 16.038 | 41.781 | 82.995 | 1 | 29.61 |
| 3089 | CD1 | ILE | 69 | 16.572 | 42.501 | 81.767 | 1 | 29.55 |
| 3090 | C | ILE | 69 | 12.649 | 41.538 | 84.811 | 1 | 32.64 |
| 3091 | O | ILE | 69 | 12.468 | 42.244 | 85.798 | 1 | 32.2 |
| 3092 | N | ALA | 70 | 11.656 | 41.083 | 84.051 | 1 | 33.39 |
| 3093 | CA | ALA | 70 | 10.258 | 41.41 | 84.312 | 1 | 35.22 |
| 3094 | CB | ALA | 70 | 9.386 | 40.918 | 83.162 | 1 | 34.73 |
| 3095 | C | ALA | 70 | 9.727 | 40.868 | 85.636 | 1 | 35.94 |
| 3096 | O | ALA | 70 | 8.885 | 41.51 | 86.266 | 1 | 37.17 |
| 3097 | N | ASP | 71 | 10.199 | 39.7 | 86.066 | 1 | 36.84 |
| 3098 | CA | ASP | 71 | 9.72 | 39.146 | 87.331 | 1 | 38.75 |
| 3099 | CB | ASP | 71 | 9.743 | 37.609 | 87.316 | 1 | 39.54 |
| 3100 | CG | ASP | 71 | 11.142 | 37.039 | 87.228 | 1 | 39.41 |
| 3101 | OD1 | ASP | 71 | 12.106 | 37.693 | 87.669 | 1 | 38.74 |
| 3102 | OD2 | ASP | 71 | 11.269 | 35.908 | 86.732 | 1 | 42.5 |
| 3103 | C | ASP | 71 | 10.519 | 39.665 | 88.519 | 1 | 39.01 |
| 3104 | O | ASP | 71 | 10.317 | 39.219 | 89.646 | 1 | 40.23 |
| 3105 | N | GLY | 72 | 11.43 | 40.599 | 88.257 | 1 | 38.37 |
| 3106 | CA | GLY | 72 | 12.226 | 41.182 | 89.322 | 1 | 37.76 |
| 3107 | C | GLY | 72 | 13.392 | 40.361 | 89.837 | 1 | 38.24 |
| 3108 | O | GLY | 72 | 13.955 | 40.675 | 90.883 | 1 | 39.38 |
| 3109 | N | SER | 73 | 13.771 | 39.311 | 89.12 | 1 | 36.99 |
| 3110 | CA | SER | 73 | 14.889 | 38.484 | 89.562 | 1 | 35.71 |
| 3111 | CB | SER | 73 | 14.876 | 37.139 | 88.837 | 1 | 35.76 |
| 3112 | OG | SER | 73 | 13.717 | 36.4 | 89.17 | 1 | 36.17 |
| 3113 | C | SER | 73 | 16.238 | 39.163 | 89.343 | 1 | 35.38 |
| 3114 | O | SER | 73 | 17.175 | 38.969 | 90.119 | 1 | 35.74 |
| 3115 | N | VAL | 74 | 16.342 | 39.962 | 88.288 | 1 | 33.87 |
| 3116 | CA | VAL | 74 | 17.598 | 40.636 | 87.994 | 1 | 32.72 |
| 3117 | CB | VAL | 74 | 18.538 | 39.679 | 87.192 | 1 | 32.82 |
| 3118 | CG1 | VAL | 74 | 17.979 | 39.444 | 85.793 | 1 | 31.76 |
| 3119 | CG2 | VAL | 74 | 19.946 | 40.242 | 87.131 | 1 | 34.65 |
| 3120 | C | VAL | 74 | 17.344 | 41.921 | 87.206 | 1 | 31.94 |
| 3121 | O | VAL | 74 | 16.289 | 42.09 | 86.597 | 1 | 31.01 |
| 3122 | N | LYS | 75 | 18.299 | 42.842 | 87.242 | 1 | 31.16 |
| 3123 | CA | LYS | 75 | 18.164 | 44.086 | 86.502 | 1 | 31.69 |
| 3124 | CB | LYS | 75 | 18.64 | 45.262 | 87.353 | 1 | 34.89 |
| 3125 | CG | LYS | 75 | 17.841 | 45.429 | 88.632 | 1 | 39.76 |
| 3126 | CD | LYS | 75 | 18.138 | 46.744 | 89.34 | 1 | 43.3 |
| 3127 | CE | LYS | 75 | 19.556 | 46.802 | 89.894 | 1 | 47.44 |
| 3128 | NZ | LYS | 75 | 20.595 | 47.115 | 88.867 | 1 | 49.67 |
| 3129 | C | LYS | 75 | 19.006 | 43.981 | 85.234 | 1 | 30.56 |
| 3130 | O | LYS | 75 | 19.945 | 43.187 | 85.179 | 1 | 28.43 |
| 3131 | N | ARG | 76 | 18.674 | 44.777 | 84.222 | 1 | 29.43 |
| 3132 | CA | ARG | 76 | 19.417 | 44.746 | 82.963 | 1 | 28.54 |
| 3133 | CB | ARG | 76 | 18.876 | 45.805 | 81.994 | 1 | 28.18 |
| 3134 | CG | ARG | 76 | 19.622 | 45.855 | 80.654 | 1 | 26.45 |
| 3135 | CD | ARG | 76 | 19.436 | 44.573 | 79.844 | 1 | 24.99 |
| 3136 | NE | ARG | 76 | 20.237 | 44.585 | 78.616 | 1 | 23.98 |
| 3137 | CZ | ARG | 76 | 21.497 | 44.164 | 78.535 | 1 | 24.93 |
| 3138 | NH1 | ARG | 76 | 22.109 | 43.688 | 79.612 | 1 | 23.32 |
| 3139 | NH2 | ARG | 76 | 22.148 | 44.218 | 77.376 | 1 | 22.71 |
| 3140 | C | ARG | 76 | 20.914 | 44.969 | 83.182 | 1 | 28.68 |
| 3141 | O | ARG | 76 | 21.749 | 44.296 | 82.574 | 1 | 28.29 |
| 3142 | N | GLU | 77 | 21.24 | 45.906 | 84.066 | 1 | 28.14 |
| 3143 | CA | GLU | 77 | 22.626 | 46.248 | 84.372 | 1 | 29.48 |
| 3144 | CB | GLU | 77 | 22.685 | 47.418 | 85.363 | 1 | 32.67 |
| 3145 | CG | GLU | 77 | 21.785 | 45.611 | 85.034 | 1 | 39.88 |
| 3146 | CD | GLU | 77 | 20.296 | 45.272 | 85.075 | 1 | 41.83 |
| 3147 | OE1 | GLU | 77 | 19.857 | 47.607 | 86.031 | 1 | 44.85 |
| 3148 | OE2 | GLU | 77 | 19.564 | 45.679 | 84.152 | 1 | 46.46 |
| 3149 | C | GLU | 77 | 23.404 | 45.071 | 84.961 | 1 | 27.25 |
| 3150 | O | GLU | 77 | 24.636 | 45.081 | 84.968 | 1 | 26.62 |
| 3151 | N | ASP | 78 | 22.701 | 44.064 | 85.468 | 1 | 25.71 |
| 3152 | CA | ASP | 78 | 23.39 | 42.917 | 86.05 | 1 | 26.07 |
| 3153 | CB | ASP | 78 | 22.765 | 42.542 | 87.391 | 1 | 27.02 |
| 3154 | CG | ASP | 78 | 23.043 | 43.589 | 88.462 | 1 | 30.21 |
| 3155 | OD1 | ASP | 78 | 24.232 | 43.88 | 88.705 | 1 | 31.27 |
| 3156 | OD2 | ASP | 78 | 22.083 | 44.12 | 89.042 | 1 | 31.19 |
| 3157 | C | ASP | 78 | 23.451 | 41.703 | 85.134 | 1 | 25.57 |
| 3158 | O | ASP | 78 | 23.765 | 40.591 | 85.562 | 1 | 25.87 |
| 3159 | N | ILE | 79 | 23.139 | 41.933 | 83.867 | 1 | 25.22 |
| 3160 | CA | ILE | 79 | 23.199 | 40.893 | 82.854 | 1 | 23.78 |
| 3161 | CB | ILE | 79 | 21.824 | 40.652 | 82.19 | 1 | 25.49 |
| 3162 | CG2 | ILE | 79 | 21.984 | 39.762 | 80.955 | 1 | 25.78 |
| 3163 | CG1 | ILE | 79 | 20.87 | 39.991 | 83.182 | 1 | 25.94 |
| 3164 | CD1 | ILE | 79 | 21.372 | 38.659 | 83.719 | 1 | 28.17 |
| 3165 | C | ILE | 79 | 24.169 | 41.408 | 81.798 | 1 | 23.9 |
| 3166 | O | ILE | 79 | 24.05 | 42.551 | 81.35 | 1 | 24.38 |
| 3167 | N | PHE | 80 | 25.143 | 40.577 | 81.435 | 1 | 20.22 |
| 3168 | CA | PHE | 80 | 26.129 | 40.927 | 80.415 | 1 | 20.04 |
| 3169 | CB | PHE | 80 | 27.55 | 40.696 | 80.951 | 1 | 19.06 |
| 3170 | CG | PHE | 80 | 28.644 | 41.179 | 80.027 | 1 | 19.61 |
| 3171 | CD1 | PHE | 80 | 29.024 | 40.429 | 78.911 | 1 | 20.29 |
| 3172 | CD2 | PHE | 80 | 29.301 | 42.38 | 80.28 | 1 | 17.72 |
| 3173 | CE1 | PHE | 80 | 30.051 | 40.869 | 78.055 | 1 | 17.94 |
| 3174 | CE2 | PHE | 80 | 30.324 | 42.829 | 79.442 | 1 | 18.63 |
| 3175 | CZ | PHE | 80 | 30.703 | 42.07 | 78.323 | 1 | 16.39 |
| 3176 | C | PHE | 80 | 25.817 | 39.977 | 79.253 | 1 | 20.87 |
| 3177 | O | PHE | 80 | 26.037 | 38.77 | 79.347 | 1 | 20.12 |
| 3178 | N | TYR | 81 | 25.273 | 40.532 | 78.176 | 1 | 19.86 |
| 3179 | CA | TYR | 81 | 24.885 | 39.751 | 77.013 | 1 | 20.51 |
| 3180 | CB | TYR | 81 | 23.43 | 40.053 | 76.605 | 1 | 19.99 |
| 3181 | CG | TYR | 81 | 23.024 | 39.287 | 75.365 | 1 | 21.37 |
| 3182 | CD1 | TYR | 81 | 22.825 | 37.913 | 75.419 | 1 | 21.54 |
| 3183 | CE1 | TYR | 81 | 22.57 | 37.17 | 74.274 | 1 | 23.14 |
| 3184 | CD2 | TYR | 81 | 22.948 | 39.915 | 74.123 | 1 | 22.65 |
| 3185 | CE2 | TYR | 81 | 22.692 | 39.181 | 72.957 | 1 | 21.42 |
| 3186 | CZ | TYR | 81 | 22.507 | 37.805 | 73.047 | 1 | 24.06 |
| 3187 | OH | TYR | 81 | 22.271 | 37.046 | 71.922 | 1 | 23.23 |
| 3188 | C | TYR | 81 | 25.795 | 40.015 | 75.826 | 1 | 20 |
| 3189 | O | TYR | 81 | 26.017 | 41.166 | 75.442 | 1 | 20.71 |
| 3190 | N | THR | 82 | 26.309 | 38.934 | 75.248 | 1 | 18.88 |
| 3191 | CA | THR | 82 | 27.203 | 39.009 | 74.095 | 1 | 19.62 |
| 3192 | CB | THR | 82 | 28.491 | 38.176 | 74.333 | 1 | 19.73 |
| 3193 | OG1 | THR | 82 | 29.211 | 38.708 | 75.45 | 1 | 20.09 |
| 3194 | CG2 | THR | 82 | 29.388 | 38.19 | 73.084 | 1 | 19.44 |
| 3195 | C | THR | 82 | 26.562 | 38.452 | 72.827 | 1 | 19.88 |
| 3196 | O | THR | 82 | 25.927 | 37.4 | 72.86 | 1 | 18.43 |
| 3197 | N | SER | 83 | 26.7 | 39.169 | 71.716 | 1 | 20.07 |
| 3198 | CA | SER | 83 | 26.222 | 38.648 | 70.442 | 1 | 19.78 |
| 3199 | CB | SER | 83 | 25.044 | 39.442 | 69.887 | 1 | 22.09 |
| 3200 | OG | SER | 83 | 24.518 | 38.772 | 68.739 | 1 | 23.46 |
| 3201 | C | SER | 83 | 27.416 | 38.75 | 69.484 | 1 | 19.72 |
| 3202 | O | SER | 83 | 28.433 | 39.372 | 69.803 | 1 | 19.32 |
| 3203 | N | LYS | 84 | 27.283 | 38.158 | 68.306 | 1 | 19.04 |
| 3204 | CA | LYS | 84 | 28.367 | 38.162 | 67.349 | 1 | 19.87 |
| 3205 | CB | LYS | 84 | 29.11 | 36.815 | 67.388 | 1 | 18.7 |
| 3206 | CG | LYS | 84 | 29.415 | 36.276 | 68.799 | 1 | 20.05 |
| 3207 | CD | LYS | 84 | 30.287 | 35.01 | 68.769 | 1 | 19.48 |
| 3208 | CE | LYS | 84 | 30.471 | 34.415 | 70.169 | 1 | 18.76 |
| 3209 | NZ | LYS | 84 | 29.297 | 33.602 | 70.612 | 1 | 17.06 |
| 3210 | C | LYS | 84 | 27.89 | 38.433 | 65.929 | 1 | 18.83 |
| 3211 | O | LYS | 84 | 26.804 | 38.005 | 65.516 | 1 | 18.7 |
| 3212 | N | LEU | 85 | 28.724 | 39.16 | 65.194 | 1 | 19.54 |
| 3213 | CA | LEU | 85 | 28.465 | 39.516 | 63.803 | 1 | 20.7 |
| 3214 | CB | LEU | 85 | 29.305 | 40.738 | 63.439 | 1 | 18.63 |
| 3215 | CG | LEU | 85 | 29.321 | 41.146 | 61.962 | 1 | 20.07 |
| 3216 | CD1 | LEU | 85 | 28.016 | 41.856 | 61.651 | 1 | 18.73 |
| 3217 | CD2 | LEU | 85 | 30.512 | 42.058 | 61.679 | 1 | 17.83 |
| 3218 | C | LEU | 85 | 28.884 | 38.337 | 62.913 | 1 | 21.17 |
| 3219 | O | LEU | 85 | 30.053 | 37.95 | 62.918 | 1 | 20.5 |
| 3220 | N | TRP | 86 | 27.948 | 37.761 | 62.164 | 1 | 21.27 |
| 3221 | CA | TRP | 86 | 28.311 | 36.637 | 61.301 | 1 | 21.06 |
| 3222 | CB | TRP | 86 | 27.092 | 35.801 | 60.879 | 1 | 21.67 |
| 3223 | CG | TRP | 86 | 27.521 | 34.565 | 60.127 | 1 | 23.15 |
| 3224 | CD2 | TRP | 86 | 28.514 | 33.619 | 60.546 | 1 | 24.43 |
| 3225 | CE2 | TRP | 86 | 28.648 | 32.664 | 59.512 | 1 | 25.79 |
| 3226 | CE3 | TRP | 86 | 29.304 | 33.485 | 61.701 | 1 | 24.11 |
| 3227 | CD1 | TRP | 86 | 27.1 | 34.157 | 58.891 | 1 | 23.02 |
| 3228 | NE1 | TRP | 86 | 27.777 | 33.018 | 58.513 | 1 | 24.76 |
| 3229 | CZ2 | TRP | 86 | 29.545 | 31.587 | 59.592 | 1 | 25.02 |
| 3230 | CZ3 | TRP | 86 | 30.195 | 32.416 | 61.783 | 1 | 25.95 |
| 3231 | CH2 | TRP | 86 | 30.308 | 31.479 | 60.731 | 1 | 26.23 |
| 3232 | C | TRP | 86 | 29.047 | 37.138 | 60.065 | 1 | 21.57 |
| 3233 | O | TRP | 86 | 28.927 | 38.299 | 59.682 | 1 | 22.04 |
| 3234 | N | SER | 87 | 29.803 | 36.241 | 59.447 | 1 | 21.45 |
| 3235 | CA | SER | 87 | 30.638 | 36.538 | 58.291 | 1 | 22.88 |
| 3236 | CB | SER | 87 | 31.524 | 35.33 | 58.017 | 1 | 22.58 |
| 3237 | OG | SER | 87 | 32.38 | 35.105 | 59.131 | 1 | 24.59 |
| 3238 | C | SER | 87 | 29.984 | 36.99 | 56.994 | 1 | 23.13 |
| 3239 | O | SER | 87 | 30.692 | 37.401 | 56.072 | 1 | 23.03 |
| 3240 | N | ASN | 88 | 28.658 | 36.916 | 56.909 | 1 | 22.33 |
| 3241 | CA | ASN | 88 | 27.968 | 37.351 | 55.7 | 1 | 23.47 |
| 3242 | CB | ASN | 88 | 26.704 | 36.502 | 55.453 | 1 | 22.73 |
| 3243 | CG | ASN | 88 | 25.702 | 36.57 | 56.601 | 1 | 25.84 |
| 3244 | OD1 | ASN | 88 | 26.029 | 36.995 | 57.706 | 1 | 25.61 |
| 3245 | ND2 | ASN | 88 | 24.476 | 36.123 | 56.339 | 1 | 24.6 |
| 3246 | C | ASN | 88 | 27.616 | 38.832 | 55.824 | 1 | 24.56 |
| 3247 | O | ASN | 88 | 26.995 | 39.41 | 54.93 | 1 | 24.77 |
| 3248 | N | SER | 89 | 28.035 | 39.452 | 56.927 | 1 | 23.6 |
| 3249 | CA | SER | 89 | 27.768 | 40.872 | 57.144 | 1 | 23.68 |
| 3250 | CB | SER | 89 | 26.697 | 41.069 | 58.229 | 1 | 24.02 |
| 3251 | OG | SER | 89 | 25.433 | 40.577 | 57.794 | 1 | 25.33 |
| 3252 | C | SER | 89 | 29.019 | 41.665 | 57.508 | 1 | 22.6 |
| 3253 | O | SER | 89 | 28.953 | 42.624 | 58.285 | 1 | 23.13 |
| 3254 | N | HIS | 90 | 30.155 | 41.28 | 56.925 | 1 | 22.35 |
| 3255 | CA | HIS | 90 | 31.434 | 41.96 | 57.177 | 1 | 22.48 |
| 3256 | CB | HIS | 90 | 32.625 | 41.071 | 56.789 | 1 | 21.12 |
| 3257 | CG | HIS | 90 | 32.979 | 40.031 | 57.809 | 1 | 21.81 |
| 3258 | CD2 | HIS | 90 | 32.449 | 39.746 | 59.022 | 1 | 20.99 |
| 3259 | ND1 | HIS | 90 | 34.03 | 39.154 | 57.634 | 1 | 20.97 |
| 3260 | CE1 | HIS | 90 | 34.135 | 38.377 | 58.697 | 1 | 20.74 |
| 3261 | NE2 | HIS | 90 | 33.189 | 38.716 | 59.556 | 1 | 21.43 |
| 3262 | C | HIS | 90 | 31.586 | 43.286 | 56.431 | 1 | 23.09 |
| 3263 | O | HIS | 90 | 32.378 | 44.146 | 56.843 | 1 | 23.05 |
| 3264 | N | ARG | 91 | 30.875 | 43.45 | 55.321 | 1 | 24.36 |
| 3265 | CA | ARG | 91 | 30.989 | 44.701 | 54.574 | 1 | 24.7 |
| 3266 | CB | ARG | 91 | 30.134 | 44.651 | 53.309 | 1 | 25.26 |
| 3267 | CG | ARG | 91 | 30.635 | 43.625 | 52.294 | 1 | 27.18 |
| 3268 | CD | ARG | 91 | 29.807 | 43.649 | 51.017 | 1 | 27.88 |
| 3269 | NE | ARG | 91 | 30.241 | 42.614 | 50.083 | 1 | 30.92 |
| 3270 | CZ | ARG | 91 | 29.71 | 42.427 | 48.879 | 1 | 31.94 |
| 3271 | NH1 | ARG | 91 | 28.724 | 43.213 | 48.456 | 1 | 32.98 |
| 3272 | NH2 | ARG | 91 | 30.155 | 41.442 | 48.107 | 1 | 33.31 |
| 3273 | C | ARG | 91 | 30.536 | 45.811 | 55.519 | 1 | 24.43 |
| 3274 | O | ARG | 91 | 29.53 | 45.678 | 56.207 | 1 | 25.45 |
| 3275 | N | PRO | 92 | 31.281 | 46.925 | 55.565 | 1 | 25.75 |
| 3276 | CD | PRO | 92 | 32.393 | 47.259 | 54.661 | 1 | 25.11 |
| 3277 | CA | PRO | 92 | 30.972 | 48.062 | 56.439 | 1 | 26.83 |
| 3278 | CB | PRO | 92 | 31.869 | 49.173 | 55.893 | 1 | 27.11 |
| 3279 | CG | PRO | 92 | 33.043 | 48.415 | 55.379 | 1 | 28.11 |
| 3280 | C | PRO | 92 | 29.516 | 48.499 | 56.533 | 1 | 26.91 |
| 3281 | O | PRO | 92 | 29.014 | 48.719 | 57.634 | 1 | 28.31 |
| 3282 | N | GLU | 93 | 28.84 | 48.615 | 55.393 | 1 | 26.36 |
| 3283 | CA | GLU | 93 | 27.453 | 49.061 | 55.369 | 1 | 27.12 |
| 3284 | CB | GLU | 93 | 27.058 | 49.479 | 53.943 | 1 | 30.41 |
| 3285 | CG | GLU | 93 | 26.975 | 48.317 | 52.955 | 1 | 31.77 |
| 3286 | CD | GLU | 93 | 28.232 | 48.126 | 52.125 | 1 | 34.21 |
| 3287 | OE1 | GLU | 93 | 29.355 | 48.302 | 52.657 | 1 | 33.1 |
| 3288 | OE2 | GLU | 93 | 28.091 | 47.782 | 50.929 | 1 | 38.91 |
| 3289 | C | GLU | 93 | 26.464 | 48.012 | 55.886 | 1 | 26.86 |
| 3290 | O | GLU | 93 | 25.286 | 48.307 | 56.07 | 1 | 26.1 |
| 3291 | N | LEU | 94 | 26.941 | 46.794 | 56.134 | 1 | 25.53 |
| 3292 | CA | LEU | 94 | 26.063 | 45.727 | 56.613 | 1 | 24.95 |
| 3293 | CB | LEU | 94 | 26.336 | 44.432 | 55.835 | 1 | 24.97 |
| 3294 | CG | LEU | 94 | 26.132 | 44.44 | 54.315 | 1 | 26.65 |
| 3295 | CD1 | LEU | 94 | 26.463 | 43.055 | 53.724 | 1 | 26.84 |
| 3296 | CD2 | LEU | 94 | 24.688 | 44.813 | 54.008 | 1 | 25.62 |
| 3297 | C | LEU | 94 | 26.211 | 45.444 | 58.112 | 1 | 25.43 |
| 3298 | O | LEU | 94 | 25.394 | 44.729 | 58.698 | 1 | 25.96 |
| 3299 | N | VAL | 95 | 27.252 | 45.999 | 58.725 | 1 | 25.39 |
| 3300 | CA | VAL | 95 | 27.503 | 45.78 | 60.146 | 1 | 26.12 |
| 3301 | CB | VAL | 95 | 28.835 | 46.43 | 60.569 | 1 | 26.07 |
| 3302 | CG1 | VAL | 95 | 29.067 | 46.242 | 62.075 | 1 | 23.48 |
| 3303 | CG2 | VAL | 95 | 29.971 | 45.804 | 59.772 | 1 | 21.76 |
| 3304 | C | VAL | 95 | 26.384 | 46.258 | 61.075 | 1 | 27.44 |
| 3305 | O | VAL | 95 | 25.841 | 45.466 | 61.852 | 1 | 27.01 |
| 3306 | N | ARG | 96 | 26.03 | 47.539 | 61.007 | 1 | 27.47 |
| 3307 | CA | ARG | 96 | 24.972 | 48.044 | 61.874 | 1 | 27.84 |
| 3308 | CB | ARG | 96 | 24.822 | 49.561 | 61.722 | 1 | 29.5 |
| 3309 | CG | ARG | 96 | 23.652 | 50.109 | 62.515 | 1 | 33.27 |
| 3310 | CD | ARG | 96 | 23.873 | 51.547 | 62.916 | 1 | 37.19 |
| 3311 | NE | ARG | 96 | 22.726 | 52.068 | 63.657 | 1 | 40.47 |
| 3312 | CZ | ARG | 96 | 22.694 | 53.273 | 64.214 | 1 | 41.33 |
| 3313 | NH1 | ARG | 96 | 23.747 | 54.074 | 64.113 | 1 | 41.57 |
| 3314 | NH2 | ARG | 96 | 21.614 | 53.677 | 64.868 | 1 | 41.86 |
| 3315 | C | ARG | 96 | 23.626 | 47.34 | 61.633 | 1 | 27.49 |
| 3316 | O | ARG | 96 | 22.909 | 47.019 | 62.577 | 1 | 26.29 |
| 3317 | N | PRO | 97 | 23.251 | 47.114 | 60.363 | 1 | 28.66 |
| 3318 | CD | PRO | 97 | 23.763 | 47.712 | 59.116 | 1 | 27.81 |
| 3319 | CA | PRO | 97 | 21.974 | 46.434 | 60.126 | 1 | 27.05 |
| 3320 | CB | PRO | 97 | 21.888 | 46.403 | 58.603 | 1 | 27.89 |
| 3321 | CG | PRO | 97 | 22.546 | 47.675 | 58.22 | 1 | 28.33 |
| 3322 | C | PRO | 97 | 21.958 | 45.026 | 60.751 | 1 | 27.06 |
| 3323 | O | PRO | 97 | 20.917 | 44.556 | 61.212 | 1 | 25.73 |
| 3324 | N | ALA | 98 | 23.109 | 44.352 | 60.765 | 1 | 25.7 |
| 3325 | CA | ALA | 98 | 23.187 | 43.012 | 61.359 | 1 | 24.62 |
| 3326 | CB | ALA | 98 | 24.565 | 42.388 | 61.11 | 1 | 23.84 |
| 3327 | C | ALA | 98 | 22.94 | 43.122 | 62.864 | 1 | 24.52 |
| 3328 | O | ALA | 98 | 22.212 | 42.315 | 63.441 | 1 | 23.17 |
| 3329 | N | LEU | 99 | 23.558 | 44.115 | 63.496 | 1 | 23.39 |
| 3330 | CA | LEU | 99 | 23.383 | 44.315 | 64.927 | 1 | 25.24 |
| 3331 | CB | LEU | 99 | 24.316 | 45.418 | 65.447 | 1 | 24.65 |
| 3332 | CG | LEU | 99 | 24.163 | 45.779 | 66.946 | 1 | 25.76 |
| 3333 | CD1 | LEU | 99 | 24.147 | 44.511 | 67.801 | 1 | 25.23 |
| 3334 | CD2 | LEU | 99 | 25.305 | 46.686 | 67.364 | 1 | 25.67 |
| 3335 | C | LEU | 99 | 21.936 | 44.672 | 65.254 | 1 | 25.15 |
| 3336 | O | LEU | 99 | 21.352 | 44.146 | 66.209 | 1 | 24.56 |
| 3337 | N | GLU | 100 | 21.351 | 45.569 | 64.463 | 1 | 26.05 |
| 3338 | CA | GLU | 100 | 19.967 | 45.976 | 64.702 | 1 | 27.38 |
| 3339 | CB | GLU | 100 | 19.564 | 47.109 | 63.75 | 1 | 28.96 |
| 3340 | CG | GLU | 100 | 20.558 | 48.256 | 63.757 | 1 | 34.26 |
| 3341 | CD | GLU | 100 | 19.999 | 49.52 | 63.15 | 1 | 36.84 |
| 3342 | OE1 | GLU | 100 | 19.211 | 49.412 | 62.187 | 1 | 39.85 |
| 3343 | OE2 | GLU | 100 | 20.361 | 50.614 | 63.632 | 1 | 39.16 |
| 3344 | C | GLU | 100 | 19.018 | 44.803 | 64.54 | 1 | 26.48 |
| 3345 | O | GLU | 100 | 17.98 | 44.734 | 65.205 | 1 | 25.47 |
| 3346 | N | ARG | 101 | 19.374 | 43.868 | 63.666 | 1 | 25.02 |
| 3347 | CA | ARG | 101 | 18.526 | 42.706 | 63.459 | 1 | 26.35 |
| 3348 | CB | ARG | 101 | 18.981 | 41.941 | 62.219 | 1 | 30.67 |
| 3349 | CG | ARG | 101 | 18.08 | 40.784 | 61.865 | 1 | 37.33 |
| 3350 | CD | ARG | 101 | 18.547 | 40.148 | 60.582 | 1 | 43.46 |
| 3351 | NE | ARG | 101 | 17.75 | 38.974 | 60.251 | 1 | 49.79 |
| 3352 | CZ | ARG | 101 | 18.031 | 38.147 | 59.249 | 1 | 52.3 |
| 3353 | NH1 | ARG | 101 | 19.092 | 38.372 | 58.484 | 1 | 52.79 |
| 3354 | NH2 | ARG | 101 | 17.258 | 37.093 | 59.019 | 1 | 53.09 |
| 3355 | C | ARG | 101 | 18.553 | 41.802 | 64.696 | 1 | 24.03 |
| 3356 | O | ARG | 101 | 17.521 | 41.265 | 65.11 | 1 | 23.87 |
| 3357 | N | SER | 102 | 19.73 | 41.626 | 65.289 | 1 | 22.85 |
| 3358 | CA | SER | 102 | 19.822 | 40.812 | 66.496 | 1 | 24.12 |
| 3359 | CB | SER | 102 | 21.285 | 40.612 | 65.917 | 1 | 24.62 |
| 3360 | OG | SER | 102 | 21.924 | 39.627 | 66.121 | 1 | 25.5 |
| 3361 | C | SER | 102 | 19.043 | 41.493 | 67.631 | 1 | 22.73 |
| 3362 | O | SER | 102 | 18.311 | 40.835 | 68.371 | 1 | 24.7 |
| 3363 | N | LEU | 103 | 19.187 | 42.808 | 67.759 | 1 | 24.11 |
| 3364 | CA | LEU | 103 | 18.491 | 43.547 | 68.82 | 1 | 25.06 |
| 3365 | CB | LEU | 103 | 18.878 | 45.032 | 68.779 | 1 | 24.73 |
| 3366 | CG | LEU | 103 | 20.332 | 45.368 | 69.153 | 1 | 27.33 |
| 3367 | CD1 | LEU | 103 | 20.622 | 46.852 | 68.923 | 1 | 28.57 |
| 3368 | CD2 | LEU | 103 | 20.578 | 45.003 | 70.622 | 1 | 27.99 |
| 3369 | C | LEU | 103 | 16.981 | 43.375 | 68.679 | 1 | 26.5 |
| 3370 | O | LEU | 103 | 16.268 | 43.275 | 69.676 | 1 | 26.41 |
| 3371 | N | LYS | 104 | 16.492 | 43.327 | 67.443 | 1 | 27.65 |
| 3372 | CA | LYS | 104 | 15.064 | 43.139 | 67.228 | 1 | 29.21 |
| 3373 | CB | LYS | 104 | 14.711 | 43.375 | 65.76 | 1 | 32.36 |
| 3374 | CG | LYS | 104 | 13.231 | 43.248 | 65.477 | 1 | 38.44 |
| 3375 | CD | LYS | 104 | 12.852 | 43.925 | 64.174 | 1 | 42.49 |
| 3376 | CE | LYS | 104 | 11.339 | 44.104 | 64.091 | 1 | 44.61 |
| 3377 | NZ | LYS | 104 | 10.914 | 44.934 | 62.926 | 1 | 46.67 |
| 3378 | C | LYS | 104 | 14.624 | 41.738 | 67.668 | 1 | 28.63 |
| 3379 | O | LYS | 104 | 13.585 | 41.577 | 68.306 | 1 | 26.48 |
| 3380 | N | ASN | 105 | 15.411 | 40.721 | 67.33 | 1 | 28.38 |
| 3381 | CA | ASN | 105 | 15.071 | 39.363 | 67.731 | 1 | 27.47 |
| 3382 | CB | ASN | 105 | 16.046 | 38.356 | 67.112 | 1 | 27.39 |
| 3383 | CG | ASN | 105 | 15.873 | 38.243 | 65.613 | 1 | 31.53 |
| 3384 | OD1 | ASN | 105 | 14.807 | 38.553 | 65.086 | 1 | 33.79 |
| 3385 | ND2 | ASN | 105 | 16.907 | 37.792 | 64.922 | 1 | 32.72 |
| 3386 | C | ASN | 105 | 15.11 | 39.26 | 69.247 | 1 | 26.84 |
| 3387 | O | ASN | 105 | 14.266 | 38.61 | 69.859 | 1 | 25.99 |
| 3388 | N | LEU | 106 | 16.091 | 39.923 | 69.847 | 1 | 25.55 |
| 3389 | CA | LEU | 106 | 16.252 | 39.909 | 71.293 | 1 | 26.49 |
| 3390 | CB | LEU | 106 | 17.669 | 40.36 | 71.668 | 1 | 25.12 |
| 3391 | CG | LEU | 106 | 18.821 | 39.41 | 71.332 | 1 | 24.02 |
| 3392 | CD1 | LEU | 106 | 20.099 | 40.203 | 71.186 | 1 | 25.04 |
| 3393 | CD2 | LEU | 106 | 18.945 | 38.34 | 72.411 | 1 | 25.07 |
| 3394 | C | LEU | 106 | 15.256 | 40.81 | 72.013 | 1 | 27.75 |
| 3395 | O | LEU | 106 | 14.825 | 40.497 | 73.117 | 1 | 27.21 |
| 3396 | N | GLN | 107 | 14.891 | 41.919 | 71.375 | 1 | 28.43 |
| 3397 | CA | GLN | 107 | 14.003 | 42.898 | 71.983 | 1 | 30.44 |
| 3398 | CB | GLN | 107 | 12.7 | 42.25 | 72.471 | 1 | 31.92 |
| 3399 | CG | GLN | 107 | 11.698 | 41.979 | 71.352 | 1 | 35.44 |
| 3400 | CD | GLN | 107 | 11.285 | 43.253 | 70.627 | 1 | 38.8 |
| 3401 | OE1 | GLN | 107 | 10.734 | 44.172 | 71.237 | 1 | 41.91 |
| 3402 | NE2 | GLN | 107 | 11.552 | 43.317 | 69.324 | 1 | 37.34 |
| 3403 | C | GLN | 107 | 14.742 | 43.572 | 73.144 | 1 | 30.8 |
| 3404 | O | GLN | 107 | 14.18 | 43.805 | 74.218 | 1 | 30.82 |
| 3405 | N | LEU | 108 | 16.022 | 43.858 | 72.921 | 1 | 29.8 |
| 3406 | CA | LEU | 108 | 16.856 | 44.55 | 73.905 | 1 | 29.54 |
| 3407 | CB | LEU | 108 | 18.066 | 43.691 | 74.291 | 1 | 29.47 |
| 3408 | CG | LEU | 108 | 17.754 | 42.432 | 75.11 | 1 | 29.63 |
| 3409 | CD1 | LEU | 108 | 19.025 | 41.629 | 75.352 | 1 | 29.41 |
| 3410 | CD2 | LEU | 108 | 17.13 | 42.844 | 76.437 | 1 | 30.16 |
| 3411 | C | LEU | 108 | 17.312 | 45.839 | 73.222 | 1 | 29.64 |
| 3412 | O | LEU | 108 | 17.398 | 45.885 | 71.999 | 1 | 29.62 |
| 3413 | N | ASP | 109 | 17.589 | 46.885 | 73.998 | 1 | 29.32 |
| 3414 | CA | ASP | 109 | 18.014 | 48.161 | 73.428 | 1 | 29.12 |
| 3415 | CB | ASP | 109 | 17.732 | 49.305 | 74.408 | 1 | 32.9 |
| 3416 | CG | ASP | 109 | 16.255 | 49.429 | 74.75 | 1 | 37.9 |
| 3417 | OD1 | ASP | 109 | 15.428 | 49.475 | 73.816 | 1 | 39.21 |
| 3418 | OD2 | ASP | 109 | 15.926 | 49.478 | 75.953 | 1 | 41.44 |
| 3419 | C | ASP | 109 | 19.49 | 48.169 | 73.045 | 1 | 28.37 |
| 3420 | O | ASP | 109 | 19.927 | 48.987 | 72.236 | 1 | 27.72 |
| 3421 | N | TYR | 110 | 20.265 | 47.259 | 73.62 | 1 | 26.92 |
| 3422 | CA | TYR | 110 | 21.687 | 47.196 | 73.301 | 1 | 25.32 |
| 3423 | CB | TYR | 110 | 22.431 | 48.35 | 73.987 | 1 | 26.74 |
| 3424 | CG | TYR | 110 | 22.411 | 48.272 | 75.504 | 1 | 27.83 |
| 3425 | CD1 | TYR | 110 | 21.44 | 48.943 | 76.258 | 1 | 28.23 |
| 3426 | CE1 | TYR | 110 | 21.391 | 48.816 | 77.656 | 1 | 28.46 |
| 3427 | CD2 | TYR | 110 | 23.337 | 47.475 | 76.182 | 1 | 27.8 |
| 3428 | CE2 | TYR | 110 | 23.301 | 47.335 | 77.563 | 1 | 26.49 |
| 3429 | CZ | TYR | 110 | 22.326 | 48.006 | 78.297 | 1 | 29.94 |
| 3430 | OH | TYR | 110 | 22.297 | 47.835 | 79.66 | 1 | 30.12 |
| 3431 | C | TYR | 110 | 22.282 | 45.877 | 73.775 | 1 | 23.75 |
| 3432 | O | TYR | 110 | 21.685 | 45.192 | 74.594 | 1 | 23.32 |
| 3433 | N | VAL | 111 | 23.442 | 45.51 | 73.235 | 1 | 22.2 |
| 3434 | CA | VAL | 111 | 24.125 | 44.305 | 73.7 | 1 | 22.01 |
| 3435 | CB | VAL | 111 | 24.64 | 43.378 | 72.542 | 1 | 20.41 |
| 3436 | CG1 | VAL | 111 | 23.464 | 42.815 | 71.749 | 1 | 19.23 |
| 3437 | CG2 | VAL | 111 | 25.606 | 44.148 | 71.635 | 1 | 19.31 |
| 3438 | C | VAL | 111 | 25.326 | 44.825 | 74.479 | 1 | 19.32 |
| 3439 | O | VAL | 111 | 25.824 | 45.905 | 74.199 | 1 | 20.9 |
| 3440 | N | ASP | 112 | 25.79 | 44.061 | 75.456 | 1 | 19.53 |
| 3441 | CA | ASP | 112 | 26.937 | 44.491 | 76.249 | 1 | 18.48 |
| 3442 | CB | ASP | 112 | 26.95 | 43.71 | 77.552 | 1 | 18.38 |
| 3443 | CG | ASP | 112 | 25.745 | 44.016 | 78.397 | 1 | 20.85 |
| 3444 | OD1 | ASP | 112 | 25.82 | 44.966 | 79.21 | 1 | 21.85 |
| 3445 | OD2 | ASP | 112 | 24.72 | 43.326 | 78.221 | 1 | 20.3 |
| 3446 | C | ASP | 112 | 28.238 | 44.291 | 75.496 | 1 | 18.23 |
| 3447 | O | ASP | 112 | 29.216 | 45.005 | 75.721 | 1 | 18.75 |
| 3448 | N | LEU | 113 | 28.239 | 43.317 | 74.594 | 1 | 16.99 |
| 3449 | CA | LEU | 113 | 29.423 | 43 | 73.821 | 1 | 17.76 |
| 3450 | CB | LEU | 113 | 30.253 | 41.934 | 74.559 | 1 | 15.82 |
| 3451 | CG | LEU | 113 | 31.566 | 41.46 | 73.91 | 1 | 16.22 |
| 3452 | CD1 | LEU | 113 | 32.526 | 42.629 | 73.681 | 1 | 16.89 |
| 3453 | CD2 | LEU | 113 | 32.209 | 40.421 | 74.821 | 1 | 16.54 |
| 3454 | C | LEU | 113 | 29.051 | 42.492 | 72.436 | 1 | 18.74 |
| 3455 | O | LEU | 113 | 28.177 | 41.63 | 72.289 | 1 | 17.72 |
| 3456 | N | TYR | 114 | 29.711 | 43.041 | 71.421 | 1 | 18.92 |
| 3457 | CA | TYR | 114 | 29.462 | 42.611 | 70.05 | 1 | 20.62 |
| 3458 | CB | TYR | 114 | 28.854 | 43.742 | 69.229 | 1 | 19.99 |
| 3459 | CG | TYR | 114 | 28.154 | 43.246 | 67.987 | 1 | 21.84 |
| 3460 | CD1 | TYR | 114 | 27.269 | 42.158 | 68.049 | 1 | 20.26 |
| 3461 | CE1 | TYR | 114 | 26.561 | 41.737 | 66.916 | 1 | 22.06 |
| 3462 | CD2 | TYR | 114 | 28.319 | 43.897 | 66.765 | 1 | 20.03 |
| 3463 | CE2 | TYR | 114 | 27.617 | 43.489 | 65.636 | 1 | 21.15 |
| 3464 | CZ | TYR | 114 | 26.74 | 42.412 | 65.713 | 1 | 22.5 |
| 3465 | OH | TYR | 114 | 26.021 | 42.015 | 64.593 | 1 | 22.68 |
| 3466 | C | TYR | 114 | 30.805 | 42.202 | 69.471 | 1 | 19.94 |
| 3467 | O | TYR | 114 | 31.77 | 42.959 | 69.521 | 1 | 21.19 |
| 3468 | N | LEU | 115 | 30.858 | 40.999 | 68.915 | 1 | 20.18 |
| 3469 | CA | LEU | 115 | 32.105 | 40.478 | 68.379 | 1 | 20.44 |
| 3470 | CB | LEU | 115 | 32.491 | 39.194 | 69.114 | 1 | 18.82 |
| 3471 | CG | LEU | 115 | 32.475 | 39.126 | 70.637 | 1 | 19.55 |
| 3472 | CD1 | LEU | 115 | 32.736 | 37.68 | 71.064 | 1 | 18.69 |
| 3473 | CD2 | LEU | 115 | 33.516 | 40.075 | 71.204 | 1 | 17.11 |
| 3474 | C | LEU | 115 | 32.098 | 40.11 | 66.909 | 1 | 19.78 |
| 3475 | O | LEU | 115 | 31.073 | 39.705 | 66.368 | 1 | 21.56 |
| 3476 | N | ILE | 116 | 33.252 | 40.243 | 66.266 | 1 | 20.57 |
| 3477 | CA | ILE | 116 | 33.353 | 39.772 | 64.893 | 1 | 20.53 |
| 3478 | CB | ILE | 116 | 34.621 | 40.29 | 64.189 | 1 | 21.9 |
| 3479 | CG2 | ILE | 116 | 34.868 | 39.468 | 62.9 | 1 | 21.88 |
| 3480 | CG1 | ILE | 116 | 34.472 | 41.786 | 63.901 | 1 | 19.88 |
| 3481 | CD1 | ILE | 116 | 35.662 | 42.422 | 63.206 | 1 | 22.71 |
| 3482 | C | ILE | 116 | 33.507 | 38.278 | 65.216 | 1 | 21.23 |
| 3483 | O | ILE | 116 | 34.426 | 37.886 | 65.936 | 1 | 20.2 |
| 3484 | N | HIS | 117 | 32.606 | 37.45 | 64.695 | 1 | 19.53 |
| 3485 | CA | HIS | 117 | 32.593 | 36.016 | 64.985 | 1 | 19.41 |
| 3486 | CB | HIS | 117 | 31.252 | 35.442 | 64.523 | 1 | 19.59 |
| 3487 | CG | HIS | 117 | 30.849 | 34.175 | 65.216 | 1 | 20.13 |
| 3488 | CD2 | HIS | 117 | 31.579 | 33.221 | 65.84 | 1 | 20.21 |
| 3489 | ND1 | HIS | 117 | 29.533 | 33.771 | 65.305 | 1 | 20.28 |
| 3490 | CE1 | HIS | 117 | 29.469 | 32.625 | 65.96 | 1 | 20.28 |
| 3491 | NE2 | HIS | 117 | 30.698 | 32.269 | 66.295 | 1 | 21.74 |
| 3492 | C | HIS | 117 | 33.724 | 35.17 | 64.404 | 1 | 20.54 |
| 3493 | O | HIS | 117 | 34.267 | 34.301 | 65.083 | 1 | 19.37 |
| 3494 | N | PHE | 118 | 34.067 | 35.432 | 63.148 | 1 | 20.26 |
| 3495 | CA | PHE | 118 | 35.109 | 34.687 | 62.446 | 1 | 21.96 |
| 3496 | CB | PHE | 118 | 34.508 | 33.45 | 61.787 | 1 | 21.65 |
| 3497 | CG | PHE | 118 | 35.52 | 32.395 | 61.462 | 1 | 24.14 |
| 3498 | CD1 | PHE | 118 | 36.152 | 31.698 | 62.488 | 1 | 24.63 |
| 3499 | CD2 | PHE | 118 | 35.865 | 32.112 | 60.141 | 1 | 25.78 |
| 3500 | CE1 | PHE | 118 | 37.12 | 30.727 | 62.219 | 1 | 27.54 |
| 3501 | CE2 | PHE | 118 | 36.841 | 31.137 | 59.85 | 1 | 26.58 |
| 3502 | CZ | PHE | 118 | 37.467 | 30.444 | 60.897 | 1 | 25.92 |
| 3503 | C | PHE | 118 | 35.677 | 35.631 | 61.386 | 1 | 22.61 |
| 3504 | O | PHE | 118 | 34.928 | 36.305 | 60.692 | 1 | 24.23 |
| 3505 | N | PRO | 119 | 37.011 | 35.684 | 61.248 | 1 | 23.82 |
| 3506 | CD | PRO | 119 | 37.979 | 34.889 | 62.026 | 1 | 23.73 |
| 3507 | CA | PRO | 119 | 37.695 | 36.56 | 60.292 | 1 | 23.42 |
| 3508 | CB | PRO | 119 | 39.131 | 36.565 | 60.81 | 1 | 22.99 |
| 3509 | CG | PRO | 119 | 39.292 | 35.183 | 61.332 | 1 | 24.47 |
| 3510 | C | PRO | 119 | 37.591 | 36.272 | 58.795 | 1 | 24.19 |
| 3511 | O | PRO | 119 | 38.026 | 37.084 | 57.981 | 1 | 26.01 |
| 3512 | N | VAL | 120 | 37.007 | 35.143 | 58.42 | 1 | 23.47 |
| 3513 | CA | VAL | 120 | 36.849 | 34.837 | 57.003 | 1 | 24.24 |
| 3514 | CB | VAL | 120 | 36.983 | 33.308 | 56.749 | 1 | 23.87 |
| 3515 | CG1 | VAL | 120 | 36.682 | 32.989 | 55.294 | 1 | 26.43 |
| 3516 | CG2 | VAL | 120 | 38.408 | 32.843 | 57.094 | 1 | 24.56 |
| 3517 | C | VAL | 120 | 35.463 | 35.34 | 56.57 | 1 | 23.37 |
| 3518 | O | VAL | 120 | 34.455 | 35.016 | 57.198 | 1 | 23.97 |
| 3519 | N | SER | 121 | 35.413 | 36.137 | 55.504 | 1 | 22.7 |
| 3520 | CA | SER | 121 | 34.143 | 36.695 | 55.028 | 1 | 23.83 |
| 3521 | CB | SER | 121 | 34.341 | 38.121 | 54.497 | 1 | 22.24 |
| 3522 | OG | SER | 121 | 35.203 | 38.894 | 55.312 | 1 | 25.56 |
| 3523 | C | SER | 121 | 33.482 | 35.885 | 53.911 | 1 | 24.3 |
| 3524 | O | SER | 121 | 34.176 | 35.29 | 53.091 | 1 | 24.53 |
| 3525 | N | VAL | 122 | 32.146 | 35.874 | 53.887 | 1 | 24.39 |
| 3526 | CA | VAL | 122 | 31.39 | 35.192 | 52.828 | 1 | 24.58 |
| 3527 | CB | VAL | 122 | 30.561 | 33.974 | 53.344 | 1 | 24.37 |
| 3528 | CG1 | VAL | 122 | 31.495 | 32.91 | 53.92 | 1 | 22.74 |
| 3529 | CG2 | VAL | 122 | 29.538 | 34.417 | 54.396 | 1 | 22.77 |
| 3530 | C | VAL | 122 | 30.432 | 36.211 | 52.195 | 1 | 27.4 |
| 3531 | O | VAL | 122 | 30.149 | 37.267 | 52.774 | 1 | 26.69 |
| 3532 | N | LYS | 123 | 29.943 | 35.893 | 51.002 | 1 | 27.42 |
| 3533 | CA | LYS | 123 | 29.036 | 36.773 | 50.267 | 1 | 29.52 |
| 3534 | CB | LYS | 123 | 28.565 | 36.072 | 48.983 | 1 | 31.01 |
| 3535 | CG | LYS | 123 | 28.977 | 36.762 | 47.688 | 1 | 39.6 |
| 3536 | CD | LYS | 123 | 30.491 | 36.807 | 47.503 | 1 | 42.6 |
| 3537 | CE | LYS | 123 | 30.862 | 37.461 | 46.18 | 1 | 46.09 |
| 3538 | NZ | LYS | 123 | 32.336 | 37.593 | 46.027 | 1 | 49.64 |
| 3539 | C | LYS | 123 | 27.801 | 37.219 | 51.052 | 1 | 29.16 |
| 3540 | O | LYS | 123 | 27.203 | 36.432 | 51.787 | 1 | 28.49 |
| 3541 | N | PRO | 124 | 27.4 | 38.492 | 50.893 | 1 | 29.8 |
| 3542 | CD | PRO | 124 | 28.141 | 39.529 | 50.148 | 1 | 30.33 |
| 3543 | CA | PRO | 124 | 26.225 | 39.07 | 51.568 | 1 | 30.53 |
| 3544 | CB | PRO | 124 | 26.174 | 40.495 | 51.012 | 1 | 30.7 |
| 3545 | CG | PRO | 124 | 27.608 | 40.814 | 50.757 | 1 | 30.68 |
| 3546 | C | PRO | 124 | 24.962 | 38.282 | 51.21 | 1 | 31.57 |
| 3547 | O | PRO | 124 | 24.876 | 37.725 | 50.119 | 1 | 31.86 |
| 3548 | N | GLY | 125 | 23.994 | 38.243 | 52.123 | 1 | 31.67 |
| 3549 | CA | GLY | 125 | 22.762 | 37.524 | 51.866 | 1 | 33.76 |
| 3550 | C | GLY | 125 | 22.178 | 36.898 | 53.122 | 1 | 36.86 |
| 3551 | O | GLY | 125 | 22.792 | 36.939 | 54.189 | 1 | 36.34 |
| 3552 | N | GLU | 126 | 20.992 | 36.311 | 53.002 | 1 | 39.29 |
| 3553 | CA | GLU | 126 | 20.342 | 35.689 | 54.147 | 1 | 42.9 |
| 3554 | CB | GLU | 126 | 18.865 | 35.434 | 53.838 | 1 | 46.46 |
| 3555 | CG | GLU | 126 | 18.069 | 36.709 | 53.588 | 1 | 51.73 |
| 3556 | CD | GLU | 126 | 16.646 | 36.453 | 53.109 | 1 | 55.45 |
| 3557 | OE1 | GLU | 126 | 15.876 | 35.77 | 53.828 | 1 | 56.9 |
| 3558 | OE2 | GLU | 126 | 16.294 | 36.944 | 52.008 | 1 | 58.09 |
| 3559 | C | GLU | 126 | 21.026 | 34.386 | 54.563 | 1 | 43.66 |
| 3560 | O | GLU | 126 | 21.092 | 34.065 | 55.755 | 1 | 44.91 |
| 3561 | N | GLU | 127 | 21.553 | 33.649 | 53.589 | 1 | 41.2 |
| 3562 | CA | GLU | 127 | 22.223 | 32.383 | 53.87 | 1 | 40.87 |
| 3563 | CB | GLU | 127 | 22.576 | 31.683 | 52.557 | 1 | 43.76 |
| 3564 | CG | GLU | 127 | 21.403 | 31.584 | 51.595 | 1 | 50.52 |
| 3565 | CD | GLU | 127 | 21.76 | 30.874 | 50.307 | 1 | 55.07 |
| 3566 | OE1 | GLU | 127 | 22.779 | 31.253 | 49.677 | 1 | 57.17 |
| 3567 | OE2 | GLU | 127 | 21.017 | 29.938 | 49.917 | 1 | 57.54 |
| 3568 | C | GLU | 127 | 23.485 | 32.565 | 54.718 | 1 | 38.27 |
| 3569 | O | GLU | 127 | 24.328 | 33.41 | 54.414 | 1 | 36.76 |
| 3570 | N | VAL | 128 | 23.611 | 31.769 | 55.779 | 1 | 35.57 |
| 3571 | CA | VAL | 128 | 24.773 | 31.854 | 56.661 | 1 | 34.51 |
| 3572 | CB | VAL | 128 | 24.544 | 31.089 | 57.991 | 1 | 35.34 |
| 3573 | CG1 | VAL | 128 | 23.59 | 31.872 | 58.878 | 1 | 38.12 |
| 3574 | CG2 | VAL | 128 | 23.973 | 29.716 | 57.714 | 1 | 38.24 |
| 3575 | C | VAL | 128 | 26.017 | 31.308 | 55.974 | 1 | 32.57 |
| 3576 | O | VAL | 128 | 27.115 | 31.817 | 56.17 | 1 | 30.06 |
| 3577 | N | ILE | 129 | 25.85 | 30.259 | 55.18 | 1 | 30.33 |
| 3578 | CA | ILE | 129 | 26.978 | 29.707 | 54.447 | 1 | 30.03 |
| 3579 | CB | ILE | 129 | 27.35 | 28.303 | 54.949 | 1 | 30.67 |
| 3580 | CG2 | ILE | 129 | 28.459 | 27.72 | 54.081 | 1 | 32.7 |
| 3581 | CG1 | ILE | 129 | 27.824 | 28.388 | 56.405 | 1 | 33.2 |
| 3582 | CD1 | ILE | 129 | 28.245 | 27.063 | 57.002 | 1 | 33.76 |
| 3583 | C | ILE | 129 | 26.568 | 29.659 | 52.976 | 1 | 28.26 |
| 3584 | O | ILE | 129 | 26.003 | 28.673 | 52.511 | 1 | 27.38 |
| 3585 | N | PRO | 130 | 26.835 | 30.744 | 52.233 | 1 | 26.68 |
| 3586 | CD | PRO | 130 | 27.563 | 31.935 | 52.693 | 1 | 26.56 |
| 3587 | CA | PRO | 130 | 26.504 | 30.868 | 50.807 | 1 | 27.04 |
| 3588 | CB | PRO | 130 | 26.872 | 32.319 | 50.475 | 1 | 25.79 |
| 3589 | CG | PRO | 130 | 27.009 | 32.992 | 51.787 | 1 | 28.71 |
| 3590 | C | PRO | 130 | 27.329 | 29.9 | 49.958 | 1 | 25.77 |
| 3591 | O | PRO | 130 | 28.552 | 29.854 | 50.088 | 1 | 24.5 |
| 3592 | N | LYS | 131 | 26.656 | 29.146 | 49.093 | 1 | 24.91 |
| 3593 | CA | LYS | 131 | 27.315 | 28.189 | 48.196 | 1 | 24.79 |
| 3594 | CB | LYS | 131 | 27.078 | 26.756 | 48.675 | 1 | 26.39 |
| 3595 | CG | LYS | 131 | 27.803 | 26.384 | 49.944 | 1 | 27.51 |
| 3596 | CD | LYS | 131 | 27.34 | 25.031 | 50.427 | 1 | 33.12 |
| 3597 | CE | LYS | 131 | 28.06 | 24.629 | 51.698 | 1 | 35.52 |
| 3598 | NZ | LYS | 131 | 27.73 | 23.226 | 52.05 | 1 | 39.05 |
| 3599 | C | LYS | 131 | 26.75 | 28.321 | 46.783 | 1 | 24.19 |
| 3600 | O | LYS | 131 | 25.568 | 28.616 | 46.623 | 1 | 23.34 |
| 3601 | N | ASP | 132 | 27.581 | 28.108 | 45.759 | 1 | 22.71 |
| 3602 | CA | ASP | 132 | 27.082 | 28.177 | 44.387 | 1 | 22.22 |
| 3603 | CB | ASP | 132 | 28.198 | 28.52 | 43.383 | 1 | 20.55 |
| 3604 | CG | ASP | 132 | 29.319 | 27.49 | 43.344 | 1 | 23.23 |
| 3605 | OD1 | ASP | 132 | 29.135 | 26.342 | 43.795 | 1 | 21.29 |
| 3606 | OD2 | ASP | 132 | 30.396 | 27.85 | 42.827 | 1 | 23.89 |
| 3607 | C | ASP | 132 | 26.437 | 26.831 | 44.045 | 1 | 23.21 |
| 3608 | O | ASP | 132 | 26.338 | 25.954 | 44.907 | 1 | 22.1 |
| 3609 | N | GLU | 133 | 25.992 | 26.67 | 42.8 | 1 | 22.41 |
| 3610 | CA | GLU | 133 | 25.321 | 25.44 | 42.376 | 1 | 23.86 |
| 3611 | CB | GLU | 133 | 24.8 | 25.588 | 40.952 | 1 | 25.55 |
| 3612 | CG | GLU | 133 | 25.876 | 25.7 | 39.897 | 1 | 28.56 |
| 3613 | CD | GLU | 133 | 25.286 | 25.997 | 38.535 | 1 | 31.04 |
| 3614 | OE1 | GLU | 133 | 24.264 | 25.368 | 38.192 | 1 | 35.64 |
| 3615 | OE2 | GLU | 133 | 25.839 | 26.842 | 37.806 | 1 | 32.06 |
| 3616 | C | GLU | 133 | 26.191 | 24.193 | 42.462 | 1 | 25.26 |
| 3617 | O | GLU | 133 | 25.685 | 23.065 | 42.418 | 1 | 24.92 |
| 3618 | N | ASN | 134 | 27.5 | 24.391 | 42.582 | 1 | 23.78 |
| 3619 | CA | ASN | 134 | 28.422 | 23.26 | 42.671 | 1 | 24.13 |
| 3620 | CB | ASN | 134 | 29.594 | 23.475 | 41.708 | 1 | 22.21 |
| 3621 | CG | ASN | 134 | 29.167 | 23.344 | 40.258 | 1 | 23.78 |
| 3622 | OD1 | ASN | 134 | 29.56 | 24.137 | 39.396 | 1 | 28.06 |
| 3623 | ND2 | ASN | 134 | 28.352 | 22.337 | 39.98 | 1 | 20.35 |
| 3624 | C | ASN | 134 | 28.921 | 22.987 | 44.086 | 1 | 23.51 |
| 3625 | O | ASN | 134 | 29.891 | 22.255 | 44.282 | 1 | 22.18 |
| 3626 | N | GLY | 135 | 28.252 | 23.58 | 45.072 | 1 | 24.03 |
| 3627 | CA | GLY | 135 | 28.63 | 23.349 | 46.458 | 1 | 22.56 |
| 3628 | C | GLY | 135 | 29.825 | 24.133 | 46.96 | 1 | 23.83 |
| 3629 | O | GLY | 135 | 30.278 | 23.916 | 48.08 | 1 | 25.17 |
| 3630 | N | LYS | 136 | 30.345 | 25.047 | 46.153 | 1 | 22.25 |
| 3631 | CA | LYS | 136 | 31.493 | 25.823 | 46.592 | 1 | 22.88 |
| 3632 | CB | LYS | 136 | 32.27 | 26.385 | 45.399 | 1 | 22.99 |
| 3633 | CG | LYS | 136 | 32.843 | 25.345 | 44.44 | 1 | 24.46 |
| 3634 | CD | LYS | 136 | 33.627 | 26.015 | 43.311 | 1 | 24.1 |
| 3635 | CE | LYS | 136 | 34.916 | 26.65 | 43.817 | 1 | 25.34 |
| 3636 | NZ | LYS | 136 | 35.74 | 27.192 | 42.702 | 1 | 25.24 |
| 3637 | C | LYS | 136 | 31.062 | 26.99 | 47.478 | 1 | 23.81 |
| 3638 | O | LYS | 136 | 30.102 | 27.697 | 47.164 | 1 | 23.75 |
| 3639 | N | ILE | 137 | 31.774 | 27.179 | 48.586 | 1 | 22.95 |
| 3640 | CA | ILE | 137 | 31.497 | 28.284 | 49.495 | 1 | 23.81 |
| 3641 | CB | ILE | 137 | 32.44 | 28.287 | 50.719 | 1 | 26.23 |
| 3642 | CG2 | ILE | 137 | 32.107 | 29.467 | 51.61 | 1 | 25.71 |
| 3643 | CG1 | ILE | 137 | 32.35 | 26.97 | 51.49 | 1 | 28.54 |
| 3644 | CD1 | ILE | 137 | 31.002 | 26.691 | 52.061 | 1 | 31.96 |
| 3645 | C | ILE | 137 | 31.809 | 29.564 | 48.732 | 1 | 23.61 |
| 3646 | O | ILE | 137 | 32.849 | 29.656 | 48.081 | 1 | 23.18 |
| 3647 | N | LEU | 138 | 30.921 | 30.547 | 48.812 | 1 | 22.95 |
| 3648 | CA | LEU | 138 | 31.145 | 31.826 | 48.143 | 1 | 23.74 |
| 3649 | CB | LEU | 138 | 29.815 | 32.423 | 47.677 | 1 | 23.47 |
| 3650 | CG | LEU | 138 | 29.035 | 31.61 | 46.628 | 1 | 25.27 |
| 3651 | CD1 | LEU | 138 | 27.66 | 32.23 | 46.394 | 1 | 26.57 |
| 3652 | CD2 | LEU | 138 | 29.836 | 31.573 | 45.335 | 1 | 25.69 |
| 3653 | C | LEU | 138 | 31.841 | 32.81 | 49.085 | 1 | 25.22 |
| 3654 | O | LEU | 138 | 31.192 | 33.526 | 49.86 | 1 | 23.71 |
| 3655 | N | PHE | 139 | 33.164 | 32.833 | 49.019 | 1 | 24.91 |
| 3656 | CA | PHE | 139 | 33.958 | 33.721 | 49.851 | 1 | 27.07 |
| 3657 | CB | PHE | 139 | 35.418 | 33.265 | 49.87 | 1 | 27.38 |
| 3658 | CG | PHE | 139 | 35.617 | 31.92 | 50.487 | 1 | 29.34 |
| 3659 | CD1 | PHE | 139 | 36 | 30.834 | 49.711 | 1 | 29.28 |
| 3660 | CD2 | PHE | 139 | 35.398 | 31.732 | 51.85 | 1 | 28.38 |
| 3661 | CE1 | PHE | 139 | 36.161 | 29.575 | 50.283 | 1 | 30.53 |
| 3662 | CE2 | PHE | 139 | 35.556 | 30.486 | 52.428 | 1 | 29.29 |
| 3663 | CZ | PHE | 139 | 35.939 | 29.398 | 51.646 | 1 | 29.9 |
| 3664 | C | PHE | 139 | 33.884 | 35.148 | 49.329 | 1 | 29.06 |
| 3665 | O | PHE | 139 | 33.672 | 35.376 | 48.137 | 1 | 27.67 |
| 3666 | N | ASP | 140 | 34.069 | 36.105 | 50.229 | 1 | 28.6 |
| 3667 | CA | ASP | 140 | 34.029 | 37.514 | 49.866 | 1 | 31.58 |
| 3668 | CB | ASP | 140 | 32.805 | 38.176 | 50.517 | 1 | 32.21 |
| 3669 | CG | ASP | 140 | 32.5 | 39.559 | 49.945 | 1 | 34.68 |
| 3670 | OD1 | ASP | 140 | 32.879 | 39.833 | 48.789 | 1 | 36.23 |
| 3671 | OD2 | ASP | 140 | 31.863 | 40.369 | 50.647 | 1 | 34.31 |
| 3672 | C | ASP | 140 | 35.329 | 38.139 | 50.361 | 1 | 32.82 |
| 3673 | O | ASP | 140 | 35.928 | 37.654 | 51.32 | 1 | 33.86 |
| 3674 | N | THR | 141 | 35.784 | 39.189 | 49.689 | 1 | 32.76 |
| 3675 | CA | THR | 141 | 37.013 | 39.86 | 50.087 | 1 | 33.56 |
| 3676 | CB | THR | 141 | 37.913 | 40.165 | 48.878 | 1 | 35.84 |
| 3677 | OG1 | THR | 141 | 38.103 | 38.967 | 48.119 | 1 | 39.77 |
| 3678 | CG2 | THR | 141 | 39.274 | 40.671 | 49.343 | 1 | 36.93 |
| 3679 | C | THR | 141 | 36.632 | 41.167 | 50.755 | 1 | 32.53 |
| 3680 | O | THR | 141 | 36.155 | 42.101 | 50.103 | 1 | 31.95 |
| 3681 | N | VAL | 142 | 36.817 | 41.216 | 52.067 | 1 | 30.52 |
| 3682 | CA | VAL | 142 | 36.49 | 42.41 | 52.82 | 1 | 27.86 |
| 3683 | CB | VAL | 142 | 35.262 | 42.188 | 53.735 | 1 | 27.77 |
| 3684 | CG1 | VAL | 142 | 34.888 | 43.496 | 54.418 | 1 | 27.54 |
| 3685 | CG2 | VAL | 142 | 34.082 | 41.656 | 52.923 | 1 | 26.68 |
| 3686 | C | VAL | 142 | 37.672 | 42.797 | 53.688 | 1 | 28.24 |
| 3687 | O | VAL | 142 | 38.298 | 41.951 | 54.323 | 1 | 27.91 |
| 3688 | N | ASP | 143 | 37.984 | 44.087 | 53.703 | 1 | 28.13 |
| 3689 | CA | ASP | 143 | 39.077 | 44.607 | 54.515 | 1 | 27.9 |
| 3690 | CB | ASP | 143 | 39.408 | 46.026 | 54.056 | 1 | 29.39 |
| 3691 | CG | ASP | 143 | 40.48 | 46.687 | 54.903 | 1 | 33.95 |
| 3692 | OD1 | ASP | 143 | 40.951 | 46.073 | 55.887 | 1 | 33.81 |
| 3693 | OD2 | ASP | 143 | 40.859 | 47.835 | 54.586 | 1 | 36.21 |
| 3694 | C | ASP | 143 | 38.536 | 44.623 | 55.945 | 1 | 26.94 |
| 3695 | O | ASP | 143 | 37.636 | 45.402 | 56.249 | 1 | 27.48 |
| 3696 | N | LEU | 144 | 39.07 | 43.768 | 56.817 | 1 | 26.33 |
| 3697 | CA | LEU | 144 | 38.581 | 43.706 | 58.195 | 1 | 26.54 |
| 3698 | CB | LEU | 144 | 39.242 | 42.554 | 58.967 | 1 | 26.97 |
| 3699 | CG | LEU | 144 | 38.81 | 41.135 | 58.56 | 1 | 29.11 |
| 3700 | CD1 | LEU | 144 | 39.538 | 40.083 | 59.394 | 1 | 28.71 |
| 3701 | CD2 | LEU | 144 | 37.308 | 41.002 | 58.733 | 1 | 27.25 |
| 3702 | C | LEU | 144 | 38.775 | 45.018 | 58.945 | 1 | 26.94 |
| 3703 | O | LEU | 144 | 38.077 | 45.287 | 59.921 | 1 | 27.91 |
| 3704 | N | CYS | 145 | 39.72 | 45.843 | 58.507 | 1 | 27.33 |
| 3705 | CA | CYS | 145 | 39.901 | 47.119 | 59.177 | 1 | 27.7 |
| 3706 | CB | CYS | 145 | 41.174 | 47.826 | 58.692 | 1 | 28.73 |
| 3707 | SG | CYS | 145 | 42.692 | 47.121 | 59.402 | 1 | 35.09 |
| 3708 | C | CYS | 145 | 38.667 | 47.99 | 58.945 | 1 | 26.94 |
| 3709 | O | CYS | 145 | 38.303 | 48.781 | 59.819 | 1 | 27.42 |
| 3710 | N | ALA | 146 | 38.017 | 47.834 | 57.788 | 1 | 26.6 |
| 3711 | CA | ALA | 146 | 36.804 | 48.599 | 57.47 | 1 | 25.31 |
| 3712 | CB | ALA | 146 | 36.475 | 48.493 | 55.96 | 1 | 24.37 |
| 3713 | C | ALA | 146 | 35.64 | 48.056 | 58.307 | 1 | 25.77 |
| 3714 | O | ALA | 146 | 34.75 | 48.806 | 58.733 | 1 | 25.16 |
| 3715 | N | THR | 147 | 35.64 | 46.743 | 58.526 | 1 | 24.18 |
| 3716 | CA | THR | 147 | 34.606 | 46.114 | 59.343 | 1 | 24.57 |
| 3717 | CB | THR | 147 | 34.794 | 44.579 | 59.399 | 1 | 23.82 |
| 3718 | OG1 | THR | 147 | 34.774 | 44.053 | 58.065 | 1 | 25.31 |
| 3719 | CG2 | THR | 147 | 33.669 | 43.927 | 60.205 | 1 | 23.43 |
| 3720 | C | THR | 147 | 34.749 | 46.678 | 60.756 | 1 | 23.55 |
| 3721 | O | THR | 147 | 33.767 | 47.071 | 61.38 | 1 | 25.57 |
| 3722 | N | TRP | 148 | 35.986 | 46.742 | 61.245 | 1 | 24.23 |
| 3723 | CA | TRP | 148 | 36.225 | 47.25 | 62.591 | 1 | 23.36 |
| 3724 | CB | TRP | 148 | 37.705 | 47.142 | 62.962 | 1 | 23.54 |
| 3725 | CG | TRP | 148 | 37.935 | 47.433 | 64.424 | 1 | 23.04 |
| 3726 | CD2 | TRP | 148 | 37.494 | 46.631 | 65.531 | 1 | 22.47 |
| 3727 | CE2 | TRP | 148 | 37.819 | 47.333 | 66.717 | 1 | 23.42 |
| 3728 | CE3 | TRP | 148 | 36.851 | 45.387 | 65.636 | 1 | 23.18 |
| 3729 | CD1 | TRP | 148 | 38.503 | 48.559 | 64.969 | 1 | 24.5 |
| 3730 | NE1 | TRP | 148 | 38.433 | 48.506 | 66.348 | 1 | 23.64 |
| 3731 | CZ2 | TRP | 148 | 37.521 | 46.833 | 67.993 | 1 | 23.27 |
| 3732 | CZ3 | TRP | 148 | 36.553 | 44.889 | 66.911 | 1 | 23.7 |
| 3733 | CH2 | TRP | 148 | 36.89 | 45.615 | 68.069 | 1 | 20.81 |
| 3734 | C | TRP | 148 | 35.749 | 48.694 | 62.756 | 1 | 25.54 |
| 3735 | O | TRP | 148 | 35.168 | 49.045 | 63.786 | 1 | 25.46 |
| 3736 | N | GLU | 149 | 35.998 | 49.533 | 61.752 | 1 | 26 |
| 3737 | CA | GLU | 149 | 35.56 | 50.919 | 61.817 | 1 | 26.33 |
| 3738 | CB | GLU | 149 | 35.969 | 51.665 | 60.544 | 1 | 28.95 |
| 3739 | CG | GLU | 149 | 37.373 | 52.251 | 60.629 | 1 | 34.18 |
| 3740 | CD | GLU | 149 | 37.939 | 52.684 | 59.289 | 1 | 37.43 |
| 3741 | OE1 | GLU | 149 | 37.156 | 53.113 | 58.417 | 1 | 40.48 |
| 3742 | OE2 | GLU | 149 | 39.175 | 52.603 | 59.122 | 1 | 39.38 |
| 3743 | C | GLU | 149 | 34.053 | 50.97 | 62.009 | 1 | 26.02 |
| 3744 | O | GLU | 149 | 33.544 | 51.756 | 62.816 | 1 | 27.1 |
| 3745 | N | ALA | 150 | 33.332 | 50.121 | 61.283 | 1 | 24.34 |
| 3746 | CA | ALA | 150 | 31.879 | 50.07 | 61.396 | 1 | 24.88 |
| 3747 | CB | ALA | 150 | 31.299 | 49.131 | 60.309 | 1 | 24.29 |
| 3748 | C | ALA | 150 | 31.473 | 49.598 | 62.801 | 1 | 25.38 |
| 3749 | O | ALA | 150 | 30.468 | 50.049 | 63.347 | 1 | 27.18 |
| 3750 | N | MET | 151 | 32.256 | 48.692 | 63.384 | 1 | 24.41 |
| 3751 | CA | MET | 151 | 31.977 | 48.193 | 64.732 | 1 | 24.44 |
| 3752 | CB | MET | 151 | 32.961 | 47.072 | 65.107 | 1 | 25.95 |
| 3753 | CG | MET | 151 | 32.815 | 45.755 | 64.328 | 1 | 25.68 |
| 3754 | SD | MET | 151 | 31.418 | 44.786 | 64.877 | 1 | 28.58 |
| 3755 | CE | MET | 151 | 32.086 | 44.047 | 66.384 | 1 | 26.24 |
| 3756 | C | MET | 151 | 32.131 | 49.347 | 65.737 | 1 | 24.56 |
| 3757 | O | MET | 151 | 31.332 | 49.488 | 66.66 | 1 | 24.19 |
| 3758 | N | GLU | 152 | 33.181 | 50.153 | 65.565 | 1 | 24.73 |
| 3759 | CA | GLU | 152 | 33.42 | 51.298 | 66.447 | 1 | 26.03 |
| 3760 | CB | GLU | 152 | 34.698 | 52.042 | 66.032 | 1 | 24.12 |
| 3761 | CG | GLU | 152 | 35.957 | 51.207 | 66.192 | 1 | 24.68 |
| 3762 | CD | GLU | 152 | 37.206 | 51.936 | 65.738 | 1 | 26.19 |
| 3763 | OE1 | GLU | 152 | 37.132 | 52.656 | 64.721 | 1 | 28.65 |
| 3764 | OE2 | GLU | 152 | 38.264 | 51.781 | 66.383 | 1 | 27.08 |
| 3765 | C | GLU | 152 | 32.228 | 52.255 | 66.422 | 1 | 26.42 |
| 3766 | O | GLU | 152 | 31.834 | 52.813 | 67.457 | 1 | 26.39 |
| 3767 | N | LYS | 153 | 31.644 | 52.436 | 65.241 | 1 | 27.2 |
| 3768 | CA | LYS | 153 | 30.493 | 53.322 | 65.109 | 1 | 26.81 |
| 3769 | CB | LYS | 153 | 30.157 | 53.553 | 63.633 | 1 | 29.05 |
| 3770 | CG | LYS | 153 | 31.166 | 54.436 | 62.922 | 1 | 30.91 |
| 3771 | CD | LYS | 153 | 30.82 | 54.608 | 61.451 | 1 | 34.68 |
| 3772 | CE | LYS | 153 | 31.836 | 55.496 | 60.749 | 1 | 38.13 |
| 3773 | NZ | LYS | 153 | 31.508 | 55.656 | 59.306 | 1 | 40.81 |
| 3774 | C | LYS | 153 | 29.285 | 52.771 | 65.843 | 1 | 26.46 |
| 3775 | O | LYS | 153 | 28.449 | 53.541 | 66.32 | 1 | 27.56 |
| 3776 | N | CYS | 154 | 29.176 | 51.443 | 65.937 | 1 | 25.25 |
| 3777 | CA | CYS | 154 | 28.055 | 50.85 | 66.664 | 1 | 25.09 |
| 3778 | CB | CYS | 154 | 27.961 | 49.342 | 66.401 | 1 | 27 |
| 3779 | SG | CYS | 154 | 27.428 | 48.921 | 64.727 | 1 | 30.45 |
| 3780 | C | CYS | 154 | 28.218 | 51.112 | 68.159 | 1 | 24.11 |
| 3781 | O | CYS | 154 | 27.233 | 51.294 | 68.873 | 1 | 24.83 |
| 3782 | N | LYS | 155 | 29.459 | 51.123 | 68.637 | 1 | 25.55 |
| 3783 | CA | LYS | 155 | 29.712 | 51.406 | 70.047 | 1 | 25.51 |
| 3784 | CB | LYS | 155 | 31.176 | 51.138 | 70.403 | 1 | 25.73 |
| 3785 | CG | LYS | 155 | 31.558 | 51.53 | 71.826 | 1 | 26.92 |
| 3786 | CD | LYS | 155 | 32.978 | 51.09 | 72.153 | 1 | 27.35 |
| 3787 | CE | LYS | 155 | 33.507 | 51.759 | 73.421 | 1 | 26.92 |
| 3788 | NZ | LYS | 155 | 32.652 | 51.476 | 74.608 | 1 | 29.16 |
| 3789 | C | LYS | 155 | 29.368 | 52.878 | 70.301 | 1 | 27.65 |
| 3790 | O | LYS | 155 | 28.662 | 53.199 | 71.257 | 1 | 27.97 |
| 3791 | N | ASP | 156 | 29.856 | 53.773 | 69.448 | 1 | 28.02 |
| 3792 | CA | ASP | 156 | 29.55 | 55.192 | 69.62 | 1 | 28.95 |
| 3793 | CB | ASP | 156 | 30.254 | 56.034 | 68.556 | 1 | 30.46 |
| 3794 | CG | ASP | 156 | 31.73 | 56.195 | 68.839 | 1 | 31.41 |
| 3795 | OD1 | ASP | 156 | 32.116 | 56.124 | 70.024 | 1 | 34.3 |
| 3796 | OD2 | ASP | 156 | 32.506 | 56.408 | 67.894 | 1 | 35 |
| 3797 | C | ASP | 156 | 28.054 | 55.468 | 69.579 | 1 | 28.87 |
| 3798 | O | ASP | 156 | 27.569 | 56.36 | 70.273 | 1 | 29.89 |
| 3799 | N | ALA | 157 | 27.322 | 54.698 | 68.776 | 1 | 27.44 |
| 3800 | CA | ALA | 157 | 25.878 | 54.874 | 68.652 | 1 | 27.79 |
| 3801 | CB | ALA | 157 | 25.371 | 54.178 | 67.384 | 1 | 28.15 |
| 3802 | C | ALA | 157 | 25.159 | 54.318 | 69.875 | 1 | 28.12 |
| 3803 | O | ALA | 157 | 23.952 | 54.493 | 70.029 | 1 | 29.4 |
| 3804 | N | GLY | 158 | 25.907 | 53.632 | 70.736 | 1 | 26.76 |
| 3805 | CA | GLY | 158 | 25.32 | 53.064 | 71.934 | 1 | 26.83 |
| 3806 | C | GLY | 158 | 24.639 | 51.713 | 71.758 | 1 | 26.48 |
| 3807 | O | GLY | 158 | 24.021 | 51.221 | 72.7 | 1 | 26.46 |
| 3808 | N | LEU | 159 | 24.751 | 51.102 | 70.578 | 1 | 24.65 |
| 3809 | CA | LEU | 159 | 24.11 | 49.804 | 70.324 | 1 | 24.26 |
| 3810 | CB | LEU | 159 | 23.961 | 49.568 | 68.813 | 1 | 24.47 |
| 3811 | CG | LEU | 159 | 23.131 | 50.604 | 68.044 | 1 | 26.75 |
| 3812 | CD1 | LEU | 159 | 23.265 | 50.386 | 66.538 | 1 | 28.57 |
| 3813 | CD2 | LEU | 159 | 21.683 | 50.501 | 68.475 | 1 | 27.65 |
| 3814 | C | LEU | 159 | 24.886 | 48.637 | 70.957 | 1 | 23.19 |
| 3815 | O | LEU | 159 | 24.311 | 47.6 | 71.281 | 1 | 22.64 |
| 3816 | N | ALA | 160 | 26.189 | 48.824 | 71.116 | 1 | 22.81 |
| 3817 | CA | ALA | 160 | 27.065 | 47.827 | 71.716 | 1 | 23.53 |
| 3818 | CB | ALA | 160 | 27.972 | 47.197 | 70.653 | 1 | 20.98 |
| 3819 | C | ALA | 160 | 27.897 | 48.587 | 72.738 | 1 | 24.49 |
| 3820 | O | ALA | 160 | 28.546 | 49.573 | 72.394 | 1 | 25.77 |
| 3821 | N | LYS | 161 | 27.874 | 48.15 | 73.994 | 1 | 23.46 |
| 3822 | CA | LYS | 161 | 28.641 | 48.84 | 75.031 | 1 | 23.07 |
| 3823 | CB | LYS | 161 | 28.213 | 48.339 | 76.414 | 1 | 25.71 |
| 3824 | CG | LYS | 161 | 26.784 | 48.701 | 76.804 | 1 | 27.04 |
| 3825 | CD | LYS | 161 | 26.609 | 50.209 | 76.937 | 1 | 32.62 |
| 3826 | CE | LYS | 161 | 25.24 | 50.561 | 77.52 | 1 | 36.11 |
| 3827 | NZ | LYS | 161 | 25.07 | 52.027 | 77.77 | 1 | 38.63 |
| 3828 | C | LYS | 161 | 30.139 | 48.63 | 74.826 | 1 | 22.81 |
| 3829 | O | LYS | 161 | 30.946 | 49.544 | 75.02 | 1 | 21.97 |
| 3830 | N | SER | 162 | 30.51 | 47.413 | 74.433 | 1 | 20.99 |
| 3831 | CA | SER | 162 | 31.908 | 47.065 | 74.173 | 1 | 20.14 |
| 3832 | CB | SER | 162 | 32.482 | 46.209 | 75.314 | 1 | 20.7 |
| 3833 | OG | SER | 162 | 32.534 | 46.928 | 76.547 | 1 | 20.56 |
| 3834 | C | SER | 162 | 31.964 | 46.249 | 72.876 | 1 | 20.23 |
| 3835 | O | SER | 162 | 30.967 | 45.65 | 72.471 | 1 | 20.14 |
| 3836 | N | ILE | 163 | 33.122 | 46.23 | 72.228 | 1 | 19.07 |
| 3837 | CA | ILE | 163 | 33.286 | 45.473 | 70.997 | 1 | 19.95 |
| 3838 | CB | ILE | 163 | 33.313 | 46.399 | 69.746 | 1 | 18.17 |
| 3839 | CG2 | ILE | 163 | 31.924 | 46.98 | 69.504 | 1 | 18.41 |
| 3840 | CG1 | ILE | 163 | 34.344 | 47.514 | 69.929 | 1 | 22.18 |
| 3841 | CD1 | ILE | 163 | 34.512 | 48.422 | 68.692 | 1 | 21.93 |
| 3842 | C | ILE | 163 | 34.575 | 44.668 | 71.085 | 1 | 19.93 |
| 3843 | O | ILE | 163 | 35.538 | 45.082 | 71.733 | 1 | 19.82 |
| 3844 | N | GLY | 164 | 34.581 | 43.496 | 70.462 | 1 | 19.44 |
| 3845 | CA | GLY | 164 | 35.766 | 42.66 | 70.506 | 1 | 18.53 |
| 3846 | C | GLY | 164 | 35.798 | 41.737 | 69.308 | 1 | 19.86 |
| 3847 | O | GLY | 164 | 35.051 | 41.931 | 68.351 | 1 | 18.58 |
| 3848 | N | VAL | 165 | 36.649 | 40.722 | 69.365 | 1 | 20.23 |
| 3849 | CA | VAL | 165 | 36.767 | 39.782 | 68.267 | 1 | 20.23 |
| 3850 | CB | VAL | 165 | 38.089 | 40.001 | 67.491 | 1 | 20.54 |
| 3851 | CG1 | VAL | 165 | 38.11 | 41.402 | 66.883 | 1 | 19.71 |
| 3852 | CG2 | VAL | 165 | 39.283 | 39.813 | 68.427 | 1 | 19.59 |
| 3853 | C | VAL | 165 | 36.728 | 38.352 | 68.775 | 1 | 21.06 |
| 3854 | O | VAL | 165 | 36.782 | 38.094 | 69.981 | 1 | 20.22 |
| 3855 | N | SER | 166 | 36.62 | 37.419 | 67.838 | 1 | 20.87 |
| 3856 | CA | SER | 166 | 36.584 | 36.013 | 68.174 | 1 | 20.65 |
| 3857 | CB | SER | 166 | 35.138 | 35.52 | 68.225 | 1 | 21.36 |
| 3858 | OG | SER | 166 | 35.07 | 34.163 | 68.626 | 1 | 22.02 |
| 3859 | C | SER | 166 | 37.351 | 35.273 | 67.09 | 1 | 22.23 |
| 3860 | O | SER | 166 | 37.302 | 35.652 | 65.922 | 1 | 21.99 |
| 3861 | N | ASN | 167 | 38.075 | 34.233 | 67.482 | 1 | 20.96 |
| 3862 | CA | ASN | 167 | 38.841 | 33.435 | 66.542 | 1 | 22.06 |
| 3863 | CB | ASN | 167 | 37.893 | 32.728 | 65.569 | 1 | 22.09 |
| 3864 | CG | ASN | 167 | 37.015 | 31.708 | 66.264 | 1 | 21.04 |
| 3865 | OD1 | ASN | 167 | 37.518 | 30.823 | 66.95 | 1 | 23.3 |
| 3866 | ND2 | ASN | 167 | 35.7 | 31.829 | 66.099 | 1 | 18.81 |
| 3867 | C | ASN | 167 | 39.902 | 34.217 | 65.774 | 1 | 23.1 |
| 3868 | O | ASN | 167 | 40.18 | 33.922 | 64.613 | 1 | 23.82 |
| 3869 | N | PHE | 168 | 40.493 | 35.214 | 66.421 | 1 | 23.08 |
| 3870 | CA | PHE | 168 | 41.555 | 35.99 | 65.794 | 1 | 24.03 |
| 3871 | CB | PHE | 168 | 41.473 | 37.47 | 66.192 | 1 | 22.46 |
| 3872 | CG | PHE | 168 | 40.657 | 38.319 | 65.247 | 1 | 22.17 |
| 3873 | CD1 | PHE | 168 | 39.416 | 37.888 | 64.79 | 1 | 21.07 |
| 3874 | CD2 | PHE | 168 | 41.126 | 39.568 | 64.835 | 1 | 21.91 |
| 3875 | CE1 | PHE | 168 | 38.648 | 38.688 | 63.932 | 1 | 20.2 |
| 3876 | CE2 | PHE | 168 | 40.373 | 40.374 | 63.982 | 1 | 20.38 |
| 3877 | CZ | PHE | 168 | 39.131 | 39.929 | 63.531 | 1 | 21.38 |
| 3878 | C | PHE | 168 | 42.886 | 35.428 | 66.259 | 1 | 24.86 |
| 3879 | O | PHE | 168 | 42.995 | 34.912 | 67.372 | 1 | 25.03 |
| 3880 | N | ASN | 169 | 43.898 | 35.513 | 65.4 | 1 | 24.63 |
| 3881 | CA | ASN | 169 | 45.225 | 35.045 | 65.77 | 1 | 24.45 |
| 3882 | CB | ASN | 169 | 45.829 | 34.148 | 64.673 | 1 | 24.35 |
| 3883 | CG | ASN | 169 | 45.875 | 34.831 | 63.313 | 1 | 25.59 |
| 3884 | OD1 | ASN | 169 | 45.86 | 36.064 | 63.219 | 1 | 26.35 |
| 3885 | ND2 | ASN | 169 | 45.953 | 34.029 | 62.252 | 1 | 25.28 |
| 3886 | C | ASN | 169 | 45.079 | 36.283 | 65.986 | 1 | 24.06 |
| 3887 | O | ASN | 169 | 45.585 | 37.406 | 65.891 | 1 | 23.88 |
| 3888 | N | HIS | 170 | 47.355 | 36.066 | 66.287 | 1 | 26.3 |
| 3889 | CA | HIS | 170 | 48.32 | 37.137 | 66.533 | 1 | 28.53 |
| 3890 | CB | HIS | 170 | 49.71 | 36.504 | 66.738 | 1 | 32.78 |
| 3891 | CG | HIS | 170 | 50.839 | 37.486 | 66.77 | 1 | 36.15 |
| 3892 | CD2 | HIS | 170 | 51.968 | 37.569 | 66.027 | 1 | 37.07 |
| 3893 | ND1 | HIS | 170 | 50.888 | 38.538 | 67.66 | 1 | 39.45 |
| 3894 | CE1 | HIS | 170 | 51.998 | 39.229 | 67.461 | 1 | 38.47 |
| 3895 | NE2 | HIS | 170 | 52.671 | 38.662 | 66.476 | 1 | 39.51 |
| 3896 | C | HIS | 170 | 48.363 | 38.167 | 65.403 | 1 | 29.3 |
| 3897 | O | HIS | 170 | 48.24 | 39.375 | 65.635 | 1 | 29.28 |
| 3898 | N | ARG | 171 | 48.536 | 37.681 | 64.179 | 1 | 29.9 |
| 3899 | CA | ARG | 171 | 48.61 | 38.542 | 63.001 | 1 | 30.1 |
| 3900 | CB | ARG | 171 | 48.852 | 37.671 | 61.761 | 1 | 33.25 |
| 3901 | CG | ARG | 171 | 48.681 | 38.377 | 60.422 | 1 | 39.56 |
| 3902 | CD | ARG | 171 | 48.92 | 37.41 | 59.258 | 1 | 43.94 |
| 3903 | NE | ARG | 171 | 47.972 | 37.627 | 58.165 | 1 | 49.18 |
| 3904 | CZ | ARG | 171 | 46.67 | 37.368 | 58.247 | 1 | 51.35 |
| 3905 | NH1 | ARG | 171 | 45.164 | 36.878 | 59.371 | 1 | 55.28 |
| 3906 | NH2 | ARG | 171 | 45.868 | 37.608 | 57.216 | 1 | 52.41 |
| 3907 | C | ARG | 171 | 47.359 | 39.408 | 62.814 | 1 | 28.39 |
| 3908 | O | ARG | 171 | 47.463 | 40.612 | 62.588 | 1 | 29.64 |
| 3909 | N | LEU | 172 | 46.178 | 38.803 | 62.914 | 1 | 26.33 |
| 3910 | CA | LEU | 172 | 44.938 | 39.557 | 62.741 | 1 | 25.38 |
| 3911 | CB | LEU | 172 | 43.747 | 38.603 | 62.663 | 1 | 24.79 |
| 3912 | CG | LEU | 172 | 43.709 | 37.762 | 61.383 | 1 | 26.34 |
| 3913 | CD1 | LEU | 172 | 42.624 | 36.711 | 61.48 | 1 | 25.89 |
| 3914 | CD2 | LEU | 172 | 43.47 | 38.669 | 60.191 | 1 | 28.91 |
| 3915 | C | LEU | 172 | 44.717 | 40.582 | 63.849 | 1 | 25.71 |
| 3916 | O | LEU | 172 | 44.188 | 41.669 | 63.606 | 1 | 25.08 |
| 3917 | N | LEU | 173 | 45.115 | 40.232 | 65.066 | 1 | 26.74 |
| 3918 | CA | LEU | 173 | 44.969 | 41.138 | 66.198 | 1 | 27.55 |
| 3919 | CB | LEU | 173 | 45.348 | 40.409 | 67.495 | 1 | 29.17 |
| 3920 | CG | LEU | 173 | 44.306 | 40.19 | 68.604 | 1 | 31.02 |
| 3921 | CD1 | LEU | 173 | 42.906 | 40.038 | 68.069 | 1 | 32.02 |
| 3922 | CD2 | LEU | 173 | 44.72 | 38.965 | 69.392 | 1 | 30.16 |
| 3923 | C | LEU | 173 | 45.889 | 42.337 | 65.956 | 1 | 29.71 |
| 3924 | O | LEU | 173 | 45.485 | 43.49 | 66.117 | 1 | 28.42 |
| 3925 | N | GLU | 174 | 47.124 | 42.058 | 65.545 | 1 | 31.33 |
| 3926 | CA | GLU | 174 | 48.094 | 43.113 | 65.27 | 1 | 33.6 |
| 3927 | CB | GLU | 174 | 49.42 | 42.503 | 64.813 | 1 | 37.04 |
| 3928 | CG | GLU | 174 | 50.62 | 43.353 | 65.18 | 1 | 45.07 |
| 3929 | CD | GLU | 174 | 50.99 | 43.222 | 66.642 | 1 | 47.57 |
| 3930 | OE1 | GLU | 174 | 51.704 | 42.248 | 66.974 | 1 | 49.93 |
| 3931 | OE2 | GLU | 174 | 50.56 | 44.079 | 67.455 | 1 | 50.39 |
| 3932 | C | GLU | 174 | 47.567 | 44.066 | 64.192 | 1 | 32.62 |
| 3933 | O | GLU | 174 | 47.746 | 45.289 | 64.28 | 1 | 32.33 |
| 3934 | N | MET | 175 | 48.918 | 43.5 | 63.178 | 1 | 31.67 |
| 3935 | CA | MET | 175 | 46.35 | 44.291 | 62.089 | 1 | 32.26 |
| 3936 | CB | MET | 175 | 48.595 | 43.379 | 61.114 | 1 | 34.26 |
| 3937 | CG | MET | 175 | 44.954 | 44.113 | 59.946 | 1 | 38.06 |
| 3938 | SD | MET | 175 | 43.842 | 43.057 | 58.977 | 1 | 44.03 |
| 3939 | CE | MET | 175 | 44.985 | 42.348 | 57.82 | 1 | 42.23 |
| 3940 | C | MET | 175 | 45.4 | 48.356 | 62.645 | 1 | 31.22 |
| 3941 | O | MET | 175 | 45.469 | 46.525 | 62.259 | 1 | 31.12 |
| 3942 | N | ILE | 176 | 44.518 | 44.948 | 63.556 | 1 | 29.2 |
| 3943 | CA | ILE | 176 | 43.56 | 45.873 | 64.169 | 1 | 29.44 |
| 3944 | CB | ILE | 176 | 42.487 | 45.126 | 65.013 | 1 | 29.2 |
| 3945 | CG2 | ILE | 176 | 41.531 | 46.13 | 65.656 | 1 | 26.57 |
| 3946 | CG1 | ILE | 176 | 41.711 | 44.145 | 64.13 | 1 | 29.65 |
| 3947 | CD1 | ILE | 176 | 41.039 | 44.797 | 62.933 | 1 | 32.17 |
| 3948 | C | ILE | 176 | 44.25 | 46.874 | 65.091 | 1 | 29.17 |
| 3949 | O | ILE | 176 | 43.992 | 48.069 | 65.013 | 1 | 29.18 |
| 3950 | N | LEU | 177 | 48.121 | 46.38 | 65.968 | 1 | 29.28 |
| 3951 | CA | LEU | 177 | 45.818 | 47.246 | 66.909 | 1 | 30.31 |
| 3952 | CB | LEU | 177 | 46.686 | 46.411 | 67.853 | 1 | 29.49 |
| 3953 | CG | LEU | 177 | 45.926 | 46.469 | 68.798 | 1 | 29.49 |
| 3954 | CD1 | LEU | 177 | 46.925 | 44.666 | 69.627 | 1 | 29.17 |
| 3955 | CD2 | LEU | 177 | 44.999 | 46.283 | 69.707 | 1 | 29.94 |
| 3956 | C | LEU | 177 | 46.678 | 48.318 | 66.241 | 1 | 32.67 |
| 3957 | O | LEU | 177 | 46.811 | 49.426 | 66.762 | 1 | 32.4 |
| 3958 | N | ASN | 178 | 47.258 | 47.993 | 65.09 | 1 | 33.5 |
| 3959 | CA | ASN | 178 | 48.109 | 48.946 | 64.387 | 1 | 34.66 |
| 3960 | CB | ASN | 178 | 49.302 | 48.227 | 63.754 | 1 | 35.62 |
| 3961 | CG | ASN | 178 | 50.198 | 47.572 | 64.788 | 1 | 39.47 |
| 3962 | OD1 | ASN | 178 | 50.574 | 48.196 | 65.781 | 1 | 41.76 |
| 3963 | ND2 | ASN | 178 | 50.546 | 46.311 | 64.563 | 1 | 39.83 |
| 3964 | C | ASN | 178 | 47.353 | 49.727 | 63.32 | 1 | 34.67 |
| 3965 | O | ASN | 178 | 47.949 | 50.492 | 62.563 | 1 | 35.38 |
| 3966 | N | LYS | 179 | 46.043 | 49.537 | 63.264 | 1 | 33.89 |
| 3967 | CA | LYS | 179 | 45.226 | 50.237 | 62.293 | 1 | 34.68 |
| 3968 | CB | LYS | 179 | 43.771 | 49.784 | 62.406 | 1 | 33.92 |
| 3969 | CG | LYS | 179 | 42.814 | 50.569 | 61.545 | 1 | 35.62 |
| 3970 | CD | LYS | 179 | 41.389 | 50.295 | 61.965 | 1 | 36.18 |
| 3971 | CE | LYS | 179 | 40.447 | 51.217 | 61.248 | 1 | 39.1 |
| 3972 | NZ | LYS | 179 | 40.762 | 52.641 | 61.55 | 1 | 38.7 |
| 3973 | C | LYS | 179 | 45.299 | 51.746 | 62.505 | 1 | 35.14 |
| 3974 | O | LYS | 179 | 45.16 | 52.239 | 63.623 | 1 | 35.3 |
| 3975 | N | PRO | 180 | 45.529 | 52.505 | 61.427 | 1 | 35.67 |
| 3976 | CD | PRO | 180 | 45.81 | 52.083 | 60.04 | 1 | 35.36 |
| 3977 | CA | PRO | 180 | 45.601 | 53.962 | 61.57 | 1 | 33.66 |
| 3978 | CB | PRO | 180 | 46.109 | 54.415 | 60.2 | 1 | 34.48 |
| 3979 | CG | PRO | 180 | 45.559 | 53.355 | 59.265 | 1 | 36.41 |
| 3980 | C | PRO | 180 | 44.23 | 54.539 | 61.922 | 1 | 32.96 |
| 3981 | O | PRO | 180 | 43.209 | 54.124 | 61.37 | 1 | 34.61 |
| 3982 | N | GLY | 181 | 44.207 | 55.485 | 62.855 | 1 | 32.23 |
| 3983 | CA | GLY | 181 | 42.949 | 56.091 | 63.252 | 1 | 32.13 |
| 3984 | C | GLY | 181 | 42.122 | 55.246 | 64.209 | 1 | 31.18 |
| 3985 | O | GLY | 181 | 40.968 | 55.571 | 64.479 | 1 | 32.43 |
| 3986 | N | LEU | 182 | 42.711 | 54.168 | 64.724 | 1 | 30.98 |
| 3987 | CA | LEU | 182 | 42.024 | 53.267 | 65.654 | 1 | 30.49 |
| 3988 | CB | LEU | 182 | 43.005 | 52.225 | 66.209 | 1 | 30.27 |
| 3989 | CG | LEU | 182 | 42.43 | 51.214 | 67.214 | 1 | 28.84 |
| 3990 | CD1 | LEU | 182 | 41.506 | 50.263 | 66.485 | 1 | 28.21 |
| 3991 | CD2 | LEU | 182 | 43.555 | 50.434 | 67.877 | 1 | 28.47 |
| 3992 | C | LEU | 182 | 41.404 | 54.028 | 66.818 | 1 | 30.66 |
| 3993 | O | LEU | 182 | 42.079 | 54.801 | 67.495 | 1 | 30.34 |
| 3994 | N | LYS | 183 | 40.119 | 53.807 | 67.059 | 1 | 30.58 |
| 3995 | CA | LYS | 183 | 39.45 | 54.491 | 68.151 | 1 | 30.53 |
| 3996 | CB | LYS | 183 | 38.082 | 54.983 | 67.688 | 1 | 31.75 |
| 3997 | CG | LYS | 183 | 37.414 | 55.943 | 68.649 | 1 | 36.09 |
| 3998 | CD | LYS | 183 | 36.183 | 56.587 | 68.024 | 1 | 38.57 |
| 3999 | CE | LYS | 183 | 35.55 | 57.61 | 68.967 | 1 | 39.87 |
| 4000 | NZ | LYS | 183 | 34.336 | 58.238 | 68.365 | 1 | 40.74 |
| 4001 | C | LYS | 183 | 39.306 | 53.584 | 69.376 | 1 | 30.95 |
| 4002 | O | LYS | 183 | 39.57 | 54.005 | 70.501 | 1 | 30.93 |
| 4003 | N | TYR | 184 | 38.904 | 52.336 | 69.16 | 1 | 29.62 |
| 4004 | CA | TYR | 184 | 38.717 | 51.395 | 70.264 | 1 | 28.68 |
| 4005 | CB | TYR | 184 | 37.223 | 51.122 | 70.467 | 1 | 28.02 |
| 4006 | CG | TYR | 184 | 36.38 | 52.35 | 70.696 | 1 | 28.43 |
| 4007 | CD1 | TYR | 184 | 36.605 | 53.166 | 71.804 | 1 | 28.38 |
| 4008 | CE1 | TYR | 184 | 35.809 | 54.279 | 72.051 | 1 | 30.14 |
| 4009 | CD2 | TYR | 184 | 35.333 | 52.681 | 69.832 | 1 | 27.4 |
| 4010 | CE2 | TYR | 184 | 34.528 | 53.802 | 70.072 | 1 | 28.51 |
| 4011 | CZ | TYR | 184 | 34.774 | 54.592 | 71.184 | 1 | 29.78 |
| 4012 | OH | TYR | 184 | 33.988 | 55.698 | 71.441 | 1 | 31.05 |
| 4013 | C | TYR | 184 | 39.392 | 50.074 | 69.962 | 1 | 28.58 |
| 4014 | O | TYR | 184 | 39.149 | 49.482 | 68.912 | 1 | 28.67 |
| 4015 | N | LYS | 185 | 40.248 | 49.584 | 70.845 | 1 | 26.76 |
| 4016 | CA | LYS | 185 | 40.812 | 48.299 | 70.508 | 1 | 29.06 |
| 4017 | CB | LYS | 185 | 42.232 | 48.14 | 71.053 | 1 | 31.4 |
| 4018 | CG | LYS | 185 | 42.447 | 48.535 | 72.458 | 1 | 33.76 |
| 4019 | CD | LYS | 185 | 43.942 | 48.577 | 72.706 | 1 | 34.8 |
| 4020 | CE | LYS | 185 | 44.251 | 49.368 | 74.171 | 1 | 36.57 |
| 4021 | NZ | LYS | 185 | 46.708 | 48.162 | 74.404 | 1 | 35.78 |
| 4022 | C | LYS | 185 | 39.873 | 47.213 | 71.011 | 1 | 26.11 |
| 4023 | O | LYS | 185 | 38.944 | 47.494 | 71.761 | 1 | 26.29 |
| 4024 | N | PRO | 186 | 40.051 | 45.973 | 70.542 | 1 | 24.89 |
| 4025 | CD | PRO | 186 | 40.938 | 45.496 | 69.463 | 1 | 25.78 |
| 4026 | CA | PRO | 186 | 39.158 | 44.904 | 71.009 | 1 | 24.29 |
| 4027 | CB | PRO | 186 | 39.651 | 43.679 | 70.241 | 1 | 23.54 |
| 4028 | CG | PRO | 186 | 40.209 | 44.261 | 68.978 | 1 | 26.3 |
| 4029 | C | PRO | 186 | 39.279 | 44.715 | 72.525 | 1 | 22.36 |
| 4030 | O | PRO | 186 | 40.38 | 44.766 | 73.067 | 1 | 22.48 |
| 4031 | N | VAL | 187 | 35.161 | 44.503 | 73.213 | 1 | 21.95 |
| 4032 | CA | VAL | 187 | 38.242 | 44.31 | 74.658 | 1 | 20.67 |
| 4033 | CB | VAL | 187 | 36.91 | 44.662 | 75.404 | 1 | 21.13 |
| 4034 | CG1 | VAL | 187 | 36.523 | 46.125 | 75.154 | 1 | 20.89 |
| 4035 | CG2 | VAL | 187 | 35.786 | 43.718 | 74.977 | 1 | 17.82 |
| 4036 | C | VAL | 187 | 38.575 | 42.847 | 74.943 | 1 | 21.82 |
| 4037 | O | VAL | 187 | 39.071 | 42.514 | 76.02 | 1 | 20.83 |
| 4038 | N | CYS | 188 | 38.312 | 41.971 | 73.975 | 1 | 20.74 |
| 4039 | CA | CYS | 188 | 38.584 | 40.55 | 74.167 | 1 | 20.17 |
| 4040 | CB | CYS | 188 | 37.429 | 39.887 | 74.933 | 1 | 20.49 |
| 4041 | SG | CYS | 188 | 35.87 | 39.834 | 73.928 | 1 | 23.03 |
| 4042 | C | CYS | 188 | 38.744 | 39.792 | 72.856 | 1 | 20.41 |
| 4043 | O | CYS | 188 | 38.411 | 40.284 | 71.772 | 1 | 21.33 |
| 4044 | N | ASN | 189 | 39.255 | 38.574 | 72.983 | 1 | 20.08 |
| 4045 | CA | ASN | 189 | 39.401 | 37.666 | 71.856 | 1 | 18.81 |
| 4046 | CB | ASN | 189 | 40.875 | 37.51 | 71.45 | 1 | 20.01 |
| 4047 | CG | ASN | 189 | 41.048 | 36.651 | 70.213 | 1 | 20.57 |
| 4048 | OD1 | ASN | 189 | 40.092 | 36.411 | 69.482 | 1 | 18.3 |
| 4049 | ND2 | ASN | 189 | 42.279 | 36.189 | 69.966 | 1 | 21.29 |
| 4050 | C | ASN | 189 | 38.835 | 36.351 | 72.38 | 1 | 19.18 |
| 4051 | O | ASN | 189 | 39.42 | 35.721 | 73.272 | 1 | 20.26 |
| 4052 | N | GLN | 190 | 37.679 | 35.95 | 71.853 | 1 | 18.43 |
| 4053 | CA | GLN | 190 | 37.053 | 34.705 | 72.29 | 1 | 17.94 |
| 4054 | CB | GLN | 190 | 35.526 | 34.805 | 72.222 | 1 | 17.13 |
| 4055 | CG | GLN | 190 | 34.827 | 33.558 | 72.778 | 1 | 18.21 |
| 4056 | CD | GLN | 190 | 33.318 | 33.569 | 72.608 | 1 | 20.51 |
| 4057 | OE1 | GLN | 190 | 32.737 | 32.648 | 72.015 | 1 | 21.14 |
| 4058 | NE2 | GLN | 190 | 32.67 | 34.598 | 73.137 | 1 | 19.04 |
| 4059 | C | GLN | 190 | 37.549 | 33.574 | 71.394 | 1 | 19.36 |
| 4060 | O | GLN | 190 | 37.243 | 33.537 | 70.201 | 1 | 19.38 |
| 4061 | N | VAL | 191 | 38.317 | 32.657 | 71.976 | 1 | 20.07 |
| 4062 | CA | VAL | 191 | 38.893 | 31.537 | 71.236 | 1 | 20.61 |
| 4063 | CB | VAL | 191 | 40.406 | 31.763 | 70.963 | 1 | 21.77 |
| 4064 | CG1 | VAL | 191 | 40.61 | 32.976 | 70.066 | 1 | 20.92 |
| 4065 | CG2 | VAL | 191 | 41.148 | 31.96 | 72.283 | 1 | 22.75 |
| 4066 | C | VAL | 191 | 38.764 | 30.219 | 71.99 | 1 | 22.39 |
| 4067 | O | VAL | 191 | 38.426 | 30.191 | 73.171 | 1 | 20.27 |
| 4068 | N | GLU | 192 | 39.037 | 29.119 | 71.296 | 1 | 22.19 |
| 4069 | CA | GLU | 192 | 38.985 | 27.808 | 71.921 | 1 | 22.65 |
| 4070 | CB | GLU | 192 | 39.198 | 26.705 | 70.879 | 1 | 23.46 |
| 4071 | CG | GLU | 192 | 39.096 | 25.296 | 71.45 | 1 | 23.93 |
| 4072 | CD | GLU | 192 | 39.269 | 24.213 | 70.392 | 1 | 27.4 |
| 4073 | OE1 | GLU | 192 | 38.971 | 24.478 | 69.205 | 1 | 26.23 |
| 4074 | OE2 | GLU | 192 | 39.692 | 23.095 | 70.759 | 1 | 28.5 |
| 4075 | C | GLU | 192 | 40.116 | 27.751 | 72.943 | 1 | 21.32 |
| 4076 | O | GLU | 192 | 41.24 | 28.144 | 72.642 | 1 | 21.7 |
| 4077 | N | CYS | 193 | 39.827 | 27.26 | 74.145 | 1 | 21.5 |
| 4078 | CA | CYS | 193 | 40.866 | 27.178 | 75.172 | 1 | 22.88 |
| 4079 | CB | CYS | 193 | 41.114 | 28.56 | 75.781 | 1 | 22.37 |
| 4080 | SG | CYS | 193 | 42.63 | 28.647 | 76.765 | 1 | 23.81 |
| 4081 | C | CYS | 193 | 40.486 | 26.175 | 76.258 | 1 | 23.67 |
| 4082 | O | CYS | 193 | 39.407 | 26.256 | 76.842 | 1 | 23.76 |
| 4083 | N | HIS | 194 | 41.382 | 25.227 | 76.518 | 1 | 23.84 |
| 4084 | CA | HIS | 194 | 41.154 | 24.183 | 77.514 | 1 | 23.63 |
| 4085 | CB | HIS | 194 | 40.15 | 23.161 | 76.986 | 1 | 24.05 |
| 4086 | CG | HIS | 194 | 40.487 | 22.638 | 75.622 | 1 | 26.96 |
| 4087 | CD2 | HIS | 194 | 41.398 | 21.722 | 75.222 | 1 | 25.41 |
| 4088 | ND1 | HIS | 194 | 39.897 | 23.119 | 74.471 | 1 | 27.77 |
| 4089 | CE1 | HIS | 194 | 40.434 | 22.523 | 73.42 | 1 | 26.58 |
| 4090 | NE2 | HIS | 194 | 41.349 | 21.671 | 73.849 | 1 | 28.41 |
| 4091 | C | HIS | 194 | 42.499 | 23.513 | 77.806 | 1 | 24.5 |
| 4092 | O | HIS | 194 | 43.505 | 23.832 | 77.166 | 1 | 23.35 |
| 4093 | N | PRO | 195 | 42.539 | 22.581 | 78.775 | 1 | 25.61 |
| 4094 | CD | PRO | 195 | 41.455 | 22.199 | 79.699 | 1 | 25.15 |
| 4095 | CA | PRO | 195 | 43.788 | 21.896 | 79.131 | 1 | 25.96 |
| 4096 | CB | PRO | 195 | 43.337 | 20.907 | 80.204 | 1 | 25.74 |
| 4097 | CG | PRO | 195 | 42.227 | 21.645 | 80.88 | 1 | 26.84 |
| 4098 | C | PRO | 195 | 44.558 | 21.219 | 77.984 | 1 | 26.11 |
| 4099 | O | PRO | 195 | 45.774 | 21.046 | 78.076 | 1 | 26.88 |
| 4100 | N | TYR | 196 | 43.866 | 20.824 | 76.919 | 1 | 26.9 |
| 4101 | CA | TYR | 196 | 44.554 | 20.189 | 75.799 | 1 | 28.74 |
| 4102 | CB | TYR | 196 | 43.668 | 19.137 | 75.132 | 1 | 29.05 |
| 4103 | CG | TYR | 196 | 43.501 | 17.855 | 75.931 | 1 | 32.1 |
| 4104 | CD1 | TYR | 196 | 44.364 | 17.538 | 76.99 | 1 | 31.26 |
| 4105 | CE1 | TYR | 196 | 44.219 | 16.349 | 77.707 | 1 | 32.71 |
| 4106 | CD2 | TYR | 196 | 42.491 | 16.947 | 75.61 | 1 | 31.29 |
| 4107 | CE2 | TYR | 196 | 42.337 | 15.759 | 76.315 | 1 | 34.06 |
| 4108 | CZ | TYR | 196 | 43.203 | 15.466 | 77.361 | 1 | 33.36 |
| 4109 | OH | TYR | 196 | 43.044 | 14.296 | 78.056 | 1 | 32.95 |
| 4110 | C | TYR | 196 | 45.021 | 21.198 | 74.755 | 1 | 29.92 |
| 4111 | O | TYR | 196 | 45.707 | 20.84 | 73.786 | 1 | 30.07 |
| 4112 | N | PHE | 197 | 44.651 | 22.457 | 74.957 | 1 | 27.87 |
| 4113 | CA | PHE | 197 | 45.037 | 23.534 | 74.052 | 1 | 28.15 |
| 4114 | CB | PHE | 197 | 44.062 | 23.578 | 72.873 | 1 | 29.53 |
| 4115 | CG | PHE | 197 | 44.614 | 24.257 | 71.654 | 1 | 32.44 |
| 4116 | CD1 | PHE | 197 | 45.608 | 23.647 | 70.894 | 1 | 35.41 |
| 4117 | CD2 | PHE | 197 | 44.156 | 25.514 | 71.276 | 1 | 34.16 |
| 4118 | CE1 | PHE | 197 | 46.142 | 24.28 | 69.772 | 1 | 36.3 |
| 4119 | CE2 | PHE | 197 | 44.681 | 26.162 | 70.156 | 1 | 35.95 |
| 4120 | CZ | PHE | 197 | 45.677 | 25.543 | 69.402 | 1 | 37.51 |
| 4121 | C | PHE | 197 | 44.936 | 24.806 | 74.897 | 1 | 27.74 |
| 4122 | O | PHE | 197 | 44.017 | 25.605 | 74.716 | 1 | 26.07 |
| 4123 | N | ASN | 198 | 45.894 | 24.994 | 75.806 | 1 | 27.1 |
| 4124 | CA | ASN | 198 | 45.853 | 26.127 | 76.729 | 1 | 27.05 |
| 4125 | CB | ASN | 198 | 46.695 | 25.824 | 77.982 | 1 | 27.21 |
| 4126 | CG | ASN | 198 | 48.183 | 25.776 | 77.706 | 1 | 26.26 |
| 4127 | OD1 | ASN | 198 | 48.71 | 26.569 | 76.933 | 1 | 28.28 |
| 4128 | ND2 | ASN | 198 | 48.873 | 24.857 | 78.368 | 1 | 26.2 |
| 4129 | C | ASN | 198 | 46.175 | 27.516 | 76.192 | 1 | 25.79 |
| 4130 | O | ASN | 198 | 46.119 | 28.483 | 76.94 | 1 | 24.41 |
| 4131 | N | GLN | 199 | 46.501 | 27.615 | 74.906 | 1 | 24.83 |
| 4132 | CA | GLN | 199 | 46.762 | 28.912 | 74.267 | 1 | 26.02 |
| 4133 | CB | GLN | 199 | 45.43 | 29.658 | 74.1 | 1 | 25.23 |
| 4134 | CG | GLN | 199 | 44.393 | 28.922 | 73.261 | 1 | 25.22 |
| 4135 | CD | GLN | 199 | 44.459 | 29.298 | 71.789 | 1 | 25.4 |
| 4136 | OE1 | GLN | 199 | 45.507 | 29.709 | 71.29 | 1 | 25.86 |
| 4137 | NE2 | GLN | 199 | 43.337 | 29.151 | 71.087 | 1 | 22.14 |
| 4138 | C | GLN | 199 | 47.742 | 29.827 | 74.99 | 1 | 26.7 |
| 4139 | O | GLN | 199 | 47.62 | 31.046 | 74.898 | 1 | 26.57 |
| 4140 | N | ARG | 200 | 48.711 | 29.252 | 75.694 | 1 | 28.09 |
| 4141 | CA | ARG | 200 | 49.685 | 30.044 | 76.448 | 1 | 28.58 |
| 4142 | CB | ARG | 200 | 50.799 | 29.127 | 76.969 | 1 | 32.25 |
| 4143 | CG | ARG | 200 | 51.638 | 29.701 | 78.105 | 1 | 37.74 |
| 4144 | CD | ARG | 200 | 53.07 | 29.962 | 77.65 | 1 | 44.51 |
| 4145 | NE | ARG | 200 | 53.199 | 31.263 | 76.995 | 1 | 48.39 |
| 4146 | CZ | ARG | 200 | 54.276 | 31.669 | 76.326 | 1 | 49.74 |
| 4147 | NH1 | ARG | 200 | 55.329 | 30.869 | 76.212 | 1 | 51.15 |
| 4148 | NH2 | ARG | 200 | 54.301 | 32.881 | 75.781 | 1 | 49.06 |
| 4149 | C | ARG | 200 | 50.286 | 31.22 | 75.664 | 1 | 28.41 |
| 4150 | O | ARG | 200 | 50.319 | 32.345 | 76.165 | 1 | 26.45 |
| 4151 | N | LYS | 201 | 50.748 | 30.978 | 74.436 | 1 | 26.72 |
| 4152 | CA | LYS | 201 | 51.342 | 32.061 | 73.649 | 1 | 28.31 |
| 4153 | CB | LYS | 201 | 51.937 | 31.532 | 72.34 | 1 | 30.68 |
| 4154 | CG | LYS | 201 | 53.237 | 30.755 | 72.486 | 1 | 37.8 |
| 4155 | CD | LYS | 201 | 53.801 | 30.401 | 71.105 | 1 | 41.9 |
| 4156 | CE | LYS | 201 | 55.059 | 29.54 | 71.184 | 1 | 45.86 |
| 4157 | NZ | LYS | 201 | 56.18 | 30.229 | 71.887 | 1 | 49.34 |
| 4158 | C | LYS | 201 | 50.361 | 33.188 | 73.329 | 1 | 26.58 |
| 4159 | O | LYS | 201 | 50.688 | 34.363 | 73.487 | 1 | 26.98 |
| 4160 | N | LEU | 202 | 49.163 | 32.828 | 72.871 | 1 | 25.85 |
| 4161 | CA | LEU | 202 | 48.147 | 33.823 | 72.53 | 1 | 23.91 |
| 4162 | CB | LEU | 202 | 46.952 | 33.153 | 71.846 | 1 | 23.65 |
| 4163 | CG | LEU | 202 | 45.819 | 34.079 | 71.381 | 1 | 23.7 |
| 4164 | CD1 | LEU | 202 | 46.368 | 35.186 | 70.498 | 1 | 25.46 |
| 4165 | CD2 | LEU | 202 | 44.773 | 33.273 | 70.623 | 1 | 24.46 |
| 4166 | C | LEU | 202 | 47.688 | 34.557 | 73.786 | 1 | 23.51 |
| 4167 | O | LEU | 202 | 47.484 | 35.773 | 73.763 | 1 | 23.97 |
| 4168 | N | LEU | 203 | 47.538 | 33.822 | 74.885 | 1 | 21.67 |
| 4169 | CA | LEU | 203 | 47.126 | 34.434 | 76.149 | 1 | 22.3 |
| 4170 | CB | LEU | 203 | 46.95 | 33.355 | 77.234 | 1 | 20.74 |
| 4171 | CG | LEU | 203 | 46.642 | 33.809 | 78.676 | 1 | 23.3 |
| 4172 | CD1 | LEU | 203 | 45.375 | 34.669 | 78.715 | 1 | 19.67 |
| 4173 | CD2 | LEU | 203 | 46.471 | 32.56 | 79.568 | 1 | 20.64 |
| 4174 | C | LEU | 203 | 48.128 | 35.5 | 76.602 | 1 | 22.71 |
| 4175 | O | LEU | 203 | 47.733 | 36.609 | 76.969 | 1 | 23.72 |
| 4176 | N | ASP | 204 | 49.425 | 35.187 | 76.577 | 1 | 23.56 |
| 4177 | CA | ASP | 204 | 50.419 | 36.192 | 76.987 | 1 | 24.12 |
| 4178 | CB | ASP | 204 | 51.842 | 35.608 | 77.013 | 1 | 26.09 |
| 4179 | CG | ASP | 204 | 52.115 | 34.791 | 78.266 | 1 | 27.99 |
| 4180 | OD1 | ASP | 204 | 51.489 | 35.077 | 79.306 | 1 | 28.64 |
| 4181 | OD2 | ASP | 204 | 52.963 | 33.875 | 78.217 | 1 | 29.69 |
| 4182 | C | ASP | 204 | 50.387 | 37.398 | 76.059 | 1 | 23.51 |
| 4183 | O | ASP | 204 | 50.542 | 38.534 | 76.503 | 1 | 23.91 |
| 4184 | N | PHE | 205 | 50.187 | 37.158 | 74.765 | 1 | 24.98 |
| 4185 | CA | PHE | 205 | 50.121 | 38.274 | 73.833 | 1 | 23.38 |
| 4186 | CB | PHE | 205 | 49.938 | 37.798 | 72.391 | 1 | 24.99 |
| 4187 | CG | PHE | 205 | 49.765 | 38.929 | 71.412 | 1 | 26.81 |
| 4188 | CD1 | PHE | 205 | 50.83 | 39.768 | 71.098 | 1 | 26.99 |
| 4189 | CD2 | PHE | 205 | 48.526 | 39.196 | 70.856 | 1 | 26.55 |
| 4190 | CE1 | PHE | 205 | 50.66 | 40.864 | 70.246 | 1 | 26.8 |
| 4191 | CE2 | PHE | 205 | 48.346 | 40.292 | 70.003 | 1 | 30.13 |
| 4192 | CZ | PHE | 205 | 49.42 | 41.124 | 69.702 | 1 | 27.73 |
| 4193 | C | PHE | 205 | 48.949 | 39.181 | 74.203 | 1 | 22.42 |
| 4194 | O | PHE | 205 | 49.099 | 40.398 | 74.291 | 1 | 22.75 |
| 4195 | N | CYS | 206 | 47.778 | 38.59 | 74.413 | 1 | 22.27 |
| 4196 | CA | CYS | 206 | 46.602 | 39.375 | 74.774 | 1 | 23.67 |
| 4197 | CB | CYS | 206 | 45.366 | 38.472 | 74.882 | 1 | 24.33 |
| 4198 | SG | CYS | 206 | 44.75 | 37.884 | 73.267 | 1 | 27.76 |
| 4199 | C | CYS | 206 | 46.808 | 40.156 | 76.07 | 1 | 22.4 |
| 4200 | O | CYS | 206 | 46.44 | 41.326 | 76.156 | 1 | 22.26 |
| 4201 | N | LYS | 207 | 47.391 | 39.513 | 77.077 | 1 | 24.23 |
| 4202 | CA | LYS | 207 | 47.652 | 40.19 | 78.346 | 1 | 24.63 |
| 4203 | CB | LYS | 207 | 48.292 | 39.22 | 79.342 | 1 | 25.13 |
| 4204 | CG | LYS | 207 | 47.317 | 38.231 | 79.935 | 1 | 25.83 |
| 4205 | CD | LYS | 207 | 48.005 | 37.272 | 80.885 | 1 | 27.27 |
| 4206 | CE | LYS | 207 | 46.982 | 36.367 | 81.564 | 1 | 27.36 |
| 4207 | NZ | LYS | 207 | 47.631 | 35.354 | 82.45 | 1 | 29.94 |
| 4208 | C | LYS | 207 | 48.568 | 41.397 | 78.161 | 1 | 24.44 |
| 4209 | O | LYS | 207 | 48.386 | 42.438 | 78.803 | 1 | 25.58 |
| 4210 | N | SER | 208 | 49.561 | 41.264 | 77.286 | 1 | 25.32 |
| 4211 | CA | SER | 208 | 50.494 | 42.367 | 77.056 | 1 | 25.46 |
| 4212 | CB | SER | 208 | 51.665 | 41.91 | 76.174 | 1 | 26.22 |
| 4213 | OG | SER | 208 | 51.275 | 41.759 | 74.818 | 1 | 28.33 |
| 4214 | C | SER | 208 | 49.78 | 43.562 | 76.416 | 1 | 26.12 |
| 4215 | O | SER | 208 | 50.264 | 44.688 | 76.494 | 1 | 26.43 |
| 4216 | N | LYS | 209 | 48.617 | 43.325 | 75.811 | 1 | 25.6 |
| 4217 | CA | LYS | 209 | 47.859 | 44.407 | 75.177 | 1 | 26.57 |
| 4218 | CB | LYS | 209 | 47.482 | 44.016 | 73.745 | 1 | 28.83 |
| 4219 | CG | LYS | 209 | 48.671 | 43.646 | 72.86 | 1 | 30.87 |
| 4220 | CD | LYS | 209 | 49.64 | 44.82 | 72.704 | 1 | 32.82 |
| 4221 | CE | LYS | 209 | 50.844 | 44.434 | 71.84 | 1 | 35.79 |
| 4222 | NZ | LYS | 209 | 51.782 | 45.579 | 71.616 | 1 | 36.64 |
| 4223 | C | LYS | 209 | 46.581 | 44.74 | 75.958 | 1 | 27.27 |
| 4224 | O | LYS | 209 | 45.757 | 45.53 | 75.506 | 1 | 27.83 |
| 4225 | N | ASP | 210 | 46.444 | 44.143 | 77.14 | 1 | 26.43 |
| 4226 | CA | ASP | 210 | 45.277 | 44.319 | 78.004 | 1 | 27.2 |
| 4227 | CB | ASP | 210 | 45.101 | 45.768 | 78.49 | 1 | 28.75 |
| 4228 | CG | ASP | 210 | 44.099 | 45.864 | 79.648 | 1 | 31.15 |
| 4229 | OD1 | ASP | 210 | 43.425 | 46.907 | 79.819 | 1 | 31.49 |
| 4230 | OD2 | ASP | 210 | 43.995 | 44.873 | 80.404 | 1 | 32.66 |
| 4231 | C | ASP | 210 | 44.001 | 43.877 | 77.295 | 1 | 25.84 |
| 4232 | O | ASP | 210 | 42.953 | 44.528 | 77.381 | 1 | 26.31 |
| 4233 | N | ILE | 211 | 44.109 | 42.772 | 76.571 | 1 | 23.64 |
| 4234 | CA | ILE | 211 | 42.969 | 42.195 | 75.879 | 1 | 22.21 |
| 4235 | CB | ILE | 211 | 43.326 | 41.807 | 74.424 | 1 | 23 |
| 4236 | CG2 | ILE | 211 | 42.18 | 41 | 73.797 | 1 | 20.67 |
| 4237 | CG1 | ILE | 211 | 43.627 | 43.078 | 73.615 | 1 | 22.51 |
| 4238 | CD1 | ILE | 211 | 44.053 | 42.802 | 72.17 | 1 | 24.95 |
| 4239 | C | ILE | 211 | 42.637 | 40.932 | 76.67 | 1 | 21.64 |
| 4240 | O | ILE | 211 | 43.522 | 40.135 | 76.956 | 1 | 22.47 |
| 4241 | N | VAL | 212 | 41.373 | 49.763 | 77.04 | 1 | 22.74 |
| 4242 | CA | VAL | 212 | 40.951 | 39.596 | 77.809 | 1 | 22.77 |
| 4243 | CB | VAL | 212 | 39.656 | 39.9 | 78.613 | 1 | 23.55 |
| 4244 | CG1 | VAL | 212 | 39.101 | 38.62 | 79.216 | 1 | 22.95 |
| 4245 | CG2 | VAL | 212 | 39.964 | 40.923 | 79.722 | 1 | 24.04 |
| 4246 | C | VAL | 212 | 40.688 | 38.412 | 76.895 | 1 | 22.87 |
| 4247 | O | VAL | 212 | 40.044 | 38.555 | 75.862 | 1 | 22.07 |
| 4248 | N | LEU | 213 | 41.203 | 37.244 | 77.262 | 1 | 21.5 |
| 4249 | CA | LEU | 213 | 40.958 | 36.055 | 76.464 | 1 | 22.56 |
| 4250 | CB | LEU | 213 | 42.175 | 35.113 | 76.506 | 1 | 22.1 |
| 4251 | CG | LEU | 213 | 42.166 | 33.952 | 75.493 | 1 | 23.39 |
| 4252 | CD1 | LEU | 213 | 43.584 | 33.488 | 75.184 | 1 | 27.12 |
| 4253 | CD2 | LEU | 213 | 41.346 | 32.807 | 76.037 | 1 | 26.06 |
| 4254 | C | LEU | 213 | 39.722 | 35.371 | 77.059 | 1 | 21.64 |
| 4255 | O | LEU | 213 | 39.646 | 35.161 | 78.27 | 1 | 22.01 |
| 4256 | N | VAL | 214 | 38.745 | 35.062 | 76.211 | 1 | 20.01 |
| 4257 | CA | VAL | 214 | 37.53 | 34.384 | 76.65 | 1 | 19.73 |
| 4258 | CB | VAL | 214 | 36.257 | 35.133 | 76.175 | 1 | 19.98 |
| 4259 | CG1 | VAL | 214 | 35.003 | 34.365 | 76.593 | 1 | 15.45 |
| 4260 | CG2 | VAL | 214 | 36.244 | 36.555 | 76.771 | 1 | 18.52 |
| 4261 | C | VAL | 214 | 37.576 | 32.99 | 76.031 | 1 | 21.65 |
| 4262 | O | VAL | 214 | 37.674 | 32.84 | 74.805 | 1 | 20.55 |
| 4263 | N | ALA | 215 | 37.505 | 31.975 | 76.882 | 1 | 19.93 |
| 4264 | CA | ALA | 215 | 37.591 | 30.594 | 76.434 | 1 | 20.81 |
| 4265 | CB | ALA | 215 | 38.322 | 29.756 | 77.495 | 1 | 20.96 |
| 4266 | C | ALA | 215 | 36.288 | 29.913 | 76.103 | 1 | 21.6 |
| 4267 | O | ALA | 215 | 35.368 | 29.899 | 76.917 | 1 | 21.53 |
| 4268 | N | TYR | 216 | 36.202 | 29.353 | 74.899 | 1 | 20.94 |
| 4269 | CA | TYR | 216 | 35.021 | 28.588 | 74.551 | 1 | 22.1 |
| 4270 | CB | TYR | 216 | 34.348 | 29.11 | 73.275 | 1 | 21.22 |
| 4271 | CG | TYR | 216 | 35.088 | 28.944 | 71.968 | 1 | 20.46 |
| 4272 | CD1 | TYR | 216 | 35.3 | 27.679 | 71.415 | 1 | 20.84 |
| 4273 | CE1 | TYR | 216 | 35.814 | 27.538 | 70.121 | 1 | 21.78 |
| 4274 | CD2 | TYR | 216 | 35.43 | 30.065 | 71.209 | 1 | 19.29 |
| 4275 | CE2 | TYR | 216 | 35.948 | 29.941 | 69.92 | 1 | 20.31 |
| 4276 | CZ | TYR | 216 | 36.127 | 28.67 | 69.379 | 1 | 20.19 |
| 4277 | OH | TYR | 216 | 36.569 | 28.535 | 68.084 | 1 | 22.59 |
| 4278 | C | TYR | 216 | 35.478 | 27.135 | 74.43 | 1 | 21.99 |
| 4279 | O | TYR | 216 | 36.676 | 26.87 | 74.339 | 1 | 21.2 |
| 4280 | N | SER | 217 | 34.524 | 26.208 | 74.468 | 1 | 23.35 |
| 4281 | CA | SER | 217 | 34.807 | 24.78 | 74.417 | 1 | 24.92 |
| 4282 | CB | SER | 217 | 35.403 | 24.39 | 73.055 | 1 | 25.83 |
| 4283 | OG | SER | 217 | 34.417 | 24.514 | 72.047 | 1 | 28.98 |
| 4284 | C | SER | 217 | 35.769 | 24.433 | 75.552 | 1 | 24.94 |
| 4285 | O | SER | 217 | 36.615 | 23.545 | 75.426 | 1 | 24.4 |
| 4286 | N | ALA | 218 | 35.615 | 25.139 | 76.673 | 1 | 24.95 |
| 4287 | CA | ALA | 218 | 36.461 | 24.939 | 77.851 | 1 | 24.36 |
| 4288 | CB | ALA | 218 | 36.197 | 26.051 | 78.875 | 1 | 25.49 |
| 4289 | C | ALA | 218 | 36.256 | 23.577 | 78.507 | 1 | 23.35 |
| 4290 | O | ALA | 218 | 37.063 | 23.16 | 79.345 | 1 | 22.95 |
| 4291 | N | LEU | 219 | 35.169 | 22.903 | 78.14 | 1 | 24.1 |
| 4292 | CA | LEU | 219 | 34.856 | 21.579 | 78.669 | 1 | 25.84 |
| 4293 | CB | LEU | 219 | 33.414 | 21.536 | 79.188 | 1 | 24.72 |
| 4294 | CG | LEU | 219 | 33.095 | 22.494 | 80.342 | 1 | 25.83 |
| 4295 | CD1 | LEU | 219 | 31.646 | 22.321 | 80.784 | 1 | 24.61 |
| 4296 | CD2 | LEU | 219 | 34.037 | 22.215 | 81.501 | 1 | 24.76 |
| 4297 | C | LEU | 219 | 35.055 | 20.482 | 77.618 | 1 | 26.63 |
| 4298 | O | LEU | 219 | 34.61 | 19.352 | 77.808 | 1 | 28.68 |
| 4299 | N | GLY | 220 | 35.714 | 20.813 | 76.51 | 1 | 28.28 |
| 4300 | CA | GLY | 220 | 35.949 | 19.815 | 75.472 | 1 | 29.4 |
| 4301 | C | GLY | 220 | 35.057 | 19.901 | 74.242 | 1 | 30.96 |
| 4302 | O | GLY | 220 | 35.138 | 19.046 | 73.353 | 1 | 30.61 |
| 4303 | N | SER | 221 | 34.202 | 20.925 | 74.21 | 1 | 30.65 |
| 4304 | CA | SER | 221 | 33.268 | 21.204 | 73.113 | 1 | 32.44 |
| 4305 | CB | SER | 221 | 33.985 | 21.168 | 71.754 | 1 | 32.4 |
| 4306 | OG | SER | 221 | 33.932 | 19.867 | 71.178 | 1 | 30.8 |
| 4307 | C | SER | 221 | 32.055 | 20.28 | 73.05 | 1 | 33.25 |
| 4308 | O | SER | 221 | 32.005 | 19.234 | 73.696 | 1 | 31.91 |
| 4309 | N | HIS | 222 | 31.075 | 20.69 | 72.256 | 1 | 34.38 |
| 4310 | CA | HIS | 222 | 29.857 | 19.919 | 72.067 | 1 | 35.94 |
| 4311 | CB | HIS | 222 | 28.831 | 20.751 | 71.299 | 1 | 37.09 |
| 4312 | CG | HIS | 222 | 29.407 | 21.482 | 70.124 | 1 | 40.16 |
| 4313 | CD2 | HIS | 222 | 29.799 | 21.046 | 68.903 | 1 | 40.46 |
| 4314 | ND1 | HIS | 222 | 29.652 | 22.839 | 70.14 | 1 | 42.05 |
| 4315 | CE1 | HIS | 222 | 30.167 | 23.208 | 68.98 | 1 | 42.17 |
| 4316 | NE2 | HIS | 222 | 30.266 | 22.137 | 68.21 | 1 | 42.81 |
| 4317 | C | HIS | 222 | 30.125 | 18.62 | 71.302 | 1 | 36.26 |
| 4318 | O | HIS | 222 | 29.291 | 17.717 | 71.302 | 1 | 34.82 |
| 4319 | N | ARG | 223 | 31.285 | 18.535 | 70.654 | 1 | 36.75 |
| 4320 | CA | ARG | 223 | 31.653 | 17.355 | 69.878 | 1 | 38.59 |
| 4321 | CB | ARG | 223 | 31.851 | 16.146 | 70.801 | 1 | 38.31 |
| 4322 | CG | ARG | 223 | 32.922 | 16.338 | 71.873 | 1 | 35.17 |
| 4323 | CD | ARG | 223 | 33.11 | 15.061 | 72.681 | 1 | 39.11 |
| 4324 | NE | ARG | 223 | 33.691 | 13.994 | 71.87 | 1 | 38.42 |
| 4325 | CZ | ARG | 223 | 34.993 | 13.854 | 71.636 | 1 | 39 |
| 4326 | NH1 | ARG | 223 | 35.864 | 14.706 | 72.16 | 1 | 37.07 |
| 4327 | NH2 | ARG | 223 | 35.422 | 12.868 | 70.856 | 1 | 39.45 |
| 4328 | C | ARG | 223 | 30.608 | 17.02 | 68.814 | 1 | 39.36 |
| 4329 | O | ARG | 223 | 30.332 | 15.852 | 68.555 | 1 | 39.26 |
| 4330 | N | GLU | 224 | 30.034 | 18.047 | 68.192 | 1 | 41.08 |
| 4331 | CA | GLU | 224 | 29.026 | 17.844 | 67.153 | 1 | 43.24 |
| 4332 | CB | GLU | 224 | 28.105 | 19.063 | 67.062 | 1 | 43.95 |
| 4333 | CG | GLU | 224 | 26.878 | 18.864 | 66.186 | 1 | 46.73 |
| 4334 | CD | GLU | 224 | 26.177 | 20.17 | 65.865 | 1 | 48.29 |
| 4335 | OE1 | GLU | 224 | 26.301 | 21.121 | 66.662 | 1 | 49.58 |
| 4338 | OE2 | GLU | 224 | 25.493 | 20.247 | 64.823 | 1 | 48.86 |
| 4337 | C | GLU | 224 | 29.67 | 17.609 | 65.786 | 1 | 43.52 |
| 4338 | O | GLU | 224 | 30.557 | 18.354 | 65.368 | 1 | 41.39 |
| 4339 | N | GLU | 225 | 29.224 | 16.568 | 65.095 | 1 | 45.73 |
| 4340 | CA | GLU | 225 | 29.742 | 16.261 | 63.765 | 1 | 47.78 |
| 4341 | CB | GLU | 225 | 29.553 | 14.78 | 63.455 | 1 | 49.4 |
| 4342 | CG | GLU | 225 | 30.413 | 13.853 | 64.289 | 1 | 53.17 |
| 4343 | CD | GLU | 225 | 30.094 | 12.389 | 64.034 | 1 | 56.08 |
| 4344 | OE1 | GLU | 225 | 29.093 | 11.888 | 64.59 | 1 | 57.71 |
| 4345 | OE2 | GLU | 225 | 30.835 | 11.744 | 63.263 | 1 | 56.97 |
| 4346 | C | GLU | 225 | 28.967 | 17.097 | 62.751 | 1 | 47.44 |
| 4347 | O | GLU | 225 | 27.796 | 17.405 | 62.97 | 1 | 47.38 |
| 4348 | N | PRO | 226 | 29.609 | 17.477 | 61.633 | 1 | 47.69 |
| 4349 | CD | PRO | 226 | 28.882 | 17.89 | 60.426 | 1 | 49.01 |
| 4350 | CA | PRO | 226 | 31.001 | 17.16 | 61.29 | 1 | 48.17 |
| 4351 | CB | PRO | 226 | 30.99 | 17.121 | 59.755 | 1 | 48.21 |
| 4352 | CG | PRO | 226 | 29.532 | 17.022 | 59.388 | 1 | 49.35 |
| 4353 | C | PRO | 226 | 31.987 | 18.208 | 61.797 | 1 | 47.52 |
| 4354 | O | PRO | 226 | 33.153 | 18.202 | 61.406 | 1 | 48.77 |
| 4355 | N | TRP | 227 | 31.523 | 19.106 | 62.658 | 1 | 46.39 |
| 4356 | CA | TRP | 227 | 32.376 | 20.167 | 63.185 | 1 | 45.73 |
| 4357 | CB | TRP | 227 | 31.542 | 21.137 | 64.022 | 1 | 47.52 |
| 4358 | CG | TRP | 227 | 30.346 | 21.651 | 63.292 | 1 | 50.07 |
| 4359 | CD2 | TRP | 227 | 30.345 | 22.544 | 62.171 | 1 | 51.14 |
| 4360 | CE2 | TRP | 227 | 28.997 | 22.727 | 61.785 | 1 | 51.86 |
| 4361 | CE3 | TRP | 227 | 31.352 | 23.205 | 61.452 | 1 | 52.61 |
| 4362 | CD1 | TRP | 227 | 29.04 | 21.34 | 63.535 | 1 | 50.49 |
| 4363 | NE1 | TRP | 227 | 28.223 | 21.982 | 62.635 | 1 | 51.12 |
| 4364 | CZ2 | TRP | 227 | 28.628 | 23.547 | 60.71 | 1 | 52.18 |
| 4365 | CZ3 | TRP | 227 | 30.984 | 24.022 | 60.381 | 1 | 53.15 |
| 4366 | CH2 | TRP | 227 | 29.632 | 24.183 | 60.023 | 1 | 52.77 |
| 4367 | C | TRP | 227 | 33.543 | 19.654 | 64.016 | 1 | 44.43 |
| 4368 | O | TRP | 227 | 34.661 | 20.156 | 63.903 | 1 | 44.61 |
| 4369 | N | VAL | 228 | 33.283 | 18.658 | 64.853 | 1 | 42.93 |
| 4370 | CA | VAL | 228 | 34.326 | 18.096 | 65.701 | 1 | 42.13 |
| 4371 | CB | VAL | 228 | 33.928 | 18.156 | 67.188 | 1 | 40.82 |
| 4372 | CG1 | VAL | 228 | 35.066 | 17.634 | 68.051 | 1 | 39.45 |
| 4373 | CG2 | VAL | 228 | 33.566 | 19.585 | 67.568 | 1 | 39.2 |
| 4374 | C | VAL | 228 | 34.596 | 16.644 | 65.333 | 1 | 42.87 |
| 4375 | O | VAL | 228 | 33.667 | 15.851 | 65.169 | 1 | 42.96 |
| 4376 | N | ASP | 229 | 35.874 | 16.304 | 65.21 | 1 | 43.32 |
| 4377 | CA | ASP | 229 | 36.285 | 14.95 | 64.862 | 1 | 44.79 |
| 4378 | CB | ASP | 229 | 37.772 | 14.942 | 64.407 | 1 | 46.03 |
| 4379 | CG | ASP | 229 | 38.312 | 13.54 | 64.255 | 1 | 48.28 |
| 4380 | OD1 | ASP | 229 | 37.528 | 12.655 | 63.854 | 1 | 48.6 |
| 4381 | OD2 | ASP | 229 | 39.525 | 13.328 | 64.462 | 1 | 48.83 |
| 4382 | C | ASP | 229 | 36.022 | 13.975 | 66.007 | 1 | 45.19 |
| 4383 | O | ASP | 229 | 36.572 | 14.121 | 67.098 | 1 | 45 |
| 4384 | N | PRO | 230 | 35.167 | 12.966 | 65.771 | 1 | 45.41 |
| 4385 | CD | PRO | 230 | 34.403 | 12.769 | 64.528 | 1 | 46.46 |
| 4386 | CA | PRO | 230 | 34.806 | 11.945 | 66.763 | 1 | 45.67 |
| 4387 | CB | PRO | 230 | 33.873 | 11.019 | 65.983 | 1 | 46.29 |
| 4388 | CG | PRO | 230 | 33.225 | 11.948 | 65.011 | 1 | 48.91 |
| 4389 | C | PRO | 230 | 36.01 | 11.2 | 67.331 | 1 | 45.59 |
| 4390 | O | PRO | 230 | 35.941 | 10.641 | 68.42 | 1 | 46.07 |
| 4391 | N | ASN | 231 | 37.112 | 11.194 | 66.588 | 1 | 46.41 |
| 4392 | CA | ASN | 231 | 38.315 | 10.516 | 67.046 | 1 | 46.32 |
| 4393 | CB | ASN | 231 | 39.207 | 10.148 | 65.864 | 1 | 49.03 |
| 4394 | CG | ASN | 231 | 38.583 | 9.087 | 64.984 | 1 | 51.81 |
| 4395 | OD1 | ASN | 231 | 38.192 | 8.02 | 65.466 | 1 | 53.21 |
| 4396 | ND2 | ASN | 231 | 38.486 | 9.37 | 63.687 | 1 | 52.71 |
| 4397 | C | ASN | 231 | 39.114 | 11.34 | 68.038 | 1 | 45.28 |
| 4398 | O | ASN | 231 | 40.059 | 10.835 | 68.641 | 1 | 45.04 |
| 4399 | N | SER | 232 | 38.747 | 12.607 | 68.207 | 1 | 43.34 |
| 4400 | CA | SER | 232 | 39.458 | 13.469 | 69.148 | 1 | 41.47 |
| 4401 | CB | SER | 232 | 38.972 | 14.919 | 69.023 | 1 | 41.41 |
| 4402 | OG | SER | 232 | 37.593 | 15.03 | 69.313 | 1 | 41.99 |
| 4403 | C | SER | 232 | 39.245 | 12.963 | 70.572 | 1 | 39.62 |
| 4404 | O | SER | 232 | 38.17 | 12.473 | 70.916 | 1 | 39.11 |
| 4405 | N | PRO | 233 | 40.276 | 13.07 | 71.421 | 1 | 38.66 |
| 4406 | CD | PRO | 233 | 41.599 | 13.665 | 71.175 | 1 | 38.21 |
| 4407 | CA | PRO | 233 | 40.162 | 12.608 | 72.807 | 1 | 38.68 |
| 4408 | CB | PRO | 233 | 41.58 | 12.789 | 73.349 | 1 | 38.53 |
| 4409 | CG | PRO | 233 | 42.087 | 13.95 | 72.578 | 1 | 37.92 |
| 4410 | C | PRO | 233 | 39.125 | 13.389 | 73.607 | 1 | 38.62 |
| 4411 | O | PRO | 233 | 38.873 | 14.56 | 73.329 | 1 | 38.88 |
| 4412 | N | VAL | 234 | 38.517 | 12.727 | 74.588 | 1 | 38.53 |
| 4413 | CA | VAL | 234 | 37.51 | 13.356 | 75.438 | 1 | 37.9 |
| 4414 | CB | VAL | 234 | 36.514 | 12.319 | 75.969 | 1 | 36.89 |
| 4415 | CG1 | VAL | 234 | 35.402 | 13.013 | 76.727 | 1 | 38.46 |
| 4416 | CG2 | VAL | 234 | 35.947 | 11.515 | 74.81 | 1 | 37.82 |
| 4417 | C | VAL | 234 | 38.206 | 14.042 | 76.608 | 1 | 37.12 |
| 4418 | O | VAL | 234 | 38.758 | 13.384 | 77.496 | 1 | 37.26 |
| 4419 | N | LEU | 235 | 38.178 | 15.37 | 76.602 | 1 | 35.97 |
| 4420 | CA | LEU | 235 | 38.834 | 16.149 | 77.647 | 1 | 35.14 |
| 4421 | CB | LEU | 235 | 38.55 | 17.647 | 77.462 | 1 | 33.23 |
| 4422 | CG | LEU | 235 | 39.205 | 18.573 | 78.5 | 1 | 32.46 |
| 4423 | CD1 | LEU | 235 | 40.711 | 18.608 | 78.271 | 1 | 31.27 |
| 4424 | CD2 | LEU | 235 | 38.618 | 19.978 | 78.409 | 1 | 30.84 |
| 4425 | C | LEU | 235 | 38.442 | 15.746 | 79.065 | 1 | 34.8 |
| 4426 | O | LEU | 235 | 39.307 | 15.472 | 79.896 | 1 | 34.25 |
| 4427 | N | LEU | 236 | 37.141 | 15.708 | 79.336 | 1 | 34.93 |
| 4428 | CA | LEU | 236 | 36.66 | 15.387 | 80.675 | 1 | 36.72 |
| 4429 | CB | LEU | 236 | 35.161 | 15.67 | 80.775 | 1 | 36.23 |
| 4430 | CG | LEU | 236 | 34.77 | 17.14 | 80.555 | 1 | 36.7 |
| 4431 | CD1 | LEU | 236 | 33.282 | 17.316 | 80.789 | 1 | 37.25 |
| 4432 | CD2 | LEU | 236 | 35.564 | 18.03 | 81.5 | 1 | 35.72 |
| 4433 | C | LEU | 236 | 36.96 | 13.976 | 81.168 | 1 | 38.46 |
| 4434 | O | LEU | 236 | 36.637 | 13.633 | 82.302 | 1 | 38.15 |
| 4435 | N | GLU | 237 | 37.583 | 13.165 | 80.32 | 1 | 39.4 |
| 4436 | CA | GLU | 237 | 37.937 | 11.809 | 80.694 | 1 | 40.78 |
| 4437 | CB | GLU | 237 | 37.581 | 10.838 | 79.563 | 1 | 42.68 |
| 4438 | CG | GLU | 237 | 36.157 | 10.326 | 79.641 | 1 | 46.16 |
| 4439 | CD | GLU | 237 | 35.772 | 9.474 | 78.45 | 1 | 49.85 |
| 4440 | OE1 | GLU | 237 | 36.627 | 8.699 | 77.971 | 1 | 51.65 |
| 4441 | OE2 | GLU | 237 | 34.608 | 9.569 | 78 | 1 | 51.49 |
| 4442 | C | GLU | 237 | 39.416 | 11.708 | 81.029 | 1 | 40.47 |
| 4443 | O | GLU | 237 | 39.905 | 10.638 | 81.375 | 1 | 40.93 |
| 4444 | N | ASP | 238 | 40.127 | 12.828 | 80.938 | 1 | 39.51 |
| 4445 | CA | ASP | 238 | 41.557 | 12.847 | 81.228 | 1 | 39.33 |
| 4446 | CB | ASP | 238 | 42.115 | 14.25 | 80.998 | 1 | 38.13 |
| 4447 | CG | ASP | 238 | 43.6 | 14.328 | 81.238 | 1 | 37.88 |
| 4448 | OD1 | ASP | 238 | 44.011 | 14.46 | 82.41 | 1 | 38.54 |
| 4449 | OD2 | ASP | 238 | 44.362 | 14.247 | 80.252 | 1 | 36.73 |
| 4450 | C | ASP | 238 | 41.826 | 12.393 | 82.662 | 1 | 39.27 |
| 4451 | O | ASP | 238 | 41.178 | 12.857 | 83.599 | 1 | 39.59 |
| 4452 | N | PRO | 239 | 42.787 | 11.469 | 82.847 | 1 | 39.96 |
| 4453 | CD | PRO | 239 | 43.592 | 10.835 | 81.787 | 1 | 40.09 |
| 4454 | CA | PRO | 239 | 43.158 | 10.928 | 84.164 | 1 | 39.09 |
| 4455 | CB | PRO | 239 | 44.359 | 10.037 | 83.845 | 1 | 38.92 |
| 4456 | CG | PRO | 239 | 44.079 | 9.576 | 82.469 | 1 | 40.57 |
| 4457 | C | PRO | 239 | 43.507 | 11.986 | 85.212 | 1 | 37.19 |
| 4458 | O | PRO | 239 | 42.994 | 11.955 | 86.327 | 1 | 37.68 |
| 4459 | N | VAL | 240 | 44.391 | 12.906 | 84.848 | 1 | 35.63 |
| 4460 | CA | VAL | 240 | 44.82 | 13.961 | 85.756 | 1 | 35.06 |
| 4461 | CB | VAL | 240 | 45.886 | 14.858 | 85.086 | 1 | 35.09 |
| 4462 | CG1 | VAL | 240 | 46.315 | 15.971 | 86.029 | 1 | 34.64 |
| 4463 | CG2 | VAL | 240 | 47.095 | 14.013 | 84.704 | 1 | 35.8 |
| 4464 | C | VAL | 240 | 43.636 | 14.811 | 86.223 | 1 | 35.64 |
| 4465 | O | VAL | 240 | 43.499 | 15.11 | 87.413 | 1 | 34.56 |
| 4466 | N | LEU | 241 | 42.777 | 15.191 | 85.282 | 1 | 35.51 |
| 4467 | CA | LEU | 241 | 41.603 | 15.989 | 85.604 | 1 | 35.92 |
| 4468 | CB | LEU | 241 | 40.846 | 16.347 | 84.319 | 1 | 36.74 |
| 4469 | CG | LEU | 241 | 40.853 | 17.811 | 83.862 | 1 | 37.91 |
| 4470 | CD1 | LEU | 241 | 42.237 | 18.414 | 83.992 | 1 | 36.91 |
| 4471 | CD2 | LEU | 241 | 40.363 | 17.886 | 82.425 | 1 | 36.48 |
| 4472 | C | LEU | 241 | 40.683 | 15.228 | 86.562 | 1 | 36.07 |
| 4473 | O | LEU | 241 | 40.156 | 15.801 | 87.518 | 1 | 34.97 |
| 4474 | N | CYS | 242 | 40.487 | 13.938 | 86.314 | 1 | 35.99 |
| 4475 | CA | CYS | 242 | 39.626 | 13.143 | 87.184 | 1 | 37.64 |
| 4476 | CB | CYS | 242 | 39.331 | 11.786 | 86.537 | 1 | 39.44 |
| 4477 | SG | CYS | 242 | 38.353 | 11.916 | 85.015 | 1 | 44.32 |
| 4478 | C | CYS | 242 | 40.251 | 12.951 | 88.568 | 1 | 37.12 |
| 4479 | O | CYS | 242 | 39.55 | 12.941 | 89.58 | 1 | 36.63 |
| 4480 | N | ALA | 243 | 41.573 | 12.812 | 88.607 | 1 | 36.6 |
| 4481 | CA | ALA | 243 | 42.286 | 12.63 | 89.867 | 1 | 37.02 |
| 4482 | CB | ALA | 243 | 43.757 | 12.314 | 89.593 | 1 | 37.21 |
| 4483 | C | ALA | 243 | 42.176 | 13.88 | 90.745 | 1 | 37.53 |
| 4484 | O | ALA | 243 | 41.897 | 13.791 | 91.941 | 1 | 36.53 |
| 4485 | N | LEU | 244 | 42.41 | 15.044 | 90.144 | 1 | 36.64 |
| 4486 | CA | LEU | 244 | 42.325 | 16.303 | 90.871 | 1 | 36.27 |
| 4487 | CB | LEU | 244 | 42.803 | 17.464 | 89.987 | 1 | 36.4 |
| 4488 | CG | LEU | 244 | 44.314 | 17.511 | 89.719 | 1 | 38.28 |
| 4489 | CD1 | LEU | 244 | 44.63 | 18.636 | 88.627 | 1 | 37.5 |
| 4490 | CD2 | LEU | 244 | 45.047 | 17.85 | 91.011 | 1 | 37.55 |
| 4491 | C | LEU | 244 | 40.893 | 16.551 | 91.336 | 1 | 35.84 |
| 4492 | O | LEU | 244 | 40.669 | 17.146 | 92.39 | 1 | 36.28 |
| 4493 | N | ALA | 245 | 39.926 | 16.084 | 90.555 | 1 | 35.74 |
| 4494 | CA | ALA | 245 | 38.52 | 16.254 | 90.9 | 1 | 36.61 |
| 4495 | CB | ALA | 245 | 37.634 | 15.803 | 89.739 | 1 | 35.59 |
| 4496 | C | ALA | 245 | 38.177 | 15.46 | 92.159 | 1 | 38.55 |
| 4497 | O | ALA | 245 | 37.514 | 15.971 | 93.061 | 1 | 37.18 |
| 4498 | N | LYS | 246 | 38.632 | 14.211 | 92.217 | 1 | 40.29 |
| 4499 | CA | LYS | 246 | 38.362 | 13.359 | 93.371 | 1 | 43.09 |
| 4500 | CB | LYS | 246 | 38.861 | 11.937 | 93.107 | 1 | 45.15 |
| 4501 | CG | LYS | 246 | 38.141 | 11.261 | 91.955 | 1 | 49.25 |
| 4502 | CD | LYS | 246 | 38.62 | 9.839 | 91.731 | 1 | 53.16 |
| 4503 | CE | LYS | 246 | 37.943 | 9.22 | 90.513 | 1 | 54.46 |
| 4504 | NZ | LYS | 246 | 38.453 | 7.848 | 90.227 | 1 | 57.81 |
| 4505 | C | LYS | 246 | 39.018 | 13.92 | 94.621 | 1 | 42.96 |
| 4506 | O | LYS | 246 | 38.445 | 13.883 | 95.707 | 1 | 44.05 |
| 4507 | N | LYS | 247 | 40.221 | 14.449 | 94.454 | 1 | 43.54 |
| 4508 | CA | LYS | 247 | 40.961 | 15.034 | 95.558 | 1 | 44.02 |
| 4509 | CB | LYS | 247 | 42.34 | 15.473 | 95.068 | 1 | 44.95 |
| 4510 | CG | LYS | 247 | 43.174 | 16.234 | 96.085 | 1 | 46.66 |
| 4511 | CD | LYS | 247 | 44.482 | 16.698 | 95.456 | 1 | 49.62 |
| 4512 | CE | LYS | 247 | 45.305 | 17.555 | 96.404 | 1 | 50.51 |
| 4513 | NZ | LYS | 247 | 46.576 | 18.026 | 95.768 | 1 | 50.78 |
| 4514 | C | LYS | 247 | 40.213 | 16.227 | 96.154 | 1 | 44.6 |
| 4515 | O | LYS | 247 | 40.183 | 16.399 | 97.371 | 1 | 44.73 |
| 4516 | N | HIS | 248 | 39.612 | 17.049 | 95.295 | 1 | 43.6 |
| 4517 | CA | HIS | 248 | 38.869 | 18.228 | 95.737 | 1 | 42.89 |
| 4518 | CB | HIS | 248 | 38.99 | 19.35 | 94.701 | 1 | 41.76 |
| 4519 | CG | HIS | 248 | 40.25 | 20.15 | 94.814 | 1 | 42.45 |
| 4520 | CD2 | HIS | 248 | 41.434 | 20.048 | 94.165 | 1 | 42.31 |
| 4521 | ND1 | HIS | 248 | 40.382 | 21.21 | 95.686 | 1 | 43.23 |
| 4522 | CE1 | HIS | 248 | 41.592 | 21.728 | 95.568 | 1 | 43.61 |
| 4523 | NE2 | HIS | 248 | 42.251 | 21.041 | 94.651 | 1 | 42.69 |
| 4524 | C | HIS | 248 | 37.396 | 17.913 | 95.954 | 1 | 43.32 |
| 4525 | O | HIS | 248 | 36.618 | 18.79 | 96.328 | 1 | 44.16 |
| 4526 | N | LYS | 249 | 37.017 | 16.661 | 95.72 | 1 | 43.16 |
| 4527 | CA | LYS | 249 | 35.63 | 16.238 | 95.867 | 1 | 43.35 |
| 4528 | CB | LYS | 249 | 35.169 | 16.403 | 97.317 | 1 | 46.59 |
| 4529 | CG | LYS | 249 | 34.79 | 15.096 | 98 | 1 | 50.78 |
| 4530 | CD | LYS | 249 | 35.993 | 14.175 | 98.176 | 1 | 53.98 |
| 4531 | CE | LYS | 249 | 36.915 | 14.663 | 99.287 | 1 | 56.73 |
| 4532 | NZ | LYS | 249 | 36.235 | 14.646 | 100.616 | 1 | 57.33 |
| 4533 | C | LYS | 249 | 34.725 | 17.049 | 94.938 | 1 | 42.13 |
| 4534 | O | LYS | 249 | 33.62 | 17.456 | 95.31 | 1 | 40.33 |
| 4535 | N | ARG | 250 | 35.21 | 17.292 | 93.724 | 1 | 39.33 |
| 4536 | CA | ARG | 250 | 34.452 | 18.044 | 92.732 | 1 | 37.2 |
| 4537 | CB | ARG | 250 | 35.188 | 19.338 | 92.368 | 1 | 36.51 |
| 4538 | CG | ARG | 250 | 35.427 | 20.276 | 93.536 | 1 | 36.98 |
| 4539 | CD | ARG | 250 | 34.129 | 20.896 | 94.037 | 1 | 35.57 |
| 4540 | NE | ARG | 250 | 34.357 | 21.705 | 95.23 | 1 | 35.89 |
| 4541 | CZ | ARG | 250 | 33.432 | 22.453 | 95.825 | 1 | 35.97 |
| 4542 | NH1 | ARG | 250 | 32.196 | 22.509 | 95.341 | 1 | 36.21 |
| 4543 | NH2 | ARG | 250 | 33.744 | 23.145 | 96.913 | 1 | 36.22 |
| 4544 | C | ARG | 250 | 34.298 | 17.174 | 91.491 | 1 | 36.43 |
| 4545 | O | ARG | 250 | 34.225 | 15.951 | 91.591 | 1 | 36.4 |
| 4546 | N | THR | 251 | 34.257 | 17.807 | 90.325 | 1 | 34.14 |
| 4547 | CA | THR | 251 | 34.115 | 17.081 | 89.068 | 1 | 33.98 |
| 4548 | CB | THR | 251 | 32.737 | 17.319 | 88.439 | 1 | 34.97 |
| 4549 | OG1 | THR | 251 | 32.598 | 18.71 | 88.119 | 1 | 34.88 |
| 4550 | CG2 | THR | 251 | 31.628 | 16.903 | 89.403 | 1 | 35.34 |
| 4551 | C | THR | 251 | 35.179 | 17.565 | 88.088 | 1 | 32.67 |
| 4552 | O | THR | 251 | 35.74 | 18.651 | 88.257 | 1 | 31.81 |
| 4553 | N | PRO | 252 | 35.486 | 16.758 | 87.059 | 1 | 31.54 |
| 4554 | CD | PRO | 252 | 34.979 | 15.399 | 86.796 | 1 | 31.98 |
| 4555 | CA | PRO | 252 | 36.493 | 17.143 | 86.065 | 1 | 30.44 |
| 4556 | CB | PRO | 252 | 36.431 | 16 | 85.054 | 1 | 31.24 |
| 4557 | CG | PRO | 252 | 36.054 | 14.827 | 85.901 | 1 | 33.24 |
| 4558 | C | PRO | 252 | 36.154 | 18.498 | 85.427 | 1 | 28.68 |
| 4559 | O | PRO | 252 | 37.037 | 19.32 | 85.187 | 1 | 29.75 |
| 4560 | N | ALA | 253 | 34.872 | 18.72 | 85.156 | 1 | 26.29 |
| 4561 | CA | ALA | 253 | 34.429 | 19.968 | 84.547 | 1 | 26.55 |
| 4562 | CB | ALA | 253 | 32.92 | 19.943 | 84.324 | 1 | 24.73 |
| 4563 | C | ALA | 253 | 34.805 | 21.156 | 85.421 | 1 | 25.8 |
| 4564 | O | ALA | 253 | 35.347 | 22.145 | 84.93 | 1 | 24.51 |
| 4565 | N | LEU | 254 | 34.515 | 21.059 | 86.716 | 1 | 24.42 |
| 4566 | CA | LEU | 254 | 34.835 | 22.149 | 87.634 | 1 | 25.34 |
| 4567 | CB | LEU | 254 | 34.308 | 21.849 | 89.041 | 1 | 24.94 |
| 4568 | CG | LEU | 254 | 32.8 | 22.087 | 89.177 | 1 | 27.46 |
| 4569 | CD1 | LEU | 254 | 32.312 | 21.595 | 90.536 | 1 | 28.96 |
| 4570 | CD2 | LEU | 254 | 32.509 | 23.582 | 89.014 | 1 | 27.74 |
| 4571 | C | LEU | 254 | 36.326 | 22.437 | 87.68 | 1 | 24.52 |
| 4572 | O | LEU | 254 | 36.739 | 23.599 | 87.744 | 1 | 25.76 |
| 4573 | N | ILE | 255 | 37.137 | 21.389 | 87.64 | 1 | 22.9 |
| 4574 | CA | ILE | 255 | 38.589 | 21.561 | 87.657 | 1 | 23.61 |
| 4575 | CB | ILE | 255 | 39.321 | 20.179 | 87.677 | 1 | 24.63 |
| 4576 | CG2 | ILE | 255 | 40.825 | 20.381 | 87.567 | 1 | 23.07 |
| 4577 | CG1 | ILE | 255 | 38.952 | 19.388 | 88.947 | 1 | 25.8 |
| 4578 | CD1 | ILE | 255 | 39.35 | 20.05 | 90.26 | 1 | 26.32 |
| 4579 | C | ILE | 255 | 39.022 | 22.339 | 86.396 | 1 | 23.34 |
| 4580 | O | ILE | 255 | 39.815 | 23.278 | 86.482 | 1 | 24.06 |
| 4581 | N | ALA | 256 | 38.492 | 21.946 | 85.238 | 1 | 22.84 |
| 4582 | CA | ALA | 256 | 38.823 | 22.589 | 83.966 | 1 | 23.88 |
| 4583 | CB | ALA | 256 | 38.142 | 21.857 | 82.814 | 1 | 23.31 |
| 4584 | C | ALA | 256 | 38.421 | 24.061 | 83.957 | 1 | 24.82 |
| 4585 | O | ALA | 256 | 39.137 | 24.899 | 83.418 | 1 | 25.84 |
| 4586 | N | LEU | 257 | 37.272 | 24.37 | 84.546 | 1 | 23.88 |
| 4587 | CA | LEU | 257 | 36.801 | 25.751 | 84.619 | 1 | 25.05 |
| 4588 | CB | LEU | 257 | 35.34 | 25.787 | 85.086 | 1 | 25.76 |
| 4589 | CG | LEU | 257 | 34.202 | 25.919 | 84.071 | 1 | 28.76 |
| 4590 | CD1 | LEU | 257 | 34.585 | 25.369 | 82.716 | 1 | 28.9 |
| 4591 | CD2 | LEU | 257 | 32.972 | 25.22 | 84.633 | 1 | 26.81 |
| 4592 | C | LEU | 257 | 37.661 | 26.583 | 85.572 | 1 | 23.75 |
| 4593 | O | LEU | 257 | 38.07 | 27.692 | 85.231 | 1 | 21.65 |
| 4594 | N | ARG | 258 | 37.939 | 26.043 | 86.758 | 1 | 23.02 |
| 4595 | CA | ARG | 258 | 38.741 | 26.761 | 87.758 | 1 | 21.98 |
| 4596 | CB | ARG | 258 | 38.861 | 25.939 | 89.046 | 1 | 20.7 |
| 4597 | CG | ARG | 258 | 39.553 | 26.693 | 90.182 | 1 | 20.23 |
| 4598 | CD | ARG | 258 | 38.684 | 27.855 | 90.707 | 1 | 19.44 |
| 4599 | NE | ARG | 258 | 39.496 | 28.867 | 91.388 | 1 | 19.08 |
| 4600 | CZ | ARG | 258 | 39.007 | 29.941 | 92.005 | 1 | 21.06 |
| 4601 | NH1 | ARG | 258 | 37.69 | 30.155 | 92.045 | 1 | 18.42 |
| 4602 | NH2 | ARG | 258 | 39.836 | 30.819 | 92.565 | 1 | 19.9 |
| 4603 | C | ARG | 258 | 40.133 | 27.078 | 87.227 | 1 | 21.91 |
| 4604 | O | ARG | 258 | 40.692 | 28.129 | 87.511 | 1 | 20.77 |
| 4605 | N | TYR | 259 | 40.687 | 26.144 | 86.463 | 1 | 22.99 |
| 4606 | CA | TYR | 259 | 42.001 | 26.311 | 85.862 | 1 | 22.35 |
| 4607 | CB | TYR | 259 | 42.273 | 25.126 | 84.929 | 1 | 22.8 |
| 4608 | CG | TYR | 259 | 43.46 | 25.281 | 84.012 | 1 | 24.23 |
| 4609 | CD1 | TYR | 259 | 44.762 | 25.283 | 84.513 | 1 | 25.11 |
| 4610 | CE1 | TYR | 259 | 45.859 | 25.403 | 83.658 | 1 | 26.19 |
| 4611 | CD2 | TYR | 259 | 43.28 | 25.406 | 82.628 | 1 | 24.17 |
| 4612 | CE2 | TYR | 259 | 44.368 | 25.527 | 81.769 | 1 | 25.4 |
| 4613 | CZ | TYR | 259 | 48.655 | 25.524 | 82.291 | 1 | 26.22 |
| 4614 | OH | TYR | 259 | 48.739 | 25.638 | 81.443 | 1 | 26.66 |
| 4615 | C | TYR | 259 | 42.047 | 27.627 | 85.075 | 1 | 21.54 |
| 4616 | O | TYR | 259 | 42.972 | 28.439 | 85.231 | 1 | 22.01 |
| 4617 | N | GLN | 260 | 41.046 | 27.847 | 84.231 | 1 | 20.08 |
| 4618 | CA | GLN | 260 | 41.017 | 29.064 | 83.424 | 1 | 20.29 |
| 4619 | CB | GLN | 260 | 39.872 | 29.011 | 82.405 | 1 | 19.38 |
| 4620 | CG | GLN | 260 | 39.926 | 27.795 | 81.474 | 1 | 22.3 |
| 4621 | CD | GLN | 260 | 41.077 | 27.86 | 80.478 | 1 | 22.95 |
| 4622 | OE1 | GLN | 260 | 41.954 | 28.715 | 80.584 | 1 | 20.97 |
| 4623 | NE2 | GLN | 260 | 41.075 | 26.949 | 79.507 | 1 | 22.46 |
| 4624 | C | GLN | 260 | 40.902 | 30.321 | 84.275 | 1 | 20.48 |
| 4625 | O | GLN | 260 | 41.575 | 31.318 | 84.014 | 1 | 20.19 |
| 4626 | N | LEU | 261 | 40.052 | 30.284 | 85.293 | 1 | 21.23 |
| 4627 | CA | LEU | 261 | 39.891 | 31.462 | 86.139 | 1 | 21.95 |
| 4628 | CB | LEU | 261 | 38.813 | 31.221 | 87.198 | 1 | 23.21 |
| 4629 | CG | LEU | 261 | 37.385 | 31.083 | 86.68 | 1 | 24.7 |
| 4630 | CD1 | LEU | 261 | 36.445 | 30.976 | 87.878 | 1 | 24.38 |
| 4631 | CD2 | LEU | 261 | 37.014 | 32.299 | 85.816 | 1 | 22.85 |
| 4632 | C | LEU | 261 | 41.177 | 31.889 | 86.826 | 1 | 21.33 |
| 4633 | O | LEU | 261 | 41.441 | 33.088 | 86.951 | 1 | 20.72 |
| 4634 | N | GLN | 262 | 41.986 | 30.926 | 87.265 | 1 | 21.12 |
| 4635 | CA | GLN | 262 | 43.217 | 31.279 | 87.964 | 1 | 22.21 |
| 4636 | CB | GLN | 262 | 43.696 | 30.12 | 88.848 | 1 | 22.11 |
| 4637 | CG | GLN | 262 | 42.623 | 29.722 | 89.843 | 1 | 24.36 |
| 4638 | CD | GLN | 262 | 43.101 | 28.888 | 91.017 | 1 | 22.45 |
| 4639 | OE1 | GLN | 262 | 42.28 | 28.295 | 91.715 | 1 | 23.73 |
| 4640 | NE2 | GLN | 262 | 44.411 | 28.852 | 91.257 | 1 | 22.82 |
| 4641 | C | GLN | 262 | 44.332 | 31.765 | 87.057 | 1 | 23.42 |
| 4642 | O | GLN | 262 | 45.327 | 32.315 | 87.535 | 1 | 23.79 |
| 4643 | N | ARG | 263 | 44.171 | 31.586 | 85.751 | 1 | 22.96 |
| 4644 | CA | ARG | 263 | 45.189 | 32.073 | 84.832 | 1 | 23.93 |
| 4645 | CB | ARG | 263 | 45.546 | 31.001 | 83.788 | 1 | 24.5 |
| 4646 | CG | ARG | 263 | 44.395 | 30.424 | 82.993 | 1 | 27.24 |
| 4647 | CD | ARG | 263 | 44.751 | 29.015 | 82.474 | 1 | 28.26 |
| 4648 | NE | ARG | 263 | 45.962 | 28.986 | 81.636 | 1 | 26.17 |
| 4649 | CZ | ARG | 263 | 45.957 | 28.917 | 80.305 | 1 | 25.85 |
| 4650 | NH1 | ARG | 263 | 44.808 | 28.869 | 79.637 | 1 | 23.72 |
| 4651 | NH2 | ARG | 263 | 47.101 | 28.886 | 79.638 | 1 | 24.17 |
| 4652 | C | ARG | 263 | 44.742 | 33.385 | 84.177 | 1 | 24.66 |
| 4653 | O | ARG | 263 | 45.36 | 33.86 | 83.227 | 1 | 25.78 |
| 4654 | N | GLY | 264 | 43.668 | 33.972 | 84.704 | 1 | 23.32 |
| 4655 | CA | GLY | 264 | 43.184 | 35.243 | 84.189 | 1 | 22.73 |
| 4656 | C | GLY | 264 | 42.282 | 35.172 | 82.972 | 1 | 22.41 |
| 4657 | O | GLY | 264 | 42.029 | 36.179 | 82.321 | 1 | 24.09 |
| 4658 | N | VAL | 265 | 41.781 | 33.985 | 82.675 | 1 | 22.3 |
| 4659 | CA | VAL | 265 | 40.904 | 33.792 | 81.53 | 1 | 22.02 |
| 4660 | CB | VAL | 265 | 41.228 | 32.417 | 80.856 | 1 | 21.01 |
| 4661 | CG1 | VAL | 265 | 40.17 | 32.057 | 79.812 | 1 | 21.44 |
| 4662 | CG2 | VAL | 265 | 42.612 | 32.467 | 80.209 | 1 | 20.75 |
| 4663 | C | VAL | 265 | 39.416 | 33.841 | 81.924 | 1 | 23.05 |
| 4664 | O | VAL | 265 | 39.044 | 33.357 | 82.986 | 1 | 24.84 |
| 4665 | N | VAL | 266 | 38.586 | 34.464 | 81.087 | 1 | 21.47 |
| 4666 | CA | VAL | 266 | 37.146 | 34.498 | 81.319 | 1 | 21.63 |
| 4667 | CB | VAL | 266 | 36.485 | 35.715 | 80.667 | 1 | 20.91 |
| 4668 | CG1 | VAL | 266 | 34.968 | 35.544 | 80.667 | 1 | 18.86 |
| 4669 | CG2 | VAL | 266 | 36.87 | 36.98 | 81.434 | 1 | 21.5 |
| 4670 | C | VAL | 266 | 36.679 | 33.212 | 80.636 | 1 | 22.82 |
| 4671 | O | VAL | 266 | 37.06 | 32.935 | 79.497 | 1 | 23.26 |
| 4672 | N | VAL | 267 | 35.856 | 32.426 | 81.315 | 1 | 20.87 |
| 4673 | CA | VAL | 267 | 35.459 | 31.154 | 80.755 | 1 | 21.74 |
| 4674 | CB | VAL | 267 | 36.025 | 30.014 | 81.664 | 1 | 23.39 |
| 4675 | CG1 | VAL | 267 | 35.306 | 30.023 | 83.015 | 1 | 23.09 |
| 4676 | CG2 | VAL | 267 | 35.9 | 28.659 | 80.98 | 1 | 23.79 |
| 4677 | C | VAL | 267 | 33.964 | 30.957 | 80.514 | 1 | 22.13 |
| 4678 | O | VAL | 267 | 33.121 | 31.389 | 81.307 | 1 | 21.27 |
| 4679 | N | LEU | 268 | 33.643 | 30.318 | 79.393 | 1 | 20.93 |
| 4680 | CA | LEU | 268 | 32.255 | 30.03 | 79.06 | 1 | 21.22 |
| 4681 | CB | LEU | 268 | 31.996 | 30.256 | 77.566 | 1 | 21.41 |
| 4682 | CG | LEU | 268 | 32.265 | 31.668 | 77.024 | 1 | 21.51 |
| 4683 | CD1 | LEU | 268 | 31.888 | 31.743 | 75.53 | 1 | 20.35 |
| 4684 | CD2 | LEU | 268 | 31.445 | 32.673 | 77.819 | 1 | 20.39 |
| 4685 | C | LEU | 268 | 32.001 | 28.573 | 79.394 | 1 | 21.7 |
| 4686 | O | LEU | 268 | 32.934 | 27.774 | 79.432 | 1 | 20.74 |
| 4687 | N | ALA | 269 | 30.74 | 28.241 | 79.649 | 1 | 22.55 |
| 4688 | CA | ALA | 269 | 30.338 | 26.871 | 79.939 | 1 | 22.53 |
| 4689 | CB | ALA | 269 | 30.433 | 26.565 | 81.44 | 1 | 22.36 |
| 4690 | C | ALA | 269 | 28.902 | 26.732 | 79.473 | 1 | 23.29 |
| 4691 | O | ALA | 269 | 28.016 | 27.485 | 79.893 | 1 | 22.71 |
| 4692 | N | LYS | 270 | 28.679 | 25.775 | 78.587 | 1 | 22.56 |
| 4693 | CA | LYS | 270 | 27.349 | 25.532 | 78.068 | 1 | 23.54 |
| 4694 | CB | LYS | 270 | 27.391 | 25.342 | 76.545 | 1 | 23.09 |
| 4695 | CG | LYS | 270 | 26.045 | 24.951 | 75.918 | 1 | 25.51 |
| 4696 | CD | LYS | 270 | 25.744 | 23.451 | 76.047 | 1 | 26.34 |
| 4697 | CE | LYS | 270 | 24.414 | 23.091 | 75.398 | 1 | 28.99 |
| 4698 | NZ | LYS | 270 | 24.153 | 21.613 | 75.422 | 1 | 28.53 |
| 4699 | C | LYS | 270 | 26.77 | 24.28 | 78.698 | 1 | 24.6 |
| 4700 | O | LYS | 270 | 27.462 | 23.273 | 78.831 | 1 | 24.26 |
| 4701 | N | SER | 271 | 25.505 | 24.357 | 79.091 | 1 | 25.36 |
| 4702 | CA | SER | 271 | 24.791 | 23.208 | 79.638 | 1 | 26.27 |
| 4703 | CB | SER | 271 | 25.179 | 22.926 | 81.088 | 1 | 26.63 |
| 4704 | OG | SER | 271 | 24.477 | 21.781 | 81.555 | 1 | 27.01 |
| 4705 | C | SER | 271 | 23.302 | 23.46 | 79.583 | 1 | 26.36 |
| 4706 | O | SER | 271 | 22.839 | 24.545 | 79.93 | 1 | 27.11 |
| 4707 | N | TYR | 272 | 22.548 | 22.461 | 79.137 | 1 | 27.1 |
| 4708 | CA | TYR | 272 | 21.1 | 22.587 | 79.081 | 1 | 29.21 |
| 4709 | CB | TYR | 272 | 20.571 | 22.186 | 77.701 | 1 | 29.79 |
| 4710 | CG | TYR | 272 | 20.606 | 23.3 | 76.677 | 1 | 30.47 |
| 4711 | CD1 | TYR | 272 | 21.471 | 24.384 | 76.824 | 1 | 31 |
| 4712 | CE1 | TYR | 272 | 21.521 | 25.398 | 75.871 | 1 | 30.76 |
| 4713 | CD2 | TYR | 272 | 19.79 | 23.258 | 75.545 | 1 | 31.83 |
| 4714 | CE2 | TYR | 272 | 19.835 | 24.265 | 74.587 | 1 | 31.65 |
| 4715 | CZ | TYR | 272 | 20.7 | 25.329 | 74.755 | 1 | 31.6 |
| 4716 | OH | TYR | 272 | 20.743 | 26.324 | 73.807 | 1 | 33.62 |
| 4717 | C | TYR | 272 | 20.481 | 21.711 | 80.157 | 1 | 30.11 |
| 4718 | O | TYR | 272 | 19.267 | 21.529 | 80.194 | 1 | 30.36 |
| 4719 | N | ASN | 273 | 21.326 | 21.184 | 81.039 | 1 | 31.22 |
| 4720 | CA | ASN | 273 | 20.878 | 20.32 | 82.135 | 1 | 32.4 |
| 4721 | CB | ASN | 273 | 21.786 | 19.092 | 82.217 | 1 | 32.56 |
| 4722 | CG | ASN | 273 | 21.39 | 18.143 | 83.331 | 1 | 35.28 |
| 4723 | OD1 | ASN | 273 | 21.717 | 18.363 | 84.497 | 1 | 33.94 |
| 4724 | ND2 | ASN | 273 | 20.665 | 17.083 | 82.975 | 1 | 37.04 |
| 4725 | C | ASN | 273 | 20.898 | 21.078 | 83.463 | 1 | 33.03 |
| 4726 | O | ASN | 273 | 21.938 | 21.594 | 83.869 | 1 | 32.74 |
| 4727 | N | GLU | 274 | 19.757 | 21.128 | 84.146 | 1 | 33.81 |
| 4728 | CA | GLU | 274 | 19.646 | 21.854 | 85.411 | 1 | 36.55 |
| 4729 | CB | GLU | 274 | 18.267 | 21.626 | 86.035 | 1 | 38.89 |
| 4730 | CG | GLU | 274 | 17.872 | 22.716 | 87.021 | 1 | 45.92 |
| 4731 | CD | GLU | 274 | 16.504 | 22.491 | 87.644 | 1 | 48.46 |
| 4732 | OE1 | GLU | 274 | 15.567 | 22.111 | 86.908 | 1 | 51.69 |
| 4733 | OE2 | GLU | 274 | 16.366 | 22.712 | 88.865 | 1 | 49.91 |
| 4734 | C | GLU | 274 | 20.725 | 21.51 | 86.437 | 1 | 36.28 |
| 4735 | O | GLU | 274 | 21.33 | 22.402 | 87.034 | 1 | 36.03 |
| 4736 | N | GLN | 275 | 20.958 | 20.219 | 86.639 | 1 | 36.36 |
| 4737 | CA | GLN | 275 | 21.962 | 19.76 | 87.591 | 1 | 36.95 |
| 4738 | CB | GLN | 275 | 22.025 | 18.226 | 87.591 | 1 | 40.69 |
| 4739 | CG | GLN | 275 | 23.169 | 17.644 | 88.423 | 1 | 47.46 |
| 4740 | CD | GLN | 275 | 23.491 | 16.198 | 88.054 | 1 | 51.47 |
| 4741 | OE1 | GLN | 275 | 23.782 | 15.888 | 86.891 | 1 | 55.24 |
| 4742 | NE2 | GLN | 275 | 23.451 | 15.309 | 89.042 | 1 | 52.45 |
| 4743 | C | GLN | 275 | 23.343 | 20.318 | 87.27 | 1 | 34.5 |
| 4744 | O | GLN | 275 | 24.019 | 20.873 | 88.139 | 1 | 34.08 |
| 4745 | N | ARG | 276 | 23.763 | 20.168 | 86.021 | 1 | 32.2 |
| 4746 | CA | ARG | 276 | 25.076 | 20.643 | 85.617 | 1 | 31.19 |
| 4747 | CB | ARG | 276 | 25.45 | 20.041 | 84.257 | 1 | 31.11 |
| 4748 | CG | ARG | 276 | 25.631 | 18.521 | 84.334 | 1 | 30.48 |
| 4749 | CD | ARG | 276 | 26.147 | 17.922 | 83.037 | 1 | 32.58 |
| 4750 | NE | ARG | 276 | 25.103 | 17.716 | 82.041 | 1 | 35.04 |
| 4751 | CZ | ARG | 276 | 24.264 | 16.685 | 82.034 | 1 | 37.39 |
| 4752 | NH1 | ARG | 276 | 24.349 | 15.756 | 82.978 | 1 | 37.43 |
| 4753 | NH2 | ARG | 276 | 23.346 | 16.58 | 81.079 | 1 | 37.84 |
| 4754 | C | ARG | 276 | 25.186 | 22.168 | 85.608 | 1 | 30.03 |
| 4755 | O | ARG | 276 | 26.242 | 22.717 | 85.931 | 1 | 29.73 |
| 4756 | N | ILE | 277 | 24.1 | 22.852 | 85.259 | 1 | 28.62 |
| 4757 | CA | ILE | 277 | 24.101 | 24.31 | 85.252 | 1 | 28.09 |
| 4758 | CB | ILE | 277 | 22.73 | 24.869 | 84.8 | 1 | 27 |
| 4759 | CG2 | ILE | 277 | 22.629 | 26.353 | 85.148 | 1 | 25.34 |
| 4760 | CG1 | ILE | 277 | 22.549 | 24.643 | 83.296 | 1 | 26.24 |
| 4761 | CD1 | ILE | 277 | 21.149 | 24.952 | 82.779 | 1 | 25.36 |
| 4762 | C | ILE | 277 | 24.405 | 24.808 | 86.666 | 1 | 29.89 |
| 4763 | O | ILE | 277 | 25.254 | 25.679 | 86.867 | 1 | 28.89 |
| 4764 | N | ARG | 278 | 23.706 | 24.238 | 87.642 | 1 | 30.34 |
| 4765 | CA | ARG | 278 | 23.886 | 24.596 | 89.047 | 1 | 32.08 |
| 4766 | CB | ARG | 278 | 22.845 | 23.879 | 89.909 | 1 | 35.91 |
| 4767 | CG | ARG | 278 | 21.436 | 24.441 | 89.8 | 1 | 42.12 |
| 4768 | CD | ARG | 278 | 21.277 | 25.672 | 90.68 | 1 | 48.14 |
| 4769 | NE | ARG | 278 | 19.927 | 26.232 | 90.608 | 1 | 52.74 |
| 4770 | CZ | ARG | 278 | 19.504 | 27.255 | 91.345 | 1 | 54.64 |
| 4771 | NH1 | ARG | 278 | 20.327 | 27.83 | 92.217 | 1 | 56.37 |
| 4772 | NH2 | ARG | 278 | 18.265 | 27.706 | 91.206 | 1 | 55.07 |
| 4773 | C | ARG | 278 | 25.282 | 24.223 | 89.528 | 1 | 30.73 |
| 4774 | O | ARG | 278 | 25.881 | 24.934 | 90.328 | 1 | 30.53 |
| 4775 | N | GLN | 279 | 25.795 | 23.103 | 89.03 | 1 | 29.46 |
| 4776 | CA | GLN | 279 | 27.118 | 22.633 | 89.405 | 1 | 28.96 |
| 4777 | CB | GLN | 279 | 27.341 | 21.219 | 88.871 | 1 | 30.93 |
| 4778 | CG | GLN | 279 | 28.733 | 20.665 | 89.13 | 1 | 33.53 |
| 4779 | CD | GLN | 279 | 29.001 | 19.399 | 88.345 | 1 | 35.97 |
| 4780 | OE1 | GLN | 279 | 28.259 | 18.42 | 88.452 | 1 | 39.87 |
| 4781 | NE2 | GLN | 279 | 30.06 | 19.412 | 87.543 | 1 | 32.87 |
| 4782 | C | GLN | 279 | 28.251 | 23.532 | 88.916 | 1 | 28.17 |
| 4783 | O | GLN | 279 | 29.224 | 23.752 | 89.636 | 1 | 27.5 |
| 4784 | N | ASN | 280 | 28.132 | 24.051 | 87.697 | 1 | 27.84 |
| 4785 | CA | ASN | 280 | 29.193 | 24.892 | 87.132 | 1 | 27.28 |
| 4786 | CB | ASN | 280 | 28.913 | 25.183 | 85.648 | 1 | 26.48 |
| 4787 | CG | ASN | 280 | 29.011 | 23.93 | 84.781 | 1 | 28.56 |
| 4788 | OD1 | ASN | 280 | 29.645 | 22.947 | 85.168 | 1 | 26.68 |
| 4789 | ND2 | ASN | 280 | 28.395 | 23.965 | 83.597 | 1 | 27.83 |
| 4790 | C | ASN | 280 | 29.467 | 26.191 | 87.89 | 1 | 26.15 |
| 4791 | O | ASN | 280 | 30.605 | 26.664 | 87.909 | 1 | 26.27 |
| 4792 | N | VAL | 281 | 28.448 | 26.765 | 88.526 | 1 | 27.49 |
| 4793 | CA | VAL | 281 | 28.661 | 27.999 | 89.282 | 1 | 27.67 |
| 4794 | CB | VAL | 281 | 27.321 | 28.695 | 89.67 | 1 | 28.69 |
| 4795 | CG1 | VAL | 281 | 26.57 | 29.113 | 88.422 | 1 | 29.15 |
| 4796 | CG2 | VAL | 281 | 26.466 | 27.758 | 90.51 | 1 | 30.98 |
| 4797 | C | VAL | 281 | 29.47 | 27.746 | 90.553 | 1 | 26.86 |
| 4798 | O | VAL | 281 | 29.916 | 28.683 | 91.202 | 1 | 27.07 |
| 4799 | N | GLN | 282 | 29.659 | 26.482 | 90.912 | 1 | 27.07 |
| 4800 | CA | GLN | 282 | 30.432 | 26.158 | 92.107 | 1 | 27.85 |
| 4801 | CB | GLN | 282 | 30.16 | 24.717 | 92.537 | 1 | 30.9 |
| 4802 | CG | GLN | 282 | 28.74 | 24.495 | 93.019 | 1 | 35.25 |
| 4803 | CD | GLN | 282 | 28.467 | 23.049 | 93.373 | 1 | 40.44 |
| 4804 | OE1 | GLN | 282 | 27.341 | 22.689 | 93.715 | 1 | 44.4 |
| 4805 | NE2 | GLN | 282 | 29.497 | 22.208 | 93.288 | 1 | 42.64 |
| 4806 | C | GLN | 282 | 31.935 | 26.361 | 91.881 | 1 | 28.17 |
| 4807 | O | GLN | 282 | 32.753 | 26.136 | 92.781 | 1 | 27.57 |
| 4808 | N | VAL | 283 | 32.296 | 26.799 | 90.679 | 1 | 24.86 |
| 4809 | CA | VAL | 283 | 33.693 | 27.037 | 90.358 | 1 | 23.9 |
| 4810 | CB | VAL | 283 | 33.851 | 27.492 | 88.883 | 1 | 22.86 |
| 4811 | CG1 | VAL | 283 | 33.185 | 28.846 | 88.68 | 1 | 23.2 |
| 4812 | CG2 | VAL | 283 | 35.324 | 27.549 | 88.513 | 1 | 24.13 |
| 4813 | C | VAL | 283 | 34.264 | 28.103 | 91.299 | 1 | 23.81 |
| 4814 | O | VAL | 283 | 35.47 | 28.18 | 91.504 | 1 | 23.23 |
| 4815 | N | PHE | 284 | 33.391 | 28.917 | 91.887 | 1 | 22.44 |
| 4816 | CA | PHE | 284 | 33.847 | 29.963 | 92.801 | 1 | 24.08 |
| 4817 | CB | PHE | 284 | 32.863 | 31.136 | 92.781 | 1 | 24.64 |
| 4818 | CG | PHE | 284 | 32.73 | 31.791 | 91.435 | 1 | 22.68 |
| 4819 | CD1 | PHE | 284 | 33.793 | 32.5 | 90.883 | 1 | 23.3 |
| 4820 | CD2 | PHE | 284 | 31.546 | 31.692 | 90.718 | 1 | 22.4 |
| 4821 | CE1 | PHE | 284 | 33.677 | 33.102 | 89.628 | 1 | 24.58 |
| 4822 | CE2 | PHE | 284 | 31.419 | 32.288 | 89.464 | 1 | 23.13 |
| 4823 | CZ | PHE | 284 | 32.487 | 32.995 | 88.92 | 1 | 21.07 |
| 4824 | C | PHE | 284 | 33.999 | 29.467 | 94.242 | 1 | 25.51 |
| 4825 | O | PHE | 284 | 34.422 | 30.22 | 95.122 | 1 | 25.17 |
| 4826 | N | GLU | 285 | 33.667 | 28.203 | 94.476 | 1 | 25.57 |
| 4827 | CA | GLU | 285 | 33.724 | 27.642 | 95.823 | 1 | 29.39 |
| 4828 | CB | GLU | 285 | 32.573 | 26.653 | 96.015 | 1 | 31.89 |
| 4829 | CG | GLU | 285 | 31.234 | 27.321 | 96.252 | 1 | 39.46 |
| 4830 | CD | GLU | 285 | 30.09 | 26.33 | 96.255 | 1 | 44.77 |
| 4831 | OE1 | GLU | 285 | 30.288 | 25.186 | 96.737 | 1 | 47.95 |
| 4832 | OE2 | GLU | 285 | 28.987 | 26.7 | 95.787 | 1 | 49.57 |
| 4833 | C | GLU | 285 | 35.027 | 26.983 | 96.246 | 1 | 28.31 |
| 4834 | O | GLU | 285 | 35.16 | 26.561 | 97.394 | 1 | 27.4 |
| 4835 | N | PHE | 286 | 35.989 | 26.889 | 95.338 | 1 | 25.7 |
| 4836 | CA | PHE | 286 | 37.254 | 26.277 | 95.696 | 1 | 25.13 |
| 4837 | CB | PHE | 286 | 37.214 | 24.758 | 95.464 | 1 | 25.68 |
| 4838 | CG | PHE | 286 | 37.153 | 24.358 | 94.015 | 1 | 25.61 |
| 4839 | CD1 | PHE | 286 | 35.969 | 24.47 | 93.29 | 1 | 24.99 |
| 4840 | CD2 | PHE | 286 | 38.29 | 23.876 | 93.369 | 1 | 26.91 |
| 4841 | CE1 | PHE | 286 | 35.917 | 24.111 | 91.943 | 1 | 25.93 |
| 4842 | CE2 | PHE | 286 | 38.248 | 23.515 | 92.02 | 1 | 27.1 |
| 4843 | CZ | PHE | 286 | 37.06 | 23.634 | 91.308 | 1 | 26.05 |
| 4844 | C | PHE | 286 | 38.384 | 26.903 | 94.897 | 1 | 26.27 |
| 4845 | O | PHE | 286 | 38.144 | 27.707 | 93.994 | 1 | 24.93 |
| 4846 | N | GLN | 287 | 39.615 | 26.533 | 95.238 | 1 | 25.23 |
| 4847 | CA | GLN | 287 | 40.791 | 27.061 | 94.563 | 1 | 27.17 |
| 4848 | CB | GLN | 287 | 41.435 | 28.157 | 95.412 | 1 | 27.41 |
| 4849 | CG | GLN | 287 | 40.492 | 29.293 | 95.778 | 1 | 27.78 |
| 4850 | CD | GLN | 287 | 41.174 | 30.349 | 96.61 | 1 | 29.71 |
| 4851 | OE1 | GLN | 287 | 42.214 | 30.877 | 96.22 | 1 | 30.3 |
| 4852 | NE2 | GLN | 287 | 40.597 | 30.666 | 97.766 | 1 | 30.43 |
| 4853 | C | GLN | 287 | 41.802 | 25.953 | 94.322 | 1 | 26.22 |
| 4854 | O | GLN | 287 | 41.761 | 24.925 | 94.99 | 1 | 26.9 |
| 4855 | N | LEU | 288 | 42.703 | 26.169 | 93.364 | 1 | 25.77 |
| 4856 | CA | LEU | 288 | 43.747 | 25.191 | 93.036 | 1 | 24.94 |
| 4857 | CB | LEU | 288 | 43.761 | 24.916 | 91.523 | 1 | 26.15 |
| 4858 | CG | LEU | 288 | 42.495 | 24.293 | 90.905 | 1 | 27.46 |
| 4859 | CD1 | LEU | 288 | 42.624 | 24.269 | 89.378 | 1 | 27.78 |
| 4860 | CD2 | LEU | 288 | 42.286 | 22.889 | 91.447 | 1 | 27.31 |
| 4861 | C | LEU | 288 | 45.104 | 25.744 | 93.469 | 1 | 23.73 |
| 4862 | O | LEU | 288 | 45.341 | 26.942 | 93.374 | 1 | 22.56 |
| 4863 | N | THR | 289 | 45.987 | 24.875 | 93.954 | 1 | 24.64 |
| 4864 | CA | THR | 289 | 47.319 | 25.304 | 94.383 | 1 | 25.36 |
| 4865 | CB | THR | 289 | 47.948 | 24.281 | 95.341 | 1 | 25.61 |
| 4866 | OG1 | THR | 289 | 48.143 | 23.04 | 94.649 | 1 | 25.09 |
| 4867 | CG2 | THR | 289 | 47.024 | 24.055 | 96.546 | 1 | 22.71 |
| 4868 | C | THR | 289 | 48.244 | 25.472 | 93.177 | 1 | 26.62 |
| 4869 | O | THR | 289 | 47.901 | 25.064 | 92.064 | 1 | 25.36 |
| 4870 | N | SER | 290 | 49.414 | 26.071 | 93.398 | 1 | 26.88 |
| 4871 | CA | SER | 290 | 50.364 | 26.267 | 92.311 | 1 | 29.96 |
| 4872 | CB | SER | 290 | 51.62 | 26.999 | 92.811 | 1 | 29.73 |
| 4873 | OG | SER | 290 | 52.346 | 26.212 | 93.745 | 1 | 32.26 |
| 4874 | C | SER | 290 | 50.751 | 24.918 | 91.69 | 1 | 30.98 |
| 4875 | O | SER | 290 | 50.92 | 24.818 | 90.476 | 1 | 31.76 |
| 4876 | N | GLU | 291 | 50.871 | 23.884 | 92.52 | 1 | 32.52 |
| 4877 | CA | GLU | 291 | 51.237 | 22.554 | 92.037 | 1 | 33.88 |
| 4878 | CB | GLU | 291 | 51.547 | 21.619 | 93.212 | 1 | 38.55 |
| 4879 | CG | GLU | 291 | 52.874 | 21.907 | 93.889 | 1 | 45.94 |
| 4880 | CD | GLU | 291 | 53.057 | 21.129 | 95.176 | 1 | 50.9 |
| 4881 | OE1 | GLU | 291 | 52.907 | 19.884 | 95.147 | 1 | 54.99 |
| 4882 | OE2 | GLU | 291 | 53.354 | 21.764 | 96.216 | 1 | 53.85 |
| 4883 | C | GLU | 291 | 50.156 | 21.932 | 91.17 | 1 | 33.08 |
| 4884 | O | GLU | 291 | 50.446 | 21.261 | 90.174 | 1 | 31.43 |
| 4885 | N | GLU | 292 | 48.903 | 22.145 | 91.543 | 1 | 30.81 |
| 4886 | CA | GLU | 292 | 47.812 | 21.589 | 90.76 | 1 | 30.36 |
| 4887 | CB | GLU | 292 | 46.506 | 21.669 | 91.553 | 1 | 30.62 |
| 4888 | CG | GLU | 292 | 46.6 | 20.987 | 92.913 | 1 | 31.48 |
| 4889 | CD | GLU | 292 | 45.324 | 21.103 | 93.725 | 1 | 31.8 |
| 4890 | OE1 | GLU | 292 | 44.779 | 22.219 | 93.816 | 1 | 31.91 |
| 4891 | OE2 | GLU | 292 | 44.874 | 20.084 | 94.283 | 1 | 32.52 |
| 4892 | C | GLU | 292 | 47.703 | 22.341 | 89.43 | 1 | 29.31 |
| 4893 | O | GLU | 292 | 47.383 | 21.748 | 88.401 | 1 | 29.71 |
| 4894 | N | MET | 293 | 47.98 | 23.641 | 89.451 | 1 | 28.6 |
| 4895 | CA | MET | 293 | 47.929 | 24.44 | 88.231 | 1 | 29.31 |
| 4896 | CB | MET | 293 | 48.135 | 25.927 | 88.545 | 1 | 28.13 |
| 4897 | CG | MET | 293 | 46.909 | 26.626 | 89.168 | 1 | 24.99 |
| 4898 | SD | MET | 293 | 45.459 | 26.71 | 88.062 | 1 | 24.87 |
| 4899 | CE | MET | 293 | 46.03 | 27.955 | 86.905 | 1 | 21.91 |
| 4900 | C | MET | 293 | 49.023 | 23.952 | 87.274 | 1 | 31.81 |
| 4901 | O | MET | 293 | 48.787 | 23.823 | 86.068 | 1 | 30.75 |
| 4902 | N | LYS | 294 | 50.21 | 23.674 | 87.812 | 1 | 32.56 |
| 4903 | CA | LYS | 294 | 51.323 | 23.185 | 86.995 | 1 | 35.27 |
| 4904 | CB | LYS | 294 | 52.593 | 23.014 | 87.837 | 1 | 38.67 |
| 4905 | CG | LYS | 294 | 53.627 | 24.11 | 87.651 | 1 | 44.7 |
| 4906 | CD | LYS | 294 | 53.13 | 25.447 | 88.174 | 1 | 49.7 |
| 4907 | CE | LYS | 294 | 54.232 | 26.508 | 88.12 | 1 | 52.73 |
| 4908 | NZ | LYS | 294 | 54.689 | 26.815 | 86.725 | 1 | 54.6 |
| 4909 | C | LYS | 294 | 50.978 | 21.852 | 85.356 | 1 | 33.58 |
| 4910 | O | LYS | 294 | 51.315 | 21.604 | 85.201 | 1 | 33.97 |
| 4911 | N | ALA | 295 | 50.308 | 20.993 | 87.116 | 1 | 32.23 |
| 4912 | CA | ALA | 295 | 49.921 | 19.686 | 86.62 | 1 | 31.87 |
| 4913 | CB | ALA | 295 | 49.215 | 18.892 | 87.716 | 1 | 31.59 |
| 4914 | C | ALA | 295 | 49.004 | 19.833 | 85.411 | 1 | 32.06 |
| 4915 | O | ALA | 295 | 49.146 | 19.111 | 84.424 | 1 | 31.65 |
| 4916 | N | ILE | 296 | 48.067 | 20.773 | 85.485 | 1 | 30.84 |
| 4917 | CA | ILE | 296 | 47.132 | 20.974 | 84.386 | 1 | 29.42 |
| 4918 | CB | ILE | 296 | 45.933 | 21.843 | 84.826 | 1 | 28.15 |
| 4919 | CG2 | ILE | 296 | 44.935 | 21.981 | 83.675 | 1 | 26.2 |
| 4920 | CG1 | ILE | 296 | 45.245 | 21.179 | 86.019 | 1 | 26.62 |
| 4921 | CD1 | ILE | 296 | 44.21 | 22.059 | 86.722 | 1 | 27.4 |
| 4922 | C | ILE | 296 | 47.816 | 21.58 | 83.163 | 1 | 30.13 |
| 4923 | O | ILE | 296 | 47.461 | 21.245 | 82.033 | 1 | 30.65 |
| 4924 | N | ASP | 297 | 48.79 | 22.461 | 83.387 | 1 | 29.89 |
| 4925 | CA | ASP | 297 | 49.539 | 23.063 | 82.291 | 1 | 32.57 |
| 4926 | CB | ASP | 297 | 50.595 | 24.05 | 82.798 | 1 | 34.19 |
| 4927 | CG | ASP | 297 | 50.013 | 25.38 | 83.218 | 1 | 36.04 |
| 4928 | OD1 | ASP | 297 | 48.93 | 25.756 | 82.727 | 1 | 37.57 |
| 4929 | OD2 | ASP | 297 | 50.664 | 26.062 | 84.034 | 1 | 37.05 |
| 4930 | C | ASP | 297 | 50.271 | 21.981 | 81.502 | 1 | 33.26 |
| 4931 | O | ASP | 297 | 50.524 | 22.146 | 80.315 | 1 | 33.27 |
| 4932 | N | GLY | 298 | 50.62 | 20.886 | 82.172 | 1 | 33.01 |
| 4933 | CA | GLY | 298 | 51.332 | 19.805 | 81.511 | 1 | 34.38 |
| 4934 | C | GLY | 298 | 50.487 | 18.934 | 80.599 | 1 | 35.29 |
| 4935 | O | GLY | 298 | 51.021 | 18.081 | 79.886 | 1 | 36.21 |
| 4936 | N | LEU | 299 | 49.175 | 19.145 | 80.603 | 1 | 34.6 |
| 4937 | CA | LEU | 299 | 48.275 | 18.354 | 79.767 | 1 | 34.47 |
| 4938 | CB | LEU | 299 | 46.869 | 18.333 | 80.371 | 1 | 34.16 |
| 4939 | CG | LEU | 299 | 46.734 | 17.714 | 81.765 | 1 | 35.32 |
| 4940 | CD1 | LEU | 299 | 45.289 | 17.803 | 82.229 | 1 | 32.85 |
| 4941 | CD2 | LEU | 299 | 47.193 | 16.259 | 81.721 | 1 | 35.04 |
| 4942 | C | LEU | 299 | 48.188 | 18.874 | 78.338 | 1 | 34.82 |
| 4943 | O | LEU | 299 | 47.619 | 18.218 | 77.466 | 1 | 34.16 |
| 4944 | N | ASN | 300 | 48.751 | 20.051 | 78.099 | 1 | 34.57 |
| 4945 | CA | ASN | 300 | 48.708 | 20.644 | 76.772 | 1 | 36.54 |
| 4946 | CB | ASN | 300 | 49.519 | 21.939 | 76.744 | 1 | 35.32 |
| 4947 | CG | ASN | 300 | 49.295 | 22.73 | 75.471 | 1 | 36.34 |
| 4948 | OD1 | ASN | 300 | 48.154 | 23.019 | 75.103 | 1 | 34.17 |
| 4949 | ND2 | ASN | 300 | 50.381 | 23.094 | 74.796 | 1 | 35.9 |
| 4950 | C | ASN | 300 | 49.232 | 19.686 | 75.708 | 1 | 38.58 |
| 4951 | O | ASN | 300 | 50.356 | 19.193 | 75.799 | 1 | 38.3 |
| 4952 | N | ARG | 301 | 48.418 | 19.431 | 74.692 | 1 | 40.03 |
| 4953 | CA | ARG | 301 | 48.818 | 18.522 | 73.628 | 1 | 42.19 |
| 4954 | CB | ARG | 301 | 48.187 | 17.146 | 73.864 | 1 | 43.22 |
| 4955 | CG | ARG | 301 | 46.685 | 17.187 | 74.009 | 1 | 45.6 |
| 4956 | CD | ARG | 301 | 46.086 | 15.795 | 74.05 | 1 | 48.02 |
| 4957 | NE | ARG | 301 | 46.388 | 15.087 | 75.288 | 1 | 51.11 |
| 4958 | CZ | ARG | 301 | 46.014 | 13.835 | 75.54 | 1 | 52.67 |
| 4959 | NH1 | ARG | 301 | 45.323 | 13.148 | 74.636 | 1 | 53.22 |
| 4960 | NH2 | ARG | 301 | 46.327 | 13.272 | 76.697 | 1 | 52.78 |
| 4961 | C | ARG | 301 | 48.428 | 19.053 | 72.255 | 1 | 42.63 |
| 4962 | O | ARG | 301 | 48.392 | 18.306 | 71.278 | 1 | 42.73 |
| 4963 | N | ASN | 302 | 48.137 | 20.347 | 72.194 | 1 | 42.33 |
| 4964 | CA | ASN | 302 | 47.755 | 21.001 | 70.949 | 1 | 43.77 |
| 4965 | CB | ASN | 302 | 48.964 | 21.121 | 70.019 | 1 | 46.96 |
| 4966 | CG | ASN | 302 | 49.916 | 22.223 | 70.447 | 1 | 50.49 |
| 4967 | OD1 | ASN | 302 | 50.467 | 22.193 | 71.547 | 1 | 52.26 |
| 4968 | ND2 | ASN | 302 | 50.112 | 23.208 | 69.574 | 1 | 52.86 |
| 4969 | C | ASN | 302 | 46.614 | 20.321 | 70.211 | 1 | 41.81 |
| 4970 | O | ASN | 302 | 46.722 | 20.03 | 69.023 | 1 | 42.49 |
| 4971 | N | VAL | 303 | 45.523 | 20.069 | 70.922 | 1 | 40.06 |
| 4972 | CA | VAL | 303 | 44.349 | 19.447 | 70.327 | 1 | 38.45 |
| 4973 | CB | VAL | 303 | 43.776 | 18.329 | 71.229 | 1 | 38.96 |
| 4974 | CG1 | VAL | 303 | 42.399 | 17.904 | 70.734 | 1 | 38.55 |
| 4975 | CG2 | VAL | 303 | 44.716 | 17.133 | 71.229 | 1 | 39.24 |
| 4976 | C | VAL | 303 | 43.271 | 20.511 | 70.123 | 1 | 37.41 |
| 4977 | O | VAL | 303 | 42.66 | 20.987 | 71.085 | 1 | 36.9 |
| 4978 | N | ARG | 304 | 43.046 | 20.879 | 68.867 | 1 | 35.2 |
| 4979 | CA | ARG | 304 | 42.042 | 21.878 | 68.524 | 1 | 32.11 |
| 4980 | CB | ARG | 304 | 42.571 | 22.781 | 67.4 | 1 | 32.3 |
| 4981 | CG | ARG | 304 | 41.675 | 23.962 | 67.063 | 1 | 30.26 |
| 4982 | CD | ARG | 304 | 42.164 | 24.706 | 65.825 | 1 | 28.43 |
| 4983 | NE | ARG | 304 | 43.37 | 25.511 | 66.014 | 1 | 26.71 |
| 4984 | CZ | ARG | 304 | 43.421 | 26.658 | 66.688 | 1 | 28.86 |
| 4985 | NH1 | ARG | 304 | 42.325 | 27.151 | 67.262 | 1 | 27.75 |
| 4986 | NH2 | ARG | 304 | 44.563 | 27.331 | 66.759 | 1 | 26.89 |
| 4987 | C | ARG | 304 | 40.795 | 21.128 | 68.067 | 1 | 31.89 |
| 4988 | O | ARG | 304 | 40.83 | 20.406 | 67.073 | 1 | 31.75 |
| 4989 | N | TYR | 305 | 39.701 | 21.275 | 68.806 | 1 | 29.48 |
| 4990 | CA | TYR | 305 | 38.454 | 20.597 | 68.455 | 1 | 30.06 |
| 4991 | CB | TYR | 305 | 37.511 | 20.533 | 69.661 | 1 | 29.82 |
| 4992 | CG | TYR | 305 | 38.005 | 19.656 | 70.789 | 1 | 32.23 |
| 4993 | CD1 | TYR | 305 | 38.144 | 18.275 | 70.623 | 1 | 32.6 |
| 4994 | CE1 | TYR | 305 | 38.617 | 17.469 | 71.66 | 1 | 34.66 |
| 4995 | CD2 | TYR | 305 | 38.349 | 20.209 | 72.021 | 1 | 32.53 |
| 4996 | CE2 | TYR | 305 | 38.821 | 19.416 | 73.063 | 1 | 33.64 |
| 4997 | CZ | TYR | 305 | 38.954 | 18.05 | 72.877 | 1 | 34.42 |
| 4998 | OH | TYR | 305 | 39.43 | 17.272 | 73.905 | 1 | 36.03 |
| 4999 | C | TYR | 305 | 37.716 | 21.261 | 67.294 | 1 | 29.99 |
| 5000 | O | TYR | 305 | 37.137 | 20.577 | 66.453 | 1 | 29.95 |
| 5001 | N | LEU | 306 | 37.721 | 22.59 | 67.26 | 1 | 29.06 |
| 5002 | CA | LEU | 306 | 37.039 | 23.324 | 66.198 | 1 | 29.57 |
| 5003 | CB | LEU | 306 | 36.143 | 24.412 | 66.794 | 1 | 31.17 |
| 5004 | CG | LEU | 306 | 34.832 | 23.902 | 67.379 | 1 | 35.1 |
| 5005 | CD1 | LEU | 306 | 34.23 | 24.953 | 68.278 | 1 | 36.46 |
| 5006 | CD2 | LEU | 306 | 33.872 | 23.544 | 66.246 | 1 | 35.02 |
| 5007 | C | LEU | 306 | 38.018 | 23.955 | 65.225 | 1 | 28.82 |
| 5008 | O | LEU | 306 | 38.512 | 25.056 | 65.458 | 1 | 28.22 |
| 5009 | N | THR | 307 | 38.294 | 23.248 | 64.134 | 1 | 28.38 |
| 5010 | CA | THR | 307 | 39.219 | 23.737 | 63.122 | 1 | 29.36 |
| 5011 | CB | THR | 307 | 39.927 | 22.568 | 62.409 | 1 | 31.79 |
| 5012 | OG1 | THR | 307 | 38.942 | 21.752 | 61.763 | 1 | 33.76 |
| 5013 | CG2 | THR | 307 | 40.713 | 21.722 | 63.409 | 1 | 33.07 |
| 5014 | C | THR | 307 | 38.488 | 24.56 | 62.068 | 1 | 29.69 |
| 5015 | O | THR | 307 | 39.082 | 25.423 | 61.421 | 1 | 30.12 |
| 5016 | N | LEU | 308 | 37.2 | 24.286 | 61.889 | 1 | 30.15 |
| 5017 | CA | LEU | 308 | 36.406 | 25.01 | 60.9 | 1 | 31.95 |
| 5018 | CB | LEU | 308 | 36.16 | 26.452 | 61.374 | 1 | 33.81 |
| 5019 | CG | LEU | 308 | 35.248 | 26.604 | 62.606 | 1 | 35.34 |
| 5020 | CD1 | LEU | 308 | 35.35 | 28.004 | 63.175 | 1 | 36.3 |
| 5021 | CD2 | LEU | 308 | 33.816 | 26.282 | 62.205 | 1 | 35.3 |
| 5022 | C | LEU | 308 | 37.143 | 25.035 | 59.562 | 1 | 31.91 |
| 5023 | O | LEU | 308 | 37.14 | 26.052 | 58.868 | 1 | 30.16 |
| 5024 | N | ASP | 309 | 37.759 | 23.91 | 59.205 | 1 | 33.18 |
| 5025 | CA | ASP | 309 | 38.537 | 23.804 | 57.971 | 1 | 34.02 |
| 5026 | CB | ASP | 309 | 39.256 | 22.455 | 57.915 | 1 | 37.49 |
| 5027 | CG | ASP | 309 | 38.383 | 21.323 | 58.372 | 1 | 40 |
| 5028 | OD1 | ASP | 309 | 37.235 | 21.222 | 57.889 | 1 | 42.35 |
| 5029 | OD2 | ASP | 309 | 38.845 | 20.528 | 59.218 | 1 | 46.49 |
| 5030 | C | ASP | 309 | 37.75 | 24.022 | 56.685 | 1 | 34.37 |
| 5031 | O | ASP | 309 | 38.343 | 24.13 | 55.618 | 1 | 33.61 |
| 5032 | N | ILE | 310 | 36.424 | 24.09 | 56.773 | 1 | 33.33 |
| 5033 | CA | ILE | 310 | 35.634 | 24.335 | 55.576 | 1 | 33.67 |
| 5034 | CB | ILE | 310 | 34.132 | 24.112 | 55.812 | 1 | 35.32 |
| 5035 | CG2 | ILE | 310 | 33.863 | 22.644 | 56.1 | 1 | 36.47 |
| 5036 | CG1 | ILE | 310 | 33.648 | 24.997 | 56.96 | 1 | 37.09 |
| 5037 | CD1 | ILE | 310 | 32.143 | 25.055 | 57.086 | 1 | 39.69 |
| 5038 | C | ILE | 310 | 35.852 | 25.788 | 55.148 | 1 | 33.45 |
| 5039 | O | ILE | 310 | 35.505 | 26.174 | 54.031 | 1 | 32.28 |
| 5040 | N | PHE | 311 | 36.429 | 26.59 | 56.044 | 1 | 30.63 |
| 5041 | CA | PHE | 311 | 36.714 | 27.988 | 55.754 | 1 | 28.67 |
| 5042 | CB | PHE | 311 | 36.357 | 28.869 | 56.954 | 1 | 31.17 |
| 5043 | CG | PHE | 311 | 34.881 | 28.975 | 57.205 | 1 | 32.87 |
| 5044 | CD1 | PHE | 311 | 34.05 | 29.586 | 56.274 | 1 | 33.44 |
| 5045 | CD2 | PHE | 311 | 34.317 | 28.442 | 58.361 | 1 | 34.61 |
| 5046 | CE1 | PHE | 311 | 32.669 | 29.667 | 56.485 | 1 | 34.53 |
| 5047 | CE2 | PHE | 311 | 32.937 | 28.515 | 58.586 | 1 | 35.78 |
| 5048 | CZ | PHE | 311 | 32.111 | 29.131 | 57.643 | 1 | 35.21 |
| 5049 | C | PHE | 311 | 38.178 | 28.186 | 55.397 | 1 | 28.06 |
| 5050 | O | PHE | 311 | 38.64 | 29.309 | 55.246 | 1 | 28.42 |
| 5051 | N | ALA | 312 | 38.903 | 27.085 | 55.264 | 1 | 29.48 |
| 5052 | CA | ALA | 312 | 40.314 | 27.14 | 54.914 | 1 | 30.76 |
| 5053 | CB | ALA | 312 | 40.959 | 25.781 | 55.143 | 1 | 31.54 |
| 5054 | C | ALA | 312 | 40.429 | 27.539 | 53.446 | 1 | 30.85 |
| 5055 | O | ALA | 312 | 39.536 | 27.259 | 52.644 | 1 | 30.36 |
| 5056 | N | GLY | 313 | 41.529 | 28.194 | 53.101 | 1 | 31.63 |
| 5057 | CA | GLY | 313 | 41.727 | 28.627 | 51.729 | 1 | 31.95 |
| 5058 | C | GLY | 313 | 42.057 | 30.1 | 51.706 | 1 | 31.33 |
| 5059 | O | GLY | 313 | 43.196 | 30.469 | 51.426 | 1 | 31.62 |
| 5060 | N | PRO | 314 | 41.077 | 30.974 | 51.999 | 1 | 30.84 |
| 5061 | CD | PRO | 314 | 39.679 | 30.671 | 52.344 | 1 | 31.01 |
| 5062 | CA | PRO | 314 | 41.305 | 32.421 | 52.008 | 1 | 31.17 |
| 5063 | CB | PRO | 314 | 39.965 | 32.982 | 52.474 | 1 | 29.75 |
| 5064 | CG | PRO | 314 | 38.993 | 31.966 | 52.028 | 1 | 29.65 |
| 5065 | C | PRO | 314 | 42.432 | 32.769 | 52.98 | 1 | 31.38 |
| 5066 | O | PRO | 314 | 42.672 | 32.055 | 53.953 | 1 | 30.86 |
| 5067 | N | PRO | 315 | 43.124 | 33.885 | 52.739 | 1 | 31.94 |
| 5068 | CD | PRO | 315 | 42.93 | 34.835 | 51.63 | 1 | 32.54 |
| 5069 | CA | PRO | 315 | 44.226 | 34.307 | 53.602 | 1 | 31.85 |
| 5070 | CB | PRO | 315 | 44.798 | 35.506 | 52.854 | 1 | 32.13 |
| 5071 | CG | PRO | 315 | 43.587 | 36.073 | 52.171 | 1 | 34.34 |
| 5072 | C | PRO | 315 | 43.864 | 34.626 | 55.059 | 1 | 31.91 |
| 5073 | O | PRO | 315 | 44.717 | 34.528 | 55.938 | 1 | 31.53 |
| 5074 | N | ASN | 316 | 42.614 | 34.989 | 55.332 | 1 | 30.71 |
| 5075 | CA | ASN | 316 | 42.233 | 35.312 | 56.706 | 1 | 29.74 |
| 5076 | CB | ASN | 316 | 41.022 | 36.243 | 56.723 | 1 | 31.09 |
| 5077 | CG | ASN | 316 | 41.393 | 37.672 | 56.399 | 1 | 31.99 |
| 5078 | OD1 | ASN | 316 | 42.564 | 38.034 | 56.441 | 1 | 32.66 |
| 5079 | ND2 | ASN | 316 | 40.395 | 38.497 | 56.086 | 1 | 34.21 |
| 5080 | C | ASN | 316 | 41.957 | 34.11 | 57.601 | 1 | 29.62 |
| 5081 | O | ASN | 316 | 41.608 | 34.281 | 58.769 | 1 | 29.58 |
| 5082 | N | TYR | 317 | 42.1 | 32.9 | 57.066 | 1 | 27.38 |
| 5083 | CA | TYR | 317 | 41.873 | 31.704 | 57.875 | 1 | 27.4 |
| 5084 | CB | TYR | 317 | 42.12 | 30.438 | 57.051 | 1 | 26.75 |
| 5085 | CG | TYR | 317 | 41.891 | 29.164 | 57.836 | 1 | 27.13 |
| 5086 | CD1 | TYR | 317 | 40.608 | 28.797 | 58.25 | 1 | 24.51 |
| 5087 | CE1 | TYR | 317 | 40.4 | 27.65 | 59.012 | 1 | 26.82 |
| 5088 | CD2 | TYR | 317 | 42.961 | 28.35 | 58.204 | 1 | 26.21 |
| 5089 | CE2 | TYR | 317 | 42.766 | 27.206 | 58.966 | 1 | 26.26 |
| 5090 | CZ | TYR | 317 | 41.489 | 26.863 | 59.365 | 1 | 26.86 |
| 5091 | OH | TYR | 317 | 41.294 | 25.735 | 60.111 | 1 | 27.26 |
| 5092 | C | TYR | 317 | 42.88 | 31.813 | 59.031 | 1 | 27.77 |
| 5093 | O | TYR | 317 | 44.087 | 31.855 | 58.81 | 1 | 28.39 |
| 5094 | N | PRO | 318 | 42.388 | 31.843 | 60.279 | 1 | 27.63 |
| 5095 | CD | PRO | 318 | 40.989 | 31.528 | 60.603 | 1 | 27.38 |
| 5096 | CA | PRO | 318 | 43.178 | 31.97 | 61.51 | 1 | 28.3 |
| 5097 | CB | PRO | 318 | 42.143 | 32.403 | 62.553 | 1 | 26.85 |
| 5098 | CG | PRO | 318 | 40.796 | 32.286 | 61.879 | 1 | 29.32 |
| 5099 | C | PRO | 318 | 44.027 | 30.842 | 62.082 | 1 | 27.97 |
| 5100 | O | PRO | 318 | 45.028 | 31.113 | 62.744 | 1 | 28.92 |
| 5101 | N | PHE | 319 | 43.641 | 29.598 | 61.843 | 1 | 28.42 |
| 5102 | CA | PHE | 319 | 44.342 | 28.487 | 62.463 | 1 | 30.82 |
| 5103 | CB | PHE | 319 | 43.32 | 27.395 | 62.797 | 1 | 28.73 |
| 5104 | CG | PHE | 319 | 42.063 | 27.923 | 63.457 | 1 | 27.08 |
| 5105 | CD1 | PHE | 319 | 42.128 | 28.942 | 64.415 | 1 | 25.83 |
| 5106 | CD2 | PHE | 319 | 40.823 | 27.395 | 63.13 | 1 | 26.76 |
| 5107 | CE1 | PHE | 319 | 40.967 | 29.426 | 65.038 | 1 | 25.52 |
| 5108 | CE2 | PHE | 319 | 39.658 | 27.865 | 63.743 | 1 | 27.67 |
| 5109 | CZ | PHE | 319 | 39.732 | 28.881 | 64.698 | 1 | 26.45 |
| 5110 | C | PHE | 319 | 45.54 | 27.901 | 61.732 | 1 | 33.58 |
| 5111 | O | PHE | 319 | 45.967 | 26.788 | 62.032 | 1 | 34.36 |
| 5112 | N | SER | 320 | 46.093 | 28.657 | 60.793 | 1 | 34.56 |
| 5113 | CA | SER | 320 | 47.256 | 28.202 | 60.051 | 1 | 37.51 |
| 5114 | CB | SER | 320 | 47.259 | 28.797 | 58.642 | 1 | 37.72 |
| 5115 | OG | SER | 320 | 46.36 | 28.1 | 57.807 | 1 | 40.24 |
| 5116 | C | SER | 320 | 48.541 | 28.59 | 60.766 | 1 | 37.79 |
| 5117 | O | SER | 320 | 49.473 | 27.796 | 60.851 | 1 | 38.27 |
| 5118 | N | ASP | 321 | 48.588 | 29.812 | 61.283 | 1 | 38.88 |
| 5119 | CA | ASP | 321 | 49.777 | 30.293 | 61.976 | 1 | 39.93 |
| 5120 | CB | ASP | 321 | 49.672 | 31.796 | 62.227 | 1 | 40.64 |
| 5121 | CG | ASP | 321 | 49.532 | 32.588 | 60.946 | 1 | 41.88 |
| 5122 | OD1 | ASP | 321 | 49.799 | 32.027 | 59.863 | 1 | 44.55 |
| 5123 | OD2 | ASP | 321 | 49.165 | 33.777 | 61.016 | 1 | 42.35 |
| 5124 | C | ASP | 321 | 50.001 | 29.572 | 63.298 | 1 | 40.65 |
| 5125 | O | ASP | 321 | 49.101 | 28.9 | 63.805 | 1 | 40.61 |
| 5126 | N | GLU | 322 | 51.205 | 29.709 | 63.848 | 1 | 40.39 |
| 5127 | CA | GLU | 322 | 51.53 | 29.073 | 65.116 | 1 | 41.97 |
| 5128 | CB | GLU | 322 | 52.98 | 29.355 | 65.502 | 1 | 44.61 |
| 5129 | CG | GLU | 322 | 53.38 | 28.734 | 66.829 | 1 | 50.18 |
| 5130 | CD | GLU | 322 | 54.791 | 29.095 | 67.243 | 1 | 53.4 |
| 5131 | OE1 | GLU | 322 | 55.099 | 30.304 | 67.33 | 1 | 56.08 |
| 5132 | OE2 | GLU | 322 | 55.589 | 28.166 | 67.483 | 1 | 56.29 |
| 5133 | C | GLU | 322 | 50.593 | 29.577 | 66.213 | 1 | 40.5 |
| 5134 | O | GLU | 322 | 50.201 | 28.817 | 67.09 | 1 | 40.87 |
| 5135 | N | TYR | 323 | 50.245 | 30.859 | 66.162 | 1 | 39.54 |
| 5136 | CA | TYR | 323 | 49.331 | 31.443 | 67.135 | 1 | 38.14 |
| 5137 | CB | TYR | 323 | 49.979 | 31.505 | 68.527 | 1 | 37.34 |
| 5138 | CG | TYR | 323 | 50.918 | 32.665 | 68.752 | 1 | 37.74 |
| 5139 | CD1 | TYR | 323 | 50.5 | 33.802 | 69.44 | 1 | 37.26 |
| 5140 | CE1 | TYR | 323 | 51.357 | 34.888 | 69.632 | 1 | 38.97 |
| 5141 | CD2 | TYR | 323 | 52.223 | 32.634 | 68.258 | 1 | 39.61 |
| 5142 | CE2 | TYR | 323 | 53.087 | 33.712 | 68.442 | 1 | 41.09 |
| 5143 | CZ | TYR | 323 | 52.647 | 34.837 | 69.13 | 1 | 41.71 |
| 5144 | OH | TYR | 323 | 53.502 | 35.906 | 69.302 | 1 | 44.4 |
| 5145 | C | TYR | 323 | 48.924 | 32.833 | 66.657 | 1 | 37.95 |
| 5146 | O | TYR | 323 | 47.966 | 33.404 | 67.224 | 1 | 37.2 |
| 5147 | OXT | TYR | 323 | 49.574 | 33.327 | 65.708 | 1 | 37.14 |
| 5148 | | | | | | | | |
| 5149 | AP | NAP | 1 | 52.419 | 49.694 | 102.271 | 1 | 25.98 |
| 5150 | AO1 | NAP | 1 | 53.278 | 48.524 | 101.978 | 1 | 24.03 |
| 5151 | AO2 | NAP | 1 | 51.09 | 49.77 | 101.716 | 1 | 25.27 |
| 5152 | AO5* | NAP | 1 | 53.318 | 50.97 | 101.846 | 1 | 29.25 |
| 5153 | AC5* | NAP | 1 | 52.909 | 52.29 | 102.189 | 1 | 30.23 |
| 5154 | AC4* | NAP | 1 | 54.039 | 53.219 | 101.868 | 1 | 31.8 |
| 5155 | AO4* | NAP | 1 | 54.201 | 53.232 | 100.438 | 1 | 31.27 |
| 5156 | AC3* | NAP | 1 | 53.762 | 54.613 | 102.308 | 1 | 32.3 |
| 5157 | AO3* | NAP | 1 | 54.958 | 55.28 | 102.766 | 1 | 35.28 |
| 5158 | AC2* | NAP | 1 | 53.241 | 55.281 | 101.067 | 1 | 32.59 |
| 5159 | AO2* | NAP | 1 | 53.547 | 56.682 | 100.997 | 1 | 30.26 |
| 5160 | AC1* | NAP | 1 | 53.986 | 54.564 | 99.946 | 1 | 31.68 |
| 5161 | AN9 | NAP | 1 | 53.225 | 54.45 | 98.65 | 1 | 33.36 |
| 5162 | AC8 | NAP | 1 | 51.869 | 54.208 | 98.446 | 1 | 32.69 |
| 5163 | AN7 | NAP | 1 | 51.537 | 54.195 | 97.183 | 1 | 33.6 |
| 5164 | AC5 | NAP | 1 | 52.709 | 54.423 | 96.504 | 1 | 33.07 |
| 5165 | AC6 | NAP | 1 | 53.008 | 54.54 | 95.114 | 1 | 33 |
| 5166 | AN6 | NAP | 1 | 52.071 | 54.44 | 94.174 | 1 | 31.6 |
| 5167 | AN1 | NAP | 1 | 54.308 | 54.77 | 94.756 | 1 | 32.37 |
| 5168 | AC2 | NAP | 1 | 55.276 | 54.886 | 95.702 | 1 | 32.87 |
| 5169 | AN3 | NAP | 1 | 55.073 | 54.811 | 97.031 | 1 | 32.45 |
| 5170 | AC4 | NAP | 1 | 53.756 | 54.576 | 97.372 | 1 | 33.11 |
| 5171 | O3 | NAP | 1 | 52.428 | 49.962 | 103.878 | 1 | 28.24 |
| 5172 | NP | NAP | 1 | 51.74 | 49.352 | 105.191 | 1 | 25.93 |
| 5173 | NO1 | NAP | 1 | 51.185 | 50.499 | 105.907 | 1 | 29.68 |
| 5174 | NO2 | NAP | 1 | 52.866 | 48.563 | 105.843 | 1 | 27.83 |
| 5175 | NO5* | NAP | 1 | 50.679 | 48.252 | 104.748 | 1 | 24.61 |
| 5176 | NC5* | NAP | 1 | 49.41 | 48.673 | 104.185 | 1 | 22.4 |
| 5177 | NC4* | NAP | 1 | 48.232 | 48.457 | 105.153 | 1 | 21.8 |
| 5178 | NO4* | NAP | 1 | 48.282 | 47.111 | 105.609 | 1 | 22.69 |
| 5179 | NC3* | NAP | 1 | 48.329 | 49.251 | 106.458 | 1 | 21.8 |
| 5180 | NO3* | NAP | 1 | 47.991 | 50.629 | 106.38 | 1 | 19.92 |
| 5181 | NC2* | NAP | 1 | 47.396 | 48.489 | 107.334 | 1 | 22.04 |
| 5182 | NO2* | NAP | 1 | 46.026 | 48.804 | 107.052 | 1 | 20.53 |
| 5183 | NC1* | NAP | 1 | 47.735 | 47.016 | 106.914 | 1 | 22.46 |
| 5184 | NN1 | NAP | 1 | 48.727 | 46.402 | 107.841 | 1 | 23.71 |
| 5185 | NC2 | NAP | 1 | 48.212 | 45.362 | 108.609 | 1 | 22.55 |
| 5186 | NC3 | NAP | 1 | 49.096 | 44.691 | 109.529 | 1 | 23.34 |
| 5187 | NC7 | NAP | 1 | 48.639 | 43.554 | 110.331 | 1 | 23.95 |
| 5188 | NO7 | NAP | 1 | 49.358 | 43.043 | 111.198 | 1 | 23.51 |
| 5189 | NN7 | NAP | 1 | 47.421 | 43.016 | 110.121 | 1 | 21.98 |
| 5190 | NC4 | NAP | 1 | 50.447 | 45.146 | 109.629 | 1 | 24.05 |
| 5191 | NC5 | NAP | 1 | 50.944 | 46.129 | 108.834 | 1 | 25.29 |
| 5192 | NC6 | NAP | 1 | 50.107 | 46.751 | 107.934 | 1 | 24.49 |
| 5193 | AP2* | NAP | 1 | 52.398 | 57.76 | 100.976 | 1 | 33.17 |
| 5194 | AOP1 | NAP | 1 | 51.433 | 57.516 | 102.079 | 1 | 30.43 |
| 5195 | AOP2 | NAP | 1 | 53.228 | 58.97 | 101.146 | 1 | 35.2 |
| 5196 | AOP3 | NAP | 1 | 51.84 | 57.625 | 99.618 | 1 | 31.14 |
| 5197 | | | | | | | | |
| 5198 | AP | NAP | 1 | 31.491 | 23.321 | 76.234 | 1 | 26.92 |
| 5199 | AO1 | NAP | 1 | 32.959 | 23.112 | 76.092 | 1 | 23.4 |
| 5200 | AO2 | NAP | 1 | 31.023 | 24.344 | 77.147 | 1 | 28.39 |
| 5201 | AO5* | NAP | 1 | 30.891 | 21.867 | 76.578 | 1 | 27.18 |
| 5202 | AC5* | NAP | 1 | 29.483 | 21.663 | 76.586 | 1 | 30.27 |
| 5203 | AC4* | NAP | 1 | 29.226 | 20.199 | 76.739 | 1 | 31.97 |
| 5204 | AO4* | NAP | 1 | 29.66 | 19.821 | 78.06 | 1 | 30.87 |
| 5205 | AC3* | NAP | 1 | 27.783 | 19.857 | 76.621 | 1 | 31.72 |
| 5206 | AO3* | NAP | 1 | 27.59 | 18.576 | 75.977 | 1 | 34.57 |
| 5207 | AC2* | NAP | 1 | 27.318 | 19.788 | 78.042 | 1 | 31.52 |
| 5208 | AO2* | NAP | 1 | 26.267 | 18.844 | 78.253 | 1 | 31.04 |
| 5209 | AC1* | NAP | 1 | 28.551 | 19.293 | 78.793 | 1 | 31.36 |
| 5210 | AN9 | NAP | 1 | 28.676 | 19.783 | 80.211 | 1 | 31.18 |
| 5211 | AC8 | NAP | 1 | 28.366 | 21.032 | 80.732 | 1 | 31.01 |
| 5212 | AN7 | NAP | 1 | 28.585 | 21.116 | 82.02 | 1 | 32.83 |
| 5213 | AC5 | NAP | 1 | 29.061 | 19.882 | 82.38 | 1 | 31.98 |
| 5214 | AC6 | NAP | 1 | 29.459 | 19.338 | 83.632 | 1 | 32.42 |
| 5215 | AN6 | NAP | 1 | 29.398 | 20.04 | 84.756 | 1 | 31.38 |
| 5216 | AN1 | NAP | 1 | 29.904 | 18.042 | 83.662 | 1 | 31.81 |
| 5217 | AC2 | NAP | 1 | 29.958 | 17.318 | 82.516 | 1 | 31.07 |
| 5218 | AN3 | NAP | 1 | 29.59 | 17.76 | 81.296 | 1 | 30.66 |
| 5219 | AC4 | NAP | 1 | 29.139 | 19.053 | 81.298 | 1 | 31.27 |
| 5220 | O3 | NAP | 1 | 30.829 | 23.475 | 74.756 | 1 | 26.84 |
| 5221 | NP | NAP | 1 | 30.744 | 24.558 | 73.599 | 1 | 26.56 |
| 5222 | NO1 | NAP | 1 | 29.325 | 24.632 | 73.216 | 1 | 30.95 |
| 5223 | NO2 | NAP | 1 | 31.715 | 24.056 | 72.551 | 1 | 28.97 |
| 5224 | NO5* | NAP | 1 | 31.381 | 25.925 | 74.121 | 1 | 24.33 |
| 5225 | NC5* | NAP | 1 | 30.623 | 26.74 | 75.053 | 1 | 24.07 |
| 5226 | NC4* | NAP | 1 | 30.044 | 28.012 | 74.415 | 1 | 21.96 |
| 5227 | NO4* | NAP | 1 | 31.116 | 28.688 | 73.763 | 1 | 21.63 |
| 5228 | NC3* | NAP | 1 | 29.051 | 27.781 | 73.281 | 1 | 21.44 |
| 5229 | NO3* | NAP | 1 | 27.731 | 27.385 | 73.666 | 1 | 20.81 |
| 5230 | NC2* | NAP | 1 | 29.091 | 29.1 | 72.586 | 1 | 21.74 |
| 5231 | NO2* | NAP | 1 | 28.314 | 30.09 | 73.265 | 1 | 20.71 |
| 5232 | NC1* | NAP | 1 | 30.618 | 29.432 | 72.663 | 1 | 21.5 |
| 5233 | NN1 | NAP | 1 | 31.326 | 29.023 | 71.419 | 1 | 24.29 |
| 5234 | NC2 | NAP | 1 | 31.797 | 30.089 | 70.659 | 1 | 22.71 |
| 5235 | NC3 | NAP | 1 | 32.496 | 29.798 | 69.439 | 1 | 24.24 |
| 5236 | NC7 | NAP | 1 | 33.068 | 30.865 | 68.614 | 1 | 24.12 |
| 5237 | NO7 | NAP | 1 | 33.584 | 30.618 | 67.517 | 1 | 24.87 |
| 5238 | NN7 | NAP | 1 | 33.082 | 32.142 | 69.046 | 1 | 20.78 |
| 5239 | NC4 | NAP | 1 | 32.651 | 28.43 | 69.054 | 1 | 23.18 |
| 5240 | NC5 | NAP | 1 | 32.232 | 27.4 | 69.831 | 1 | 25.36 |
| 5241 | NC6 | NAP | 1 | 31.575 | 27.679 | 71.012 | 1 | 24.89 |
| 5242 | AP2* | NAP | 1 | 24.846 | 19.298 | 78.758 | 1 | 31.12 |
| 5243 | AOP1 | NAP | 1 | 24.346 | 20.449 | 77.935 | 1 | 29.71 |
| 5244 | AOP2 | NAP | 1 | 24.129 | 18.035 | 78.559 | 1 | 33.02 |
| 5245 | AOP3 | NAP | 1 | 25.095 | 19.605 | 80.174 | 1 | 30.62 |
| 5246 | | | | | | | | |
| 5247 | OH2 | TIP | 1 | 45.001 | 40.378 | 118.313 | 1 | 15.26 |
| 5248 | OH2 | TIP | 2 | 32.516 | 38.304 | 123.853 | 1 | 14.81 |
| 5249 | OH2 | TIP | 3 | 32.919 | 34.403 | 108.28 | 1 | 15.52 |
| 5250 | OH2 | TIP | 4 | 41.837 | 41.292 | 105.623 | 1 | 18.99 |
| 5251 | OH2 | TIP | 5 | 30.917 | 36.384 | 75.698 | 1 | 20.4 |
| 5252 | OH2 | TIP | 6 | 30.328 | 45.984 | 78.024 | 1 | 20.83 |
| 5253 | OH2 | TIP | 7 | 33.466 | 41.619 | 90.824 | 1 | 19.67 |
| 5254 | OH2 | TIP | 8 | 37.501 | 44.346 | 125.316 | 1 | 20.26 |
| 5255 | OH2 | TIP | 9 | 37.393 | 32.625 | 101.943 | 1 | 18.16 |
| 5256 | OH2 | TIP | 10 | 40.909 | 45.982 | 122.694 | 1 | 18.11 |
| 5257 | OH2 | TIP | 11 | 44.528 | 38.79 | 126.817 | 1 | 23.06 |
| 5258 | OH2 | TIP | 12 | 53.521 | 36.063 | 107.923 | 1 | 19.33 |
| 5259 | OH2 | TIP | 13 | 55.414 | 39.924 | 108.941 | 1 | 18.8 |
| 5260 | OH2 | TIP | 14 | 24.457 | 39.716 | 64.921 | 1 | 23.35 |
| 5261 | OH2 | TIP | 15 | 32.145 | 36.847 | 61.428 | 1 | 18.79 |
| 5262 | OH2 | TIP | 16 | 41.349 | 44.99 | 127.363 | 1 | 19.2 |
| 5263 | OH2 | TIP | 18 | 31.821 | 40.243 | 104.727 | 1 | 19.6 |
| 5264 | OH2 | TIP | 19 | 40.383 | 45.522 | 119.941 | 1 | 17.85 |
| 5265 | OH2 | TIP | 20 | 41.038 | 43.926 | 105.074 | 1 | 21.25 |
| 5266 | OH2 | TIP | 21 | 52.798 | 38.523 | 108.612 | 1 | 21.6 |
| 5267 | OH2 | TIP | 22 | 24.578 | 34.991 | 52.277 | 1 | 27.2 |
| 5268 | OH2 | TIP | 23 | 43.566 | 46.144 | 116.665 | 1 | 18.79 |
| 5269 | OH2 | TIP | 24 | 38.407 | 48.16 | 96.072 | 1 | 21.13 |
| 5270 | OH2 | TIP | 25 | 27.004 | 35.105 | 64.406 | 1 | 19.85 |
| 5271 | OH2 | TIP | 26 | 35.011 | 48.336 | 73.361 | 1 | 19.97 |
| 5272 | OH2 | TIP | 27 | 32.296 | 48.154 | 124.555 | 1 | 21.12 |
| 5273 | OH2 | TIP | 28 | 25.256 | 38.826 | 62.122 | 1 | 20.05 |
| 5274 | OH2 | TIP | 29 | 39.109 | 24.774 | 80.543 | 1 | 21.81 |
| 5275 | OH2 | TIP | 30 | 42.063 | 39.776 | 126.839 | 1 | 22.87 |
| 5276 | OH2 | TIP | 31 | 46.745 | 46.451 | 128.813 | 1 | 23.39 |
| 5277 | OH2 | TIP | 32 | 50.371 | 47.093 | 101.553 | 1 | 25.02 |
| 5278 | OH2 | TIP | 34 | 40.126 | 44.155 | 77.856 | 1 | 23.06 |
| 5279 | OH2 | TIP | 35 | 42.375 | 29.781 | 68.382 | 1 | 25.35 |
| 5280 | OH2 | TIP | 36 | 38.086 | 47.952 | 120.461 | 1 | 22.59 |
| 5281 | OH2 | TIP | 37 | 24.488 | 38.637 | 59.36 | 1 | 25.22 |
| 5282 | OH2 | TIP | 38 | 33.449 | 37.051 | 74.61 | 1 | 20.61 |
| 5283 | OH2 | TIP | 39 | 31.1 | 36.961 | 121.752 | 1 | 22.46 |
| 5284 | OH2 | TIP | 40 | 22.204 | 25.909 | 36.991 | 1 | 23.75 |
| 5285 | OH2 | TIP | 41 | 39.567 | 26.563 | 67.439 | 1 | 22.52 |
| 5286 | OH2 | TIP | 42 | 41.986 | 22.37 | 117.586 | 1 | 25.15 |
| 5287 | OH2 | TIP | 43 | 39.491 | 29.267 | 68.378 | 1 | 22.32 |
| 5288 | OH2 | TIP | 44 | 32.736 | 26.776 | 117.39 | 1 | 21.24 |
| 5289 | OH2 | TIP | 45 | 52.291 | 33.953 | 109.25 | 1 | 24.65 |
| 5290 | OH2 | TIP | 46 | 28.534 | 35.148 | 85.678 | 1 | 21.99 |
| 5291 | OH2 | TIP | 47 | 38.513 | 51.843 | 118.035 | 1 | 29.58 |
| 5292 | OH2 | TIP | 48 | 58.9 | 36.787 | 94.782 | 1 | 26.72 |
| 5293 | OH2 | TIP | 49 | 45.997 | 29.479 | 122.242 | 1 | 25.6 |
| 5294 | OH2 | TIP | 50 | 37.564 | 23.032 | 108.863 | 1 | 31.22 |
| 5295 | OH2 | TIP | 51 | 53.34 | 43.038 | 96.412 | 1 | 25.97 |
| 5296 | OH2 | TIP | 52 | 38.245 | 46.005 | 93.947 | 1 | 26.92 |
| 5297 | OH2 | TIP | 53 | 33.17 | 26.364 | 77.101 | 1 | 23.27 |
| 5298 | OH2 | TIP | 54 | 30.792 | 35.957 | 87.521 | 1 | 25.27 |
| 5299 | OH2 | TIP | 55 | 34.833 | 27.951 | 47.706 | 1 | 26.42 |
| 5300 | OH2 | TIP | 56 | 28.786 | 46.17 | 103.396 | 1 | 23.32 |
| 5301 | OH2 | TIP | 57 | 44.266 | 34.136 | 98.337 | 1 | 26.71 |
| 5302 | OH2 | TIP | 58 | 53.209 | 26.911 | 111.536 | 1 | 27.08 |
| 5303 | OH2 | TIP | 59 | 38.28 | 46.235 | 117.616 | 1 | 20.29 |
| 5304 | OH2 | TIP | 60 | 45.846 | 29.481 | 68.533 | 1 | 25.82 |
| 5305 | OH2 | TIP | 61 | 39.477 | 24.73 | 123.6 | 1 | 24.13 |
| 5306 | OH2 | TIP | 62 | 43.143 | 32.069 | 67.027 | 1 | 28.39 |
| 5307 | OH2 | TIP | 63 | 33.587 | 31.372 | 107.145 | 1 | 25.01 |
| 5308 | OH2 | TIP | 64 | 44.207 | 34.772 | 130.949 | 1 | 27.02 |
| 5309 | OH2 | TIP | 65 | 55.271 | 33.285 | 106.717 | 1 | 26.54 |
| 5310 | OH2 | TIP | 66 | 51.566 | 52.972 | 106.675 | 1 | 34.06 |
| 5311 | OH2 | TIP | 67 | 40.855 | 48.113 | 126.206 | 1 | 39.94 |
| 5312 | OH2 | TIP | 68 | 39.093 | 47.851 | 123.163 | 1 | 26.75 |
| 5313 | OH2 | TIP | 69 | 54.844 | 31.2 | 103.265 | 1 | 28.75 |
| 5314 | OH2 | TIP | 70 | 19.433 | 36.954 | 65.541 | 1 | 30.63 |
| 5315 | OH2 | TIP | 71 | 41.513 | 46.621 | 82.033 | 1 | 29.13 |
| 5316 | OH2 | TIP | 72 | 39.688 | 35.104 | 85.306 | 1 | 32.16 |
| 5317 | OH2 | TIP | 73 | 42.495 | 49.189 | 124.242 | 1 | 22.77 |
| 5318 | OH2 | TIP | 74 | 27.176 | 49.553 | 59.439 | 1 | 29.39 |
| 5319 | OH2 | TIP | 75 | 32.193 | 21.851 | 46.155 | 1 | 34.18 |
| 5320 | OH2 | TIP | 76 | 31.155 | 26.326 | 40.579 | 1 | 33.16 |
| 5321 | OH2 | TIP | 77 | 53.856 | 31.665 | 108.599 | 1 | 28.69 |
| 5322 | OH2 | TIP | 78 | 45.803 | 31.563 | 67.006 | 1 | 30.59 |
| 5323 | OH2 | TIP | 79 | 37.812 | 46.835 | 90.383 | 1 | 32.74 |
| 5324 | OH2 | TIP | 80 | 34.283 | 25.635 | 49.031 | 1 | 30.24 |
| 5325 | OH2 | TIP | 81 | 48.808 | 44.792 | 79.805 | 1 | 28.33 |
| 5326 | OH2 | TIP | 82 | 47.473 | 28.776 | 92.601 | 1 | 29.5 |
| 5327 | OH2 | TIP | 83 | 38.546 | 38.877 | 131.989 | 1 | 27.52 |
| 5328 | OH2 | TIP | 84 | 40.089 | 25.09 | 97.779 | 1 | 24.84 |
| 5329 | OH2 | TIP | 85 | 30.793 | 29.787 | 118.751 | 1 | 29.98 |
| 5330 | OH2 | TIP | 86 | 47.286 | 55.22 | 112.543 | 1 | 31.6 |
| 5331 | OH2 | TIP | 87 | 42.753 | 37.02 | 79.784 | 1 | 39.13 |
| 5332 | OH2 | TIP | 88 | 35.057 | 16.558 | 77.386 | 1 | 31.6 |
| 5333 | OH2 | TIP | 89 | 53.713 | 38.681 | 131.57 | 1 | 33.22 |
| 5334 | OH2 | TIP | 90 | 29.118 | 41.306 | 54.163 | 1 | 32 |
| 5335 | OH2 | TIP | 91 | 12.943 | 37.742 | 72.249 | 1 | 30.26 |
| 5336 | OH2 | TIP | 92 | 51.078 | 31.946 | 123.26 | 1 | 27.8 |
| 5337 | OH2 | TIP | 93 | 27.2 | 23.212 | 73.011 | 1 | 34.31 |
| 5338 | OH2 | TIP | 94 | 22.033 | 35.651 | 87.698 | 1 | 32.6 |
| 5339 | OH2 | TIP | 95 | 20.237 | 42.493 | 89.338 | 1 | 36.3 |
| 5340 | OH2 | TIP | 96 | 60.838 | 48.524 | 101.659 | 1 | 30.33 |
| 5341 | OH2 | TIP | 97 | 51.931 | 24.631 | 101.105 | 1 | 35.53 |
| 5342 | OH2 | TIP | 98 | 28.693 | 34.432 | 88.763 | 1 | 39.11 |
| 5343 | OH2 | TIP | 99 | 23.52 | 28.524 | 54.874 | 1 | 38.75 |
| 5344 | OH2 | TIP | 100 | 24.19 | 45.506 | 81.007 | 1 | 28.77 |
| 5345 | OH2 | TIP | 101 | 56.073 | 53.112 | 92.754 | 1 | 34.65 |
| 5346 | OH2 | TIP | 102 | 48.516 | 30.266 | 71.62 | 1 | 34.65 |
| 5347 | OH2 | TIP | 103 | 23.686 | 42.794 | 57.713 | 1 | 32.93 |
| 5348 | OH2 | TIP | 104 | 31.063 | 35.267 | 91.149 | 1 | 31.56 |
| 5349 | OH2 | TIP | 105 | 27.649 | 34.013 | 105.836 | 1 | 30.01 |
| 5350 | OH2 | TIP | 106 | 34.803 | 30.893 | 97.929 | 1 | 37.48 |
| 5351 | OH2 | TIP | 107 | 34.287 | 53.842 | 96.009 | 1 | 34.32 |
| 5352 | OH2 | TIP | 108 | 35.036 | 22.425 | 62.625 | 1 | 35.66 |
| 5353 | OH2 | TIP | 109 | 37.983 | 18.029 | 65.397 | 1 | 32.91 |
| 5354 | OH2 | TIP | 110 | 47.128 | 33.366 | 123.654 | 1 | 35.1 |
| 5355 | OH2 | TIP | 111 | 36.675 | 21.183 | 113.083 | 1 | 30.61 |
| 5356 | OH2 | TIP | 112 | 29.181 | 34.831 | 124.926 | 1 | 45.05 |
| 5357 | OH2 | TIP | 113 | 23.584 | 22.96 | 38.857 | 1 | 36.5 |
| 5358 | OH2 | TIP | 114 | 16.564 | 46.517 | 84.468 | 1 | 36.53 |
| 5359 | OH2 | TIP | 115 | 27.043 | 49.684 | 108.942 | 1 | 32.55 |
| 5360 | OH2 | TIP | 116 | 33.094 | 58.091 | 113.339 | 1 | 28.91 |
| 5361 | OH2 | TIP | 117 | 49.645 | 33.751 | 80.775 | 1 | 34.36 |
| 5362 | OH2 | TIP | 118 | 30.066 | 42.223 | 103.534 | 1 | 27.54 |
| 5363 | OH2 | TIP | 119 | 35.876 | 16.599 | 74.461 | 1 | 32.92 |
| 5364 | OH2 | TIP | 120 | 26.7 | 38.417 | 110.27 | 1 | 27.97 |
| 5365 | OH2 | TIP | 121 | 59.389 | 50.34 | 99.207 | 1 | 31.84 |
| 5366 | OH2 | TIP | 122 | 32.506 | 16.893 | 84.875 | 1 | 32.8 |
| 5367 | OH2 | TIP | 123 | 28.477 | 49.93 | 119.857 | 1 | 33.02 |
| 5368 | OH2 | TIP | 124 | 28.226 | 46.593 | 79.868 | 1 | 31.4 |
| 5369 | OH2 | TIP | 125 | 47.312 | 22.624 | 79.641 | 1 | 32.14 |
| 5370 | OH2 | TIP | 126 | 43.274 | 38.481 | 93.349 | 1 | 36.38 |
| 5371 | OH2 | TIP | 127 | 24.394 | 39.895 | 54.703 | 1 | 31.22 |
| 5372 | OH2 | TIP | 128 | 62.576 | 44.383 | 109.622 | 1 | 38.29 |
| 5373 | OH2 | TIP | 129 | 39.313 | 53.697 | 63.477 | 1 | 35.69 |
| 5374 | OH2 | TIP | 130 | 56.034 | 30.407 | 114.992 | 1 | 35.51 |
| 5375 | OH2 | TIP | 131 | 58.55 | 44.64 | 114.05 | 1 | 33.72 |
| 5376 | OH2 | TIP | 132 | 26.613 | 36.991 | 107.639 | 1 | 33.13 |
| 5377 | OH2 | TIP | 133 | 28.155 | 52.064 | 73.725 | 1 | 40.93 |
| 5378 | OH2 | TIP | 134 | 26.29 | 22.128 | 38.289 | 1 | 35.09 |
| 5379 | OH2 | TIP | 135 | 41.992 | 44.005 | 85.943 | 1 | 38.29 |
| 5380 | OH2 | TIP | 136 | 23.79 | 53.196 | 102.275 | 1 | 35.55 |
| 5381 | OH2 | TIP | 137 | 38.259 | 49.291 | 73.759 | 1 | 33.97 |
| 5382 | OH2 | TIP | 138 | 31.186 | 39.8 | 53.512 | 1 | 28.3 |
| 5383 | OH2 | TIP | 139 | 49.832 | 29.79 | 94.84 | 1 | 37.52 |
| 5384 | OH2 | TIP | 140 | 51.065 | 28.329 | 72.978 | 1 | 46.98 |
| 5385 | OH2 | TIP | 141 | 46.66 | 53.496 | 98.395 | 1 | 31.75 |
| 5386 | OH2 | TIP | 142 | 46.597 | 30.116 | 64.655 | 1 | 32.73 |
| 5387 | OH2 | TIP | 143 | 25.375 | 27.537 | 35.355 | 1 | 33.43 |
| 5388 | OH2 | TIP | 144 | 21.898 | 26.93 | 69.113 | 1 | 38.87 |
| 5389 | OH2 | TIP | 145 | 52.835 | 55.056 | 106.028 | 1 | 36.26 |
| 5390 | OH2 | TIP | 146 | 37.772 | 32.55 | 96.88 | 1 | 38.37 |
| 5391 | OH2 | TIP | 147 | 55.816 | 31.008 | 110.284 | 1 | 35.26 |
| 5392 | OH2 | TIP | 148 | 42.571 | 41.266 | 84.444 | 1 | 39.27 |
| 5393 | OH2 | TIP | 149 | 57.062 | 30.146 | 101.179 | 1 | 34.53 |
| 5394 | OH2 | TIP | 150 | 19.557 | 23.851 | 68.74 | 1 | 38.35 |
| 5395 | OH2 | TIP | 151 | 53.208 | 33.684 | 91.837 | 1 | 35.29 |
| 5396 | OH2 | TIP | 152 | 26.885 | 25.98 | 82.305 | 1 | 28.44 |
| 5397 | OH2 | TIP | 153 | 60.656 | 41.047 | 133.574 | 1 | 43.29 |
| 5398 | OH2 | TIP | 154 | 50.192 | 35.856 | 83.36 | 1 | 30.89 |
| 5399 | OH2 | TIP | 155 | 46.431 | 31.285 | 59.935 | 1 | 38.03 |
| 5400 | OH2 | TIP | 156 | 58.677 | 32.63 | 120.469 | 1 | 39.94 |
| 5401 | OH2 | TIP | 157 | 26.809 | 34.112 | 97.918 | 1 | 41.53 |
| 5402 | OH2 | TIP | 158 | 33.884 | 52.712 | 77.023 | 1 | 35.19 |
| 5403 | OH2 | TIP | 159 | 38.057 | 49.152 | 93.115 | 1 | 41.66 |
| 5404 | OH2 | TIP | 160 | 45.468 | 33.014 | 90.44 | 1 | 46.45 |
| 5405 | OH2 | TIP | 161 | 11.785 | 42.754 | 80.598 | 1 | 37.64 |
| 5406 | OH2 | TIP | 162 | 24.311 | 36.374 | 91.676 | 1 | 43.49 |
| 5407 | OH2 | TIP | 163 | 42.904 | 31.446 | 93.489 | 1 | 37.26 |
| 5408 | OH2 | TIP | 164 | 19.008 | 46.343 | 76.831 | 1 | 32.64 |
| 5409 | OH2 | TIP | 165 | 26.792 | 37.766 | 121.303 | 1 | 45.31 |
| 5410 | OH2 | TIP | 166 | 49.187 | 35.043 | 63.624 | 1 | 31.95 |
| 5411 | OH2 | TIP | 167 | 36.331 | 32.254 | 93.605 | 1 | 44.09 |
| 5412 | OH2 | TIP | 168 | 35.186 | 26.215 | 102.521 | 1 | 43.2 |
| 5413 | OH2 | TIP | 169 | 48.889 | 58.577 | 112.794 | 1 | 49.41 |
| 5414 | OH2 | TIP | 170 | 52.692 | 49.72 | 130.355 | 1 | 41.72 |
| 5415 | OH2 | TIP | 171 | 50.466 | 50.762 | 125.316 | 1 | 39.53 |
| 5416 | OH2 | TIP | 172 | 39.495 | 33.029 | 94.392 | 1 | 34.89 |
| 5417 | OH2 | TIP | 173 | 41.34 | 52.958 | 115.729 | 1 | 31.01 |
| 5418 | OH2 | TIP | 174 | 34.57 | 29.202 | 41.202 | 1 | 45.68 |
| 5419 | OH2 | TIP | 175 | 46.718 | 51.893 | 65.788 | 1 | 41.59 |
| 5420 | OH2 | TIP | 176 | 28.069 | 32.273 | 93.383 | 1 | 41.61 |
| 5421 | OH2 | TIP | 177 | 32.318 | 58.463 | 117.063 | 1 | 43.39 |
| 5422 | OH2 | TIP | 178 | 44.4 | 43.106 | 133.577 | 1 | 43.84 |
| 5423 | OH2 | TIP | 179 | 46.226 | 56.794 | 110.966 | 1 | 41.8 |
| 5424 | OH2 | TIP | 180 | 40.422 | 60.788 | 94.759 | 1 | 37.26 |
| 5425 | OH2 | TIP | 181 | 48.451 | 28.607 | 83.392 | 1 | 32.96 |
| 5426 | OH2 | TIP | 182 | 24.358 | 56.785 | 99.593 | 1 | 44.73 |
| 5427 | OH2 | TIP | 183 | 41.25 | 41.877 | 55.915 | 1 | 42.35 |
| 5428 | OH2 | TIP | 184 | 45.973 | 35.217 | 86.966 | 1 | 42.98 |
| 5429 | OH2 | TIP | 185 | 24.234 | 20.35 | 90.823 | 1 | 41.63 |
| 5430 | OH2 | TIP | 186 | 20.05 | 26.911 | 71.116 | 1 | 44.24 |
| 5431 | OH2 | TIP | 187 | 25.517 | 47.644 | 116.084 | 1 | 40.22 |
| 5432 | OH2 | TIP | 188 | 38.148 | 55.74 | 125.016 | 1 | 50.57 |
| 5433 | OH2 | TIP | 189 | 17.415 | 19.187 | 83.179 | 1 | 51.35 |
| 5434 | OH2 | TIP | 190 | 40.72 | 36.135 | 53.695 | 1 | 41.2 |
| 5435 | OH2 | TIP | 191 | 58.834 | 41.888 | 125.21 | 1 | 39.88 |
| 5436 | OH2 | TIP | 192 | 29.41 | 29.727 | 93.858 | 1 | 48.74 |
| 5437 | OH2 | TIP | 193 | 49.842 | 27.949 | 80.741 | 1 | 35.45 |
| 5438 | OH2 | TIP | 194 | 37.814 | 39.274 | 54.566 | 1 | 39.68 |
| 5439 | OH2 | TIP | 195 | 20.41 | 33.596 | 67.244 | 1 | 47.88 |
| 5440 | OH2 | TIP | 196 | 56.399 | 38.41 | 78.303 | 1 | 47.48 |
| 5441 | OH2 | TIP | 197 | 18.546 | 44.981 | 60.403 | 1 | 43.73 |
| 5442 | OH2 | TIP | 198 | 23.994 | 49.638 | 108.354 | 1 | 49.51 |
| 5443 | OH2 | TIP | 199 | 22.908 | 22.771 | 41.59 | 1 | 48.79 |
| 5444 | OH2 | TIP | 200 | 51.09 | 17.174 | 84.254 | 1 | 51.21 |
| 5445 | OH2 | TIP | 201 | 21.801 | 39.4 | 59.941 | 1 | 38.04 |
| 5446 | OH2 | TIP | 202 | 59.191 | 44.537 | 116.947 | 1 | 46.96 |
| 5447 | OH2 | TIP | 203 | 53.282 | 35.753 | 73.561 | 1 | 46.48 |
| 5448 | OH2 | TIP | 204 | 41.701 | 43.375 | 82.55 | 1 | 42.46 |
| 5449 | OH2 | TIP | 205 | 30.079 | 46.916 | 125.452 | 1 | 44.25 |
| 5450 | OH2 | TIP | 206 | 52.385 | 19.609 | 89.868 | 1 | 41.23 |
| 5451 | OH2 | TIP | 207 | 26.106 | 20.765 | 73.523 | 1 | 40.03 |
| 5452 | OH2 | TIP | 208 | 47.982 | 60.086 | 89.854 | 1 | 35.91 |
| 5453 | OH2 | TIP | 209 | 45.575 | 38.685 | 84.376 | 1 | 41.02 |
| 5454 | OH2 | TIP | 210 | 50.34 | 34.715 | 124.626 | 1 | 42.79 |
| 5455 | OH2 | TIP | 211 | 38.796 | 34.422 | 92.168 | 1 | 45.42 |
| 5456 | OH2 | TIP | 212 | 57.681 | 33.9 | 101.661 | 1 | 43.35 |
| 5457 | OH2 | TIP | 213 | 54.846 | 32.481 | 93.809 | 1 | 39.9 |
| 5458 | OH2 | TIP | 214 | 18.125 | 49.991 | 70.464 | 1 | 46.3 |
| 5459 | OH2 | TIP | 215 | 21.97 | 39.651 | 62.727 | 1 | 33.34 |
| 5460 | OH2 | TIP | 216 | 28.842 | 43.327 | 125.062 | 1 | 45.51 |
| 5461 | OH2 | TIP | 217 | 16.7 | 46.812 | 66.283 | 1 | 44.74 |
| 5462 | OH2 | TIP | 218 | 30.326 | 44.411 | 91.916 | 1 | 36.28 |
| 5463 | OH2 | TIP | 220 | 38.045 | 36.433 | 53.954 | 1 | 37.35 |
| 5464 | OH2 | TIP | 221 | 27.518 | 52.423 | 95.841 | 1 | 36.13 |
| 5465 | OH2 | TIP | 222 | 45.658 | 34.782 | 133.488 | 1 | 37.65 |
| 5466 | OH2 | TIP | 223 | 40.129 | 53.923 | 117.587 | 1 | 38.41 |
| 5467 | OH2 | TIP | 226 | 41.548 | 42.117 | 133.921 | 1 | 33.86 |
| 5468 | OH2 | TIP | 227 | 14.866 | 46.35 | 70.169 | 1 | 47.73 |
| 5469 | OH2 | TIP | 228 | 31.95 | 29.491 | 106.796 | 1 | 45.5 |
| 5470 | OH2 | TIP | 229 | 21.037 | 21.047 | 41.409 | 1 | 47.05 |
| 5471 | OH2 | TIP | 230 | 28.445 | 51.038 | 61.644 | 1 | 40.98 |
| 5472 | OH2 | TIP | 231 | 43.854 | 34.88 | 90.224 | 1 | 49.28 |
| 5473 | OH2 | TIP | 232 | 28.047 | 27.942 | 34.429 | 1 | 38.25 |
| 5474 | OH2 | TIP | 233 | 12.984 | 24.147 | 67.59 | 1 | 47.73 |
| 5475 | OH2 | TIP | 234 | 55.247 | 63.389 | 98.594 | 1 | 51.45 |
| 5476 | OH2 | TIP | 235 | 60.396 | 46.828 | 118.097 | 1 | 43.58 |
| 5477 | OH2 | TIP | 236 | 29.373 | 23.257 | 113.788 | 1 | 49.82 |
| 5478 | OH2 | TIP | 237 | 51.361 | 59.242 | 106.004 | 1 | 44.98 |
| 5479 | OH2 | TIP | 238 | 28.732 | 34.199 | 91.896 | 1 | 40.79 |
| 5480 | OH2 | TIP | 239 | 47.85 | 15.242 | 77.784 | 1 | 47.04 |
| 5481 | OH2 | TIP | 240 | 35.188 | 24.83 | 51.425 | 1 | 37.83 |
| 5482 | OH2 | TIP | 241 | 53.575 | 29.228 | 116.275 | 1 | 50.9 |
| 5483 | OH2 | TIP | 242 | 47.746 | 22.817 | 114.979 | 1 | 36.7 |
| 5484 | OH2 | TIP | 243 | 47.14 | 30.391 | 90.416 | 1 | 51.56 |
| 5485 | OH2 | TIP | 244 | 7.671 | 43.941 | 85.116 | 1 | 41.02 |
| 5486 | OH2 | TIP | 245 | 31.61 | 30.446 | 99.001 | 1 | 38.13 |
| 5487 | OH2 | TIP | 246 | 34.582 | 30.877 | 124.231 | 1 | 38.93 |
| 5488 | OH2 | TIP | 247 | 49.066 | 21.06 | 96.132 | 1 | 42.69 |
| 5489 | OH2 | TIP | 248 | 49.506 | 41.872 | 61.108 | 1 | 42.5 |
| 5490 | OH2 | TIP | 249 | 32.245 | 34.942 | 125.464 | 1 | 39.7 |
| 5491 | OH2 | TIP | 250 | 39.287 | 56.63 | 116.779 | 1 | 46.55 |
| 5492 | OH2 | TIP | 251 | 47.334 | 21.723 | 101.703 | 1 | 49.53 |
| 5493 | OH2 | TIP | 252 | 14.215 | 43.763 | 87.46 | 1 | 41.94 |
| 5494 | OH2 | TIP | 253 | 52.694 | 63.517 | 94.843 | 1 | 46.38 |
| 5495 | OH2 | TIP | 254 | 57.488 | 29.835 | 125.415 | 1 | 44.6 |
| 5496 | OH2 | TIP | 255 | 47.278 | 48.74 | 79.314 | 1 | 47.95 |
| 5497 | OH2 | TIP | 256 | 27.087 | 51.852 | 116.357 | 1 | 47.55 |
| 5498 | OH2 | TIP | 257 | 41.014 | 51.312 | 87.598 | 1 | 40.75 |
| 5499 | OH2 | TIP | 258 | 48.855 | 64.637 | 94.988 | 1 | 46.51 |
| 5500 | OH2 | TIP | 259 | 34.326 | 51.133 | 57.401 | 1 | 49.68 |
| 5501 | OH2 | TIP | 260 | 38.502 | 38.24 | 91.604 | 1 | 37.64 |
| 5502 | OH2 | TIP | 261 | 24.129 | 45.02 | 113.403 | 1 | 48.05 |
| 5503 | OH2 | TIP | 262 | 35.286 | 54.125 | 63.327 | 1 | 45.56 |
| 5504 | OH2 | TIP | 263 | 30.663 | 27.704 | 120.762 | 1 | 45.17 |
| 5505 | OH2 | TIP | 264 | 35.871 | 54.187 | 124.066 | 1 | 50.41 |
| 5506 | OH2 | TIP | 265 | 21.159 | 41.64 | 58.64 | 1 | 41.93 |
| 5507 | OH2 | TIP | 266 | 19.788 | 36.679 | 50.156 | 1 | 51.04 |
| 5508 | OH2 | TIP | 267 | 40.486 | 59.384 | 116.678 | 1 | 55.91 |
| 5509 | OH2 | TIP | 268 | 57.2 | 30.639 | 89.246 | 1 | 50.27 |
| 5510 | OH2 | TIP | 269 | 40.547 | 51.106 | 73.462 | 1 | 54.93 |
| 5511 | OH2 | TIP | 270 | 34.237 | 52.021 | 91.663 | 1 | 44.61 |
| 5512 | OH2 | TIP | 271 | 25.491 | 31.111 | 92.578 | 1 | 50.81 |
| 5513 | OH2 | TIP | 272 | 39.613 | 40.278 | 90.384 | 1 | 42.78 |
| 5514 | OH2 | TIP | 273 | 34.485 | 12.602 | 83.204 | 1 | 45.2 |
| 5515 | OH2 | TIP | 274 | 35.981 | 24.594 | 99.268 | 1 | 56.4 |
| 5516 | OH2 | TIP | 275 | 29.035 | 64.519 | 103.226 | 1 | 47.65 |
| 5517 | OH2 | TIP | 276 | 51.698 | 23.931 | 95.739 | 1 | 55.55 |
| 5518 | OH2 | TIP | 277 | 33.44 | 19.305 | 115.417 | 1 | 57.07 |
| 5519 | OH2 | TIP | 278 | 56.627 | 51.75 | 100.165 | 1 | 51.89 |
| 5520 | OH2 | TIP | 279 | 43.5 | 49.937 | 132.076 | 1 | 47.67 |
| 5521 | OH2 | TIP | 280 | 60.676 | 35.007 | 83.418 | 1 | 52.33 |
| 5522 | OH2 | TIP | 281 | 14.648 | 51.474 | 77.578 | 1 | 59.77 |
| 5523 | OH2 | TIP | 283 | 30.345 | 47.707 | 82.813 | 1 | 36.04 |
| 5524 | OH2 | TIP | 284 | 36.004 | 46.202 | 52.162 | 1 | 41.13 |
| 5525 | OH2 | TIP | 285 | 32.03 | 29.957 | 42.962 | 1 | 44.61 |
| 5526 | OH2 | TIP | 286 | 48.798 | 35.044 | 85.737 | 1 | 47.24 |
| 5527 | OH2 | TIP | 287 | 32.321 | 33.202 | 127.513 | 1 | 43.68 |
| 5528 | OH2 | TIP | 288 | 22.068 | 39.15 | 56.277 | 1 | 50.31 |
| 5529 | OH2 | TIP | 289 | 21.987 | 24.918 | 66.146 | 1 | 47.47 |
| 5530 | OH2 | TIP | 290 | 15.872 | 33.363 | 67.16 | 1 | 51.12 |
| 5531 | OH2 | TIP | 291 | 26.09 | 55.172 | 95.12 | 1 | 52.07 |
| 5532 | OH2 | TIP | 292 | 27.583 | 56.238 | 65.392 | 1 | 49.45 |
| 5533 | OH2 | TIP | 293 | 34.26 | 23.299 | 47.224 | 1 | 49.03 |
| 5534 | OH2 | TIP | 294 | 57.408 | 31.747 | 105.896 | 1 | 46.8 |
| 5535 | OH2 | TIP | 295 | 59.962 | 39.089 | 123.57 | 1 | 44.87 |
| 5536 | OH2 | TIP | 296 | 65.976 | 39.468 | 95.276 | 1 | 44.84 |
| 5537 | OH2 | TIP | 297 | 48.396 | 28.207 | 68.765 | 1 | 46.48 |
| 5538 | OH2 | TIP | 298 | 42.283 | 19.509 | 65.087 | 1 | 46.86 |
| 5539 | OH2 | TIP | 299 | 59.227 | 33.147 | 109.151 | 1 | 51.7 |
| 5540 | OH2 | TIP | 300 | 29.15 | 30.207 | 106.468 | 1 | 50.01 |
| 5541 | OH2 | TIP | 301 | 46.663 | 66.616 | 95.455 | 1 | 54.65 |
| 5542 | OH2 | TIP | 302 | 26.271 | 35.337 | 122.528 | 1 | 48.4 |
| 5543 | OH2 | TIP | 303 | 18.858 | 27.274 | 88.356 | 1 | 48.53 |
| 5544 | OH2 | TIP | 304 | 51.926 | 65.647 | 96.319 | 1 | 53.14 |
| 5545 | OH2 | TIP | 305 | 20.25 | 51.904 | 72.655 | 1 | 56.14 |
| 5546 | OH2 | TIP | 306 | 34.709 | 22.106 | 59.241 | 1 | 47.81 |
| 5547 | OH2 | TIP | 307 | 40.433 | 17.704 | 66.527 | 1 | 52.34 |
| 5548 | OH2 | TIP | 308 | 32.003 | 18.215 | 77.58 | 1 | 52.34 |
| 5549 | OH2 | TIP | 309 | 23.422 | 28.55 | 92.607 | 1 | 43.14 |
| 5550 | OH2 | TIP | 310 | 47.257 | 20.159 | 98.331 | 1 | 50.52 |
| 5551 | OH2 | TIP | 311 | 24.448 | 42.609 | 110.078 | 1 | 49.23 |
| 5552 | OH2 | TIP | 312 | 33.517 | 30.6 | 45.341 | 1 | 51.64 |
| 5553 | OH2 | TIP | 313 | 42.127 | 21.444 | 98.747 | 1 | 54.04 |
| 5554 | OH2 | TIP | 314 | 18.919 | 43.54 | 58.143 | 1 | 51.11 |
| 5555 | OH2 | TIP | 315 | 23.164 | 43.23 | 115.621 | 1 | 47.26 |
| 5556 | OH2 | TIP | 316 | 16.176 | 45.107 | 61.852 | 1 | 53.28 |
| 5557 | OH2 | TIP | 317 | 27.877 | 27.144 | 109.844 | 1 | 50.1 |
| 5558 | OH2 | TIP | 318 | 67.95 | 50.314 | 103.16 | 1 | 52.74 |
| 5559 | OH2 | TIP | 319 | 31.95 | 21.574 | 113.159 | 1 | 41.29 |
| 5560 | OH2 | TIP | 320 | 26.046 | 58.454 | 106.489 | 1 | 47.06 |
| 5561 | | | | | | | | |
| 5562 | OH2 | TIP | 321 | 31.82 | 45.915 | 98.812 | 1 | 39.55 |
| 5563 | OH2 | TIP | 322 | 41.201 | 12.29 | 77.413 | 1 | 43.87 |
| 5564 | OH2 | TIP | 323 | 27.105 | 33.922 | 95.152 | 1 | 48.88 |
| 5565 | OH2 | TIP | 324 | 44.9 | 20.824 | 97.326 | 1 | 46.5 |
| 5566 | OH2 | TIP | 325 | 45.613 | 40.37 | 134.348 | 1 | 43.36 |
| 5567 | OH2 | TIP | 326 | 30.431 | 42.419 | 100.809 | 1 | 41.71 |
| 5568 | OH2 | TIP | 327 | 21.97 | 36.04 | 58.009 | 1 | 49.22 |
| 5569 | OH2 | TIP | 328 | 26.728 | 41.92 | 84.56 | 1 | 45.66 |
| 5570 | OH2 | TIP | 329 | 44.324 | 37.558 | 86.373 | 1 | 48.13 |
| 5571 | OH2 | TIP | 330 | 13.618 | 27.035 | 82.769 | 1 | 46.71 |
| 5572 | OH2 | TIP | 331 | 28.796 | 43.32 | 84.297 | 1 | 51.64 |
| 5573 | OH2 | TIP | 332 | 36.988 | 64.849 | 99.594 | 1 | 51.26 |
| 5574 | OH2 | TIP | 333 | 39.844 | 50.346 | 81.834 | 1 | 51.93 |
| 5575 | OH2 | TIP | 334 | 42.475 | 38.953 | 83.449 | 1 | 49.18 |
| 5576 | OH2 | TIP | 335 | 27.186 | 48.853 | 80.722 | 1 | 43.66 |
| 5577 | OH2 | TIP | 336 | 11.416 | 39.187 | 68.638 | 1 | 49.42 |
| 5578 | OH2 | TIP | 337 | 22.473 | 55.81 | 101.424 | 1 | 49.76 |
| 5579 | OH2 | TIP | 338 | 32.198 | 34.546 | 45.862 | 1 | 48.14 |
| 5580 | OH2 | TIP | 339 | 34.936 | 51.874 | 84.774 | 1 | 48.39 |
| 5581 | OH2 | TIP | 340 | 38.076 | 38.607 | 88.497 | 1 | 43.68 |
| 5582 | OH2 | TIP | 341 | 30.584 | 45.209 | 101.344 | 1 | 44.22 |
| 5583 | OH2 | TIP | 342 | 37.067 | 49.443 | 90.254 | 1 | 54.48 |

1. A method for identifying an inhibitor to the human type III 3a-HSD enzyme, comprising the steps of
a) designing or selecting computationally a potential inhibitor by using the atomic coordinates of the human type III 3a-HSD enzyme or co-complexes, e.g. as described in Table 1; and/or
b) obtaining a potential inhibitor by performing a NMR screen with the human type 1113a-HSD and candidate compounds, e.g. from a library of compounds; and/or
c) obtaining a potential inhibitor by performing a NMR reporter screen with the human type III 3a-HSD, a first inhibitor candidate to human type III 3a-HSD with a dissociation constant smaller than 2 mM which is either already known or found by a method according to step a) or b) and further a second candidate compound, e.g. from a library of compounds; and/or d) determining the activity of the potential inhibitor from step a), b) or c) at the human type III 3a-HSD enzyme.

2. The method of claim 1, comprising a further step d), said step comprises the step of docking said potential inhibitor to the three-dimensional structure of human type III 3a-HSD enzyme or co-complexes, as described in Table 1, and employing the obtained three-dimensional structure relation to design or select further inhibitors.

3. The method of claim 1, comprising a further step e), said step comprises the step of crystallizing the in step a), b) or c) obtained potential inhibitor with human type III 3a-HSD enzyme or co-complexes, e.g. as described in Table 1, determining the X-ray structure, comparing the three-dimensional structure of the so obtained new co-complex with the formerly known structures of human type III 3a-HSD enzyme and employing the obtained three-dimensional structure difference to design or select further potential inhibitors.

4. The method according to claim 1, wherein said potential inhibitor is designed de novo.

5. The method according to claim 1, wherein said potential inhibitor is designed from a known inhibitor.

6. The method according to claim 1, wherein said potential inhibitor is a competitive inhibitor of human type III 3a-HSD.

7. The method according to claim 1, wherein said step of employing a three-dimensional structure to design or select said compound comprises the steps of:
a) identifying chemical entities or fragments capable of associating with said enzyme; and
b) assembling the identified chemical entities or fragments into a single molecule to provide the structure of said potential inhibitor.

8. The method according to claim 1, wherein said first inhibitor candidate in step c) is 2-acetylbenzofuran.

9. A crystalline complex of human type III 3a-HSD and NADP exhibiting essentially the atomic coordinates listed in Table 1.

10. A process for the production of a crystalline complex according to claim 9, comprising the step of growing the crystalline complex in 50-200 mM ammonium sulfate or ammonium acetate, 25-200 mM MES, pH 6.0 or 25-200 mM sodium citrate, 20-30% PEG monomethylether 2000 or 5000 or 20-30% PEG 2000, 4000, 6000, or 8000; 0-10% additives, and 0-20 mM DTT.


SEQUENCE LISTING (5 sequences)

SEQ ID NO: 1 - DNA, 972 bp, Homo Sapiens
  CDS (1)...(972)
atggattcgaaataccagtgtgtgaagctgaatgatggtcacttcatgcctgtcctgggatttggcacctatgcgcctgcagaggttcctaaaagtaaagctctagaggccgtcaaattggcaatagaagccgggttccaccatattgattctgcacatgtttacaataatgaggagcaggttggactggccatccgaagcaagattgcagatggcagtgtgaagagagaagacatattctacacttcaaagctttggagcaattcccatcgaccagagttggtccgaccagccttggaaaggtcactgaaaaatcttcaattggactatgttgacctctatcttattcattttccagtgtctgtaaagccaggtgaggaagtgatcccaaaagatgaaaatggaaaaatactatttgacacagtggatctctgtgccacatgggaggccatggagaagtgtaaagatgcaggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaa

SEQ ID NO: 2 - protein, 323 aa, Homo Sapiens
MDSKYQCVKLNDGHFMPVLGFGTYAPAEVPKSKALEAVKLAIEAGFHHIDSAHVYNNEEQVGLAIRSKIADGSVKREDIFYTSKLWSNSHRPELVRPALERSLKNLQLDYVDLYLIHFPVSVKPGEEVIPKDENGKILFDTVDLCATWEAMEKCKDAGLAKSIGVSNFNHRLLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 3 - DNA, 72 bp, Artificial Sequence
  Sense gene specific primer
atacatatgcaccaccaccaccaccacctcgaggttctgttccagggtccggattcgaaataccagtgtgtg

SEQ ID NO: 4 - DNA, 30 bp, Artificial Sequence
  Sense gene specific primer
tttcatatggattcgaaataccagtgtgtg

SEQ ID NO: 5 - DNA, 30 bp, Artificial Sequence
  Anti-sense gene specific primer
agagcggccgctgttaatattcatcagaaa